(19) United States
(12) Patent Application Publication
(10) Pub. No.: US 2023/0303710 A1
(43) Pub. Date: Sep. 28, 2023

(54) METHODS AND COMPOSITIONS FOR INHIBITION OF DIHYDROOROTATE DEHYDROGENASE IN COMBINATION WITH AN ANTI-CD38 THERAPEUTIC AGENT

(71) Applicants: OHIO STATE INNOVATION FOUNDATION, Columbus, OH (US); HENDRIX COLLEGE, Conway, AR (US)

(72) Inventors: John C. BYRD, Columbus, OH (US); Erin HERTLEIN, Columbus, OH (US); Ola A. ELGAMAL, Columbus, OH (US); Mariah L. JOHNSON, Columbus, OH (US); Susheela TRIDANDAPANI, Dublin, OH (US); Chad E. BENNETT, Columbus, OH (US); Sandip Madhukar VIBHUTE, Columbus, OH (US); Thomas E. GOODWIN, Conway, AR (US)

(21) Appl. No.: 17/789,187

(22) PCT Filed: Dec. 26, 2020
(86) PCT No.: PCT/US2020/067074

§ 371 Date: Jun. 25, 2022

Related U.S. Application Data
(60) Provisional application No. 62/953,860, filed on Dec. 26, 2019.

(51) Int. Cl. C07K 16/28; A61P 35/00; A61K 31/472

(52) U.S. Cl. CPC C07K 16/2896; A61K 31/472; A61P 35/00

(57) ABSTRACT

Disclosed are pharmaceutical combinations and methods of treating a clinical condition, e.g., AML, by administering to a subject a pharmaceutical combination comprising a DHODH inhibitor and an anti-CD38 therapeutic agent such as a anti-CD38 antibody. The pharmaceutical combination can further comprise one or more additional therapeutic agents. This abstract is intended as a scanning tool for purposes of searching in the particular art and is not intended to be limiting of the present disclosure.

### CROSS-REFERENCE TO RELATED APPLICATIONS

This Application claims the benefit of U.S. Provisional Application No. 62/953,860, filed on Dec. 26, 2019, which is incorporated herein by reference in its entirety.

### BACKGROUND

Proliferating cells require a supply of nucleotides for replication of DNA and transcription of genes to RNA, as well as for a variety of other metabolic processes. Cells can supply such nucleotides by de novo nucleotide synthesis pathways. An important step in the de novo synthesis pathway of pyrimidine nucleotides is the oxidation of dihydroorotate to form orotate. That reaction is catalyzed by dihydroorotate dehydrogenase (DHODH) and that step is one of the rate-limiting steps in the pyrimidine nucleotide synthesis pathway. DHODH has a sub-cellular location in the mitochondrial membrane and uses cytochrome C in the electron transport chain as an electron acceptor for the oxidation of dihydroorotate to orotate.

Under normal circumstances the intracellular pool of pyrimidine nucleotides can be replenished by a salvage pathway in which pyrimidine nucleotides are recycled. Although this DHODH-independent mechanism is sufficient for resting lymphocytes, ‘activated’ and proliferating lymphocytes need to substantially increase the available pyrimidine and so become dependent on de novo pyrimidine synthesis. Since orotate is a necessary intermediate in pyrimidine nucleotide synthesis, and since pyrimidine nucleotides are required for DNA replication, gene expression, and carbohydrate metabolism, inhibition of the DHODH enzyme can inhibit cell growth.

Moreover, rapidly proliferating cells require pyrimidines not only for cellular growth, but also for protein glycosylation, membrane lipid biosynthesis and strand break repair (e.g., see Fairbanks, et al., J. Biol. Chem. 270:29682-29689 (1995)). Under such conditions, in order to meet the increased demand, substantial quantities of pyrimidine nucleotides must be produced in rapidly proliferating cells. Accordingly, DHODH inhibitors are attractive candidates for treating proliferative disorders (e.g., see Liu, S., et al., Structure 8:25-31 (2000)), and various studies have shown that DHODH inhibitors can stop the proliferation of tumor cells in some circumstances (e.g., see Loffler, Eur. J. Biochem. 107:207-215 (1980)).

Other circumstances in which DHODH inhibitors have been identified as candidates for the clinical control of rapid cell division include activated immune cells, diseased skin cells, cancers, and infectious agents. Examples of DHODH inhibitors used or being developed for proliferative disorders include brequinar, leflunomide, and teriflunomide. Inhibitors of DHODH have further been disclosed for the treatment or prevention of autoimmune diseases, immune and inflammatory diseases, angioplastic-related disorders, viral, bacterial, and protozoic diseases.

Although DHODH is an attractive target for therapeutic intervention for a varieity of clinical conditions, including cancer, there remain significant issues with currently described compounds. For example, many of these compounds, including brequinar, suffer from being associated with poor bioavailability, due in part to the poor aqueous solubility and GI uptake. Accordingly, currently described DHODH inhibitors can have limited pharmaceutical efficacy due to such bioavailability issues.

Despite advances in research directed towards effective and therapeutically useful DHODH inhibitors, there remain a scarcity of compounds that are both efficacious and have the appropriate bioavailability properties. These needs and other needs are satisfied by the present disclosure.

### SUMMARY

In accordance with the purpose(s) of the disclosure, as embodied and broadly described herein, the disclosure, in one aspect, relates to pharmaceutical combinations and methods of treating a clinical condition, e.g., AML, by administering to a subject a pharmaceutical combination comprising a DHODH inhibitor and an anti-CD38 antibody. The pharmaceutical combination can further comprise one or more additional therapeutic agents. Other clinical conditions that can be treated by the disclosed pharmaceutical compositions, i.e., a combination therapy comprising a DHODH inhibitor and an anti-CD38 antibody, and disclosed methods of combination therapy includes, but is not limited to, chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, and immunomodulation for solid tumors. Certain non-malignant clinical conditions can also be treated by the disclosed pharmaceutical compositions and methods of treatment include, but is not limited to aplastic anemia, depletion of malignant myeloid derived suppressor cells, and Immunoglobulin light chain amyloidosis (AL).

Disclosed herein are pharmaceutical combinations comprising an antibody specifically recognizing CD38 and at least DHODH inhibitor compound as disclosed herein, a pharmaceutically acceptable salt thereof, and a pharmaceutically acceptable carrier; wherein the antibody specifically recognizing CD38 is capable of killing a CD38+ cell by antibody dependent cell-mediated phagocytosis (ADCP), cellular fratricide, apoptosis, antibody-dependent cell-mediated cytotoxicity (ADCC) and/or complement-dependent cytotoxicity (CDC).

A disclosed DHODH inhibitor can be any DHODH inhibitor as disclosed in Intl. Pat. Appl. No. PCT/US19/38622, which is incorporated herein by reference. An exemplary DHODH inhibitor as disclosed there is:

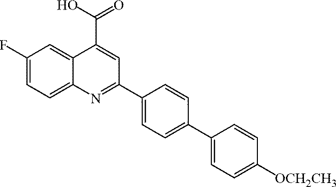 [SMILES: CCOc1ccc(-c2ccc(-c3cc(C(=O)O)c4cc(F)ccc4n3)cc2)cc1]

2-(4′-ethoxy-[1,1′-biphenyl]-4-yl)-6-fluoroquinoline-4-carboxylic acid (Cpd3)

Disclosed DHODH inhibitors can have a formula represented by a structure:

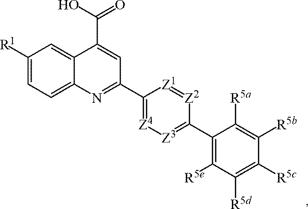

wherein each of Z¹, Z², Z³, and Z⁴ is independently selected from CH and N; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰, -R³⁰-A¹-R⁴⁰, -A¹-R⁴⁰, -A¹-R³⁰-A²-R⁴⁰, or -A¹-R³⁰-A²-R³¹-A³-R⁴⁰; wherein A¹ is selected from —O— and -NR⁵⁰-; wherein R⁵⁰ is selected from hydrogen, -C1-C10 alkyl, -C1-C10 aminoalkyl, and -C1-C10 hydroxyalkyl; wherein A² is selected from —O— and -NR⁶⁰-; wherein R⁶⁰ is selected from hydrogen, -C1-C10 alkyl, -C1-C10 aminoalkyl, and -C1-C10 hydroxyalkyl; wherein A³ is selected from —O—and -NR⁷⁰-; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, and -C1-C10 alkoxy; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; or a pharmaceutically acceptable salt thereof.

Disclosed DHODH inhibitors can have a formula represented by a structure:

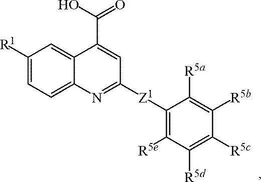

wherein Z¹ is a five-membered heterocyclic diyl; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰ -R³⁰-A¹-R⁴⁰, -A¹-R⁴⁰, -A¹-R³⁰-A²-R⁴⁰, or -A¹-R³⁰-A²-R³¹-A³-R⁴⁰; > wherein A¹ is selected from —O— and -NR⁵⁰-; wherein R⁵⁰ is selected from hydrogen, -C1-C10 alkyl, -C1-C10 aminoalkyl, and -C1-C10 hydroxyalkyl; wherein A² is selected from —O— and -NR⁶⁰-; wherein R⁶⁰ is selected from hydrogen, -C1-C10 alkyl, -C1-C10 aminoalkyl, and -C1-C10 hydroxyalkyl; wherein A³ is selected from —O— and -NR⁷⁰-; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Disclosed DHODH inhibitors can have a formula represented by a structure:

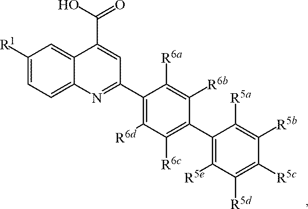

wherein R¹ is selected from hydrogen, halogen, —SF₅, —CN, —N₃, —OH, —NH₂, —CF₃, and -CF₂CF₃; wherein one of R^{5a}, R^{5b}, R^{5c}, R^{5d}, and R^{5e} is selected from a group having formula represented by a structure: -R²⁰, -R³⁰-A¹-R⁴⁰, -A¹-R⁴⁰, -A¹-R³⁰-A²-R⁴⁰, or -A¹-R³⁰-A²-R³¹-A³-R⁴⁰; wherein A¹ is selected from —O— and -NR⁵⁰-; wherein R⁵⁰ is selected from hydrogen, -C1-C10 alkyl, -C1-C10 aminoalkyl, and -C1-C10 hydroxyalkyl; wherein A² is selected from —O— and -NR⁶⁰-; wherein R⁶⁰ is selected from hydrogen, -C1-C10 alkyl, -C1-C10 aminoalkyl, and -C1-C10 hydroxyalkyl; wherein A³ is selected from —O—and -NR⁷⁰-; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and -(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

Also disclosed are methods for the treatment of a disease or disorder in a mammal comprising the step of administering to the mammal a therapeutically effective amount of a disclosed pharmaceutical combination.

Also disclosed are methods for the treatment of a disease or disorder in a mammal comprising the step of administering to the mammal a therapeutically effective amount of disclosed pharmaceutical combination.

Also disclosed are methods for the treatment of a cancer in a mammal comprising the step of administering to the mammal a therapeutically effective amount of at least one a therapeutically effective amount of disclosed pharmaceutical combination.

Also disclosed are methods for the treatment of a disease or disorder associated with T-cell proliferation in a mammal comprising the step of administering to the mammal a therapeutically effective amount of at least one disclosed compound, or a pharmaceutically acceptable salt thereof, or a disclosed pharmaceutical composition.

Also disclosed are kits comprising a therapeutically effective amount of at least one disclosed compound, or a pharmaceutically acceptable salt thereof, or a disclosed pharmaceutical composition; and: (a) at least one agent known to treat a cancer, a host-versus-graft-disease, and/or a disorder associated with T-cell proliferation; and (b) instructions for treating a cancer, a host-versus-graft-disease, and/or a disorder associated with T-cell proliferation.

Also disclosed are methods for manufacturing a medicament comprising combining a therapeutically effective amount of disclosed pharmaceutical combination with a pharmaceutically acceptable carrier or diluent.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the disclosure.

FIGS. 1A-1B show representative data for the effect of a representative DHODH inhibitor on the expression of CD38 in AML cells. Briefly, MV-411 cells with wild type (WT) p53 (FIG. 1A) or mutant p53 (FIG. 1B) were treated with brequinar (BRQ, 1 µM), or Cpd3 (1 µM) for 72 hours, and then the CD38 surface expression was determined by flow cytometry. The CD38 mean fluorescent intensity (MFI) is calculated based on the X-median expression.

FIGS. 2A-2D show representative data for the effect of a representative DHODH inhibitor in combination with an anti-CD38 antibody in a mouse xenograft model. FIG. 2A show representative data for percent survival versus time post-engraftment. Briefly, NSG mice were injected with 0.3 × 10⁶ spleen cells from an MV4-11 engrafted leukemic donor mouse (N=7 per group). Starting at day 7 post-engraftment, mice were treated with vehicle; 50 mg/kg Cpd3 (PO; indicated as “Cpd3 PO” in the figure) on a dosing schedule of Monday/Wednesday/Friday (MWF); 1 µg/g daratumumab (IP; indicated as “Dara IP” in the figure) on a dosing schedule of Tuesday and Friday (TF); or Cpd3 and daratumumab combination (indicated as “Cpd3+Dara” in the figure). The Cpd3 and daratumumab combination treatment comprised dosing with 50 mg/kg Cpd3 on a dosing scheduled of Monday/Wednesday/Friday and dosing with 1 µg/g daratumumab on a dosing schedule of Tuesday and Friday (TF). Mice were monitored until reaching early/end removal criteria (ERC) and overall survival was calculated using Kapler Meyer analysis. The arrow indicates start of treatment. The data show that treatment with either Cpd3 or daratumumab alone showed a modest improvement in survival compared to the vehicle treatment control group, whereas a combination treatment, i.e., Cpd3 in combination with the CD38 antibody daratumumab, dramatically improved survival compared to other treatment groups indicating a synergistic effect of the combination treatment. FIG. 2B shows representative photographic images of spleens isolated from the animals from each treatment group as they reached ERC. The treatment type is indicated below each photographic image as outlined for FIG. 2A. The data show that Cpd3 in combination with the CD38 antibody daratumumab decreased tumor burden, i.e., a smaller spleen size, compared to other treatment groups. FIG. 2C shows reprsentative weight loss data for each treatment group versus time post-engraftment. Weight loss is typically associated with leukemia progression and has also been associated with DHODH inhibitor treatment. The data show that decrease in weight corresponded with removal criteria and was delayed in the Cpd3+Dara group. FIG. 2D shows representative data for percentage of human CD45+ (gated on live cells) for vehicle and combination treatment groups. Briefly, Spleens were isolated from the animals from each treatment group as they reached ERC. The percentage of human CD45+ cells in the spleen were determined by flow cytometry. The data show that combination treatment (Cpd3+Dara) decreased tumor burden in the spleen from treated mice compared to the vehicle treatment group.

FIGS. 3A-3C show representative data for the effect of a representative DHODH inhibitor on the expression of CD38 in primary AML cells. Briefly, primary AML cells were treated with vehicle (DMSO), brequinar (BRQ, 1 µM), or Cpd3 (1 µM) for 72 hours, and then the CD11b and CD38 surface expression were determined by flow cytometry. FIG. 3A shows flow cytometry data for primary AM L cells following treatment with vehicle. FIG. 3B shows flow cytometry data for primary AML cells following treatment with brequinar. FIG. 3C shows flow cytometry data for primary AML cells following treatment with Cpd3.

FIGS. 4A-4B show plots of data obtained from the flow cytometry studies of the type shown in FIGS. 3A-3C. Briefly, primary AML cells were treated with vehicle (DMSO), brequinar (BRQ, 1 µM), BAY2402234 (BAY), or Cpd3 (1 µM) for 72 hours, and then the CD11b and CD38 surface expression were determined by flow cytometry. Mean fluorescence intensity (MFI) plots were normalized to vehicle (DMSO). FIG. 4A shows expression levels of CD11b after three and seven days of treatment with the indicated agent. FIG. 4B shows expression levels of CD38 after three and seven days of treatment with the indicated agent.

FIG. 5 shows representative data obtained from treatment of six AML cell-lines with different mutational backgrounds. The cell lines were treated with the indicated compound at the indicated concentrations for three days. BRQ = Brequinar; BAY = BAY2402234.

FIGS. 6A-6B show representative data for the effect of a representative DHODH inhibitor in combination with an anti-CD38 antibody in a mouse xenograft model using a TIWK (MWF) dosing regiment for the DHODH inhibitor, Cpd4, as described herein belo. FIG. 6A shows data for the indicated therapeutic agents, including combination treatment with DHODH inhibitor, Cpd4, with daratumumab (Dara). FIG. 6B shows data for the indicated therapeutic agents, including combination treatment with DHODH inhibitor, Cpd4, with isatuximab (Isa).

FIGS. 7A-7B show representative data for the effect of a representative DHODH inhibitor in combination with an anti-CD38 antibody in a mouse xenograft model using a daily dosing regiment for the DHODH inhibitor, Cpd4, as described herein belo. FIG. 7A shows data for the indicated therapeutic agents, including combination treatment with DHODH inhibitor, Cpd4, with daratumumab (Dara). FIG. 7B shows data for the indicated therapeutic agents, including combination treatment with DHODH inhibitor, Cpd4, with isatuximab (Isa).

FIGS. 8A-8B show representative data for the effect of a representative DHODH inhibitor in combination with an anti-CD38 antibody in a mouse xenograft model using a daily dosing regiment for the DHODH inhibitor, BAY2402234, as described herein belo. FIG. 8A shows data for the indicated therapeutic agents, including combination treatment with DHODH inhibitor, BAY2402234, with daratumumab (Dara). FIG. 8B shows data for the indicated therapeutic agents, including combination treatment with DHODH inhibitor, BAY2402234, with isatuximab (Isa).

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a unimolecular nanoparticle,” “a nanocluster,” or “a biomimetic vesicle,” including, but not limited to, two or more such unimolecular nanoparticles, nanoclusters, or biomimetic vesicles, including combinations of unimolecular nanoparticles, nanoclusters, or biomimetic vesicles, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/- 10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “dihydroorotate dehydrogenase” and “DHODH” can be used interchangeably, and refer to an enzyme encoded by a gene in humans with a cytogenetic location of 16q22.2 and a molecular location of base pairs 72,008,744 to 72,025,417 on chromosome 16 (Homo sapiens Annotation Release 109, GRCh38.p12). The gene structure in humans comprises 9 exons. DHODH has an EC classification of 1.3.1.1; an intracellular location within the mitochondria; and catalyzes the fourth enzymatic step in de novo pyrimidine biosynthesis. DHODH has also been referred to as DHOdehase; dihydroorotate dehydrogenase, mitochondrial; dihydroorotate dehydrogenase, mitochondrial precursor; dihydroorotate oxidase; human complement of yeast URA1; POADS; PYRD_HUMAN; and URA1.

The terms “inhibits”, “inhibiting”, or “inhibitor” of DHODH, as used herein, refer to inhibition of the enzyme DHODH, unless otherwise specified.

“Synergy”, “synergism” or“synergistic”, as used herein, refers to an effect that is more than the expected additive effect of a combination.

The term “in combination with” as used herein means that two or more therapeutics can be administered to a subject together in a mixture, concurrently as single agents or sequentially as single agents in any order.

As used herein, “IC₅₀,” is intended to refer to the concentration of a substance (e.g., a compound or a drug) that is required for 50% inhibition of a biological process, enzymatic reaction, or component of a biological or enzymatic process. For example, IC₅₀ refers to the half maximal (50%) inhibitory concentration (IC) of a substance as determined in a suitable assay. For example, an IC₅₀ for DHODH activity can be determined in an in vitro enzymatic assay using the methods described herein. Alternatively, an activity can be determined in a cell-based assay, including measurement of an activity or function associated with inhibition of the target process or enzyme. That is, DHODH activity can be indirectly determined in a cell-based assay of cell proliferation. It is believed that DHODH inhibition can lead to growth arrest or inhibition in suitable cell types. DHODH activity can be determined in a suitable cell, such as a primary AML cell or a AML cell-line, using an assay for cell-proliferation, such as an MTS assay as described herein, or a cell-colony forming assay as described herein. Suitable cell lines are described herein below.

As used herein, the term “immune” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells and natural killer cells.

As used herein, the term “DHODH inhibitor” is meant a compound that inhibits the normal enzymatic function of DHODH in converting dihydroorotate to orotate. Alternatively, a DHODH inhibitor inhibits transcription or translation of the DHODH gene. In a particular aspect, the DHODH inhibitor is an oligonucleotide that represses DHODH gene expression or product activity by, for example, binding to and inhibiting DHODH nucleic acid (i.e. DNA or mRNA). In a particular aspect, the DHODH inhibitor is an oligonucleotide e.g. an antisense oligonucleotide, shRNA, siRNA, microRNA or an aptamer. In an aspect the DHODH inhibitor is a small molecule that binds to and modulates DHODH enzymatic function. Examples of DHODH inhibitors include brequinar, leflunomide, redoxal, vidofludimas, S-2678, 2-(3,5-difluoro-3′methoxybiphenyl-4-ylamino)nicotinic acid (also known as ASLAN003), and teriflunomide.

As used herein, “brequinar” and “BQR,” which can be used interchangeably, refer to the compound having a structure represented by the following formula:

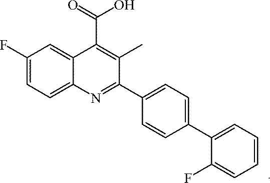

Brequinar can also be referred to by the IUPAC chemical name, or 6-fluoro-2-(2′-fluoro-1,1′-biphenyl-4-yl)-3-methyl-4-quinolinecarboxylic acid. Common salt forms are brequinar potassium and brequinar sodium (also referred to herein as BQR Na), which are the alkali metal salts of the conjugate base of the carboxylic acid. Brequinar is sometimes referred as DuP-785 or NSC-368390.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians’ Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Waals forces, dipole-dipole interactions, dipole-induced-dipole interactions, London dispersion forces, hydrogen bonding, halogen bonding, electromagnetic interactions, π-π interactions, cation-π interactions, anion-π interactions, polar rr-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof. It is understood that a vertebrate can be mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Moreover, adult and newborn subjects, as well as fetuses, whether male or female, are intended to be covered. A patient refers to a subject afflicted with a clinical condition, disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer, a disorder or disease associated with T-cell proliferation, or a graft-versus-host-disease. The effect can be therapeutic in terms of a partial or complete cure of a disease, condition, symptom or adverse effect attributed to the disease, disorder, or condition. The term “treatment” as used herein can include any treatment of a cancer, a disorder or disease associated with T-cell proliferation, or a graft-versus-host-disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

In the present disclosure, it is understood that in some cases, an effective amount or dose of a disclosed compound is the amount of the composition that is capable of inhibiting DHODH to provide a clinically meaningful decrease in the signs, symptoms, or causes of a disease, or any other desired alteration of a biological system, as a result of inhibiting DHODH. For example, an “effective amount” for therapeutic uses. In some aspects, an appropriate “effective” amount in any individual case is determined using techniques, such as a dose escalation study.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1 -to-C 6 alkyl esters and C 5 -to-C 7 cycloalkyl esters, although C 1 -to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to-C 6 alkyl amines and secondary C 1 -to-C 6 dialkyl amines. In the case of secondary amines, the amine can also be in the form of a 5- or 6-membered heterocycle containing one nitrogen atom. Amides derived from ammonia, C 1 -to-C 3 alkyl primary amides and C 1 -to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

It is understood, that unless otherwise specified, temperatures referred to herein are based on atmospheric pressure (i.e. one atmosphere).

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. Similarly, “Ar¹,” “Ar²,” “Ar³,” and “Ar⁴” are used herein as generic symbols to represent various specific aryl substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

As used herein “aminoalkyl” refers to a straight or branched chain alkyl group in which at least one hydrogen is replaced with an amino group, generally 1-3 amino groups. Non-limiting examples of aminoalkyl groups include —CH₂NH₂, —(CH₂)₂NH₂, —CHCH₃NH₂, —(CH₂)₂CHCH₃NH₂, —(CH₂)₂CHNH₂CH₂CH₃, —CHCH₃(CH₂)₂NH₂, and the like.

As used herein “alkylamino” refers to an amino group have at least one hydrogen replaced with an alkyl group. Thus, alkylamino refers to the group —NR^{a}R^{a}, wherein R^{a}and R^{b} are independently selected form H and alkyl, provided at least one of R^{a} or R^{b} is an alkyl. Non-limiting examples of alkylamino groups include —NHCH₃, —NHCH₂CH₃, —NH(CH₂)₂CH₃, —N(CH₃)₂, —N(CH₃)CH₂CH₃, —N(CH₃)(CH₂)₂CH₃, and the like.

As used herein “hydroxyalkyl” refers to a straight or branched chain alkyl group in which at least one hydrogen is replaced with an hydroxy group, generally 1-3 hydroxy groups. Non-limiting examples of hydroxyalkyl groups include —CH₂OH, —(CH₂)₂OH, —CHCH₃OH, —(CH₂)₂CHCH₃OH, —(CH₂)₂CHOHCH₂CH₃, —CHCH₃(CH₂)₂OH, and the like.

The term “alkanediyl”, as used herein, unless otherwise indicated, means bivalent straight and branched chained saturated hydrocarbon radicals having carbon atoms. For example, “C1-C6 alkanediyl” would refer to bivalent straight and branched chained saturated hydrocarbon radicals having 1 to 6 carbon atoms, such as, for example, methylene, 1,2-ethanediyl (—CH₂CH₂—), propanediyl or 1,3-propanediyl (—(CH₂)₃—), butanediyl or 1,4-butanediyl (—(CH₂)₄—), pentanediyl or 1,5-pentanediyl (—(CH₂)₅—), hexanediyl or 1,6-hexanediyl (—(CH₂)₆—) and the branched isomers thereof (e.g., isopropanediyl (— CHCH₃CH₂—)). Alkanediyl groups can be further substituted, e.g., aminoalkanediyl or hydroxyalkanediyl.

As used herein, “aminoalkanediyl” refers to a straight or branched chain alkanediyl group in which at least one hydrogen is replaced with an amino group, generally 1-3 amino groups. Non-limiting examples of aminoalkanediyl groups include —CH₂NH—, —(CH₂)₂NH— —CHCH₃NH—, —(CH₂)₂CHCH₃NH—, —(CH₂)₂CHNH₂(CH₂)₂—, —CH₂CHNH₂(CH₂)₂—, —CH₂NH(CH₂)₂—, —(CH₂)₂NH(CH₂)₂—, —CHCH₃(CH₂)₂NH—, and the like.

As used herein, “hydroxyalkanediyl” refers to a straight or branched chain alkanediyl group in which at least one hydrogen is replaced with a hydroxy group, generally 1-3 hydroxy groups. Non-limiting examples of hydroxyalkanediyl groups include —CHOH—, —CH₂CHOH—, —CCH₃OH—, —(CH₂)₂CCH₃OH—, —(CH₂)₂CHOH(CH₂)₂₋, —CH₂CHOH(CH₂)₂—, —CHOH(CH₂)₂—, —CH₂CHOH(CH₂)₂—, —CHCH₃CH₂CHOH—, and the like.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or—OA¹—(OA²)ₐ— OA³, where “a” is an integer of from 1 to 200 and A¹, A², and A³ are alkyl and/or cycloalkyl groups.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “heterocycle” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “carboxylic acid” as used herein is represented by the formula —C(O)OH.

The terms “halo,” “halogen” or “halide,” as used herein can be used interchangeably and refer to F, Cl, Br, or I.

The term “hydroxyl” or “hydroxy” as used herein is represented by the formula —OH.

The term “nitro” as used herein is represented by the formula —NO₂.

The term “nitrile” or “cyano” as used herein is represented by the formula —CN.

“R¹,” “R²,” “R³,” ... “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

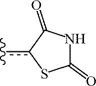

regardless of whether thiazolidinedione is used to prepare the compound. In some aspects the radical (for example an alkyl) can be further modified (i.e., substituted alkyl) by having bonded thereto one or more “substituent radicals.” The number of atoms in a given radical is not critical to the present disclosure unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some aspects, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfonyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the disclosure includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (-) are employed to designate the sign of rotation of plane-polarized light by the compound, with (-) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non- isotopically labeled reagent.

The compounds described in the disclosure can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the disclosure to form solvates and hydrates. Unless stated to the contrary, the disclosure includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser’s Reagents for Organic Synthesis, Volumes 1-17 (John Wiley and Sons, 1991); Rodd’s Chemistry of Carbon Compounds, Volumes 1-5 and Supplementals (Elsevier Science Publishers, 1989); Organic Reactions, Volumes 1-40 (John Wiley and Sons, 1991); March’s Advanced Organic Chemistry, (John Wiley and Sons, 4th Edition); and Larock’s Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Combination Therapy - Treatment With a DHODH Inhibitor and an Anti-CD38 Antibody

The present disclosure pertains to a pharmaceutical combination comprising at leaset one compound that can inhibit dihydroorotate dehydrogenase (DHODH), i.e., DHODH inhibitors, and an anti-CD38 antibody. Also described herein are methods of administering the disclosed pharmaceutical combinations to a subject in need thereof. In some aspects, the subject can have a disease or disorder associated with DHODH activity, such as a cancer (blood or solid), autoimmune diseases, depetion of cancer associated MDSC, a disorder or disease associated with T-cell proliferation, or a graft-versus-host-disease, including, but is not limited to, chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, and immunomodulation for solid tumors. Certain non-malignant clinical conditions can also be treated by the disclosed pharmaceutical compositions and methods of treatment include, but is not limited to aplastic anemia, depletion of malignant myeloid derived suppressor cells, and Immunoglobulin light chain amyloidosis (AL).

In particular, as disclosed herein below in the Examples, treatment of AML cells with a representative DHODH inhibitor can be associated with an upregulation in CD38 expression in these same cells. CD38 has been associated with differentiation and response to differentiation therapy in AML (see Prus et al, Leukemia and Lymphoma 2003, vol 44: issue 4). It has also been previously observed that targeting CD38 with a therapeutic antibody, such as daratumumab, can be an effective when done in combination with a differentiating agent, all-trans retinoic acid (“ATRA”; Buteyn et al, International Immunology 2018, vol. 30 no. 8). Specifically, these studies showed that demonstrated ATRA induced CD38 expression in MV4-11 cell line was able to cause AML cellular fratricide with daratumumab.

It is disclosed herein that a combination of DHODH inhibitor and a therapeutic antibody that binds CD38 has a profound effect on median survival days as determined in a mouse xenograft model. Without wishing to be bound by a particular theory, it is possible that a combination of DHODH inhibitor with an anti-CD38 antibody is able to cause, in part, an AML cellular fratricide, leading to improved survival and decreased tumor burden.

The present disclosure pertains to pharmaceutical compositions comprising a combination of a DHODH inhibitor and an anti-CD38 antibody. It is understood that “combination” can be a combination such as a co-formulated pharmaceutical composition. Alternatively, “combination” can be in the form of co-packaging such that both therapeutic agents, i.e., the DHODH inhibitor and the anti-CD38 antibody, are packaged in a manner such they can be simultaneously dispensed together, dispensed sequentially, dispensed on a fixed scheduled relative to one another, or combinations thereof. In some aspects, the dose can also be sequenced to enhance the expression in CD38 on tumor cells before administering CD38 antibody.

“Synergy” or as used herein with respect to the clinical condition treating effects, e.g., tumor-treating effects, of the combination of a DHODH inhibitor and an anti-CD38 antibody, comprises tumor growth inhibition, including tumor suppression, tumor growth or re-growth delay, and/or substantial elimination of established tumors, and including inhibition of re-establishment of the tumor following cessation of the treatment, that is significantly greater in terms of the amount, degree, extent of inhibition, and/or rate, and/or significantly longer significantly longer in terms of the time of inhibited re-establishment relative to the tumor-treating effects of a DHODH inhibitor or the anti-CD38 antibody alone, or relative to an additive tumor treating effect of the agents in isolation. Thus, a “synergistically effective amount” of a DHODH inhibitor or a “synergistically effective amount” of an anti-CD38 antibody is an amount at which “synergy” of the DHODH inhibitior and an anti-CD38 antibody occurs, including an amount at which both agents synergize to substantially inhibit, delay, or suppress tumor growth, substantially eliminate established tumors, and/or substantially inhibit, delay, or suppress tumor re-establishment.

A pharmaceutical combination comprising an anti-CD38 antibody a DHODH inhibitor may be used, for example, to inhibit, reduce, decrease, block, or prevent proliferation of a cell that expresses CD38 on its surface. A combination therapy comprising an anti-CD38 antibodywith a DHODH inhibitor may be used, for example, to induce, facilitate, or enhance apoptosis of a cell that expresses CD38 on its surface. The cell that expresses CD38 may be a lymphocyte, an autoimmune lymphocyte, or a tumor cell such as a leukemia cell, a multiple myeloma cell, or a lymphoma cell.

The present disclosure further pertains to methods of treating a clinical condition, e.g., AML, by administering to a subject a combination therapy comprising a DHODH inhibitor and an anti-CD38 antibody. The combination therapy can further comprise one or more additional therapeutic agents. Other clinical conditions that can be treated by the disclosed pharmaceutical compositions, i.e., a combination therapy comprising a DHODH inhibitor and an anti-CD38 antibody, and disclosed methods of combination therapy includes, but is not limited to, chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, and immunomodulation for solid tumors. Certain non-malignant clinical conditions can also be treated by the disclosed pharmaceutical compositions and methods of treatment include, but is not limited to aplastic anemia, depletion of malignant myeloid derived suppressor cells, and Immunoglobulin light chain amyloidosis (AL).

Since the activity of the combination pharmaceutical combination depends on the doses used, it is thus possible to use lower doses and to increase the activity while decreasing the toxicity phenomena in view of the synergistic aspects of the combination disclosed herein. The improved efficacy of a combination according to the present disclosure may be demonstrated by determination of the therapeutic synergy. A combination manifests therapeutic synergy if it is therapeutically superior to the best agent of the study used alone at its maximum tolerated dose or at its highest dose tested when toxicity cannot be reached in the animal species.

The constituents of which the disclosed pharmaceutical combination may be administered simultaneously, semi-simultaneously, separately, or spaced out over a period of time so as to obtain the maximum efficacy of the combination; it being possible for each administration to vary in its duration from a rapid administration to a continuous perfusion.

As a result, for the purposes of the present disclosure, the combinations are not exclusively limited to those which are obtained by physical association of the constituents, but also to those which permit a separate administration, which can be simultaneous or spaced out over a period of time.

The pharmaceutical combinations according to the present disclosure are preferably compositions which can be administered parentally. However, these compositions may be administered orally, subcutaneously or intraperitoneally in the case of localized regional therapies.

The compositions for parental administration are generally pharmaceutically acceptable, sterile solutions or suspensions which may optionally be prepared as required at the time of use. For the preparation of non-aqueous solutions or suspensions, natural vegetable oils such as olive oil, sesame oil or liquid petroleum or injectable organic esters such as ethyl oleate may be used. The sterile aqueous solutions can consist of a solution of the product in water. The aqueous solutions are suitable for intravenous administration provided the pH is appropriately adjusted and the solution is made isotonic, for example with a sufficient amount of sodium chloride or glucose. The sterilization may be carried out by heating or by any other means which does not adversely affect the composition. The combinations may also take the form of liposomes or the form of an association with carriers as cyclodextrins or polyethylene glycols.

Thus, the present disclosure also encompasses the use of the above pharmaceutical combinations for the manufacture of a medicament for the treatment of a disclosed clinical condition or disorder, including, but is not limited to, chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, and immunomodulation for solid tumors.

Another aspect of the present disclosure is an article of manufacture comprising: (a) a packaging material; (b) a combination of an antibody specifically recognizing CD38 and at least one DHODH inhibitor, wherein said antibody is capable of killing a CD38+ cell by apoptosis, antibody-dependent cell-mediated cytotoxicity (ADCC), and complement-dependent cytoxicity (CDC); and (c) a label or package insert contained within said packaging material indicting that said combination thereof is effective for treating a disclosed clinical condition or disorder, including, but is not limited to, chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, and immunomodulation for solid tumors.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Anti-CD38 Therapeutic Agents

In the disclosed pharmaceutical compositions and methods of treating a clinical condition, a DHODH inhibitor is used with an anti-CD38 therapeutic agent. In various aspects, the anti-CD38 therapeutic agent comprises a therapeutic agent that decreases the number of CD38 expressing cells and/or level of cell membrane concentration of CD38 protein.

In various aspects, a suitable anti-CD38 therapeutic agent can be one of the anti-CD38 antibodies as disclosed herein, or any other suitable anti-CD38 antibody as know to the skilled artisan. As used herein, an “anti-CD38 antibody” refers to any antibody recognizing a CD38 epitope, including, but not limited to, chimeric or humanized antibody, an antibody fragment, an antibody-drug conjugate, a radioimmune therapy antibody conjugate (e.g., a radionuclide labelled anti-CD38 antibody), a nanobody, a bispecific antibody, a trispecific antibody, a single variable-domain antibody, or combinations thereof.

In various aspects, the anti-CD38 therapeutic agent can comprise a cellular therapy, e.g., an antigen-specific adoptive cell therapy, including, but not limited to, T cells expressing CAR (i.e., a CAR T based cellular therapy comprising a CAR T cell having at least partial specificity for an antigen such as CD-38). In a further aspect, the anti-CD38 therapeutic agent comprises a CAR-T therapeutic agent target CD38 expressing cells. In some instances, the CAR-T therapeutic agent induces apoptosis of CD38 positive cells.

As used herein, the terms“T lymphocyte” and“T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells), and the like. Additional types of helper T cells include cells such as Th3 (Treg), Thl7, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

CD4+ T cells″ refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10.“CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

Suitable anti-CD38 CAR T cells for use as an anti-CD38 therapeutic agent can comprise those that have been engineered through retroviral vector-mediated transduction of the transmembrane domain of CD8α, the intracellular domains of 4-1BB and CD3ζ and anti-CD38 single-chain variable domain (scFv) such as those described by Mihara and co-workers (Leukemia. 2012 Feb; 26(2):365-7); Drent et al. (Mol Ther. 2017 Aug 2; 25(8):1946-1958) and Drent, et al. (Haematologica. 2016 May; 101(5):616-25).

In various aspects, the anti-CD38 therapeutic agent can be a CD38 CAR-T, CD38 DAR-T, and/or CD38 antibody-drug conjugate as previously described by Sorrento Therapeutics.

As used herein, “CAR T” refers to a chimeric antigen receptor-T cell for adoptive cellular immunotherapy.

As used herein, “DAR T” refers to a dimeric antigen receptor-T cell, e.g., express a dimeric antigen receptor into T-cell receptor (TCR) alpha chain constant region (TRAC). In this manner, TRAC is knocked out and antigen is knocked into its locus. The Dimeric Antigen Receptor (DAR) can utilize a Fab instead of the scFv used by traditional Chimeric Antigen Receptor (CAR) T cells.

Further exemplary, but non-limiting, anti-CD38 therapeutic agents useful in the disclosed pharmaceutical compositions and methods include those listed herein below in Tables 1-3.

TABLE 1

Anti-CD38 Therapeutic Agents

| | | |
|---|---|---|
| Therapy | Company | Technology Type |
| ISB-1342 | Ichnos Sciences Inc | Bispecific mAb |
| Y-150 | Wuhan YZY Biopharma Co Ltd | Bispecific mAb |
| CD38/CD3 BsAb | Sorrento | Bispecific mAb |
| | Therapeutics Inc | |
| ISB-1908 | Ichnos Sciences Inc | Bispecific mAb |
| KPMW-101 | Kleo Pharmaceuticals Inc | Bispecific mAb |
| CD38 x CD3 T cell Engager | IGM Biosciences Inc | Bispecific mAb |
| anti-CD3-PLGA-anti-CD38 | Benhealth Biopharmaceutical (Shenzhen) Co Ltd | Bispecific mAb |
| AMG-424 | Xencor Inc | Bispecific mAb |
| XmAb-13243 | Amgen Inc | Bispecific mAb |
| XmAb-13551 | Amgen Inc | Bispecific mAb |
| Dual Specificity CD38 and bcma CAR-T Cells | China PLA General Hospital | Cellular Immunotherapy |
| 4SCAR-CD22/CD123/CD38/CD10/CD20/TSLPR | Shenzhen Geno-Immune Medical Institute | Cellular Immunotherapy |
| CD33/CD38/CD56/CD117/CD123-specific gene-engineered T cells | Shenzhen Geno-Immune Medical Institute | Cellular Immunotherapy |
| multi-CAR T cells (anti CD38 and CD269) | Shenzhen Geno-Immune Medical Institute | Cellular Immunotherapy |
| CD38 CAR-T | China PLA General Hospital | Cellular Immunotherapy |
| BCMA-CD38 bispecific CAR-T cells | Huazhong University of Science & Technology | Cellular Immunotherapy |
| BCMA and CD38 CAR-T cells | Tianjin First Central Hospital | Cellular Immunotherapy |
| CD38 CAR T-Cells | Soochow University Hospital 1 | Cellular Immunotherapy |
| CAR-T therapy in Relapsed/Refractory multiple myeloma | Wenzhou Medical University | Cellular Immunotherapy |
| T-007 | Celularity Inc | Cellular Immunotherapy |
| CD38 CAR-NK | Celularity Inc | Cellular Immunotherapy |
| anti-BCMA CAR-T and anti-CD38 CAR-T therapy | Affiliated Hospital of Xuzhou Medical University | Cellular Immunotherapy |
| CYT-104 | Cytovia Therapeutics Inc | Cellular Immunotherapy |
| BCMA/CD-38 Dual STAR-T | China Immunotech (Beijing) Biotechnology Co Ltd | Cellular Immunotherapy |
| CD38 CAR-T | Shanghai YaKe Biotechnology Inc | Cellular Immunotherapy |
| CAR2 Anti-CD38 A2 CAR-T Cells for Multiple Myeloma | Sorrento Therapeutics Inc | Cellular Immunotherapy |
| Anti-CD38-CAR T Cells for ATLL | Hiroshima University | Cellular Immunotherapy |
| CYT-538 | Cytovia Therapeutics Inc | Cellular Immunotherapy |
| UCART-38 | Cellectis SA | Cellular Immunotherapy |
| ONKT-102 | ONK Therapeutics Ltd | Cellular Immunotherapy; mAb |
| A-145D | Takeda Pharmaceutical Co Ltd | Fusion Protein |
| daratumumab | Janssen Biotech Inc | mAb |
| isatuximab | Sanofi-Aventis US LLC | mAb |
| felzartamab | I-Mab | mAb |
| daratumumab biosimilar | Hengenix Biotech Inc | mAb |
| GEN-3014 | Genmab AS | mAb |
| Monoclonal Antibodies to Inhibit CD38 for Multiple Myeloma | Ligand Pharmaceuticals Inc | mAb |
| KP-1196 | Kleo Pharmaceuticals Inc | Small Molecule |

TABLE 2

Anti-CD38 Therapeutic Agents

| | | |
|---|---|---|
| Agent (developer) | Mechanism of action | Tumor type and phase |
| Daratumumab (Darzalex) (Janssen Biotech) | Fully human IgG1 CD38 mAb | Phase 3 studies in MRD+ MM, newly diagnosed MM, and high-risk smoldering MM Phase 2 in R/R Waldenström macroglobulinemia, RR TP53-mutant CLL, R/R AML or high-risk MDS, pediatric ALL,or lymphoblastic lymphoma Phase 1 in CLL, high-risk localized prostate cancer |
| Isatuximab-irfc (Sorclisa) (Sanofi-Aventis) | Humanized IgG1 CD38 mAb | Phase 3 in high-risk smoldering MM, RR MM, newly diagnosed MM Phase 2 in pediatric hematologic malignancies, RR mCRC, advanced solid tumors |
| TJ202 (I-Mab Biopharma) | Fully human IgG1 CD38 mAb | Phase 2 and 3 in R/R MM |
| GBR 1342 (Glenmark Pharmaceuticals) | Bispecific mAb targeting CD38 and CD3 | Phase 1 /2 in previously treated MM |
| AMG 424 (Amgen) | Bispecific mAb targeting CD38 and CD3 | Phase 1 in R/R MM |
| TAK-573 (Takeda Oncology) | Fully human IgG1 CD38 mAb | Phase ½ in R/R MM Phase 1 in metastatic and advanced solid tumors |
| TAK-169 (Takeda Oncology) | Engineered toxin body; CD38 scFv fused to enzymatically active deimmunized SLTA | Phase 1 in R/R MM |
| CD38 CAR T cells (various companies) | CD38-tarqeted adoptive cell therapy | Phase ½ in R/R AML. R/R MM. R/R B-cell ALL after CD19+ CART-cell therapy |
| BM38 (Cellyan Therapeutics) | Dual CD38- and BCMA- targeted adoptive cell therapy (CAR T) | Phase 1 /2 in R/R MM |

ALL, acute tymphobfastic leukemia: AML. acute myetoid leukemia; CAR, chimeric antigen receptor; CD, cluster of differentiation; CLL, chronic lymphocytic leukemia; IgG, immunoglobulin G; mAb, monoclonal antibody; mCRC, metastatic colorectal cancer; MDS, myelodysplastic syndrome; MM, multipe myeloma; MRD, minimal residual disease; R/R, relapsed or refractory; scFV, single-chain variable fragment; SLTA, Shiga-like toxin A.

TABLE 3

Anti-CD38 Therapeutic Agents: CAR T Therapeutic Agents

| | | | |
|---|---|---|---|
| | Clinical Trial | Interventions | Locations |
| 1 | A Feasibility and Safety Study of CD38 CAR-T Cell Immunotherapy for Relapsed B-cell Acute Lymphoblastic Leukemia After CD19 CAR-T Adoptive Cellular Immunotherapy | • Biological: Specificity CD38 CAR-T Cells | • Biotherapeutic Department and Hematology Department of Chinese PLA, Beijing, Beijing, China |
| 2 | CAR-T Cells Therapy in Relapsed/Refractory Acute Myeloid Leukemia | • Biological: CART therapy in Acute myeloid leukemia(AML) | • Southern Medical University Zhujiang Hospital, Guangdong, Guangdong, China |
| 3 | Study to Evaluate the Safety and Efficacy of Anti-CD38 CAR-T in Relapsed or Refractory Multiple Myeloma Patients | • Biological: CAR2 Anti-CD38 A2 CAR-T Cells | • Mayo Clinic Florida, Jacksonville, Florida, United States |
| | | | • Mayo Clinic Minnesota, Rochester, Minnesota, United States |
| | | | • Icahn School of Medicine, New York, New York, United States |
| | | | • University of Pennsylvania, Abramson Cancer Center, Philadelphia, Pennsylvania, United States |
| 4 | CAR-T Cells Therapy in Relapsed/Refractor y Multiple Myeloma | • Biological: CART therapy in Relapsed/ Refractory multiple myeloma | • Southern Medical University Zhujiang Hospital, Guangdong, Guangdong, China |
| 5 | Integrin #7, BCMA, CS1, CD38 and CD138 as the Single or Compound Targets for the Fourth Genenation of CAR-T Cells | • Biological: CAR-T therapy in Relapsed/ Refractory multiple myeloma | • Jinhong Jiang, Lishui, Zhejiang, China |
| | | | •The Sixth Affiliated Hospital of Wenzhou Medical University, Lishui, Zhejiang, China |
| | | | •Zhejiang QiXin Biotech, Wenzhou, Zhejiang, China |
| 6 | A Feasibility and Safety Study of Dual Specificity CD38 and BCMA CAR-T Cell Immunotherapy for Relapsed or Refractory Multiple Myeloma | • Biological: Dual Specificity CD38 and bcma CAR-T Cells | •Biotherapeutic Department and Hematology Department of Chinese PLA General Hospital, Beijing, China |
| 7 | CD38-targeted Chimeric Antigen Receptor T Cell (CART) in Relapesd or Refractory Acute Myeloid Leukemia | • Biological: CART-38 | • The First Affiliated Hospital of Soochow University, Suzhou, Jiangsu, China |
| 8 | CAR-T Immunotherapy Targeting CD19- ALL | • Biological: 4SCAR-CD22/CD123/CD38/ CD10/CD20/TSLPR | • Zhujiang Hospital of Southern Medical University, Guangzhou, Guangdong, China |
| | | | •Shenzhen Geno-immune Medical Institute, Shenzhen, Guangdong, China |
| | | | •Zhongxi Children Hospital, Shijiazhuang, Hebei, China |
| 9 | Combination CAR-T Cell Therapy Targeting Hematological Malignancies | • Biological: 4SCAR19 and 4SCAR22 Biological: 4SCAR19 and 4SCAR38 | • Zhujiang Hospital of Southern Medical University, Guangzhou, Guangdong, China |
| | | • Biological: 4SCAR19 and 4SCAR20 | • Zhujiang Hospital of Southern Medical University, Guangzhou, Guangdong, China |
| | | • Biological: 4SCAR19 and 4SCAR123 | • Shenzhen Geno-immune Medical Institute, Shenzhen, Guangdong, China |
| | | • Biological: 4SCAR19 and 4SCAR70 | •Yunnan Cancer Hospital & The Third Affiliated Hospital of Kunming Medical University & Yunnan Cancer Center, Kunming, Yunnan, China |
| | | • Biological: 4SCAR19 and 4SCAR30 | |
| 10 | Multi-CAR T Cell Therapy for Acute Myeloid Leukemia | • Biological: Muc1/CLL1/CD33/CD3 8/CD56/ CD123-specific gene-engineered T cells | • Zhujiang Hospital of Southern Medical University, Guangzhou, Guangdong, China |
| | | | • Shenzhen Geno-immune Medical Institute, Shenzhen, Guangdong, China |
| | | | • Yunnan Cancer Hospital & The Third Affiliated Hospital of Kunming Medical University & Yunnan Cancer Center, KunMing, Yunnan, China |
| 11 | 4SCAR-T Therapy Post CD19-targeted Immunotherapy | • Biological: Infusion of 4SCAR-T specific to CD22/CD123/CD38/ CD10/CD20 | •Shenzhen Children’s Hospital, Shenzhen, Guangdong, China |
| | | | • The Seventh Affilliated Hospital, Sun Yat-Sen University, Shenzhen, Guangdong, China |
| | | | • Shijiazhuang Zhongxi Children Hospital, Shijiazhuang, Hebei, China |

### Anti-CD38 Antibodies

In the disclosed pharmaceutical compositions and methods of treating a clinical condition, a DHODH inhibitor is used with an anti-CD38 antibody. A suitable anti-CD38 antibody can be one of the anti-CD38 antibodies as disclosed herein, or any other suitable anti-CD38 antibody as know to the skilled artisan. The the antibody recognizing CD38 can be capable of killing a CD38+ cell by antibody dependent cell-mediated phagocytosis (ADCP), cellular fratricide, apoptosis, antibody-dependent cell-mediated cytotoxicity (ADCC) and/or complement-dependent cytotoxicity (CDC).

By “ADCC” or “antibody dependent cell-mediated cytotoxicity” as used herein is meant the cell-mediated reaction wherein nonspecific cytotoxic cells that express FcyRs recognize bound antibody on a target cell and subsequently cause lysis of the target cell. ADCC is correlated with binding to FcyRllla; increased binding to FcyRllla leads to an increase in ADCC activity.

By “ADCP” or antibody dependent cell-mediated phagocytosis as used herein is meant the cell-mediated reaction wherein nonspecific cytotoxic cells that express FcyRs recognize bound antibody on a target cell and subsequently cause phagocytosis of the target cell.

In various aspecdts, the antibody recognizing CD38 is selected from daratumumab, isatuximab (SAR650984), felzartamab, ISB-1342, Y-150, ISB-1908, KPMW-101, AMG-424, XmAb-13243, XmAb-13551, MOR202 (MorphoSys AG), TAK-079, TAK-169, KP-1196, BM38, TJ202, and combinations thereof. In a further aspect, antibody recognizing CD38 is selected from an antibody described in Tables 1 and 2 above, including combinations of such antibodies.

Exemplary useful antibodies include monoclonal antibodies 38SB13, 38SB18, 38SB19, 38SB30, 38SB31, and 38SB39, which specifically recognize CD38, which are described in PCT application WO2008/047242, which is incorporated by reference herein in its entirety. These anti-CD38 antibodies are capable of killing CD38+ cells by three different cytotoxic mechanisms, induction of apoptosis, antibody-dependent cell-mediated cytotoxicity (ADCC), and complement-dependent cytotoxicity (CDC). In addition, these antibodies are able to directly induce apoptosis of CD38+ cells, even without the presence of stroma cells or stroma-derived cytokines. The hybridoma cell lines producing the 38SB13, 38SB18, 38SB19, 38SB30, 38SB31, and 38SB39 murine anti-CD38 antibodies have been deposited at the American Type Culture Collection (10801 University Bld, Manassas, Va., 20110-2209, USA), on Jun. 21, 2006, under the deposit numbers PTA-7667, PTA-7669, PTA-7670, PTA-7666, PTA-7668, and PTA-7671, respectively (as described in WO2008/047242). Further exemplary useful antibodies for the disclosed pharmaceutical combinations include those disclosed in U.S. Pat. Application No. 2018/0066069.

Antibodies against CD38, such as daratumumab, MOR202, or SAR650984, have been, and are currently being evaluated in the clinic for their efficacy to treat hematological malignancies and plasma cell disorders, including multiple myeloma. Each antibody has been found to bind to a different portion of the extracellular region of CD38, and each demonstrates different clinical responses (e.g., anti -tumor effects). Daratumumab, available from Johnson & Johnson (Janssen Biotech)/Genmab as Darzalex® is described in the publication to de Weers et. al, “Daratumumab, a Novel Therapeutic Human CD38 Monoclonal Antibody, Induces Killing of Multiple Myeloma and Other Hematological Tumors,” j Immunology, 2010, 186(3): 1840-1848; MOR202, available from Celgene Corp./Morphosys, is described in U.S. Pat. No. 8,877,899; and SAR650984, available from Sanofi/lmmunogen as Isatuximab, is described in the publication to Park et al, “SAR650984: A Potent Anti-CD38 Therapeutic Antibody with Three Mechanisms of Action (Apoptosis, ADCC, CDC) for Hematological Malignancies,” BLOOD, vol. 1 12, No. 11, November 2008, p. 951, and Deckert et el, “SAR650984, A Novel Humanized CD38-Targeting Antibody, Demonstrates Potent Antitumor Activity in Models of Multiple Myeloma and Other CD38+ Hematologic Malignancies,” Clin Cancer Res 2014; 20:4574-83, and U.S. Pat. No. 8, 153,765. Table 1 below lists a number of antibodies, or fragments thereof, that bind CD38, and their epitope (binding site) on the CD38 molecule, some or all of which may be useful according to various aspects of the present disclosure. In various aspects, the disclosed anti-CD38 antibody used in the disclosed pharmaceutical combinations is capable of killing a CD38+ cell by antibody dependent cell-mediated phagocytosis (ADCP), cellular fratricide, apoptosis, antibody-dependent cell-mediated cytotoxicity (ADCC) and complement-dependent cytotoxicity (CDC). The anti-CD38 antibody can be selected from daratumumab, isatuximab (SAR650984), MOR202 (MorphoSys AG), and TAK-079.

In various further aspects, a suitable anti-CD38 antibody useful in the disclosed pharmaceutical compositions and methods comprise thos0e described herein above in Tables 1 and 2.

In a still further aspect, a suitable anti-CD38 antibody useful in the disclosed pharmaceutical compositions and methods comprise trispecific antibodies such as those described in Sanofi Phase 1 : CD3xCD28xCD38 asset SAR442257 “ClinicalTrials.gov Identifier: NCT04401020”.

Accordingly, the present disclosure provides isolated anti-CD38 antibodies that specifically bind human CD38 protein (and, as described below, additionally and preferably specifically bind primate CD38 protein). Thus, reference to an anti-CD38 antibody is an antibody as defined in the foregoing that is capable of binding CD38. CD38 is a 45 kD type II transmembrane glycoprotein with a long C-terminal extracellular domain and a short N-terminal cytoplasmic domain. That is, in general, CD38 possesses a short intracytoplasmic tail, a transmembrane domain, and an extracellular domain, in specific aspects, the antibodies of the disclosure bind to the extracellular part of the CD38 protein. The CD38 protein is a bifunctional ectoenzyme that can catalyze the conversion of NAD+ into cyclic ADP-ribose (cADPR) and also hydrolyze cADPR into ADP-ribose. CD38 is upregulated and has been implicated in many hematopoietic malignancies.

As is known in the art, CD38 proteins are found in a number of species. Of particular use in the present disclosure are antibodies that bind to both the human and primate CD38 proteins, particularly primates used in clinical testing, such as cynomolgus (Macaca fascicularis, Crab eating macaque, sometimes referred to herein as “cyno”) monkeys. By “human CD38” or “human CD38 antigen” refers to the protein of SEQ ID NO:1 or a functional fraction, such as an epitope, as defined herein.

In some aspects, the anti-CD38 antibodies of the disclosure interact with CD38 at a number of amino acid residues including K121, F135, Q139, D141, M142, D202, V203, H205, Q236, E239, W241, 5274, C275, K276, F284, C287, V288, K289, N290, P291, E292, D293.

In some aspects, the anti-CD38 antibodies of the present disclosure optionally (and in some cases preferably) do not bind to other members of the CD38 family such as CD157.

The term “antibody” is used herein in the broadest sense and specifically covers monoclonal antibodies (including full length monoclonal antibodies) of any isotype such as IgG, IgM, IgA, IgD and IgE, polyclonal antibodies, multispecific antibodies, chimeric antibodies, and antibody fragments. A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions outside of the CDRs are called the “framework regions”. An “antibody” includes monoclonal, polyclonal, bispecific, multispecific, murine, chimeric, fragments, humanized and human antibodies.

A “naked antibody” is an antibody or antigen binding fragment thereof that is not attached to a therapeutic or diagnostic agent. The Fc portion of an intact naked antibody can provide effector functions, such as complement fixation and ADCC (see, e.g., Markrides, Pharmacol Rev 50:59-87, 1998). Other mechanisms by which naked antibodies induce cell death may include apoptosis. (Vaswani and Hamilton, Ann Allergy Asthma Immunol 81: 105-119, 1998.)

An “antibody fragment” is a portion of an intact antibody such as F(ab′)2, F(ab)2, Fab′, Fab, Fv, scFv, dAb and the like. Regardless of structure, an antibody fragment binds with the same antigen that is recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies”, often abbreviated as “scFv” consist of a polypeptide chain that comprises both a VH and a VL domain which interact to form an antigen-binding site. The VH and VL domains are usually linked by a peptide of 1 to 25 amino acid residues. Antibody fragments also include diabodies, triabodies and single domain antibodies (dAb).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody. To maintain binding activity, a limited number of FR amino acid residues from the parent (e.g., murine) antibody may be substituted for the corresponding human FR residues.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A human antibody also can be constructed by genetic or chromosomal transfection methods, as well as phage display technology, all of which are known in the art. (See, e.g., McCafferty et al., 1990, Nature 348:552-553 for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

As used herein, the term “antibody fusion protein” is a recombinantly produced antigen-binding molecule in which an antibody or antibody fragment is linked to another protein or peptide, such as the same or different antibody or antibody fragment or a DDD or AD peptide. The fusion protein may comprise a single antibody component, a multivalent or multispecific combination of different antibody components or multiple copies of the same antibody component. The fusion protein may additionally comprise an antibody or an antibody fragment and a therapeutic agent. Examples of therapeutic agents suitable for such fusion proteins include immunomodulators and toxins. One preferred toxin comprises a ribonuclease (RNase), preferably a recombinant RNase. A preferred immunomodulator might be an interferon, such as interferon-α, interferon-β or interferon-λ.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgG, is bivalent because it has two binding arms but is monospecific because it binds to one epitope. Multispecific, multivalent antibodies are constructs that have more than one binding site of different specificity.

A “bispecific antibody” is an antibody that can bind simultaneously to two targets which are of different structure. Bispecific antibodies (bsAb) and bispecific antibody fragments (bsFab) may have at least one arm that specifically binds to, for example, a T cell, an NK cell, a monocyte or a neutrophil, and at least one other arm that specifically binds to an antigen produced by or associated with a diseased cell, tissue, organ or pathogen, for example a tumor-associated antigen. A variety of bispecific antibodies can be produced using molecular engineering.

An antibody preparation, or a composition described herein, is said to be administered in a “therapeutically effective amount” if the amount administered is physiologically significant. An agent is physiologically significant if its presence results in a detectable change in the physiology of a recipient subject. In particular embodiments, an antibody preparation is physiologically significant if its presence invokes an antitumor response or mitigates the signs and symptoms of an infectious disease state. A physiologically significant effect could also be the evocation of a humoral and/or cellular immune response in the recipient subject leading to growth inhibition or death of target cells.

In various aspects, an antibody disclosed herein comprises single domain antibodies, e.g., a single domain antibody is derived from camelids. In the family of “camelids,” immunoglobulins devoid of light polypeptide chains are found. “Camelids” comprise old-world camelids (Camelus bactrianus and Camelus dromaderius) and new world camelids (for example, Lama paccos, Lama glama and Lama vicugna).

It should be noted that the term “nanobody” as used herein in its broadest sense is not limited to a specific biological source or to a specific method of preparation. For example, the nanobodies hereof can generally be obtained: (1) by isolating the V_{H}H domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized V_{H}H domain; (4) by “camelization” of a naturally occurring VH domain from any animal species and, in particular, from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing. One preferred class of nanobodies corresponds to the V_{H}H domains of naturally occurring heavy chain antibodies directed against BACE1. As further described herein, such V_{H}H sequences can generally be generated or obtained by suitably immunizing a species of camelid with BACE1 (i.e., so as to raise an immune response and/or heavy chain antibodies directed against BACE1), by obtaining a suitable biological sample from the camelid (such as a blood sample, serum sample or sample of B-cells), and by generating V_{H}H sequences directed against BACE1, starting from the sample, using any suitable technique known per se. Such techniques will be clear to the skilled person.

Alternatively, such naturally occurring V_{H}H domains against BACE1 can be obtained from naive libraries of Camelid V_{H}H sequences, for example, by screening such a library using BACE1 or at least one part, fragment, antigenic determinant or epitope thereof using one or more known screening techniques per se. Such libraries and techniques are, for example, described in WO9937681, WO0190190, WO03025020 and WO03035694. Alternatively, improved synthetic or semi-synthetic libraries derived from naive V_{H}H libraries may be used, such as V_{H}H libraries obtained from naive V_{H}H libraries by techniques such as random mutagenesis and/or CDR shuffling, such as, for example, described in WO0043507. Yet another technique for obtaining V_{H}H sequences directed against BACE1 involves suitably immunizing a transgenic mammal that is capable of expressing heavy chain antibodies (i.e., so as to raise an immune response and/or heavy chain antibodies directed against BACE1), obtaining a suitable biological sample from the transgenic mammal (such as a blood sample, serum sample or sample of B-cells), and then generating V_{H}H sequences directed against BACE1 starting from the sample, using any suitable technique known per se. For example, for this purpose, the heavy chain antibody-expressing mice and the further methods and techniques described in WO02085945 and in WO04049794 can be used.

A particularly preferred class of nanobodies hereof comprises nanobodies with an amino acid sequence that corresponds to the amino acid sequence of a naturally occurring V_{H}H domain, but that has been “humanized,” i.e., by replacing one or more amino acid residues in the amino acid sequence of the naturally occurring V_{H}H sequence (and, in particular, in the framework sequences) by one or more of the amino acid residues that occur at the corresponding position(s) in a VH domain from a conventional four-chain antibody from a human being. This can be performed in a manner known per se, which will be clear to the skilled person, for example, on the basis of the further description herein and the prior art on humanization referred to herein. Again, it should be noted that such humanized nanobodies of the invention can be obtained in any suitable manner known per se (i.e., as indicated under points (1)-(8) above) and, thus, are not strictly limited to polypeptides that have been obtained using a polypeptide that comprises a naturally occurring V_{H}H domain as a starting material.

Another particularly preferred class of nanobodies of the invention comprises nanobodies with an amino acid sequence that corresponds to the amino acid sequence of a naturally occurring VH domain, but that has been “camelized,” i.e., by replacing one or more amino acid residues in the amino acid sequence of a naturally occurring VH domain from a conventional four-chain antibody by one or more of the amino acid residues that occur at the corresponding position(s) in a V_{H}H domain of a heavy chain antibody. Such “camelizing” substitutions are preferably inserted at amino acid positions that form and/or are present at the VH-VL interface, and/or at the so-called Camelidae hallmark residues, as defined herein (see, for example, WO9404678). Preferably, the VH sequence that is used as a starting material or starting point for generating or designing the camelized nanobody is preferably a VH sequence from a mammal, more preferably, the VH sequence of a human being, such as a VH3 sequence. However, it should be noted that such camelized nanobodies of the invention can be obtained in any suitable manner known per se (i.e., as indicated under points (1)-(8) above) and, thus, are not strictly limited to polypeptides that have been obtained using a polypeptide that comprises a naturally occurring VH domain as a starting material. For example, both “humanization” and “camelization” can be performed by providing a nucleotide sequence that encodes a naturally occurring V_{H}H domain or VH domain, respectively, and then changing, in a manner known per se, one or more codons in the nucleotide sequence in such a way that the new nucleotide sequence encodes a “humanized” or “camelized” nanobody of the invention, respectively. This nucleic acid can then be expressed in a manner known per se, so as to provide the desired nanobody of the invention.

Alternatively, based on the amino acid sequence of a naturally occurring V_{H}H domain or VH domain, respectively, the amino acid sequence of the desired humanized or camelized nanobody of the invention, respectively, can be designed and then synthesized de novo using techniques for peptide synthesis known per se. Also, based on the amino acid sequence or nucleotide sequence of a naturally occurring V_{H}H domain or VH domain, respectively, a nucleotide sequence encoding the desired humanized or camelized nanobody hereof, respectively, can be designed and then synthesized de novo using techniques for nucleic acid synthesis known per se, after which the nucleic acid thus obtained can be expressed in a manner known per se, so as to provide the desired nanobody of the invention. Other suitable methods and techniques for obtaining the nanobodies hereof and/or nucleic acids encoding the same, starting from naturally occurring VH sequences or preferably V_{H}H sequences, will be clear from the skilled person, and may, for example, comprise combining one or more parts of one or more naturally occurring VH sequences (such as one or more FR sequences and/or CDR sequences), one or more parts of one or more naturally occurring V_{H}H sequences (such as one or more FR sequences or CDR sequences), and/or one or more synthetic or semi-synthetic sequences, in a suitable manner, so as to provide a nanobody hereof or a nucleotide sequence or nucleic acid encoding the same.

A molecule such as an antibody has been “isolated” if it has been altered and/or removed from its natural environment by human intervention. An isolated antibody that specifically binds to an epitope, isoform or variant of CD38, e.g., human CD38 or cynomolgus CD38 may, however, have cross-reactivity to other related antigens, for instance from other species, such as CD38 species homologs. Moreover, an isolated antibody may be substantially free of other cellular material and/or chemicals.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Structures that still rely

In one aspect, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

By “target antigen” or “epitope” as used herein, can be used interchangeably, is meant the molecule that is bound specifically by the variable region of a given antibody. A target antigen may be a protein, carbohydrate, lipid, or other chemical compound. A wide number of suitable target antigens are described below. Thus, the anti-CD38 antibodies, as disclosed herein, have as a target antigen one or more portions of CD38 such as amino acid and carbohydrate portions of CD38, including both contiguous and non-contiguous portions of the CD38 molecule as defined by the primary sequence of the CD38 molecule. That is, a CD38 target antigen can comprise a secondary or tertiary structure in a CD38 molecule comprising one or more amino acid components, one or more carbohydrate components, and combinations thereof.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD or dissociation constant for an antigen or epitope found in CD38 of at least about 10⁻⁴ M, at least about 10⁻⁵ M, at least about 10⁻⁶ M, at least about 10-⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope found in CD38 can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

In some aspects, the antibody can be a mixture from different species, e.g. a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies.

In some instances, the anti-CD38 antibody of the present disclosure is a humanized antibody. The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modelling, statistical analysis and mutagenesis to alter the non-CDR surfaces of antibody variable regions to resemble the surfaces of known antibodies of the target host. The CDR grafting technology involves substituting the complementarity determining regions of, for example, a mouse antibody, into a human framework domain, e.g., see WO 92/22653. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

Humanized antibodies may also comprise residues which are found in neither the human antibody or in the non-human antibody. A humanized antibody may be a super-humanized antibody, e.g., as described in U.S. Pat. No. 7,732,578. The antibodies may be humanized chimeric antibodies. Humanized antibodies also include antibodies with constant region sequences, e.g., variable region framework sequences, that are artificial consensus sequences based on multiple human antibodies.

Fully human antibodies are those where the whole molecule is human or otherwise of human origin, or includes an amino acid sequence identical to or substantially identical to human antibody sequences. Fully human antibodies include those obtained from a human V gene library, for example, where human genes encoding variable regions of antibodies are recombinantly expressed. Fully human antibodies may be expressed in other organisms (e.g., mice and xenomouse technology) or cells from other organisms transformed with genes encoding human antibodies. Fully human antibodies may nevertheless include amino acid residues not encoded by human sequences, e.g., mutations introduced by random or site directed mutations.

The anti-CD38 antibodies may be full length antibodies of any class, for example, IgGI, IgG2 or IgG4. In particular aspects the anti-CD38 antibodies are full-length IgG4 antibodies. The constant domains of such antibodies are preferably human. The variable regions of such antibodies may be of non-human origin, or preferably are human in origin or are humanized. Antibody fragments may also be used in place of the full length antibodies.

In some aspects, the anti-CD38 antibodies may comprise non-immunoglobulin derived protein frameworks. For example, reference may be made to (Ku & Schutz, Proc. Natl. Acad. Sci. USA 92: 6552-6556, 1995) which describes a four-helix bundle protein cytochrome b562 having two loops randomized to create CDRs, which have been selected for antigen binding.

Natural sequence variations may exist among heavy and light chains and the genes encoding them, and therefore, persons having ordinary skill in the art would expect to find some level of variation within the amino acid sequences, or the genes encoding them, of the antibodies described and exemplified herein. Encompassed within the term antibody are sequence variants which maintain CD38 binding specificity and which preferably substantially maintain the affinity of the parent antibody. Such an expectation is due in part to the degeneracy of the genetic code, as well as to the known evolutionary success of conservative amino acid sequence variations, which do not appreciably alter the nature of the encoded protein. Accordingly, such variants and homologs are considered substantially the same as one another and are included within the scope of the disclosure. The antibodies thus include variants having single or multiple amino acid substitutions, deletions, additions, or replacements that retain the biological properties (e.g., binding specificity and binding affinity) of the parent antibodies. The variants are preferably conservative, but may be non-conservative.

Amino acid positions assigned to complementarity determining regions (CDRs) and framework regions (FRs) may be defined according to Kabat Sequences of Proteins of Immunological Interest, National Institutes of Health, Bethesda, Md., 1987 and 1991 (also referred to herein as the Kabat numbering system). In addition, the amino acid positions assigned to CDRs and FRs may be defined according to the Enhanced Chothia Numbering Scheme (http://www.bioinfo.org.uk/mdex.html). The heavy chain constant region of an antibody can be defined by the EU numbering system (Edelman, GM et al. (1969)., Proc. Natl. Acad. USA, 63, 78-85).

According to the numbering system of Kabat, VH FRs and CDRs may be positioned as follows: residues 1-30 (FR1), 31-35 (CDR1), 36-49 (FR2), 50-65 (CDR2), 66-94 (FR3), 95-102 (CDR3) and 103- 113 (FR4), and VL FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). In some instances, variable regions may increase in length and according to the Kabat numbering system some amino acids may be designated by a number followed by a letter. This specification is not limited to FWRs and CDRs as defined by the Kabat numbering system, but includes all numbering systems, including the canonical numbering system or of Chothia et al. (1987) J. Mol. Biol. 196:901-17; Chothia et al. (1989) Nature 342:877-83; and/or Al-Lazikani et al. (1997) J. Mol. Biol. 273:927-48; the numbering system of Honnegher et al. (2001) J. Mol. Biol., 309:657-70; or the IMGT system discussed in Giudicelli et al., (1997) Nucleic Acids Res. 25:206-11. In some aspects, the CDRs are defined according to the Kabat numbering system.

In some particular aspects, for any of the heavy chain CDR2 subdomains described herein, according to the Kabat numbering system, the five C-terminal amino acids may not participate directly in antigen binding, and accordingly, it will be understood that any one or more of these five C-terminal amino acids may be substituted with another naturally- occurring amino acid without substantially adversely affecting antigen binding. In some aspects, for any of the light chain CDR1 subdomains described herein, according to the Kabat numbering system, the four N-terminal amino acids may not participate directly in antigen binding, and accordingly, it will be understood that any one or more of these four amino acids may be substituted with another naturally-occurring amino acid without substantially adversely affecting antigen binding. For example, as described by Padlan et al. (1995) FASEB J. 9:133-139, the five C terminal amino acids of heavy chain CDR2 and/or the four N-terminal amino acids of light chain CDR1 may not participate in antigen binding. In some aspects, both the heavy chain CDR2 and the light chain CDR1 do not directly participate in antigen binding.

In some aspects, chemical analogues of amino acids may be used in the antibodies described and/or exemplified herein. The use of chemical analogues of amino acids is useful, for example, for stabilizing the molecules such as if required to be administered to a subject. The analogues of the amino acids contemplated herein include, but are not limited to, modifications of side chains, incorporation of unnatural amino acids and/or their derivatives during peptide, polypeptide or protein synthesis and the use of crosslinkers and other methods which impose conformational constraints on the proteinaceous molecule or their analogues. The anti-CD38 antibodies may comprise post-translational modifications or moieties, which may impact antibody activity or stability. These modifications or moieties include, but are not limited to, methylated, acetylated, glycosylated, sulfated, phosphorylated, carboxylated, and amidated moieties and other moieties that are well known in the art. Moieties include any chemical group or combinations of groups commonly found on immunoglobulin molecules in nature or otherwise added to antibodies by recombinant expression systems, including prokaryotic and eukaryotic expression systems.

Covalent modifications of antibodies are included within the scope of this disclosure, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues. Examples of side chain modifications contemplated by the disclosure include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal. The carboxyl group may be modified by carbodiimide activation via 0- acylisourea formation followed by subsequent derivation, for example, to a corresponding amide. Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4- chloromercuribenzoate, 4-chloromercuriphenylsulphonic acid, phenyl mercury chloride, 2- chloromercuri-4-nitrophenol and other mercurials; carbamoylation with cyanate at alkaline pH. Tryptophan residues may be modified by, for example, oxidation with N- bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate.

Crosslinkers may be used, for example, to stabilize 3D conformations of the anti- CD38 antibodies using homo-bifunctional crosslinkers such as the bifunctional imido esters having (CH₂)ₙ spacer groups with n=1 to n=6, glutaraldehyde, N-hydroxysuccinimide esters and hetero- bifunctional reagents which usually contain an amino-reactive moiety such as N-hydroxysuccinimide and another group specific-reactive moiety such as maleimido or dithio moiety (SH) or carbodiimide (COOH). In some aspects, the antibodies may be derivatized by known protecting/blocking groups to prevent proteolytic cleavage or enhance activity or stability.

The anti-CD38 antibodies may be affinity matured, or may comprise amino acid changes that decrease immunogenicity, for example, by removing predicted MHC class II-binding motifs. The therapeutic utility of the antibodies described herein may be further enhanced by modulating their functional characteristics, such as antibody-dependent cell-mediated cytotoxicity (ADCC), complement-dependent cytotoxicity (CDC), serum half-life, biodistribution and binding to Fc receptors or the combination of any of these. This modulation can be achieved by protein-engineering, glyco-engineering or chemical methods. Depending on the therapeutic application required, it could be advantageous to either increase or decrease any of these activities. An example of glyco-engineering used the Potelligent® method as described in Shinkawa T. et al. (2003) J. Biol. Chem. 278: 3466- 73.

Another type of covalent modification is alterations in glycosylation. In another aspect, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g. 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zurich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

The anti-CD38 antibodies may include modifications that modulate its serum half-life and biodistribution, including modifications that modulate the antibody’s interaction with the neonatal Fc receptor (FcRn), a receptor with a key role in protecting IgG from catabolism, and maintaining high serum antibody concentration. Serum half-life modulating modifications may occur in the Fc region of IgGI or IgG4, including the triple substitution of M252Y/S254T/T256E (Numbering according to the EU numbering system (Edelman, G.M. et al. (1969) Proc. Natl. Acad. USA 63, 78-85)), (e.g., SEO ID NO: 13, SEO ID NO: 14, SEO ID NO: 15, SEO ID NO: 16), as described in U.S. Pat. No. 7,083,784. Other substitutions may occur at positions 250 and 428, see e.g., U.S. Pat. No 7,217,797, as well as at positions 307, 380 and 434, see, e.g., WO 00/42072. Examples of constant domain amino acid substitutions which modulate binding to Fc receptors and subsequent function mediated by these receptors, including FcRn binding and serum half-life, are described in U.S. Publ. Nos. 2009/0142340, 2009/0068175, and 2009/0092599. Naked antibodies may have the heavy chain C-terminal lysine omitted or removed to reduce heterogeneity. The substitution of S228P (EU numbering) in the human IgG4 can stabilize antibody Fab-arm exchange in vivo (Labrin et al. (2009) Nature Biotechnology 27:8; 767-773).

The glycans linked to antibody molecules are known to influence interactions of antibody with Fc receptors and glycan receptors and thereby influence antibody activity, including serum half-life. Hence, certain glycoforms that modulate desired antibody activities can confer therapeutic advantage. Methods for generating engineered glycoforms include but are not limited to those described in U.S. Pat. Nos. 6,602,684, 7,326,681, and 7,388,081 and PCT Publ. No. WO 08/006554. Alternatively, the antibody sequences may be modified to remove relevant glycoform-attachment sites.

The anti-CD38 antibodies preferably have a binding affinity for an epitope on CD38 that includes a dissociation constant (Kd) of less than about 1 × 10⁻⁴ M. In some aspects, the Kd is less than about 1 × 10⁻⁵ M. In still other aspects, the Kd is less than about 1 × 10⁻⁶ M. In other aspects, the Kd is less than about 1 × 10⁻⁷ M. In other aspects, the Kd is less than about 1 × 10⁻⁸ M. In other aspects, the Kd is less than about 1 × 10⁻⁹ M. In other aspects, the Kd is less than about 1 × 10⁻¹⁰ M. In still other aspects, the Kd is less than about 1 × 10⁻¹¹ M. In some aspects, the Kd is less than about 1 × 10⁻¹² M. In other aspects, the Kd is less than about 1 × 10⁻¹³ M. In other aspects, the Kd is less than about 1 × 10⁻¹⁴ M. In still other aspects, the Kd is less than about 1 × 10⁻¹⁵ M. Affinity values refer to those obtained by standard methodologies, including surface plasmon resonance such as Biacore ™ analyses or analysis using an Octet® Red 96 (Forte Bio) Dip-and-Read system.

The anti-CD38 antibodies are preferably capable of binding to CD38-positive cells. The antibody may bind to a CD38-positive cell with an EC50 value of less than about 100 nM. The antibody may bind to a CD38-positive cell with an EC50 value of less than about 75 nM. The antibody may bind to a CD38-positive cell with an EC50 value of less than about 50 nM. The antibody may bind to a CD38-positive cell with an EC50 value of less than about 30 nM. The antibody may bind to a CD38-positive cell with an EC50 value of less than about 25 nM. The antibody may bind to a CD38-positive cell with an EC50 value of less than about 20 nM. The antibody may bind to a CD38-positive cell with an EC50 value of less than about 18 nM. The antibody may bind to a CD38-positive cell with an EC50 value of less than about 15 nM. The antibody may bind to a CD38-positive cell with an EC50 value of less than about 13 nM. The antibody may bind to a CD38-positive cell with an EC50 value of less than about 10 nM. Variants of such anti-CD38 antibodies can be engineered and expressed such that the antibodies have reduced immunogenicity, enhanced stability, and enhanced half life in circulation without a significant loss of specificity or affinity of the antibody to the CD38 antigen. These variant antibodies can be fused to an attenuated interferon.

Strategies and methods for the resurfacing of antibodies, and other methods for reducing immunogenicity of antibodies within a different host, are disclosed in U.S. Pat. No. 5,639,641, which is hereby incorporated in its entirety by reference. Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5): 489-498; Studnicka G. M. et al., 1994, Protein Engineering, 7(6): 805-814; Roguska M. A. et al., 1994, PNAS, 91: 969-973), chain shuffling (U.S. Pat. No. 5,565,332), and identification of flexible residues (PCT/US2008/074381). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

### DHODH Inhibitor Compounds

In the disclosed pharmaceutical compositions and methods of treating a clinical condition, a DHODH inhibitor is used with an anti-CD38 antibody. A suitable DHODH inhibitor can be one of the DHODH inhibitors as disclosed herein, or any other DHODH inhibitor as know to the skilled artisan.

Exemplary disclosed DHODH inhibitors can have a formula represented by a structure:

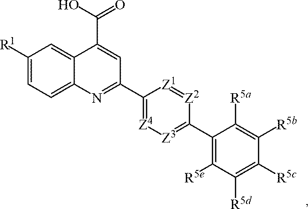

wherein each of Z¹, Z², Z³, and Z⁴ is independently selected from CH and N; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, -R³⁰-A ¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O—and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, and -C1-C10 alkoxy; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; or a pharmaceutically acceptable salt thereof.

Further exemplary disclosed DHODH inhibitors DHODH inhibitors can have a formula represented by a structure:

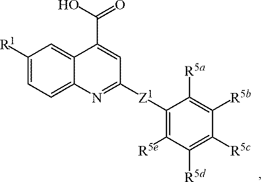

wherein Z¹ is a five-membered heterocyclic diyl; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰ —R³⁰—A¹—R⁴⁰, —A¹—R⁴⁰, —A¹—R³⁰—A²—R⁴⁰, or -A¹-R³⁰-A²-R³¹-A³-R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and -C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and —C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Further exemplary disclosed DHODH inhibitors can have a formula represented by a structure:

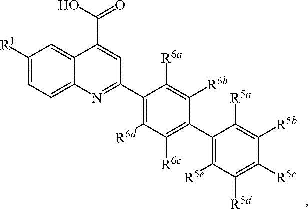

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, -R³⁰-A¹₋Ra⁴⁰, -A¹-R⁴⁰, —A¹—R³⁰—A²—R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from -O-and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

These and other exemplary disclosed DHODH inhibitors are described in further detailed herein below by reference to DHODH Inhibitor Compounds - Groups I, II, III, IV, and V.

### DHODH Inhibitor Compounds - Group I

A disclosed DHODH inhibitor can be any DHODH inhibitor as disclosed in Intl. Pat. Appl. No. PCT/US19/38622, which is incorporated herein by reference, and further described herein. For convenience, compounds of this structural type will be referred to as DHODH Inhibitor Compounds - Group I.

Disclosed are DHODH Inhibitor Compounds - Group I compounds having a formula represented by a structure:

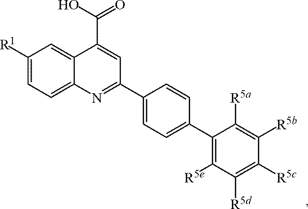

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R⁴⁰—A³—R⁴¹; wherein A¹ is selected from —O— and —NR⁵⁰—; wherein R⁵⁰ is selected from -C1-C10 aminoalkyl, -C1-C10 alkylamino, and —C1-C10 hydroxyalkyl; wherein A² is selected from —O— and —NR⁶⁰—; wherein R⁶⁰ is selected from -C1-C10 aminoalkyl, -C1-C10 alkylamino, and —C1-C10 hydroxyalkyl; wherein A³ is selected from —O— and —NR⁷⁰—; wherein R⁷⁰ is selected from -C1-C10 aminoalkyl, -C1-C10 alkylamino, and —C1-C10 hydroxyalkyl; wherein R²⁰ is selected from halogen, —C1-C10 alkylamino and -C1-C10 alkoxy; wherein R³⁰ is selected from -C1-C10 alkanediyl, -C1-C10 aminoalkanediyl, and -C1-C10 hydroxyalkanediyl; and wherein each of R⁴⁰ and R⁴¹ is independently selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 1, 2, or 3 groups independently selected from halogen, —SF₅, —CN, —N₃, —OH, —NH₂, from -C1-C3 alkyl, -C1-C3 alkoxy, -C1-C3 haloalkyl, -C1-C3 aminoalkyl, -C1-C3 alkylamino, -C1-C3 haloalkylamino, -C1-C3 hydroxyalkyl, -C1-C3 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Also disclosed are DHODH Inhibitor Compounds - Group I compounds having a formula represented by a structure:

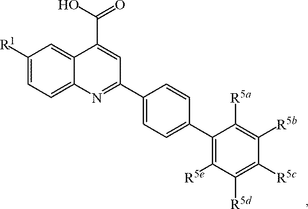

wherein R¹ is selected from hydrogen, halogen, —SF₅, —CN, —N₃, —OH, —NH₂, —CF₃, and —CF₂CF₃; wherein R^{5a} is selected from a group having formula represented by a structure: —R²⁰, —R³⁰—A¹—R⁴⁰, —A¹—R⁴⁰, —A¹—R³⁰—A²—R⁴⁰, or-A¹-R³⁰-A²-R⁴⁰-A³-R⁴¹; wherein A¹ is selected from —O— and—NR⁵⁰—; wherein R⁵⁰ is selected from -C1-C10 aminoalkyl, -C1-C10 alkylamino, and —C1-C10 hydroxyalkyl; wherein A² is selected from —O— and —NR⁶⁰—; wherein R⁶⁰ is selected from -C1-C10 aminoalkyl, -C1-C10 alkylamino, and -C1-C10 hydroxyalkyl; wherein A³ is selected from —O— and —NR⁷⁰—; wherein R⁷⁰ is selected from -C1-C10 aminoalkyl, -C1-C10 alkylamino, and -C1-C10 hydroxyalkyl; wherein R²⁰ is selected from halogen, —C1-C10 alkylamino and -C1-C10 alkoxy; wherein R³⁰ is selected from -C1-C10 alkanediyl, -C1-C10 aminoalkanediyl, and -C1-C10 hydroxyalkanediyl; and wherein each of R⁴⁰ and R⁴¹ is independently selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 1, 2, or 3 groups independently selected from halogen, —SF₅, —CN, —N₃, —OH, —NH₂, from -C1-C3 alkyl, -C1-C3 alkoxy, -C1-C3 haloalkyl, -C1-C3 aminoalkyl, -C1-C3 alkylamino, -C1-C3 haloalkylamino, -C1-C3 hydroxyalkyl, -C1-C3 halohydroxyalkyl, cycloalkyl, and heterocycloalkyl; and wherein each of R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Also disclosed are DHODH Inhibitor Compounds - Group I compounds having a formula represented by a structure:

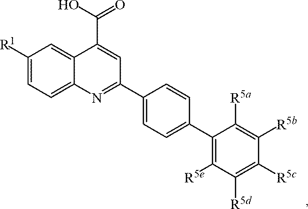

wherein R¹ is selected from hydrogen, halogen, —SF₅, —CN, —N₃, —OH, —NH₂, —CF₃, and —CF₂CF₃; wherein R^{5b} is selected from a group having formula represented by a structure: —R²⁰, —R³⁰—A¹—R⁴⁰, —A¹—R⁴⁰, —A¹—R³⁰—A²—R⁴⁰, or -A¹-R³⁰-A²-R⁴⁰-A³-R⁴¹; wherein A¹ is selected from —O— and—NR⁵⁰—; wherein R⁵⁰ is selected from -C1-C10 aminoalkyl, -C1-C10 alkylamino, and —C1-C10 hydroxyalkyl; wherein A² is selected from —O— and —NR⁶⁰—; wherein R⁶⁰ is selected from -C1-C10 aminoalkyl, -C1-C10 alkylamino, and —C1-C10 hydroxyalkyl; wherein A³ is selected from —O— and —NR⁷⁰—; wherein R⁷⁰ is selected from -C1-C10 aminoalkyl, -C1-C10 alkylamino, and -C1-C10 hydroxyalkyl; wherein R²⁰ is selected from halogen, —C1-C10 alkylamino and -C1-C10 alkoxy; wherein R³⁰ is selected from -C1-C10 alkanediyl, -C1-C10 aminoalkanediyl, and -C1-C10 hydroxyalkanediyl; and wherein each of R⁴⁰ and R⁴¹ is independently selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 1, 2, or 3 groups independently selected from halogen, —SF₅, —CN, —N₃, —OH, —NH₂, from -C1-C3 alkyl, -C1-C3 alkoxy, -C1-C3 haloalkyl, -C1-C3 aminoalkyl, -C1-C3 alkylamino, -C1-C3 haloalkylamino, -C1-C3 hydroxyalkyl, -C1-C3 halohydroxyalkyl, cycloalkyl, and heterocycloalkyl; and wherein each of R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Also disclosed are DHODH Inhibitor Compounds - Group I compounds having a formula represented by a structure:

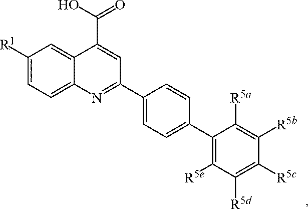

wherein R¹ is selected from hydrogen, halogen, —SF₅, —CN, —N₃, —OH, —NH₂, —CF₃, and —CF₂CF₃; wherein R^{5c} is selected from a group having formula represented by a structure: —R^{20,} —R³⁰—A¹—R⁴⁰, —A¹—R⁴⁰, —A¹—R³⁰—A²—R⁴⁰, or-A¹-R³⁰-A²-R⁴⁰-A³-R⁴¹; wherein A¹ is selected from —O— and—NR⁵⁰—; wherein R⁵⁰ is selected from -C1-C10 aminoalkyl, -C1-C10 alkylamino, and —C1-C10 hydroxyalkyl; wherein A² is selected from —O— and —NR⁶⁰—; wherein R⁶⁰ is selected from -C1-C10 aminoalkyl, -C1-C10 alkylamino, and —C1-C10 hydroxyalkyl; wherein A³ is selected from —O— and —NR⁷⁰—; wherein R⁷⁰ is selected from -C1-C10 aminoalkyl, -C1-C10 alkylamino, and —C1-C10 hydroxyalkyl; wherein R²⁰ is selected from halogen, —C1-C10 alkylamino and -C1-C10 alkoxy; wherein R³⁰ is selected from -C1-C10 alkanediyl, -C1-C10 aminoalkanediyl, and -C1-C10 hydroxyalkanediyl; and wherein each of R⁴⁰ and R⁴¹ is independently selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 1, 2, or 3 groups independently selected from halogen, —SF₅, —CN, —N₃, —OH, —NH₂, from -C1-C3 alkyl, -C1-C3 alkoxy, -C1-C3 haloalkyl, -C1-C3 aminoalkyl, -C1-C3 alkylamino, -C1-C3 haloalkylamino, -C1-C3 hydroxyalkyl, -C1-C3 halohydroxyalkyl, cycloalkyl, and heterocycloalkyl; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Also disclosed are DHODH Inhibitor Compounds - Group I compounds having a formula represented by a structure:

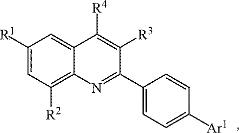

wherein Ar¹ is a phenyl independently substituted with 1, 2, or 3 groups selected from halogen, —OH, -O(C1-C7 alkyl), -(C1-C7 alkanediyl)-OH, -O(C1-C7 alkanediyl)-OH, -CH₂O(C1-C7 alkyl), -(CH₂)₂O(C1-C7 alkyl), C1-C7 haloalkyl, -O(C1-C7 haloalkyl), and C1-C7 hydroxyalkyl; wherein each of R¹ and R² are each independently selected from hydrogen, halogen, —SF₅, —CN, —N₃, —OH, —NH₂, —CF₃, —CF₂CF₃, and Ar²; wherein Ar² is a phenyl independently substituted with 1, 2, or 3 groups selected from halogen, —SF₅, —CN, —N₃, —OH, —NH₂,—CF₃, and —CF₂CF₃; and wherein at least one of R¹ and R² is not hydrogen; wherein R³ is selected from hydrogen and C1-C7 alkyl; wherein R⁴ is —S(O)ⱼR¹⁰, —(C═O)OR¹¹, and —(C═O)NR^{12a}R^{12b}; and wherein j is an integer selected from 0, 1, and 2; wherein R¹⁰ is selected from hydrogen, C1-C3 alkyl, C1-C3 hydroxyalkyl, and C1-C3 haloalkyl; wherein R¹¹ is selected from hydrogen, C1-C3 alkyl, C1-C3 hydroxyalkyl, and C1-C3 haloalkyl; and wherein each of R^{12a} and R^{12b} is independently selected from hydrogen, C1-C3 alkyl, C1-C3 hydroxyalkyl, and C1-C3 haloalkyl; or a pharmaceutically acceptable salt thereof.

Also disclosed are DHODH Inhibitor Compounds - Group I compounds having a formula represented by a structure:

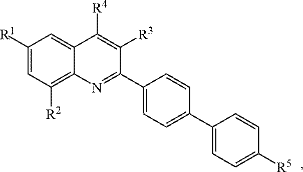

wherein each of R¹ and R² are each independently selected from hydrogen, halogen, —SF₅, —CN, —N₃, —OH, —NH₂, —CF₃, —CF₂CF₃, and Ar²; wherein Ar² is a phenyl independently substituted with 1, 2, or 3 groups selected from halogen, —SF₅, —CN, —N₃, —OH, —NH₂,—CF₃, and —CF₂CF₃; and wherein at least one of R¹ and R² is not hydrogen; wherein R³ is selected from hydrogen and C1-C7 alkyl; wherein R⁴ is —S(O)ⱼR¹⁰, —(C═O)OR¹¹, and —(C═O)NR^{12a}R^{12b}; and wherein j is an integer selected from 0, 1, and 2; wherein R¹⁰ is selected from hydrogen, C1-C3 alkyl, C1-C3 hydroxyalkyl, and C1-C3 haloalkyl; wherein R¹¹ is selected from hydrogen, C1-C3 alkyl, C1-C3 hydroxyalkyl, and C1-C3 haloalkyl; and wherein each of R^{12a} and R^{12b} is independently selected from hydrogen, C1-C3 alkyl, C1-C3 hydroxyalkyl, and C1-C3 haloalkyl; wherein R⁵ is selected from —OH, -O(C1-C7 alkyl), -(C1-C7 alkanediyl)-OH, -CH₂O(C1-C7 alkyl), -(CH₂)₂O(C1-C7 alkyl), and C1-C7 hydroxyalkyl; or a pharmaceutically acceptable salt thereof.

Also disclosed are DHODH Inhibitor Compounds - Group I compounds having a formula represented by a structure:

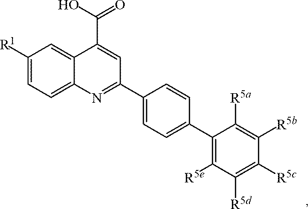

wherein R¹ is selected from halogen, —SF₅, —CN, —N₃, —OH, —NH₂, —CF₃, and —CF₂CF₃; wherein each of R^{5b} and R^{5c} is independently selected from —R²⁰, hydrogen, halogen, —SF₅, —CN, —N₃, —OH, —NH₂, —CF₃, and —CF₂CF₃; wherein R²⁰ is selected from -C1-C10 alkylamino and -C1-C10 alkoxy; provided that one of R^{5b} and R^{5c} is —R²⁰; and wherein each R^{5a}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Also disclosed are DHODH Inhibitor Compounds - Group I compounds having a formula represented by a structure:

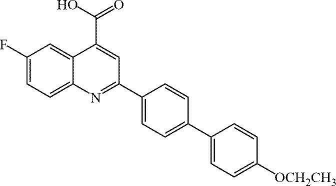 [SMILES: CCOc1ccc(-c2ccc(-c3cc(C(=O)O)c4cc(F)ccc4n3)cc2)cc1]

2-(4′-ethoxy-[1,1′-biphenyl]-4-yl)-6-fluoroquinoline-4-carboxylic acid (Cpd3).

Also disclosed are DHODH Inhibitor Compounds - Group I compounds having a formula represented by a structure:

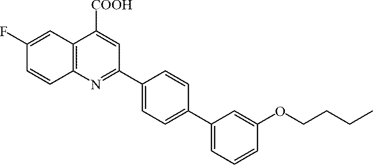 [SMILES: CCCCOc1cccc(-c2ccc(-c3cc(C(=O)O)c4cc(F)ccc4n3)cc2)c1]

2-(3′-butoxy-[1,1′-biphenyl]-4-yl)-6-fluoroquinoline-4-carboxylic acid (Cpd4).

It is understood that the disclosed DHODH inhibitor compounds comprise salt forms, e.g., a DHODH Inhibitor Compounds - Group I compound, can be in the sodium salt form such as:

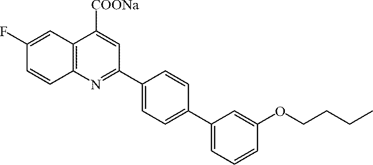

Sodium 2-(3′-butoxy-[1,1′-biphenyl]-4-yl)-6-fluoroquinoline-4-carboxylate (Cpd4Na).

The following listing of exemplary aspects supports and is supported by the disclosure provided herein for DHODH Inhibitor Compounds - Group I.

Aspect 1. A compound having a formula represented by a structure:

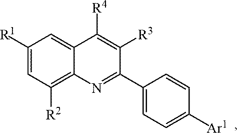

wherein Ar¹ is a phenyl independently substituted with 1, 2, or 3 groups selected from halogen, —OH, -O(C1-C7 alkyl), -(C1-C7 alkanediyl)-OH, -O(C1-C7 alkanediyl)-OH, -CH₂O(C1-C7 alkyl), -(CH₂)₂O(C1-C7 alkyl), -C1-C7 haloalkyl, -O(C1-C7 haloalkyl), and -C1-C7 hydroxyalkyl; wherein each of R¹ and R² are each independently selected from hydrogen, halogen, —SF₅, —CN, —N₃, —OH, —NH₂, —CF₃, —CF₂CF₃, and Ar²; wherein Ar² is a phenyl independently substituted with 1, 2, or 3 groups selected from halogen, —SF₅, —CN, —N₃, —OH, —NH₂,—CF₃, and —CF₂CF₃; and wherein at least one of R¹ and R² is not hydrogen; wherein R³ is selected from hydrogen and C1-C7 alkyl; wherein R⁴ is -S(O)ⱼR¹⁰, -(C=O)OR¹¹, and -(C=O)NR^{12a}R^{12b}; and wherein j is an integer selected from 0, 1, and 2; wherein R¹⁰ is selected from hydrogen, C1-C3 alkyl, C1-C3 hydroxyalkyl, and C1-C3 haloalkyl; wherein R¹¹ is selected from hydrogen, C1-C3 alkyl, C1-C3 hydroxyalkyl, and C1-C3 haloalkyl; and wherein each of R^{12a} and R^{12b} is independently selected from hydrogen, C1-C3 alkyl, C1-C3 hydroxyalkyl, and C1-C3 haloalkyl; or a pharmaceutically acceptable salt thereof.

Aspect 2. The compound of Aspect 1, having a formula represented by a structure:

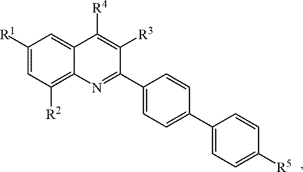

wherein R⁵ is selected from halogen, —OH, -O(C1-C7 alkyl), -(C1-C7 alkanediyl)-OH, -O(C1-C7 alkanediyl)-OH, -CH₂O(C1-C7 alkyl), -(CH₂)₂O(C1-C7 alkyl), C1-C7 haloalkyl, -O(C1-C7 haloalkyl), and C1-C7 hydroxyalkyl; or a pharmaceutically acceptable salt thereof.

Aspect 3. The compound of Aspect 2, wherein R⁵ is halogen, C1-C7 haloalkyl, or -O(C1-C7 haloalkyl).

Aspect 4. The compound of Aspect 3, wherein R⁵ is halogen.

Aspect 5. The compound of Aspect 4, wherein R⁵ is F.

Aspect 6. The compound of Aspect 3, wherein R⁵ is —OCF₃, —OCH₂CF₃, or —OCF₂CF₃.

Aspect 7. The compound of Aspect 2, wherein R⁵ is —OH, -O(C1-C7 alkyl), -(C1-C7 alkanediyl)-OH, -O(C1-C7 alkanediyl)-OH, -CH₂O(C1-C7 alkyl), -(CH₂)₂O(C1-C7 alkyl), or C1-C7 hydroxyalkyl.

Aspect 8. The compound of Aspect 7, wherein R⁵ is -O(C1-C7 alkyl), -(C1-C7 alkanediyl)-OH, -O(C1-C7 alkanediyl)-OH, -CH₂O(C1-C7 alkyl), or-(CH₂)₂O(C1-C7 alkyl).

Aspect 9. The compound of Aspect 8, wherein R⁵ is —OCH₃, —OCH₂CH₃, —O(CH₂)₂CH₃, —OCH(CH₃)₂, —O(CH₂)₃CH₃, —OCH₂CH(CH₃)₂, —OCH(CH₂CH₃)(CH₃), —CH₂OH, —(CH₂)₂OH, —(CH₂)₃OH, —(CH₂)₄OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or—(CH₂)₂OCH(CH₂CH₃)₂(CH₃).

Aspect 10. The compound of Aspect 8, wherein R⁵ is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or —(CH₂)₂OCH(CH₂CH₃)₂(CH₃).

Aspect 11. The compound of Aspect 8, wherein R⁵ is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —(CH₂)₂OCH₃, or—(CH₂)₂OCH₂CH₃. Aspect 12. The compound of Aspect 8, wherein R⁵ is —OCH₃, —OCH₂CH₃, —CH₂OH, —(CH₂)₂OH, —CH₂OCH₃, or—CH₂OCH₂CH₃.

Aspect 13. The compound of Aspect 8, wherein R⁵ is —OCH₃ or —OCH₂CH₃.

Aspect 14. The compound of any one of 1-Aspect 13, wherein R¹ is selected from halogen, —SF₅, —CF₃, and —CF₂CF₃.

Aspect 15. The compound of Aspect 14, wherein R¹ is halogen.

Aspect 16. The compound of Aspect 15, wherein R¹ is F or Cl.

Aspect 17. The compound of Aspect 15, wherein R¹ is F.

Aspect 18. The compound of Aspect 14, wherein R¹ is selected from —SF₅, —CF₃, and —CF₂CF₃.

Aspect 19. The compound of Aspect 14, wherein R¹ is —SF₅.

Aspect 20. The compound of any one of 1-Aspect 19, wherein R² is selected from halogen, —SF₅, —CF₃, and —CF₂CF₃.

Aspect 21. The compound of Aspect 20, wherein R² is halogen.

Aspect 22. The compound of Aspect 21, wherein R² is F or Cl.

Aspect 23. The compound of Aspect 21, wherein R² is F.

Aspect 24. The compound of Aspect 20, wherein R² is selected from —SF₅, —CF₃, and —CF₂CF₃.

Aspect 25. The compound of Aspect 20, wherein R² is —SF₅.

Aspect 26. The compound of any one of 1-Aspect 25, wherein R³ is selected from hydrogen and C1-C3 alkyl.

Aspect 27. The compound of Aspect 26, wherein R³ is hydrogen or methyl.

Aspect 28. The compound of Aspect 26, wherein R³ is hydrogen.

Aspect 29. The compound of Aspect 26, wherein R³ is methyl.

Aspect 30. The compound of any one of 1-Aspect 29, wherein R⁴ is -S(O)ⱼR¹⁰.

Aspect 31. The compound of Aspect 30, wherein j is 1 or 2.

Aspect 32. The compound of Aspect 30 or Aspect 31, wherein R¹⁰ is hydrogen or C1-C3 alkyl.

Aspect 33. The compound of Aspect 30 or Aspect 31, wherein R¹⁰ is hydrogen.

Aspect 34. The compound of Aspect 30 or Aspect 31, wherein R¹⁰ is C1-C3 alkyl.

Aspect 35. The compound of Aspect 34, wherein R¹⁰ is methyl or ethyl.

Aspect 36. The compound of Aspect 34, wherein R¹⁰ is methyl.

Aspect 37. The compound of Aspect 30, wherein R⁴ is —SO₂H, or —SO₂CH₃.

Aspect 38. The compound of Aspect 30 or Aspect 31, wherein R¹⁰ is C1-C3 alkyl, C1-C3 hydroxyalkyl, and C1-C3 haloalkyl.

Aspect 39. The compound of any one of 1-Aspect 25, wherein R⁴ is -(C=O)OR¹¹.

Aspect 40. The compound of Aspect 39, wherein R¹¹ is selected from hydrogen, methyl, and ethyl.

Aspect 41. The compound of Aspect 39, wherein R¹¹ is hydrogen.

Aspect 42. The compound of Aspect 41, wherein the compound is a pharmaceutically acceptable salt of R⁴.

Aspect 43. The compound of Aspect 42, pharmaceutically acceptable salt of R⁴ is a lithium, sodium, or potassium salt thereof.

Aspect 44. The compound of Aspect 42, pharmaceutically acceptable salt of R⁴ is a sodium salt thereof.

Aspect 45. The compound of Aspect 39, wherein R¹¹ is selected from C1-C3 alkyl, C1-C3 hydroxyalkyl, and C1-C3 haloalkyl.

Aspect 46. The compound of Aspect 45, wherein R¹¹ is selected from methyl, ethyl, —CHF₂, —CH₂F, —CF₃, —CHCl₂, —CH₂Cl, —CCl₃, —CH₂CH₂F, —CH₂CHF₂, —CH₂CF₃, —CH₂CH₂Cl, —CH₂CHCl₂, —CH₂CCl₃, —CH₂OH, and —(CH₂)₂OH. Aspect 47. The compound of Aspect 45, wherein R¹¹ is selected from methyl, ethyl, —CHF₂, —CH₂F, —CF₃, —CHCl₂, —CH₂Cl, —CCl₃, —CH₂CH₂F, —CH₂CHF₂, —CH₂CF₃, —CH₂CH₂Cl, —CH₂CHCl₂, and —CH₂CCl₃. Aspect 48. The compound of Aspect 45, wherein R¹¹ is selected from methyl, ethyl, —CHF₂, —CH₂F, —CF₃, —CH₂CH₂F, —CH₂CHF₂, and —CH₂CF₃,

Aspect 49. The compound of Aspect 45, wherein R¹¹ is selected from methyl and ethyl.

Aspect 50. The compound of Aspect 45, wherein R¹¹ is selected from methyl, —CHF₂, —CH₂F, —CF₃, —CHCl₂, —CH₂Cl, —CCl₃, and —CH₂OH.

Aspect 51. The compound of any one of Aspect 1-Aspect 25, wherein R⁴ is —(C═O)NR^{12a}R¹²b.

Aspect 52. The compound of Aspect 51, wherein each of R^{12a} and R^{12b} is independently selected from hydrogen and C1-C3 alkyl.

Aspect 53. The compound of Aspect 51, wherein each of R^{12a} and R^{12b} is hydrogen.

Aspect 54. The compound of Aspect 51, wherein R^{12a} is hydrogen and R^{12b} is hydrogen or C1-C3 alkyl.

Aspect 55. The compound of Aspect 51, wherein R^{12a} is hydrogen and R^{12b} is C1-C3 alkyl.

Aspect 56. The compound of Aspect 1, having a structure represented by a formula:

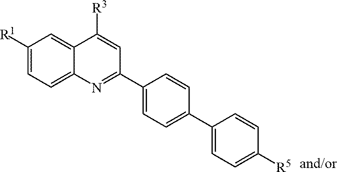

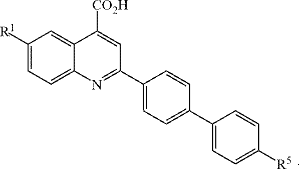

Aspect 57. The compound of Aspect 56, wherein the compound is a pharmaceutically acceptable salt thereof.

Aspect 58. The compound of Aspect 57, wherein the pharmaceutically acceptable salt is a sodium, potassium, or lithium salt.

Aspect 59. The compound of Aspect 1, having a structure represented by a formula:

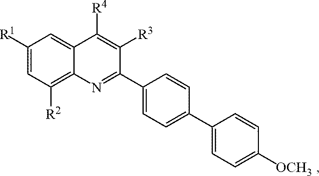

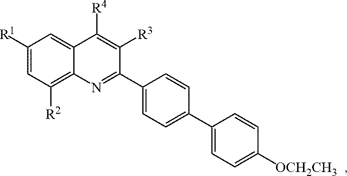

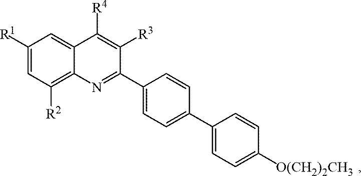

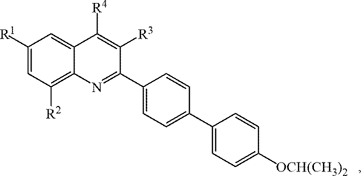

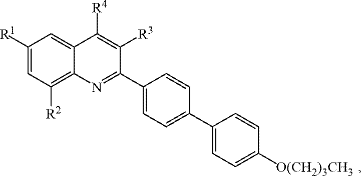

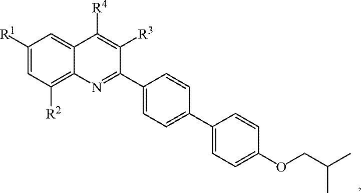

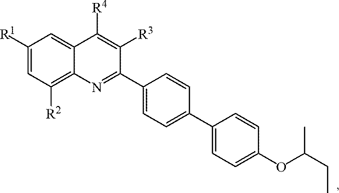

or combinations thereof.

Aspect 60. The compound of Aspect 1, having a structure represented by a formula:

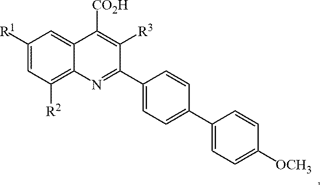

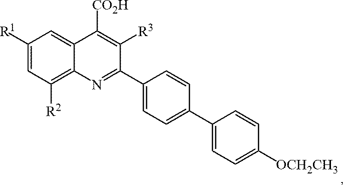

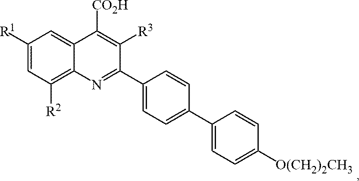

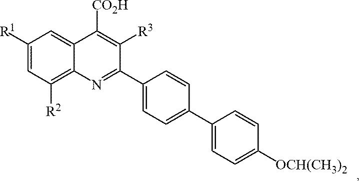

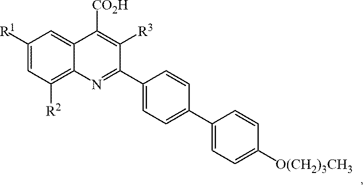

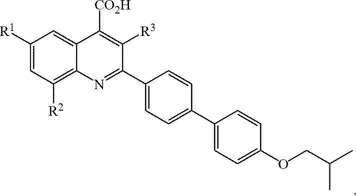

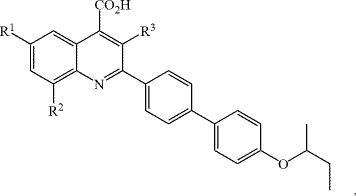

or combinations thereof.

Aspect 61. The compound of Aspect 59 or Aspect 60, wherein the compound is a pharmaceutically acceptable salt thereof.

Aspect 62. The compound of Aspect 61, wherein the pharmaceutically acceptable salt is a sodium, potassium, or lithium salt.

Aspect 63. The compound of Aspect 1, having a structure represented by a formula:

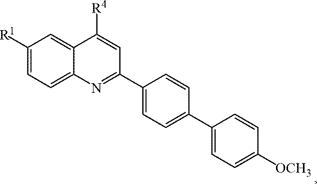

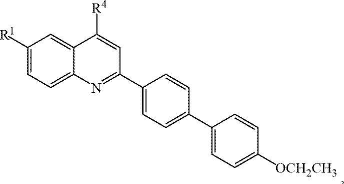

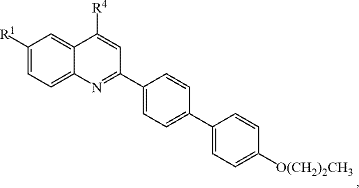

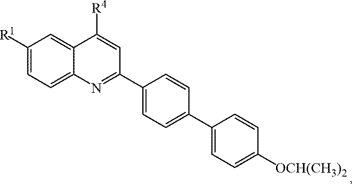

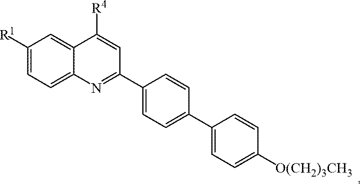

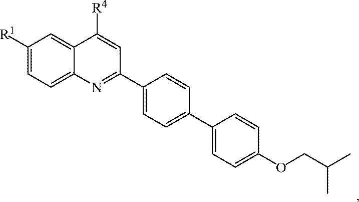

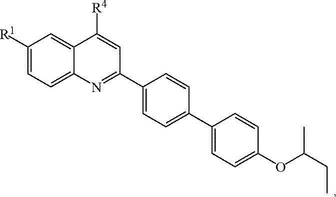

or combinations thereof.

Aspect 64. The compound of Aspect 1, having a structure represented by a formula:

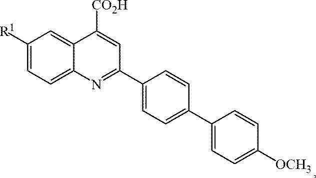

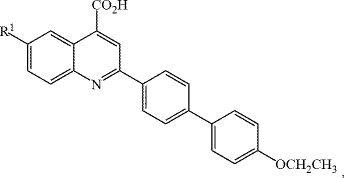

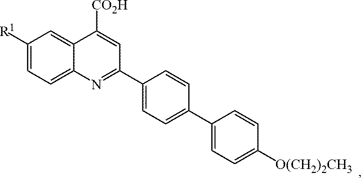

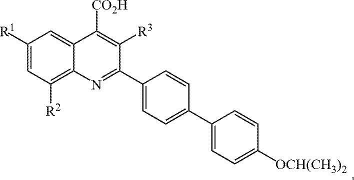

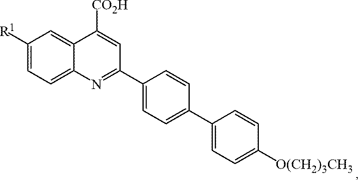

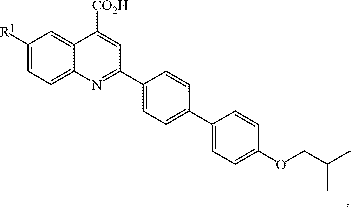

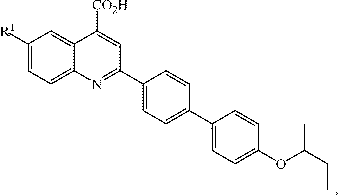

or combinations thereof.

Aspect 65. The compound of Aspect 63 or Aspect 64, wherein the compound is a pharmaceutically acceptable salt thereof.

Aspect 66. The compound of Aspect 65, wherein the pharmaceutically acceptable salt is a sodium, potassium, or lithium salt.

Aspect 67. The compound of Aspect 1, having a structure represented by a formula:

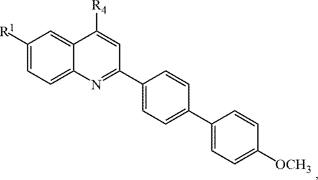

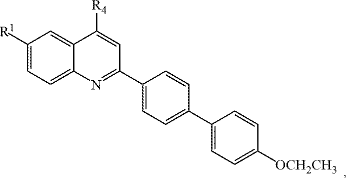

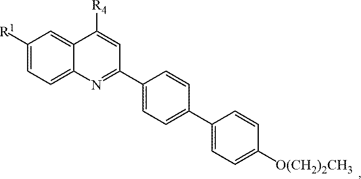

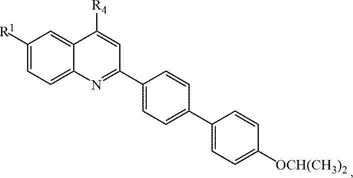

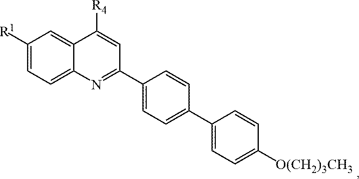

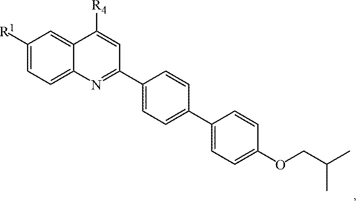

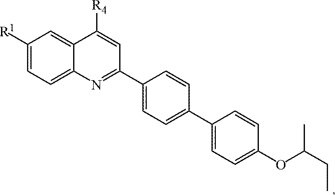

or combinations thereof.

Aspect 68. The compound of Aspect 1, having a structure represented by a formula:

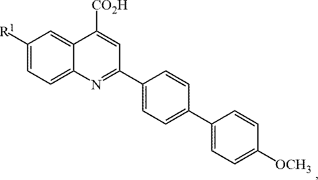

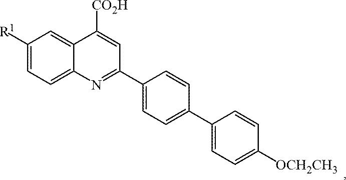

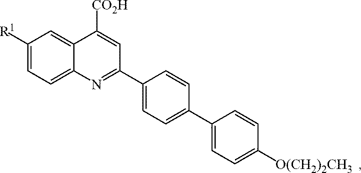

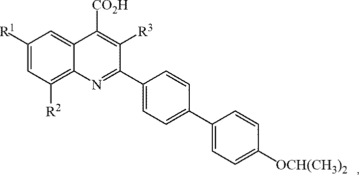

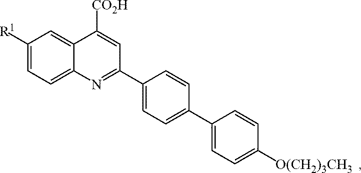

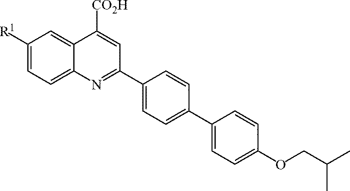

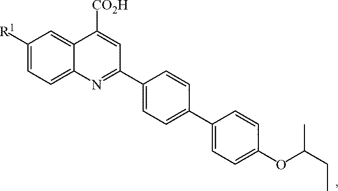

or combinations thereof.

Aspect 69. The compound of Aspect 67 or Aspect 68, wherein the compound is a pharmaceutically acceptable salt thereof.

Aspect 70. The compound of Aspect 69, wherein the pharmaceutically acceptable salt is a sodium, potassium, or lithium salt.

Aspect 71. The compound of Aspect 1, having a structure represented by a formula:

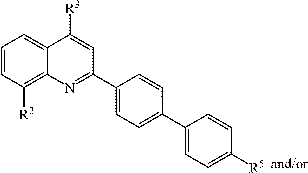

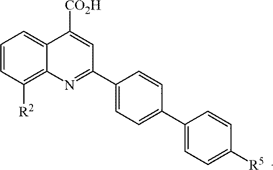

Aspect 72. The compound of Aspect 71, wherein the compound is a pharmaceutically acceptable salt thereof.

Aspect 73. The compound of Aspect 72, wherein the pharmaceutically acceptable salt is a sodium, potassium, or lithium salt.

Aspect 74. The compound of Aspect 1, having a structure represented by a formula:

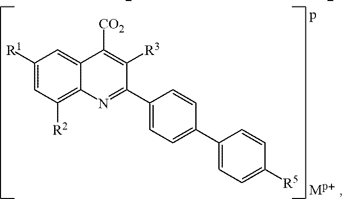

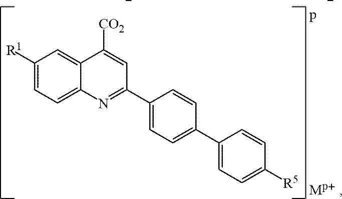

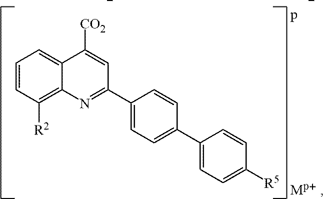

or combinations thereof.

wherein M^{p+} represents a counter ion or a moiety which forms a pharmaceutically acceptable salt; and wherein p is an integer having a value of 1, 2, or 3.

Aspect 75. The compound of Aspect 74, having a structure represented by a formula:

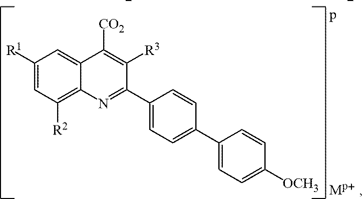

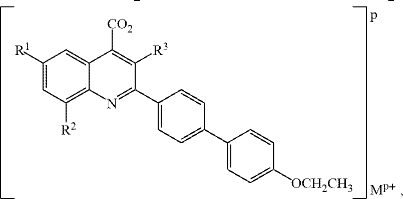

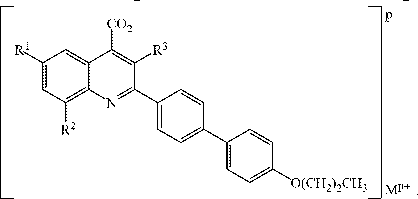

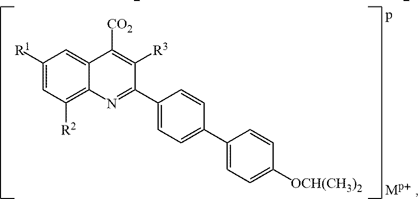

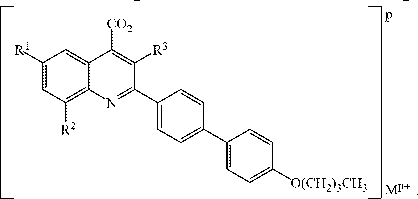

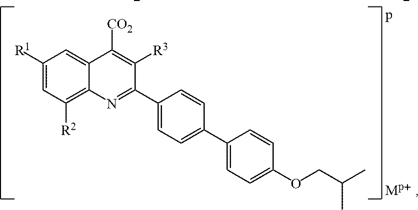

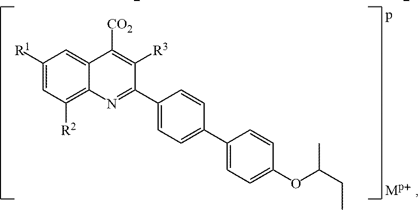

or combinations thereof.

Aspect 76. The compound of Aspect 74, having a structure represented by a formula:

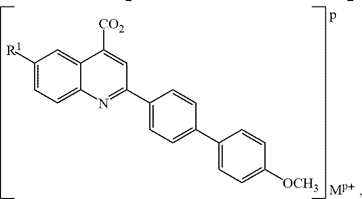

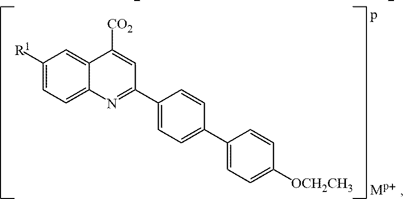

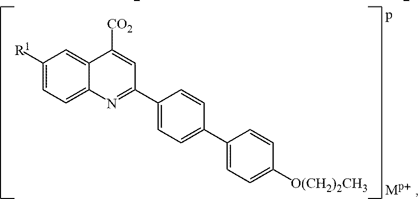

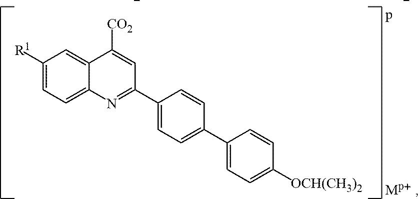

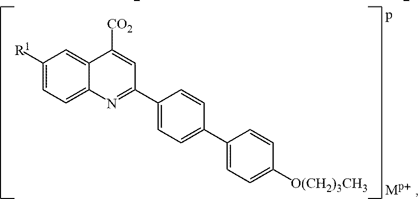

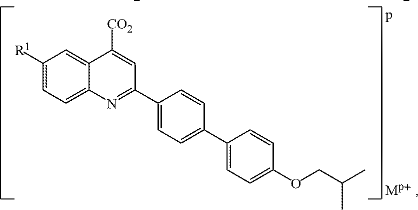

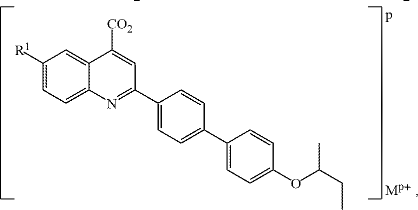

or combinations thereof.

Aspect 77. The compound of Aspect 74, having a structure represented by a formula:

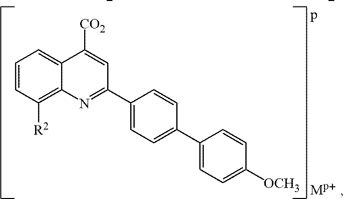

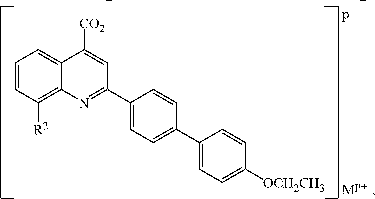

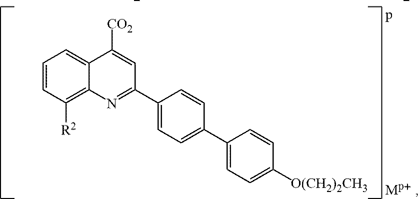

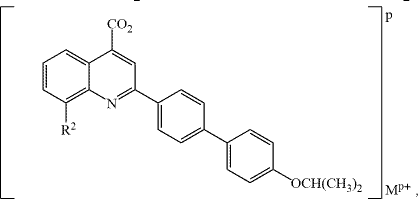

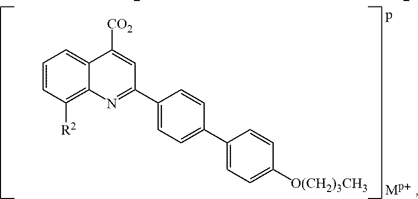

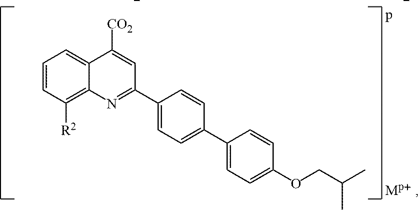

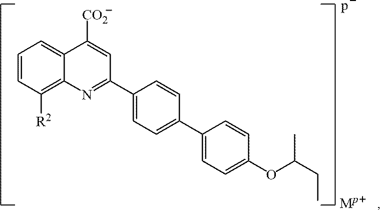

or combinations thereof.

Aspect 78. The compound of any one of Aspect 74-Aspect 77, wherein M^{p+} is selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.

Aspect 79. The compound of Aspect 78, M⁺ is Na⁺.

Aspect 80. The compound of Aspect 1, present as:

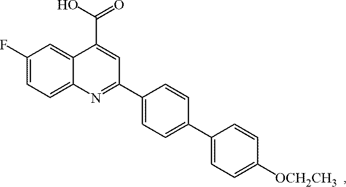

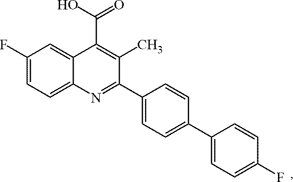

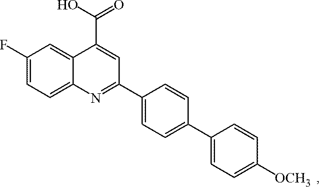

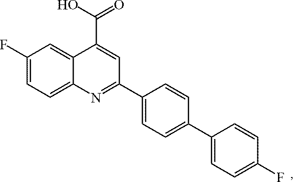

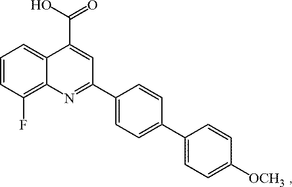

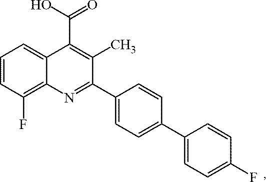

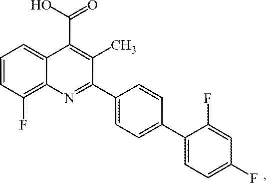

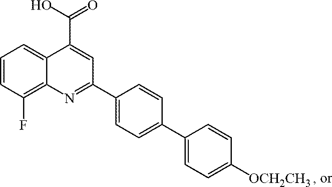

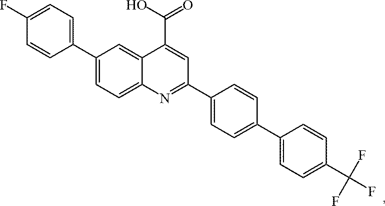

or a subgroup thereof.

Aspect 81. The compound of 21, present as:

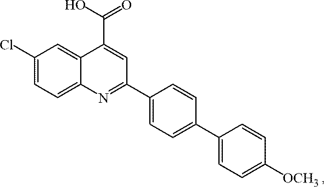

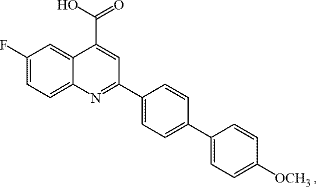

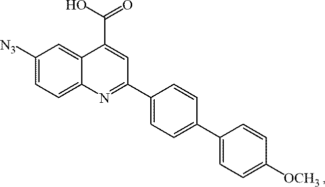

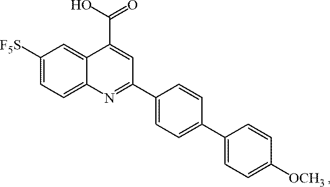

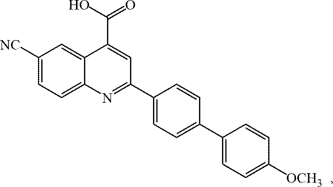

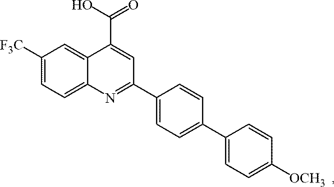

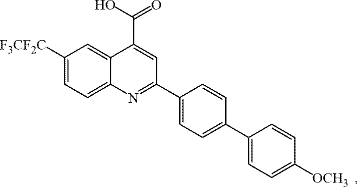

or a subgroup thereof.

Aspect 82. The compound of 21, present as:

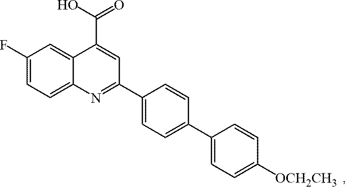

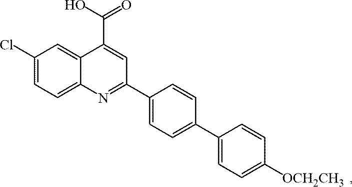

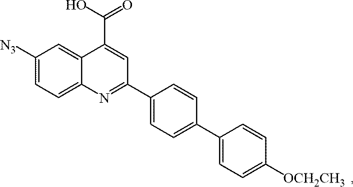

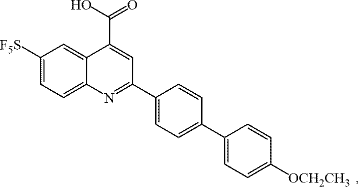

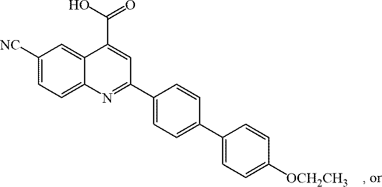

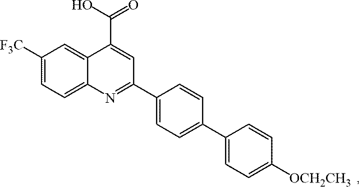

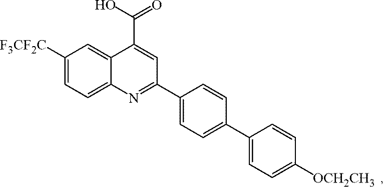

or a subgroup thereof.

Aspect 83. The compound of 21, present as:

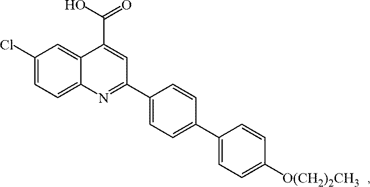

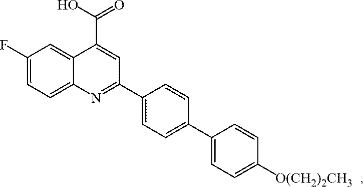

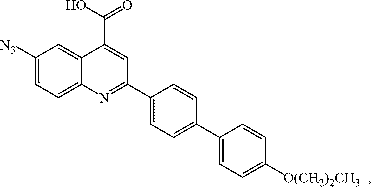

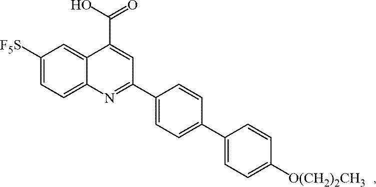

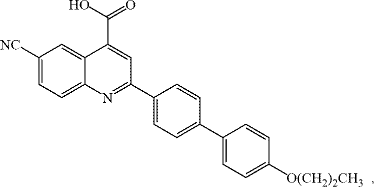

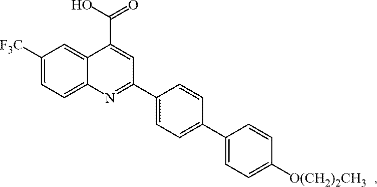

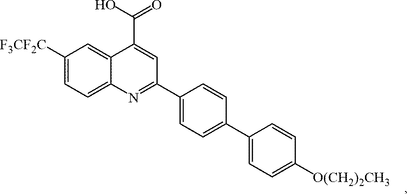

or a subgroup thereof.

Aspect 84. The compound of 21, present as:

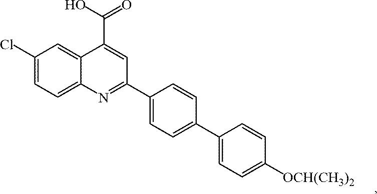

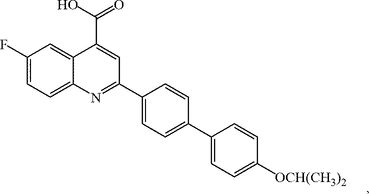

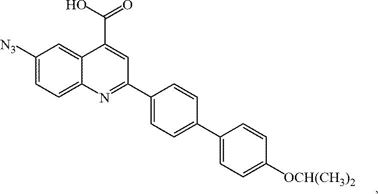

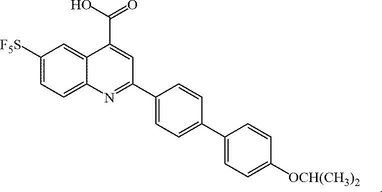

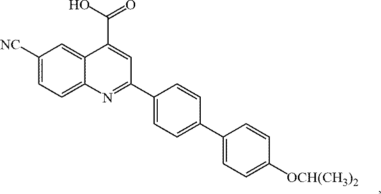

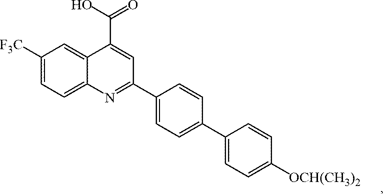

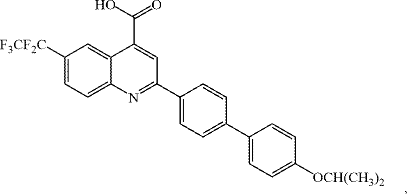

or a subgroup thereof.

Aspect 85. The compound of any one of Aspect 81-Aspect 84, wherein the compound is a pharmaceutically acceptable salt thereof comprising the conjugate base form of the compound, and a counter ion selected from selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.

Aspect 86. The compound of Aspect 82, wherein the counter ion is Na⁺.

Aspect 87. A compound having a formula represented by a structure:

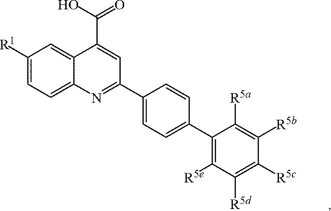

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R⁴⁰—A³—R⁴¹; wherein A¹ is selected from —O— and —NR⁵⁰—; wherein R⁵⁰ is selected from -C1-C10 aminoalkyl, -C1-C10 alkylamino, and —C1-C10 hydroxyalkyl; wherein A² is selected from —O— and —NR⁶⁰—; wherein R⁶⁰ is selected from -C1-C10 aminoalkyl, -C1-C10 alkylamino, and —C1-C10 hydroxyalkyl; wherein A³ is selected from —O— and —NR⁷⁰—; wherein R⁷⁰ is selected from -C1-C10 aminoalkyl, -C1-C10 alkylamino, and -C1-C10 hydroxyalkyl; wherein R²⁰ is selected from halogen, —C1-C10 alkylamino and -C1-C10 alkoxy; wherein R³⁰ is selected from -C1-C10 alkanediyl, -C1-C10 aminoalkanediyl, and -C1-C10 hydroxyalkanediyl; and wherein each of R⁴⁰ and R⁴¹ is independently selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 1, 2, or 3 groups independently selected from halogen, —SF₅, —CN, —N₃, —OH, —NH₂, from -C1-C3 alkyl, -C1-C3 alkoxy, -C1-C3 haloalkyl, -C1-C3 aminoalkyl, -C1-C3 alkylamino, -C1-C3 haloalkylamino, -C1-C3 hydroxyalkyl, -C1-C3 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Aspect 88. The compound of Aspect 87, wherein R^{5a} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R⁴⁰—A³—R⁴¹; and wherein each of R^{5b}, R^{5c}, R^{5d}, and R^{5e} is independently selected from hydrogen, halogen, —SF₅, —CN, —N₃, —OH, —NH₂, —CF₃, and —CF₂CF₃.

Aspect 89. The compound of Aspect 88, wherein R^{5a} is R²⁰.

Aspect 90. The compound of any one of Aspect 88 or Aspect 89, wherein R²⁰ is selected from -C2-C7 alkylamino and —C2-C7 alkoxy.

Aspect 91. The compound of any one of Aspect 88 or Aspect 89, wherein R²⁰ is halogen.

Aspect 92. The compound of any one of Aspect 87-Aspect 91, wherein each of R^{5b}, R^{5c}, R^{5d}, and R^{5e} is selected from halogen and hydrogen.

Aspect 93. The compound of Aspect 92, wherein each of R^{5b}, R^{5c}, R^{5d}, and R^{5e} is hydrogen.

Aspect 94. The compound of any one of Aspect 88-Aspect 93, wherein R¹ is halogen.

Aspect 95. The compound of Aspect 94, wherein R¹ is fluoro.

Aspect 96. The compound of Aspect 87, wherein R^{5b} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R⁴⁰—A³—R⁴¹; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.

Aspect 97. The compound of Aspect 96, wherein R^{5b} is R²⁰.

Aspect 98. The compound of Aspect 96 or Aspect 97, wherein R²⁰ is selected from -C2-C7 alkylamino and —C2-C7 alkoxy.

Aspect 99. The compound of Aspect 96 or Aspect 97, wherein R²⁰ is halogen.

Aspect 100. The compound of any one of Aspect 96-Aspect 99, wherein each of R^{5a}, R^{5c}, R^{5d}, and R^{5e} is selected from halogen and hydrogen.

Aspect 101. The compound of Aspect 100, wherein each of R^{5a}, R^{5c}, R^{5d}, and R^{5e} is hydrogen.

Aspect 102. The compound of any one of Aspect 96-Aspect 101, wherein R¹ is halogen.

Aspect 103. The compound of Aspect 102, wherein R¹ is fluoro.

Aspect 104. The compound of Aspect 87, wherein R^{5c} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R⁴⁰—A³—R⁴¹; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.

Aspect 105. The compound of Aspect 104, wherein R^{5c} is R²⁰.

Aspect 106. The compound of Aspect 104 or Aspect 105, wherein R²⁰ is selected from —C2-C7 alkylamino and —C2-C7 alkoxy.

Aspect 107. The compound of Aspect 104 or Aspect 105, wherein R²⁰ is halogen.

Aspect 108. The compound of anyone of Aspect 104-Aspect 107, wherein each of R^{5a}, R^{5b}, R^{5d}, and R^{5e} is selected from halogen and hydrogen.

Aspect 109. The compound of Aspect 108, wherein each of R^{5a}, R^{5b}, R^{5d}, and R^{5e} is hydrogen.

Aspect 110. The compound of anyone of Aspect 104-Aspect 109, wherein R¹ is halogen.

Aspect 111. The compound of Aspect 110, wherein R¹ is fluoro.

Aspect 112. The compound of Aspect 87, present as:

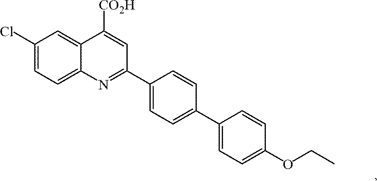

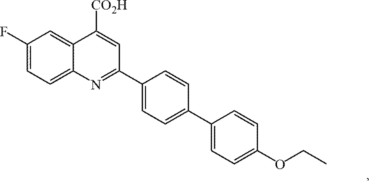

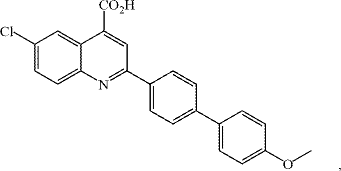

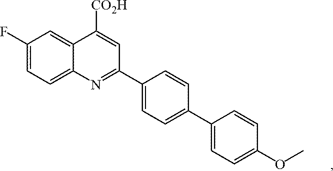

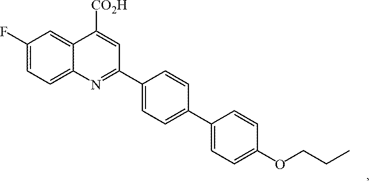

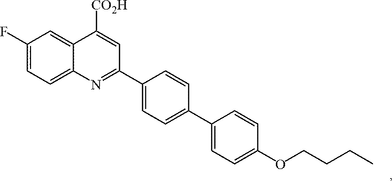

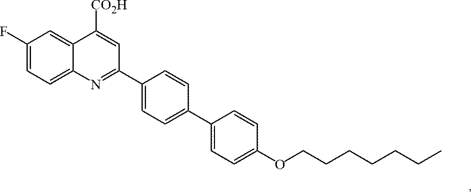

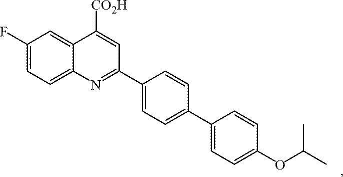

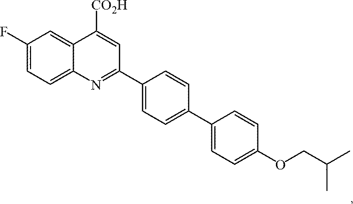

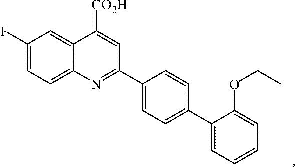

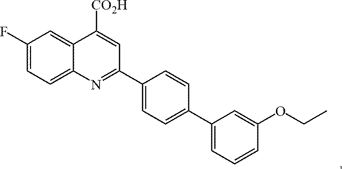

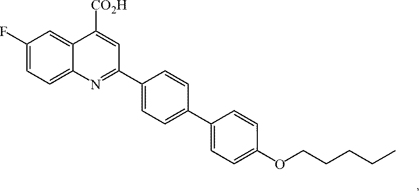

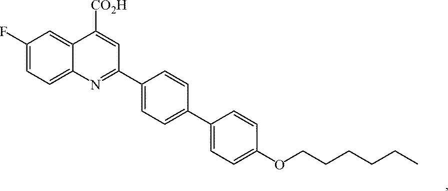

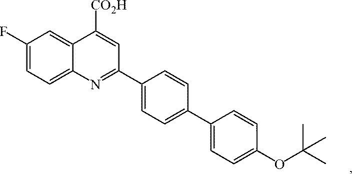

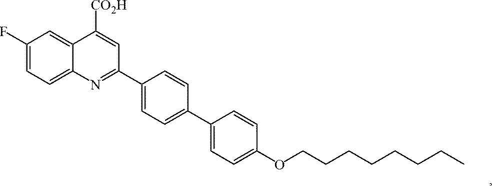

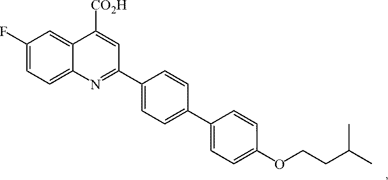

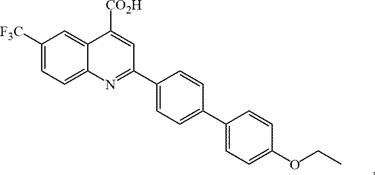

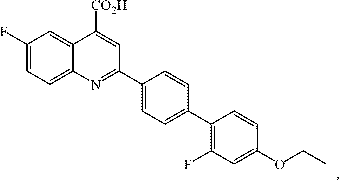

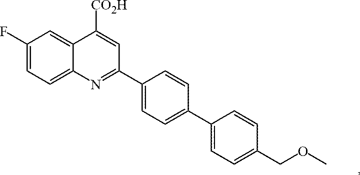

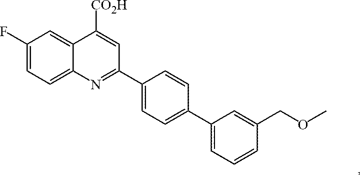

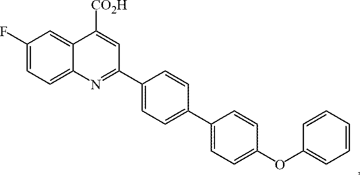

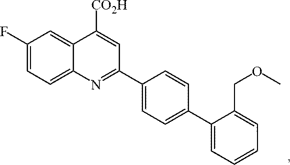

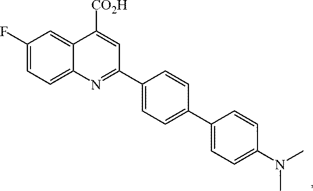

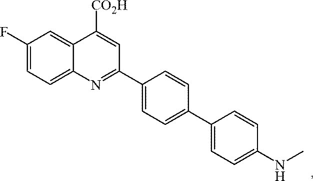

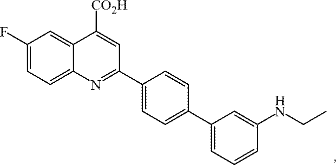

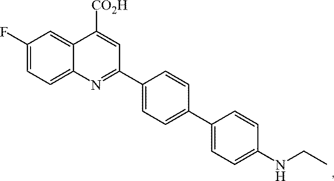

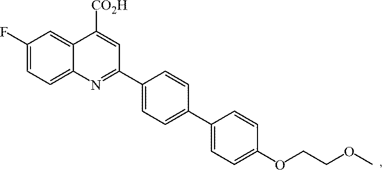

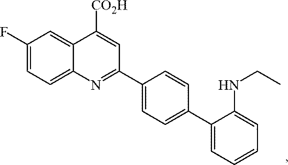

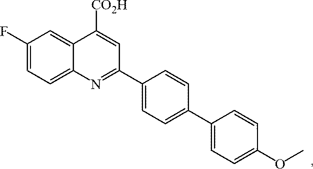

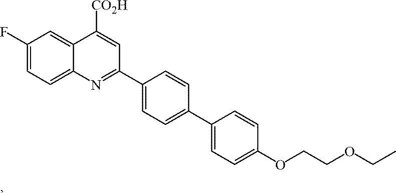

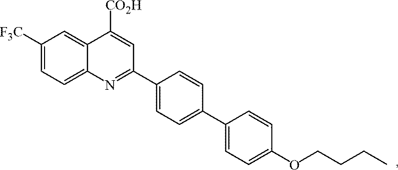

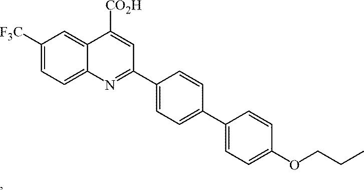

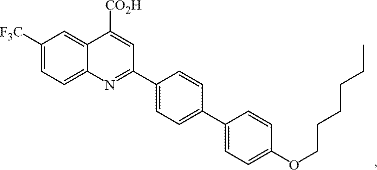

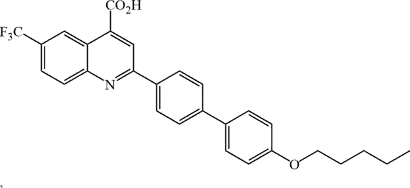

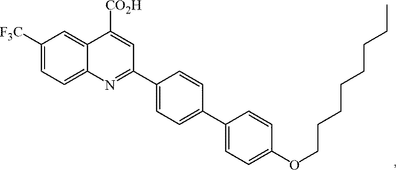

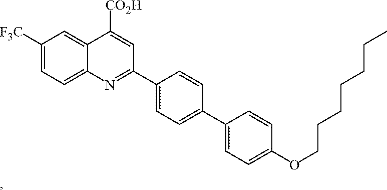

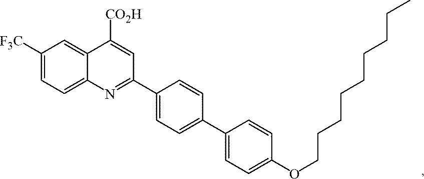

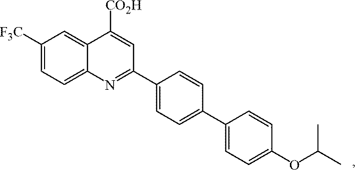

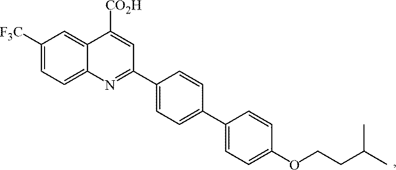

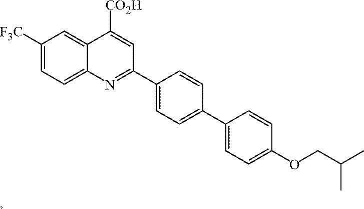

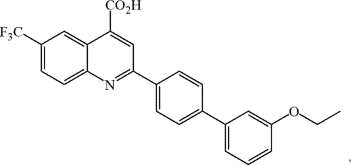

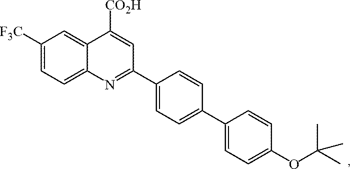

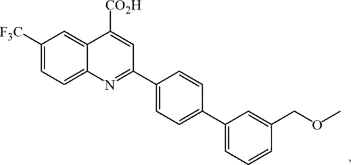

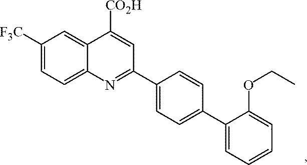

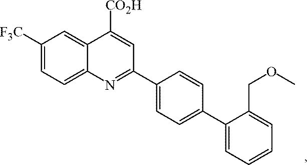

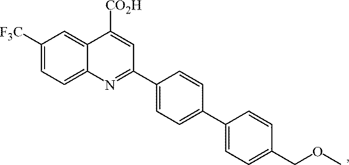

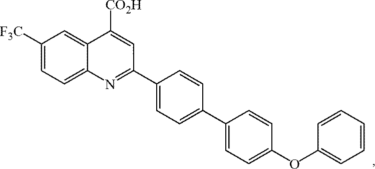

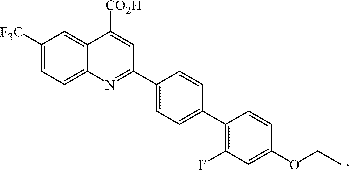

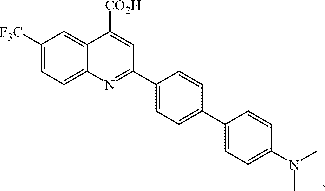

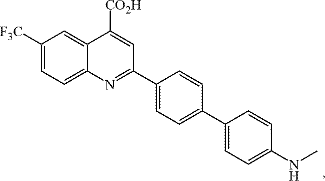

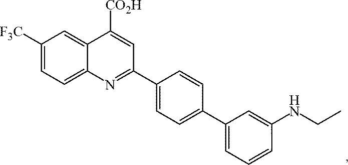

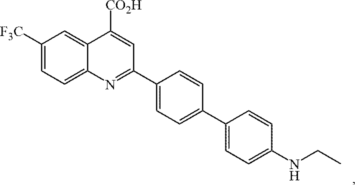

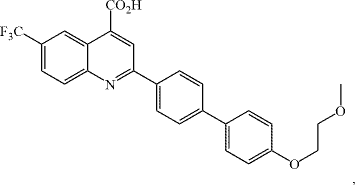

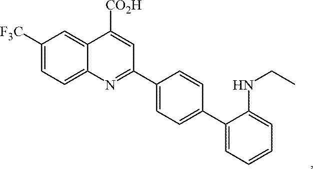

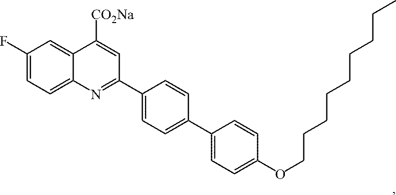

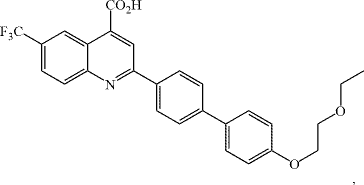

or a subgroup thereof.

Aspect 113. The compound of Aspect 87, present as:

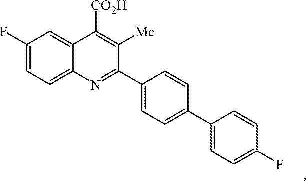

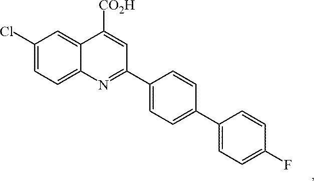

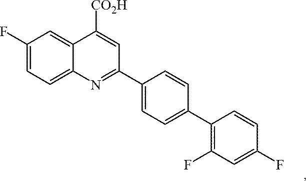

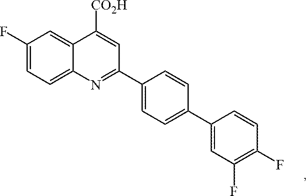

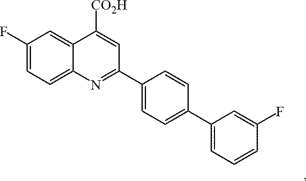

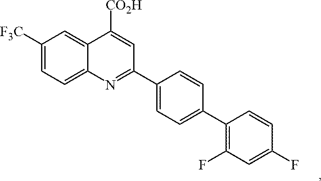

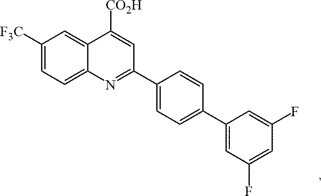

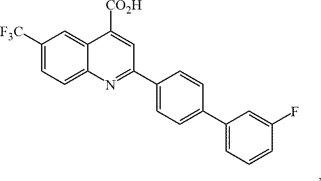

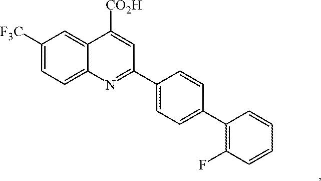

or a subgroup thereof.

Aspect 114. The compound of anyone of Aspect 87-Aspect 113, wherein the compound is a pharmaceutically acceptable salt thereof comprising the conjugate base form of the compound, and a counter ion selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.

Aspect 115. The compound of Aspect 114, wherein the counter ion is Na⁺.

### DHODH Inhibitor Compounds - Group II

A disclosed DHODH inhibitor can be any DHODH inhibitor as disclosed in Intl. Pat. Appl. No. PCT/US20/66682, which is incorporated herein by reference, and further described herein. For convenience, compounds of this structural type will be referred to as DHODH Inhibitor Compounds - Group II.

Disclosed are DHODH Inhibitor Compounds - Group II compounds having a formula represented by a structure:

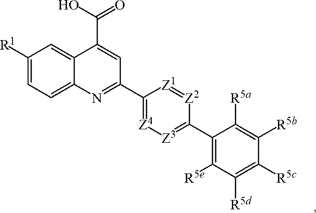

wherein each of Z¹, Z², Z³, and Z⁴ is independently selected from CH and N; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R³¹—A³—R⁴⁰; wherein A¹ is selected from —O— and —NR⁵⁰—; wherein R⁵⁰ is selected from hydrogen, —C1-C10 alkyl, —C1-C10 aminoalkyl, and —C1-C10 hydroxyalkyl; wherein A² is selected from —O— and —NR⁶⁰—; wherein R⁶⁰ is selected from hydrogen, —C1-C10 alkyl, —C1-C10 aminoalkyl, and —C1-C10 hydroxyalkyl; wherein A³ is selected from —O— and —NR⁷⁰—; wherein R⁷⁰ is selected from hydrogen, —C1-C10 alkyl, —C1-C10 aminoalkyl, and —C1-C10 hydroxyalkyl; wherein R²⁰ is selected from halogen, —C1-C10 alkyl, —C1-C10 haloalkyl, —C1-C10 hydroxyalkyl, —C1-C10 alkylamino, and —C1-C10 alkoxy; wherein each of R³⁰ and R³¹ is independently selected from —C1-C10 alkanediyl, —C1-C10 haloalkanediyl, —C1-C10 aminoalkanediyl, and —C1-C10 hydroxyalkanediyl; and wherein R⁴⁰ is selected from —C1-C10 alkyl, —C1-C10 haloalkyl, —C1-C10 aminoalkyl, —C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, —C1-C4 alkyl, —C1-C4 alkoxy, —C1-C4 haloalkyl, —C1-C4 aminoalkyl, —C1-C4 alkylamino, —C1-C4 haloalkylamino, —C1-C4 hydroxyalkyl, —C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; or a pharmaceutically acceptable salt thereof.

The following listing of exemplary aspects supports and is supported by the disclosure provided herein for DHODH Inhibitor Compounds - Group II.

Aspect 1. A compound having a formula represented by a structure:

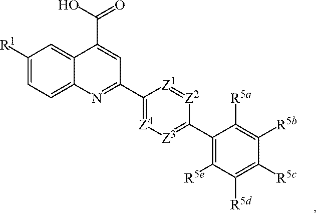

wherein each of Z¹, Z², Z³, and Z⁴ is independently selected from CH and N; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R³¹—A³—R⁴⁰; wherein A¹ is selected from —O— and —NR⁵⁰—; wherein R⁵⁰ is selected from hydrogen, —C1-C10 alkyl, —C1-C10 aminoalkyl, and —C1-C10 hydroxyalkyl; wherein A² is selected from —O— and —NR⁶⁰—; wherein R⁶⁰ is selected from hydrogen, —C1-C10 alkyl, —C1-C10 aminoalkyl, and —C1-C10 hydroxyalkyl; wherein A³ is selected from —O— and —NR⁷⁰—; wherein R⁷⁰ is selected from hydrogen, —C1-C10 alkyl, —C1-C10 aminoalkyl, and —C1-C10 hydroxyalkyl; wherein R²⁰ is selected from halogen, —C1-C10 alkyl, —C1-C10 haloalkyl, —C1-C10 hydroxyalkyl, —C1-C10 alkylamino, and —C1-C10 alkoxy; wherein each of R³⁰ and R³¹ is independently selected from —C1-C10 alkanediyl, —C1-C10 haloalkanediyl, —C1-C10 aminoalkanediyl, and —C1-C10 hydroxyalkanediyl; and wherein R⁴⁰ is selected from —C1-C10 alkyl, —C1-C10 haloalkyl, —C1-C10 aminoalkyl, —C1-C10 hydroxyalkyl, and —(CH₂)ₙAR¹; wherein n is an integer selected from 1, 2, and 3; and wherein AR¹ is a phenyl group substituted with 0,1, 2, 3, 4, or 5 groups independently selected from halogen, —SF₅, —CN, —N₃, —OH, —NH₂, —C1-C4 alkyl, —C1-C4 alkoxy, —C1-C4 haloalkyl, —C1-C4 aminoalkyl, —C1-C4 alkylamino, —C1-C4 haloalkylamino, —C1-C4 hydroxyalkyl, —C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; or a pharmaceutically acceptable salt thereof.

Aspect 2. The compound of Aspect 1, wherein R¹ is selected from halogen, —SF₅, —CF₃, and —CF₂CF₃.

Aspect 3. The compound of Aspect 2, wherein R¹ is halogen.

Aspect 4. The compound of Aspect 3, wherein R¹ is F or Cl.

Aspect 5. The compound of Aspect 3, wherein R¹ is F.

Aspect 6. The compound of Aspect 2, wherein R¹ is selected from —SF₅, —CF₃, and —CF₂CF₃.

Aspect 7. The compound of Aspect 2, wherein R¹ is —SF₅.

Aspect 8. The compound of any one of Aspect 1-Aspect 7, wherein R^{5c} is halogen, C1-C7 haloalkyl, or -O(C1-C7 haloalkyl).

Aspect 9. The compound of Aspect 8, wherein R^{5c} is halogen.

Aspect 10. The compound of Aspect 9, wherein R^{5c} is F.

Aspect 11. The compound of Aspect 8, wherein R^{5c} is —OCF₃, —OCH₂CF₃, or —OCF₂CF₃.

Aspect 12. The compound of any one of Aspect 1-Aspect 7, wherein R^{5c} is —OH, -O(C1-C7 alkyl), -C1-C7 hydroxyalkyl, -O-(C1-C7 hydroxyalkyl), -CH₂O(C1-C7 alkyl), or -(CH₂)₂O(C1-C7 alkyl).

Aspect 13. -O(C1-C7 alkyl), -(C1-C7 alkanediyl)-OH, -O(C1-C7 alkanediyl)-OH, -CH₂O(C1-C7 alkyl), or -(CH₂)₂O(C1-C7 alkyl).

Aspect 14. The compound of Aspect 13, wherein R^{5c} is —OCH₃, —OCH₂CH₃, —O(CH₂)₂CH₃, —OCH(CH₃)₂, —O(CH₂)₃CH₃, —OCH₂CH(CH₃)₂, —OCH(CH₂CH₃)(CH₃), —CH₂OH, —(CH₂)₂OH, —(CH₂)₃OH, —(CH₂)₄OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or —(CH₂)₂OCH(CH₂CH₃)₂(CH₃).

Aspect 15. The compound of Aspect 13, wherein R^{5c} is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or —(CH₂)₂OCH(CH₂CH₃)₂(CH₃).

Aspect 16. The compound of Aspect 13, wherein R^{5c} is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —(CH₂)₂OCH₃, or—(CH₂)₂OCH₂CH₃.

Aspect 17. The compound of Aspect 13, wherein R^{5c} is —OCH₃, —OCH₂CH₃, —CH₂OH, —(CH₂)₂OH, —CH₂OCH₃, or —CH₂OCH₂CH₃.

Aspect 18. The compound of Aspect 13, wherein R^{5c} is —OCH₃ or —OCH₂CH₃.

Aspect 19. The compound of any one of Aspect 12-Aspect 18, wherein each of R^{5a}, R^{5b}, R^{5d}, and R^{5e} is hydrogen.

Aspect 20. The compound of any one of Aspect 1-Aspect 7, wherein R^{5a} is selected from a group having formula represented by a structure: -R²⁰, —R³⁰—A¹—R⁴⁰, —A¹—R⁴⁰, —A¹—R³⁰—A²—R⁴⁰, or-A¹-R³⁰-A²-R³¹-A³-R⁴¹; and wherein each of R^{5b}, R^{5c}, R^{5d}, and R^{5e} is independently selected from hydrogen, halogen, —SF₅, —CN, —N₃, —OH, —NH₂, —CF₃, and —CF₂CF₃.

Aspect 21. The compound of Aspect 20, wherein R^{5a} is R²⁰.

Aspect 22. The compound of any one of Aspect 20 or Aspect 21, wherein R²⁰ is selected from -C2-C7 alkylamino and —C2-C7 alkoxy.

Aspect 23. The compound of any one of Aspect 20 or Aspect 21, wherein R²⁰ is halogen.

Aspect 24. The compound of any one of Aspect 1-Aspect 23, wherein each of R^{5b}, R^{5c}, R^{5d}, and R^{5e} is selected from halogen and hydrogen.

Aspect 25. The compound of Aspect 24, wherein each of R^{5b}, R^{5c}, R^{5d}, and R^{5e} is hydrogen.

Aspect 26. The compound of any one of Aspect 1-Aspect 7, wherein R^{5b} is selected from a group having formula represented by a structure: -R²⁰, —R³⁰—A¹—R⁴⁰, —A¹—R⁴⁰, —A¹—R³⁰—A²—R⁴⁰, or-A¹-R³⁰-A²-R³¹-A³-R⁴¹; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.

Aspect 27. The compound of Aspect 26, wherein R^{5b} is R²⁰.

Aspect 28. The compound of Aspect 26 or Aspect 27, wherein R²⁰ is selected from -C2-C7 alkylamino and —C2-C7 alkoxy.

Aspect 29. The compound of Aspect 26 or Aspect 27, wherein R²⁰ is halogen.

Aspect 30. The compound of any one of Aspect 26-Aspect 29, wherein each of R^{5a}, R^{5c}, R^{5d}, and R^{5e} is selected from halogen and hydrogen.

Aspect 31. The compound of Aspect 30, wherein each of R^{5a}, R^{5c}, R^{5d}, and R^{5e} is hydrogen.

Aspect 32. The compound of any one of Aspect 1-Aspect 7, wherein R^{5c} is selected from a group having formula represented by a structure: -R²⁰, -R³⁰-A¹-R⁴⁰, -A¹-R⁴⁰, —A¹—R³⁰—A²—R⁴⁰, or —A¹—R³⁰—A²—R³¹—A³—R⁴¹; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.

Aspect 33. The compound of Aspect 32, wherein R^{5c} is R²⁰.

Aspect 34. The compound of Aspect 32 or Aspect 33, wherein R²⁰ is selected from -C2-C7 alkylamino and -C2-C7 alkoxy.

Aspect 35. The compound of Aspect 32 or Aspect 33, wherein R²⁰ is halogen.

Aspect 36. The compound of anyone of Aspect 32-Aspect 35, wherein each of R^{5a}, R^{5b}, R^{5d}, and R^{5e} is selected from halogen and hydrogen.

Aspect 37. The compound of Aspect 36, wherein each of R^{5a}, R^{5b}, R^{5d}, and R^{5e} is hydrogen.

The compound of Aspect 1, having a structure represented by a formula:

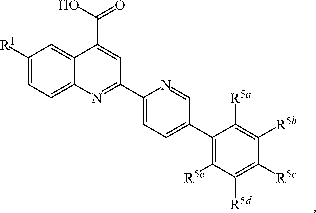

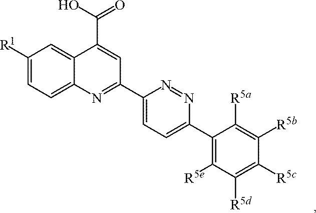

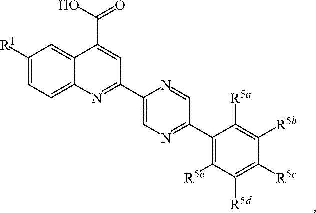

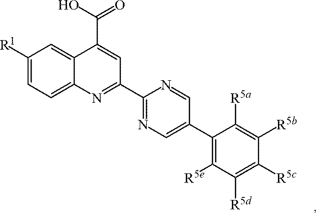

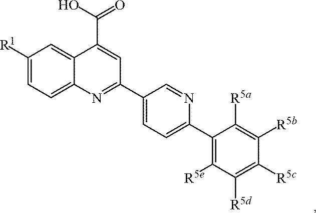

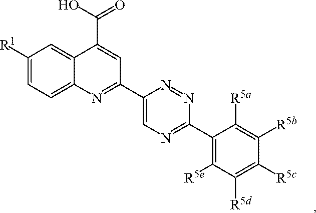

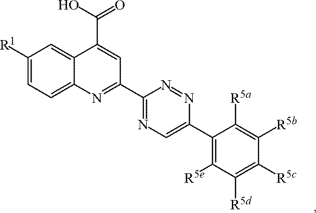

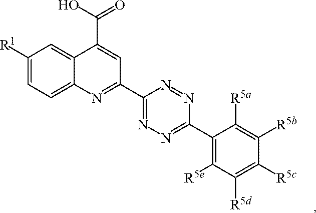

or a subgroup thereof.

Aspect 39. The compound of Aspect 1, having a structure represented by a formula:

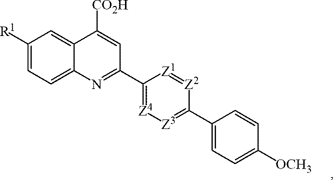

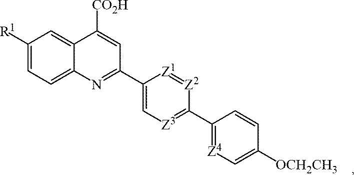

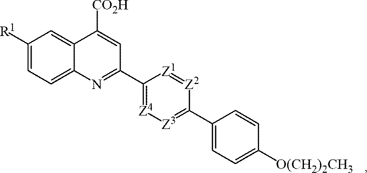

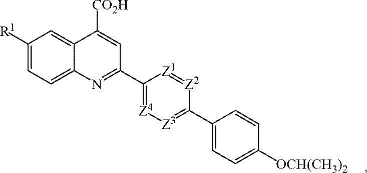

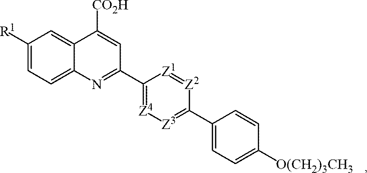

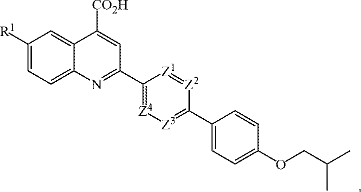

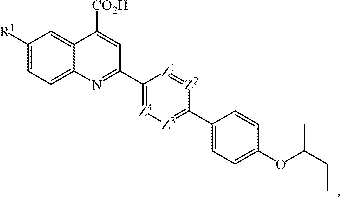

or a subgroup thereof.

Aspect 40. The compound of Aspect 1, present as:

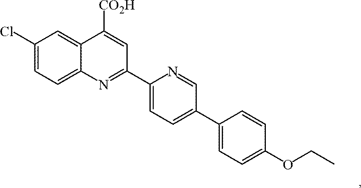

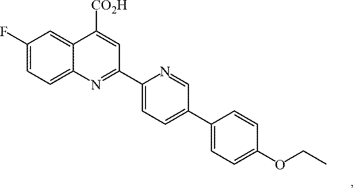

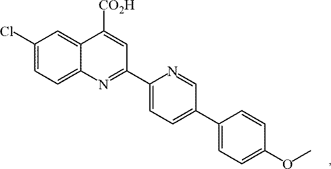

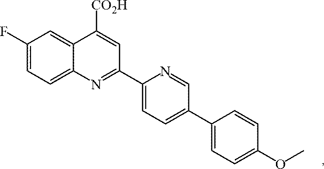

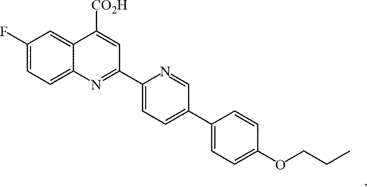

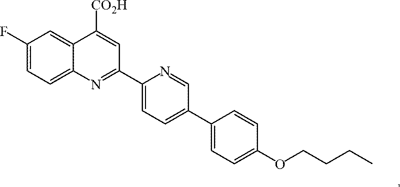

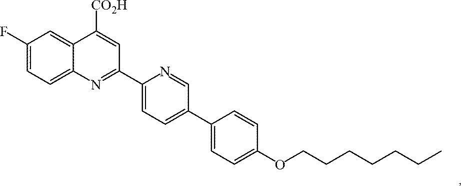

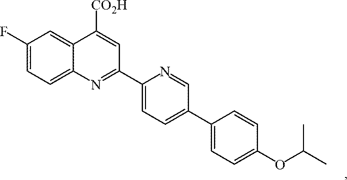

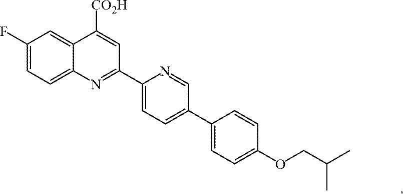

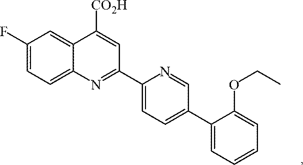

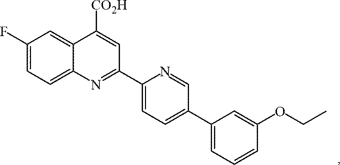

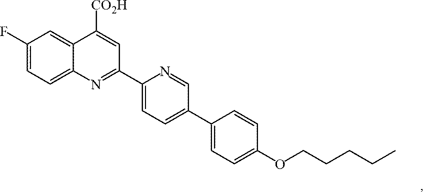

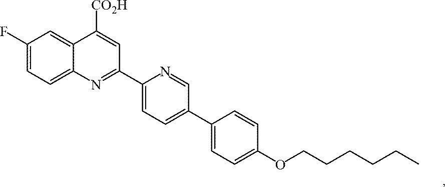

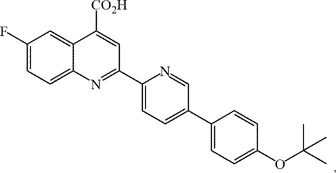

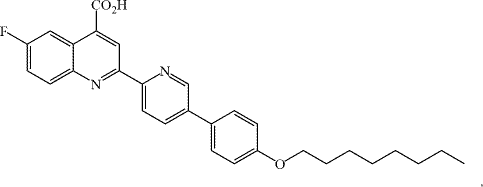

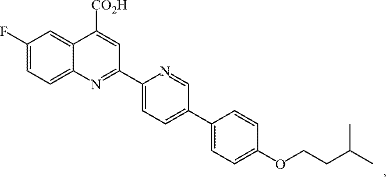

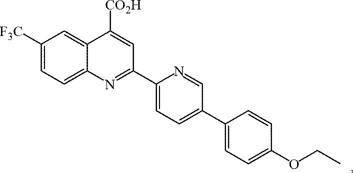

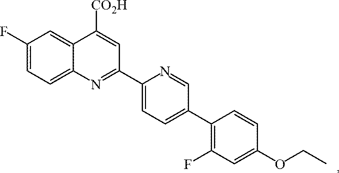

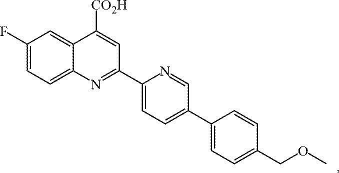

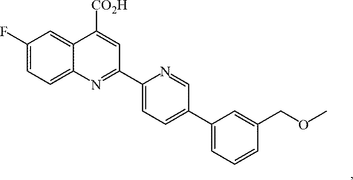

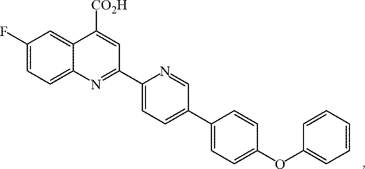

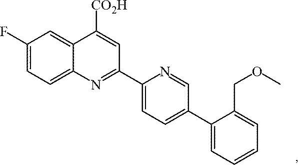

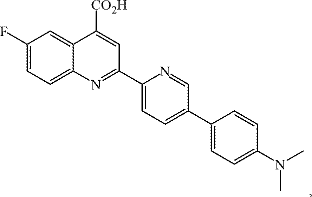

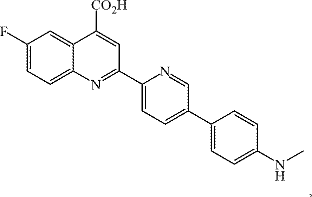

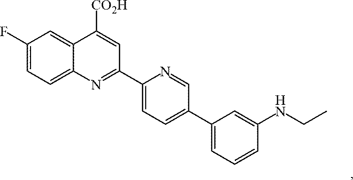

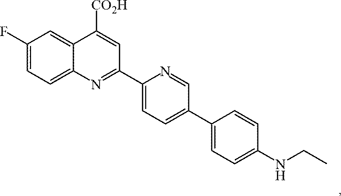

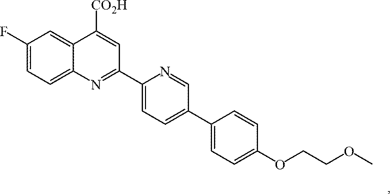

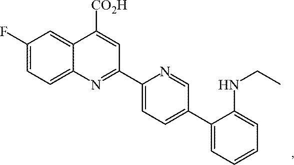

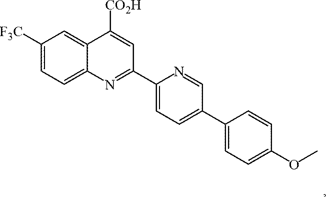

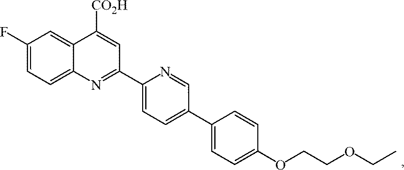

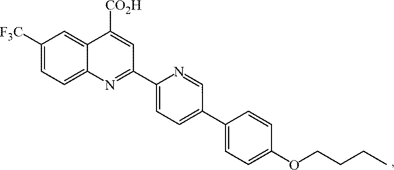

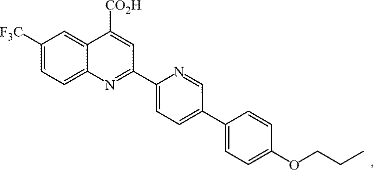

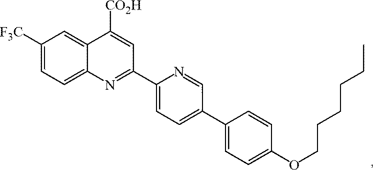

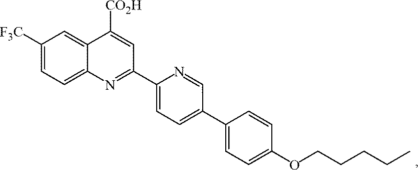

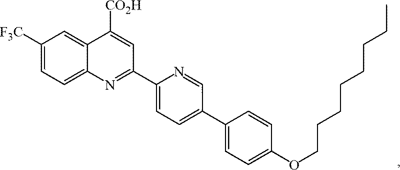

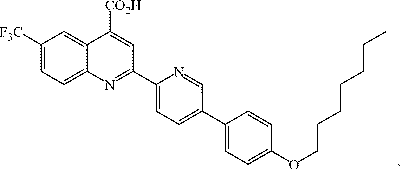

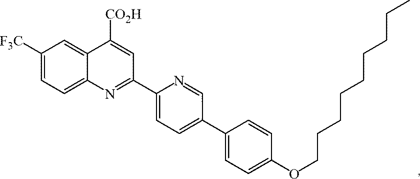

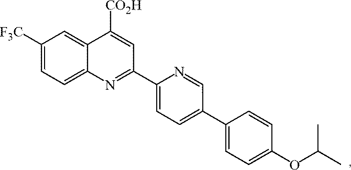

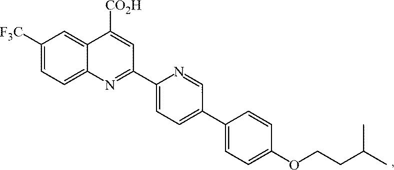

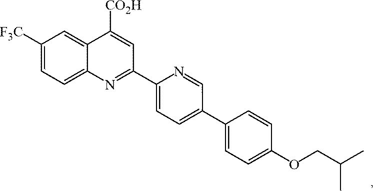

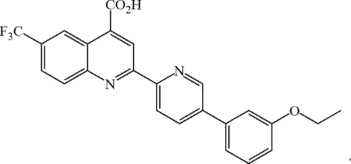

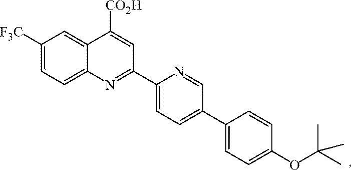

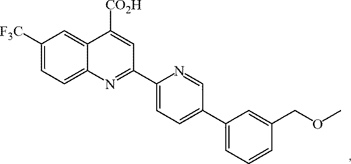

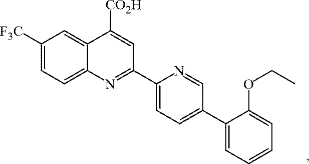

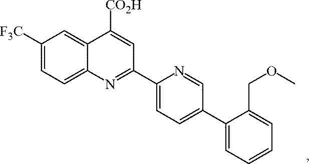

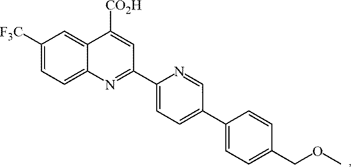

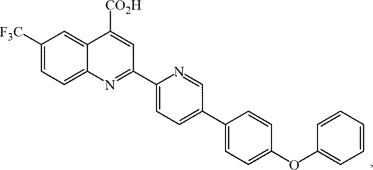

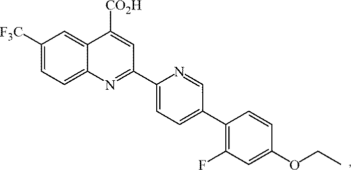

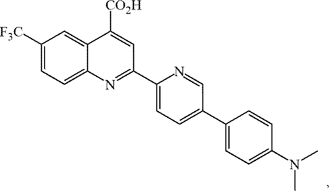

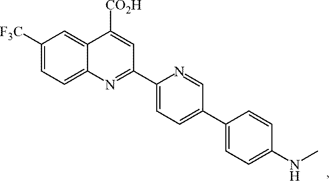

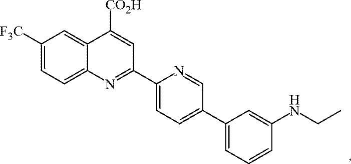

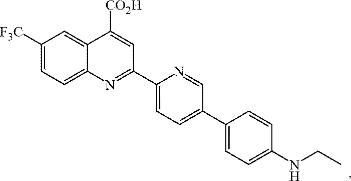

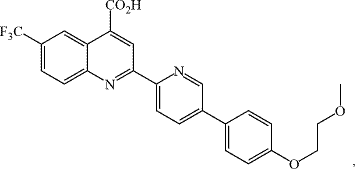

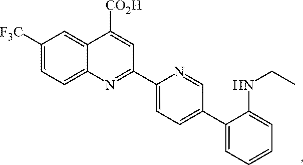

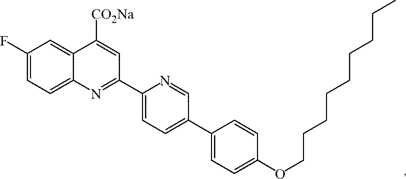

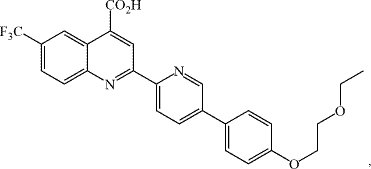

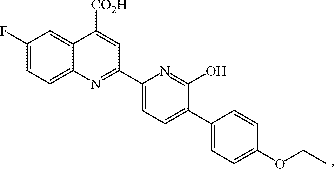

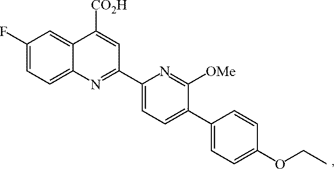

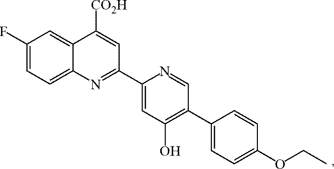

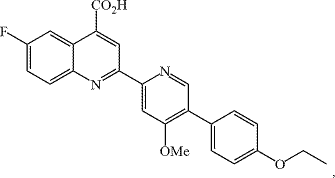

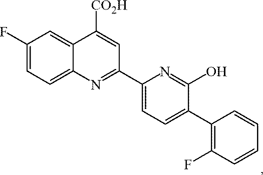

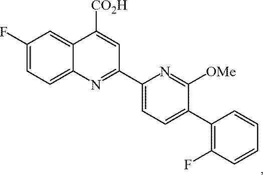

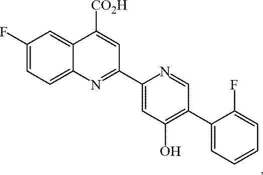

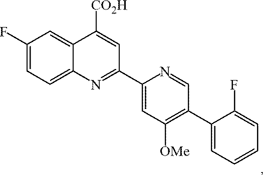

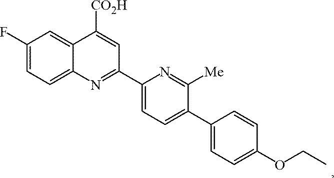

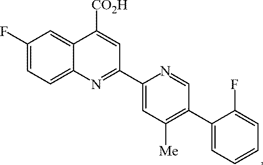

or a subgroup thereof.

Aspect 41. The compound of Aspect 1, present as:

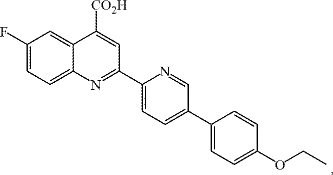

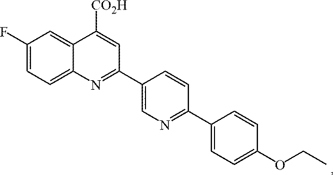

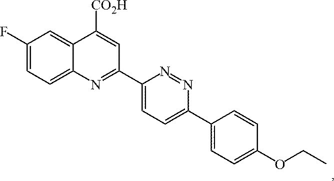

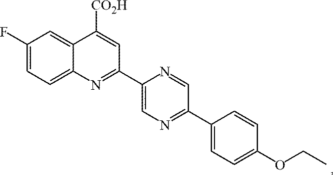

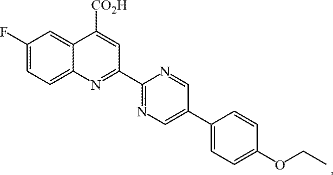

or a subgroup thereof.

Aspect 42. The compound of any one of Aspect 1-Aspect 41, wherein the compound is a pharmaceutically acceptable salt thereof comprising the conjugate base form of the compound, and a counter ion selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.

Aspect 43. The compound of Aspect 42, wherein the counter ion is Na+.

### DHODH Inhibitor Compounds - Group III

A disclosed DHODH inhibitor can be any DHODH inhibitor as disclosed in Intl. Pat. Appl. No. PCT/US20/66684, which is incorporated herein by reference, and further described herein. For convenience, compounds of this structural type will be referred to as DHODH Inhibitor Compounds - Group III.

Disclosed are DHODH Inhibitor Compounds - Group III compounds having a formula represented by a structure:

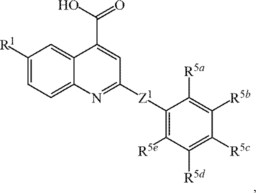

wherein Z¹ is a five-membered heterocyclic diyl; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰, —R³⁰—A¹—R⁴⁰, -A¹-R⁴⁰, —A¹—R³⁰—A²—R⁴⁰, or -A¹-R³⁰-A²-R³¹-A³-R⁴⁰; wherein A¹ is selected from —O— and -NR⁵⁰-; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and -C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and —C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

The following listing of exemplary aspects supports and is supported by the disclosure provided herein for DHODH Inhibitor Compounds - Group III.

Aspect 1. A compound having a formula represented by a structure:

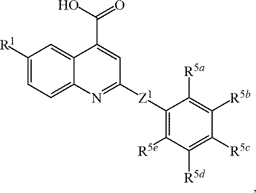

wherein Z¹ is a five-membered heterocyclic diyl; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: - R²⁰, —R³⁰—A¹—R⁴⁰, —A¹—R⁴⁰, —A¹—R³⁰—A²—R⁴⁰, or -A¹-R³⁰-A²-R³¹-A³-R⁴⁰; wherein A¹ is selected from —O— and -NR⁵⁰-; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and -C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and —C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙA R¹; wherein n is an integer selected from 1, 2, and 3; and wherein A R¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.

Aspect 2. The compound of 1, wherein Z1 has a formula represented by a structure:

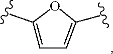

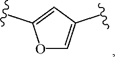

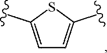

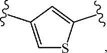

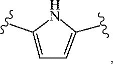

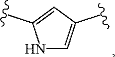

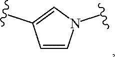

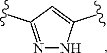

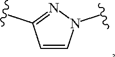

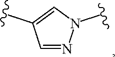

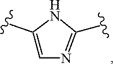

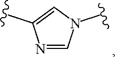

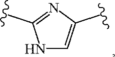

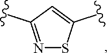

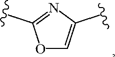

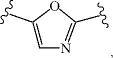

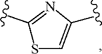

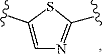

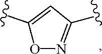

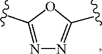

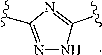

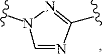

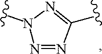

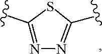

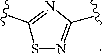

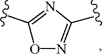

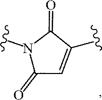

or subgroups thereof.

Aspect 3. The compound of 1, wherein R¹ is selected from halogen, —SF₅, —CF₃, and —CF₂CF₃.

Aspect 4. The compound of Aspect 3, wherein R¹ is halogen.

Aspect 5. The compound of Aspect 4, wherein R¹ is F or Cl.

Aspect 6. The compound of Aspect 4, wherein R¹ is F.

Aspect 7. The compound of Aspect 3, wherein R¹ is selected from —SF₅, —CF₃, and —CF₂CF₃.

Aspect 8. The compound of Aspect 3, wherein R¹ is —SF₅.

Aspect 9. The compound of any one of 1-Aspect 8, wherein R^{5c} is halogen, C1-C7 haloalkyl, or -O(C1-C7 haloalkyl).

Aspect 10. The compound of Aspect 9, wherein R^{5c} is halogen.

Aspect 11. The compound of Aspect 10, wherein R^{5c} is F.

Aspect 12. The compound of Aspect 9, wherein R^{5c} is —OCF₃, —OCH₂CF₃, or —OCF₂CF₃.

Aspect 13. The compound of any one of 1-Aspect 8, wherein R^{5c} is —OH, -O(C1-C7 alkyl), -C1-C7 hydroxyalkyl, -O-(C1-C7 hydroxyalkyl), -CH2O(C1-C7 alkyl), or -(CH2)2O(C1-C7 alkyl).

Aspect 14. The compound of Aspect 13, wherein R^{5c} is -O(C1-C7 alkyl), -(C1-C7 alkanediyl)-OH, -O(C1-C7 alkanediyl)-OH, -CH2O(C1-C7 alkyl), or-(CH2)2O(C1-C7 alkyl).

Aspect 15. The compound of Aspect 14, wherein R^{5c} is —OCH₃, —OCH₂CH₃, —O(CH₂)₂CH₃, —OCH(CH₃)₂, —O(CH₂)₃CH₃, —OCH₂CH(CH₃)₂, —OCH(CH₂CH₃)(CH₃), —CH₂OH, —(CH₂)₂OH, —(CH₂)₃OH, —(CH₂)₄OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or —(CH₂)₂OCH(CH₂CH₃)₂(CH₃).

Aspect 16. The compound of Aspect 14, wherein R^{5c} is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —CH₂O(CH₂)₂CH₃, —CH₂OCH(CH₃)₂, —CH₂OCH(CH₂CH₃)₂(CH₃), —(CH₂)₂OCH₃, —(CH₂)₂OCH₂CH₃, —(CH₂)₂O(CH₂)₂CH₃, —(CH₂)₂OCH(CH₃)₂, or —(CH₂)₂OCH(CH₂CH₃)₂(CH₃).

Aspect 17. The compound of Aspect 14, wherein R^{5c} is —OCH₃, —OCH₂CH₃, —O(CH₂)₂CH₃, —OCH(CH₃)₂, —CH₂OH, —(CH₂)₂OH, —(CH₂)₃OH, —CH₂OCH₃, —CH₂OCH₂CH₃, —(CH₂)₂OCH₃, or —(CH₂)₂OCH₂CH₃.

Aspect 18. The compound of Aspect 14, wherein R^{5c} is —OCH₃, —OCH₂CH₃, —CH₂OH, —(CH₂)₂OH, —CH₂OCH₃, or —CH₂OCH₂CH₃.

Aspect 19. The compound of Aspect 14, wherein R^{5c} is —OCH₃ or—OCH₂CH₃.

Aspect 20. The compound of any one of Aspect 13-Aspect 19, wherein each of R^{5a} R^{5b}, R^{5d}, and R^{5e} is hydrogen.

Aspect 21. The compound of any one of 1-Aspect 8, wherein R^{5a} is selected from a group having formula represented by a structure: -R²⁰, —R³⁰—A¹—R⁴⁰, -A¹-R⁴⁰, —A¹—R³⁰—A²—R⁴⁰, or —A¹—R³⁰—A²—R³¹—A³—R⁴¹; and wherein each of R^{5b}, R^{5c}, R^{5d}, and R^{5e} is independently selected from hydrogen, halogen, —SF₅, —CN, —N₃, —OH, —NH₂, —CF₃, and —CF₂CF₃.

Aspect 22. The compound of Aspect 21, wherein R^{5a} is R²⁰.

Aspect 23. The compound of any one of Aspect 21 or Aspect 22, wherein R²⁰ is selected from -C2-C7 alkylamino and —C2-C7 alkoxy.

Aspect 24. The compound of any one of Aspect 21 or Aspect 22, wherein R²⁰ is halogen.

Aspect 25. The compound of any one of 1-Aspect 24, wherein each of R^{5b}, R^{5c}, R^{5d}, and R^{5e} is selected from halogen and hydrogen.

Aspect 26. The compound of Aspect 25, wherein each of R^{5b}, R^{5c}, R^{5d}, and R^{5e} is hydrogen.

Aspect 27. The compound of any one of 1-Aspect 8, wherein R^{5b} is selected from a group having formula represented by a structure: —R²⁰, —R³⁰—A¹—R⁴⁰, —A¹—R⁴⁰, —A¹—R³⁰—A²—R⁴⁰, or —A¹—R³⁰—A²—R³¹—A³—R⁴¹; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.

Aspect 28. The compound of Aspect 27, wherein R^{5b} is R²⁰.

Aspect 29. The compound of Aspect 27 or Aspect 28, wherein R²⁰ is selected from -C2-C7 alkylamino and -C2-C7 alkoxy.

Aspect 30. The compound of Aspect 27 or Aspect 28, wherein R²⁰ is halogen.

Aspect 31. The compound of any one of Aspect 27-Aspect 30, wherein each of R^{5a}, R^{5c}, R^{5d}, and R^{5e} is selected from halogen and hydrogen.

Aspect 32. The compound of Aspect 31, wherein each of R^{5a}, R^{5c}, R^{5d}, and R^{5e} is hydrogen.

Aspect 33. The compound of any one of 1-Aspect 8, wherein R^{5c} is selected from a group having formula represented by a structure: -R²⁰, -R³⁰-A¹-R⁴⁰, -A¹-R⁴⁰, -A¹-R³⁰-A²-R⁴⁰, or -A¹-R³⁰-A²-R³¹-A³-R⁴¹; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.

Aspect 34. The compound of Aspect 33, wherein R^{5c} is R²⁰.

Aspect 35. The compound of Aspect 33 or Aspect 34, wherein R²⁰ is selected from -C2-C7 alkylamino and -C2-C7 alkoxy.

Aspect 36. The compound of Aspect 33 or Aspect 34, wherein R²⁰ is halogen.

Aspect 37. The compound of anyone of Aspect 33-Aspect 36, wherein each of R^{5a}, R^{5b}, R^{5d}, and R^{5e} is selected from halogen and hydrogen.

Aspect 38. The compound of Aspect 37, wherein each of R^{5a}, R^{5b}, R^{5d}, and R^{5e} is hydrogen.

Aspect 39. The compound of 1, present as:

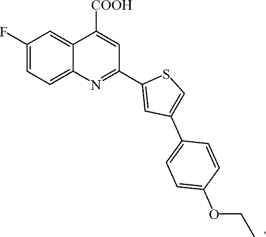

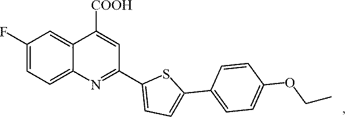

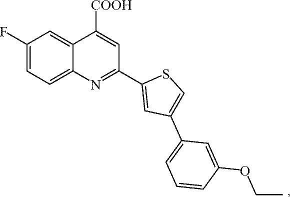

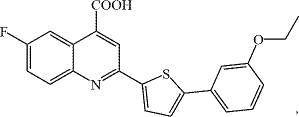

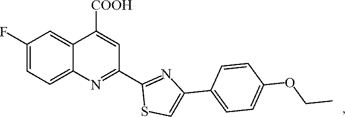

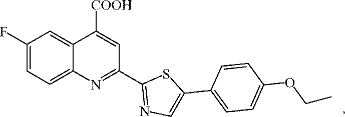

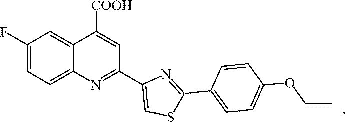

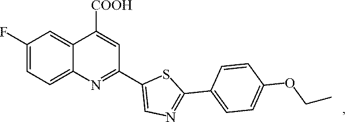

or a subgroup thereof.

Aspect 40. The compound of anyone of 1-Error! Reference source not found., wherein the compound is a pharmaceutically acceptable salt thereof comprising the conjugate base form of the compound, and a counter ion selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.

Aspect 41. The compound of Aspect 114, wherein the counter ion is Na⁺.

### DHODH Inhibitor Compounds - Group IV

A disclosed DHODH inhibitor can be any DHODH inhibitor as disclosed in Intl. Pat. Appl. PCT/US20/67065, which is incorporated herein by reference, and further described herein. For convenience, compounds of this structural type will be referred to as DHODH Inhibitor Compounds - Group IV.

Disclosed are DHODH Inhibitor Compounds - Group IV compounds having a formula represented by a structure:

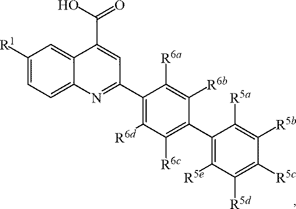

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰, -R³⁰-A¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, -C1-C10 alkoxy, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

Disclosed herein are compounds having a formula represented by a structure:

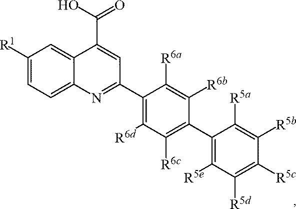

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰, -R³⁰-A¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

Disclosed herein are compounds having a formula represented by a structure:

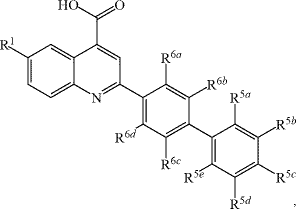

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: -R²⁰, -R³⁰-A¹-R⁴⁰, -A¹-R⁴⁰, -A¹-R³⁰-A²-R⁴⁰, or -A¹-R³⁰-A²-R³¹-A³-R⁴¹; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, and -C1-C10 alkoxy; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

In some aspects, each of R^{6a}, R^{6b}, R^{6c}, and R^{6d} can be independently selected from hydrogen, halogen, C1-C10 alkyl, C1-C10 alkoxy, and C1-C10 haloalkyl. In another aspect, R^{6a} and R^{6b} are independently selected from hydrogen and halogen. In still another aspect, R^{6a} is fluoro, or R^{6b} is fluoro, or a combination thereof. In another aspect, R^{6c} and R^{6d} can be hydrogen.

The following listing of exemplary aspects supports and is supported by the disclosure provided herein for DHODH Inhibitor Compounds - Group IV.

Aspect 1. A compound having a formula represented by a structure:

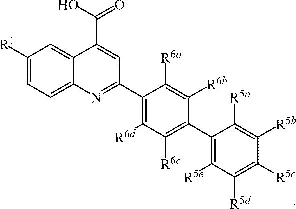

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d,} and R^{5e} is selected from a group having formula represented by a structure: -R²⁰, -R³⁰-A¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and —C1-C10 hydroxyalkyl; wherein R²⁰ is selected from halogen, -C1-C10 alkyl, -C1-C10 haloalkyl, -C1-C10 hydroxyalkyl, -C1-C10 alkylamino, -C1-C10 alkoxy, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.

Aspect 2. A compound having a formula represented by a structure:

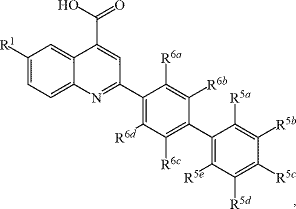

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d,} and R^{5e} is selected from a group having formula represented by a structure: —R²⁰, -R³⁰-A¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, and -C1-C10 alkoxy; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen;or a pharmaceutically acceptable salt thereof.

Aspect 3. The compound of Aspect 1 or Aspect 2, wherein R¹ is selected from halogen, —SF₅, —CF₃, and —CF₂CF₃.

Aspect 4. The compound of Aspect 3, wherein R¹ is halogen or —SF₅.

Aspect 5. The compound of Aspect 4, wherein R¹ is -F or -Cl.

Aspect 6. The compound of Aspect 4, wherein R¹ is -F.

Aspect 7. The compound of Aspect 4, wherein R¹ is -Cl.

Aspect 8. The compound of Aspect 4, wherein R¹ is —SF₅.

Aspect 9. The compound of Aspect 2, wherein R¹ is selected from —SF₅, —CF₃, and —CF₂CF₃.

Aspect 10. The compound of Aspect 9, wherein R¹ is —SF₅.

Aspect 11. The compound of Aspect 9, wherein R¹ is selected from —CF₃ and —CF₂CF₃.

Aspect 12. The compound of any one of 1-Aspect 11, wherein R^{5c} is halogen, C1-C7 haloalkyl, or -O(C1-C7 haloalkyl).

Aspect 13. The compound of Aspect 12, wherein R^{5c} is halogen.

Aspect 14. The compound of Aspect 13, wherein R^{5c} is F.

Aspect 15. The compound of Aspect 12, wherein R^{5c} is —OCF₃, —OCH2CF₃, or —OCF₂CF₃.

Aspect 16. The compound of any one of Aspect 1-Aspect 15, wherein R^{5c} is —OH, -O(C1-C7 alkyl), -C1-C7 hydroxyalkyl, -O-(C1-C7 hydroxyalkyl), -CH2O(C1-C7 alkyl), or -(CH2)2O(C1-C7 alkyl).

Aspect 17. The compound of Aspect 16, wherein R^{5c} is -O(C1-C7 alkyl), -(C1-C7 alkanediyl)-OH, -O(C1-C7 alkanediyl)-OH, -CH2O(C1-C7 alkyl), or-(CH2)2O(C1-C7 alkyl).

Aspect 18. The compound of Aspect 17, wherein R^{5c} is —OCH₃ or—OCH2CH₃.

Aspect 19. The compound of any one of Aspect 16-Aspect 18, wherein each of R^{5a}, R^{5b}, R^{5d}, and R^{5e} is hydrogen.

Aspect 20. The compound of any one of Aspect 1-Aspect 19, wherein R^{5a} is selected from a group having formula represented by a structure: -R²⁰, -R³⁰-A¹-R⁴⁰, -A¹-R⁴⁰, -A¹-R³⁰-A²-R⁴⁰, or—A¹—R³⁰—A²—R³¹—A³—R⁴⁰; and wherein each of R^{5b}, R^{5c}, R^{5d,} and R^{5e} is independently selected from hydrogen, halogen, —SF₅, —CN, —N₃, —OH, —NH₂, —CF₃, and —CF₂CF₃.

Aspect 21. The compound of Aspect 20, wherein R^{5a} is R²⁰.

Aspect 22. The compound of any one of Aspect 20 or Aspect 21, wherein R²⁰ is selected from -C2-C7 alkylamino and -C2-C7 alkoxy.

Aspect 23. The compound of any one of Aspect 20 or Aspect 21, wherein R²⁰ is halogen.

Aspect 24. The compound of any one of Aspect 1-Aspect 23, wherein each of R^{5b}, R^{5c}, R^{5d}, and R^{5e} is selected from halogen and hydrogen.

Aspect 25. The compound of Aspect 24, wherein each of R^{5b}, R^{5c}, R^{5d}, and R^{5e} is hydrogen.

Aspect 26. The compound of any one of Aspect 1-Aspect 25, wherein R^{5b} is selected from a group having formula represented by a structure: -R²⁰, -R³⁰-A¹-R⁴⁰, -A¹-R⁴⁰, -A¹-R³⁰-A²-R⁴⁰, or—A¹—R³⁰—A²—R³¹—A³—R⁴⁰; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.

Aspect 27. The compound of Aspect 26, wherein R^{5b} is R²⁰.

Aspect 28. The compound of Aspect 26 or Aspect 27, wherein R²⁰ is selected from -C2-C7 alkylamino and -C2-C7 alkoxy.

Aspect 29. The compound of Aspect 26 or Aspect 27, wherein R²⁰ is halogen.

Aspect 30. The compound of any one of Aspect 26-Aspect 29, wherein each of R^{5a}, R^{5c}, R^{5d}, and R^{5e} is selected from halogen and hydrogen.

Aspect 31. The compound of Aspect 30, wherein each of R^{5a}, R^{5c}, R^{5d,} and R^{5e} is hydrogen.

Aspect 32. The compound of any one of Aspect 1-Aspect 31, wherein R^{5c} is selected from a group having formula represented by a structure: -R²⁰, -R³⁰-A¹-R⁴⁰, -A¹-R⁴⁰, -A¹-R³⁰-A²-R⁴⁰, or -A¹-R³⁰-A²-R³¹-A³-R⁴⁰; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.

Aspect 33. The compound of Aspect 32, wherein R^{5c} is R²⁰.

Aspect 34. The compound of Aspect 32 or Aspect 33, wherein R²⁰ is selected from -C2-C7 alkylamino and -C2-C7 alkoxy.

Aspect 35. The compound of Aspect 32 or Aspect 33, wherein R²⁰ is halogen.

Aspect 36. The compound of anyone of Aspect 32-Aspect 35, wherein each of R^{5a}, R^{5b}, R^{5d,} and R^{5e} is selected from halogen and hydrogen.

Aspect 37. The compound of Aspect 36, wherein each of R^{5a}, R^{5b}, R^{5d,} and R^{5e} is hydrogen.

Aspect 38. The compound of any one of Aspect 1-Aspect 37, wherein each of R^{6a}, R^{6b}, R^{6c}, and R^{6d} is independently selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl, provided that at least one of R^{6a}, R^{6b}, R^{6c}, and R^{6d} is not hydrogen.

Aspect 39. The compound of Aspect 38, wherein R^{6a} and R^{6b} are independently selected from hydrogen, halogen, —SF₅, —CN, —N₃, —OH, —NH₂, —CHF₂, —CH₂F, and —CF₃.

Aspect 40. The compound of Aspect 39, wherein R^{6a} and R^{6b} are independently selected from halogen, —SF₅, —CN, —N₃, —OH, —NH₂, —CHF₂, —CH₂F, and —CF₃.

Aspect 41. The compound of Aspect 40, wherein R^{6a} and R^{6b} are independently selected from —F, —Cl, —SF₅, —CN, —N₃, —OH, —NH₂, —CHF₂, —CH₂F, and —CF₃.

Aspect 42. The compound of Aspect 38, wherein R^{6a} and R^{6c} are independently selected from hydrogen, halogen, —SF₅, —CN, —N₃, —OH, —NH₂, —CHF₂, —CH₂F, and —CF₃.

Aspect 43. The compound of Aspect 42, wherein R^{6a} and R^{6c} are independently selected from halogen, —SF₅, —CN, —N₃, —OH, —NH₂, —CHF₂, —CH₂F, and —CF₃.

Aspect 44. The compound of Aspect 43, wherein R^{6a} and R^{6c} are independently selected from —F, —Cl, —SF₅, —CN, —N₃, —OH, —NH₂, —CHF₂, —CH₂F, and —CF₃.

Aspect 45. The compound of Aspect 38, wherein R^{6a} and R^{6d} are independently selected from hydrogen, halogen, —SF₅, —CN, —N₃, —OH, —NH₂, —CHF₂, —CH₂F, and —CF₃.

Aspect 46. The compound of Aspect 45, wherein R^{6a} and R^{6d} are independently selected from halogen, —SF₅, —CN, —N₃, —OH, —NH₂, —CHF₂, —CH₂F, and —CF₃.

Aspect 47. The compound of Aspect 46, wherein R^{6a} and R^{6d} are independently selected from —F, —Cl, —SF₅, —CN, —N₃, —OH, —NH₂, —CHF₂, —CH₂F, and —CF₃.

Aspect 48. The compound of Aspect 38, wherein R^{6a} is selected from —F, —Cl, —SF₅, —CN, —N₃, —OH, and —NH₂.

Aspect 49. The compound of Aspect 38, wherein R^{6a} is selected from —F, —SF₅, —CN, —N₃, —OH, and —NH₂.

Aspect 50. The compound of Aspect 38, wherein R^{6b} is selected from —F, —Cl, —SF₅, —CN, —N₃, —OH, and —NH₂.

Aspect 51. The compound of Aspect 38, wherein R^{6a} is selected from —F, —SF₅, —CN, —N₃, —OH, and —NH₂.

Aspect 52. The compound of any of Aspect 1-Aspect 51, wherein each of R^{6c} and R^{6d} are hydrogen.

Aspect 53. The compound of any one of Aspect 1-Aspect 37, wherein R^{6a} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b}, R^{6c}, and R^{6d} is hydrogen.

Aspect 54. The compound of Aspect 53, wherein R^{6a} is selected from halogen, —SF₅, —CN, —N₃, —OH, —NH₂, —CHF₂, —CH₂F, and —CF₃.

Aspect 55. The compound of Aspect 54, wherein R^{6a} is selected from —F, —Cl, —SF₅, —CN, —N₃, —OH, —NH₂, —CHF₂, —CH₂F, and —CF₃.

Aspect 56. The compound of Aspect 55, wherein R^{6a} is —F.

Aspect 57. The compound of any one of Aspect 1-Aspect 37, wherein R^{6b} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a}, R^{6c}, and R^{6d} is hydrogen.

Aspect 58. The compound of Aspect 57, wherein R^{6b} is selected from halogen, —SF₅, —CN, —N₃, —OH, —NH₂, —CHF₂, —CH₂F, and —CF₃.

Aspect 59. The compound of Aspect 58, wherein R^{6b} is selected from —F, —Cl, —SF₅, —CN, —N₃, —OH, —NH₂, —CHF₂, —CH₂F, and —CF₃.

Aspect 60. The compound of Aspect 59, wherein R^{6b} is —F.

Aspect 61. The compound of any one of Aspect 1-Aspect 37, wherein each of R^{6a} and R^{6b} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6c} and R^{6d} is hydrogen.

Aspect 62. The compound of Aspect 38, wherein each of R^{6a} and R^{6b} is independently selected from halogen, —SF₅, —CN, —N₃, —OH, —NH₂, —CHF₂, —CH₂F, and —CF₃.

Aspect 63. The compound of Aspect 39, wherein each of R^{6a} and R^{6b} is independently selected from —F, —Cl, —SF₅, —CN, —N₃, —OH, —NH₂, —CHF₂, —CH₂F, and —CF₃.

Aspect 64. The compound of Aspect 40, wherein each of R^{6a} and R^{6b} is —F.

Aspect 65. The compound of any one of Aspect 1-Aspect 37, wherein each of R^{6a} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6d} is hydrogen.

Aspect 66. The compound of Aspect 38, wherein each of R^{6a} and R^{6c} is independently selected from halogen, —SF₅, —CN, —N₃, —OH, —NH₂, —CHF₂, —CH₂F, and —CF₃.

Aspect 67. The compound of Aspect 39, wherein each of R^{6a} and R^{6c} is independently selected from —F, —Cl, —SF₅, —CN, —N₃, —OH, —NH₂, —CHF₂, —CH₂F, and —CF₃.

Aspect 68. The compound of Aspect 40, wherein each of R^{6a} and R^{6c} is —F.

Aspect 69. The compound of any one of Aspect 1-Aspect 37, wherein each of R^{6a} and R^{6d} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6c} is hydrogen.

Aspect 70. The compound of Aspect 38, wherein each of R^{6a} and R^{6d} is independently selected from halogen, —SF₅, —CN, —N₃, —OH, —NH₂, —CHF₂, —CH₂F, and —CF₃.

Aspect 71. The compound of Aspect 39, wherein each of R^{6a} and R^{6d} is independently selected from —F, —Cl, —SF₅, —CN, —N₃, —OH, —NH₂, —CHF₂, —CH₂F, and —CF₃.

Aspect 72. The compound of Aspect 40, wherein each of R^{6a} and R^{6d} is —F.

Aspect 73. The compound of any one of Aspect 1-Aspect 37, wherein each of R^{6b} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a} and R^{6d} is hydrogen.

Aspect 74. The compound of Aspect 38, wherein each of R^{6b} and R^{6c} is independently selected from halogen, —SF₅, —CN, —N₃, —OH, —NH₂, —CHF₂, —CH₂F, and —CF₃.

Aspect 75. The compound of Aspect 39, wherein each of R^{6b} and R^{6c} is independently selected from —F, —Cl, —SF₅, —CN, —N₃, —OH, —NH₂, —CHF₂, —CH₂F, and —CF₃.

Aspect 76. The compound of Aspect 40, wherein each of R^{6b} and R^{6c} is —F.

Aspect 77. The compound of any one of Aspect 1-Aspect 76, wherein R²⁰ is selected from hydrogen, -C6-C10 alkyl, -C6-C10 aminoalkyl, and -C6-C10 hydroxyalkyl.

Aspect 78. The compound of any one of Aspect 1-Aspect 76, wherein R²⁰ is selected from hydrogen, -C6-C8 alkyl, -C6-C8 aminoalkyl, and -C6-C8 hydroxyalkyl.

Aspect 79. The compound of any one of Aspect 1-Aspect 76, wherein R²⁰ is selected from hydrogen, -C5-C10 alkyl, -C5-C10 aminoalkyl, and -C5-C10 hydroxyalkyl.

Aspect 80. The compound of any one of Aspect 1-Aspect 76, wherein R²⁰ is selected from hydrogen, -C5-C8 alkyl, -C5-C8 aminoalkyl, and -C5-C8 hydroxyalkyl.

Aspect 81. The compound of any one of Aspect 1-Aspect 76, wherein R²⁰ is selected from hydrogen, -C5-C6 alkyl, -C5-C6 aminoalkyl, and -C5-C6 hydroxyalkyl.

Aspect 82. The compound of any one of Aspect 1-Aspect 76, wherein R²⁰ is selected from hydrogen, -C4-C10 alkyl, —C4-C10 aminoalkyl, and —C4-C10 hydroxyalkyl.

Aspect 83. The compound of any one of Aspect 1-Aspect 76, wherein R²⁰ is selected from hydrogen, -C4-C8 alkyl, -C4-C8 aminoalkyl, and —C4-C8 hydroxyalkyl.

Aspect 84. The compound of any one of Aspect 1-Aspect 76, wherein R²⁰ is selected from hydrogen, -C4-C6 alkyl, -C4-C6 aminoalkyl, and —C4-C6 hydroxyalkyl.

Aspect 85. The compound of any one of Aspect 1-Aspect 76, wherein R²⁰ is selected from hydrogen, -C4-C5 alkyl, -C4-C5 aminoalkyl, and —C4-C5 hydroxyalkyl.

Aspect 86. The compound of any one of Aspect 1-Aspect 76, wherein R²⁰ is selected from hydrogen, -C3-C10 alkyl, -C3-C10 aminoalkyl, and -C3-C10 hydroxyalkyl.

Aspect 87. The compound of any one of Aspect 1-Aspect 76, wherein R²⁰ is selected from hydrogen, -C3-C8 alkyl, -C3-C8 aminoalkyl, and -C3-C8 hydroxyalkyl.

Aspect 88. The compound of any one of Aspect 1-Aspect 76, wherein R²⁰ is selected from hydrogen, -C3-C6 alkyl, -C3-C6 aminoalkyl, and -C3-C6 hydroxyalkyl.

Aspect 89. The compound of any one of Aspect 1-Aspect 76, wherein R²⁰ is selected from hydrogen, -C3-C5 alkyl, -C3-C5 aminoalkyl, and —C3-C5 hydroxyalkyl.

Aspect 90. The compound of any one of Aspect 1-Aspect 76, wherein R²⁰ is selected from hydrogen, -C3-C4 alkyl, -C3-C4 aminoalkyl, and —C3-C4 hydroxyalkyl.

Aspect 91. The compound of any one of Aspect 1-Aspect 76, wherein R²⁰ is selected from hydrogen, -C2-C10 alkyl, -C2-C10 aminoalkyl, and —C2-C10 hydroxyalkyl.

Aspect 92. The compound of any one of Aspect 1-Aspect 76, wherein R²⁰ is selected from hydrogen, -C2-C8 alkyl, -C2-C8 aminoalkyl, and -C2-C8 hydroxyalkyl.

Aspect 93. The compound of any one of Aspect 1-Aspect 76, wherein R²⁰ is selected from hydrogen, -C2-C6 alkyl, -C3-C6 aminoalkyl, and -C2-C6 hydroxyalkyl.

Aspect 94. The compound of any one of Aspect 1-Aspect 76, wherein R²⁰ is selected from hydrogen, —C2-C5 alkyl, -C2-C5 aminoalkyl, and —C2-C5 hydroxyalkyl.

Aspect 95. The compound of any one of Aspect 1-Aspect 76, wherein R²⁰ is selected from hydrogen, —C2-C4 alkyl, —C2-C4 aminoalkyl, and —C2-C4 hydroxyalkyl.

Aspect 96. The compound of any one of Aspect 1-Aspect 76, wherein R²⁰ is selected from hydrogen, —C2-C3 alkyl, —C2-C3 aminoalkyl, and —C2-C3 hydroxyalkyl.

Aspect 97. The compound of any one of Aspect 1-Aspect 76, wherein each of R³⁰ and R³¹ is independently selected from hydrogen, —C6-C10 alkanediyl, —C6-C10 aminoalkanediyl, and —C6-C10 hydroxyalkanediyl.

Aspect 98. The compound of any one of Aspect 1-Aspect 76, wherein each of R³⁰ and R³¹ is independently selected from hydrogen, -C6-C8 alkanediyl, -C6-C8 aminoalkanediyl, and -C6-C8 hydroxyalkanediyl.

Aspect 99. The compound of any one of Aspect 1-Aspect 76, wherein each of R³⁰ and R³¹ is independently selected from hydrogen, -C5-C10 alkanediyl, -C5-C10 aminoalkanediyl, and —C5-C10 hydroxyalkanediyl.

Aspect 100. The compound of any one of Aspect 1-Aspect 76, wherein each of R³⁰ and R³¹ is independently selected from hydrogen, -C5-C8 alkanediyl, -C5-C8 aminoalkanediyl, and -C5-C8 hydroxyalkanediyl.

Aspect 101. The compound of any one of Aspect 1-Aspect 76, wherein each of R³⁰ and R³¹ is independently selected from hydrogen, —C5-C6 alkanediyl, -C5-C6 aminoalkanediyl, and -C5-C6 hydroxyalkanediyl.

Aspect 102. The compound of any one of Aspect 1-Aspect 76, wherein each of R³⁰ and R³¹ is independently selected from hydrogen, —C4-C10 alkanediyl, —C4-C10 aminoalkanediyl, and —C4-C10 hydroxyalkanediyl.

Aspect 103. The compound of any one of Aspect 1-Aspect 76, wherein each of R³⁰ and R³¹ is independently selected from hydrogen, -C4-C8 alkanediyl, -C4-C8 aminoalkanediyl, and -C4-C8 hydroxyalkanediyl.

Aspect 104. The compound of any one of Aspect 1-Aspect 76, wherein each of R³⁰ and R³¹ is independently selected from hydrogen, -C4-C6 alkanediyl, -C4-C6 aminoalkanediyl, and -C4-C6 hydroxyalkanediyl.

Aspect 105. The compound of any one of Aspect 1-Aspect 76, wherein each of R³⁰ and R³¹ is independently selected from hydrogen, -C4-C5 alkanediyl, -C4-C5 aminoalkanediyl, and -C4-C5 hydroxyalkanediyl.

Aspect 106. The compound of any one of Aspect 1-Aspect 76, wherein each of R³⁰ and R³¹ is independently selected from hydrogen, —C3-C10 alkanediyl, —C3-C10 aminoalkanediyl, and —C3-C10 hydroxyalkanediyl.

Aspect 107. The compound of any one of Aspect 1-Aspect 76, wherein each of R³⁰ and R³¹ is independently selected from hydrogen, -C3-C8 alkanediyl, -C3-C8 aminoalkanediyl, and -C3-C8 hydroxyalkanediyl.

Aspect 108. The compound of any one of Aspect 1-Aspect 76, wherein each of R³⁰ and R³¹ is independently selected from hydrogen, -C3-C6 alkanediyl, -C3-C6 aminoalkanediyl, and -C3-C6 hydroxyalkanediyl.

Aspect 109. The compound of any one of Aspect 1-Aspect 76, wherein each of R³⁰ and R³¹ is independently selected from hydrogen, -C3-C5 alkanediyl, -C3-C5 aminoalkanediyl, and -C3-C5 hydroxyalkanediyl.

Aspect 110. The compound of any one of Aspect 1-Aspect 76, wherein each of R³⁰ and R³¹ is independently selected from hydrogen, -C3-C4 alkanediyl, -C3-C4 aminoalkanediyl, and -C3-C4 hydroxyalkanediyl.

Aspect 111. The compound of any one of Aspect 1-Aspect 76, wherein each of R³⁰ and R³¹ is independently selected from hydrogen, -C2-C10 alkanediyl, -C2-C10 aminoalkanediyl, and —C2-C10 hydroxyalkanediyl.

Aspect 112. The compound of any one of Aspect 1-Aspect 76, wherein each of R³⁰ and R³¹ is independently selected from hydrogen, —C2-C8 alkanediyl, —C2-C8 aminoalkanediyl, and —C2-C8 hydroxyalkanediyl.

Aspect 113. The compound of any one of Aspect 1-Aspect 76, wherein each of R³⁰ and R³¹ is independently selected from hydrogen, —C2-C6 alkanediyl, -C3-C6 aminoalkanediyl, and -C2-C6 hydroxyalkanediyl.

Aspect 114. The compound of any one of Aspect 1-Aspect 76, wherein each of R³⁰ and R³¹ is independently selected from hydrogen, —C2-C5 alkanediyl, -C2-C5 aminoalkanediyl, and -C2-C5 hydroxyalkanediyl.

Aspect 115. The compound of any one of Aspect 1-Aspect 76, wherein each of R³⁰ and R³¹ is independently selected from hydrogen, —C2-C4 alkanediyl, —C2-C4 aminoalkanediyl, and —C2-C4 hydroxyalkanediyl.

Aspect 116. The compound of any one of Aspect 1-Aspect 76, wherein each of R³⁰ and R³¹ is independently selected from hydrogen, —C2-C3 alkanediyl, -C2-C3 aminoalkanediyl, and —C2-C3 hydroxyalkanediyl.

Aspect 117. The compound of any one of Aspect 1-Aspect 116, wherein R⁴⁰ is selected from hydrogen, -C6-C10 alkyl, -C6-C10 aminoalkyl, and —C6-C10 hydroxyalkyl.

Aspect 118. The compound of any one of Aspect 1-Aspect 116, wherein R⁴⁰ is selected from hydrogen, -C6-C8 alkyl, -C6-C8 aminoalkyl, and -C6-C8 hydroxyalkyl.

Aspect 119. The compound of any one of Aspect 1-Aspect 116, wherein R⁴⁰ is selected from hydrogen, -C5-C10 alkyl, -C5-C10 aminoalkyl, and —C5-C10 hydroxyalkyl.

Aspect 120. The compound of any one of Aspect 1-Aspect 116, wherein R⁴⁰ is selected from hydrogen, -C5-C8 alkyl, -C5-C8 aminoalkyl, and -C5-C8 hydroxyalkyl.

Aspect 121. The compound of any one of Aspect 1-Aspect 116, wherein R⁴⁰ is selected from hydrogen, -C5-C6 alkyl, -C5-C6 aminoalkyl, and -C5-C6 hydroxyalkyl.

Aspect 122. The compound of any one of Aspect 1-Aspect 116, wherein R⁴⁰ is selected from hydrogen, -C4-C10 alkyl, -C4-C10 aminoalkyl, and -C4-C10 hydroxyalkyl.

Aspect 123. The compound of any one of Aspect 1-Aspect 116, wherein R⁴⁰ is selected from hydrogen, -C4-C8 alkyl, -C4-C8 aminoalkyl, and —C4-C8 hydroxyalkyl.

Aspect 124. The compound of any one of Aspect 1-Aspect 116, wherein R⁴⁰ is selected from hydrogen, -C4-C6 alkyl, -C4-C6 aminoalkyl, and —C4-C6 hydroxyalkyl.

Aspect 125. The compound of any one of Aspect 1-Aspect 116, wherein R⁴⁰ is selected from hydrogen, -C4-C5 alkyl, -C4-C5 aminoalkyl, and —C4-C5 hydroxyalkyl.

Aspect 126. The compound of any one of Aspect 1-Aspect 116, wherein R⁴⁰ is selected from hydrogen, -C3-C10 alkyl, -C3-C10 aminoalkyl, and -C3-C10 hydroxyalkyl.

Aspect 127. The compound of any one of Aspect 1-Aspect 116, wherein R⁴⁰ is selected from hydrogen, -C3-C8 alkyl, -C3-C8 aminoalkyl, and -C3-C8 hydroxyalkyl.

Aspect 128. The compound of any one of Aspect 1-Aspect 116, wherein R⁴⁰ is selected from hydrogen, -C3-C6 alkyl, -C3-C6 aminoalkyl, and —C3-C6 hydroxyalkyl.

Aspect 129. The compound of any one of Aspect 1-Aspect 116, wherein R⁴⁰ is selected from hydrogen, -C3-C5 alkyl, -C3-C5 aminoalkyl, and —C3-C5 hydroxyalkyl.

Aspect 130. The compound of any one of Aspect 1-Aspect 116, wherein R⁴⁰ is selected from hydrogen, -C3-C4 alkyl, -C3-C4 aminoalkyl, and —C3-C4 hydroxyalkyl.

Aspect 131. The compound of any one of Aspect 1-Aspect 116, wherein R⁴⁰ is selected from hydrogen, -C2-C10 alkyl, -C2-C10 aminoalkyl, and -C2-C10 hydroxyalkyl.

Aspect 132. The compound of any one of Aspect 1-Aspect 116, wherein R⁴⁰ is selected from hydrogen, -C2-C8 alkyl, -C2-C8 aminoalkyl, and -C2-C8 hydroxyalkyl.

Aspect 133. The compound of any one of Aspect 1-Aspect 116, wherein R⁴⁰ is selected from hydrogen, —C2-C6 alkyl, -C3-C6 aminoalkyl, and —C2-C6 hydroxyalkyl.

Aspect 134. The compound of any one of Aspect 1-Aspect 116, wherein R⁴⁰ is selected from hydrogen, —C2-C5 alkyl, -C2-C5 aminoalkyl, and —C2-C5 hydroxyalkyl.

Aspect 135. The compound of any one of Aspect 1-Aspect 116, wherein R⁴⁰ is selected from hydrogen, —C2-C4 alkyl, —C2-C4 aminoalkyl, and —C2-C4 hydroxyalkyl.

Aspect 136. The compound of any one of Aspect 1-Aspect 116, wherein R⁴⁰ is selected from hydrogen, -C2-C3 alkyl, -C2-C3 aminoalkyl, and -C2-C3 hydroxyalkyl.

Aspect 137. The compound of any one of Aspect 1-Aspect 136, wherein R⁵⁰ is selected from hydrogen, -C6-C10 alkyl, -C6-C10 aminoalkyl, and —C6-C10 hydroxyalkyl.

Aspect 138. The compound of any one of Aspect 1-Aspect 136, wherein R⁵⁰ is selected from hydrogen, -C6-C8 alkyl, -C6-C8 aminoalkyl, and -C6-C8 hydroxyalkyl.

Aspect 139. The compound of any one of Aspect 1-Aspect 136, wherein R⁵⁰ is selected from hydrogen, -C5-C10 alkyl, -C5-C10 aminoalkyl, and -C5-C10 hydroxyalkyl.

Aspect 140. The compound of any one of Aspect 1-Aspect 136, wherein R⁵⁰ is selected from hydrogen, -C5-C8 alkyl, -C5-C8 aminoalkyl, and -C5-C8 hydroxyalkyl.

Aspect 141. The compound of any one of Aspect 1-Aspect 136, wherein R⁵⁰ is selected from hydrogen, -C5-C6 alkyl, -C5-C6 aminoalkyl, and -C5-C6 hydroxyalkyl.

Aspect 142. The compound of any one of Aspect 1-Aspect 136, wherein R⁵⁰ is selected from hydrogen, -C4-C10 alkyl, —C4-C10 aminoalkyl, and —C4-C10 hydroxyalkyl.

Aspect 143. The compound of any one of Aspect 1-Aspect 136, wherein R⁵⁰ is selected from hydrogen, -C4-C8 alkyl, -C4-C8 aminoalkyl, and —C4-C8 hydroxyalkyl.

Aspect 144. The compound of any one of Aspect 1-Aspect 136, wherein R⁵⁰ is selected from hydrogen, -C4-C6 alkyl, -C4-C6 aminoalkyl, and —C4-C6 hydroxyalkyl.

Aspect 145. The compound of any one of Aspect 1-Aspect 136, wherein R⁵⁰ is selected from hydrogen, -C4-C5 alkyl, -C4-C5 aminoalkyl, and —C4-C5 hydroxyalkyl.

Aspect 146. The compound of any one of Aspect 1-Aspect 136, wherein R⁵⁰ is selected from hydrogen, -C3-C10 alkyl, -C3-C10 aminoalkyl, and -C3-C10 hydroxyalkyl.

Aspect 147. The compound of any one of Aspect 1-Aspect 136, wherein R⁵⁰ is selected from hydrogen, -C3-C8 alkyl, -C3-C8 aminoalkyl, and -C3-C8 hydroxyalkyl.

Aspect 148. The compound of any one of Aspect 1-Aspect 136, wherein R⁵⁰ is selected from hydrogen, -C3-C6 alkyl, -C3-C6 aminoalkyl, and -C3-C6 hydroxyalkyl.

Aspect 149. The compound of any one of Aspect 1-Aspect 136, wherein R⁵⁰ is selected from hydrogen, -C3-C5 alkyl, -C3-C5 aminoalkyl, and —C3-C5 hydroxyalkyl.

Aspect 150. The compound of any one of Aspect 1-Aspect 136, wherein R⁵⁰ is selected from hydrogen, -C3-C4 alkyl, -C3-C4 aminoalkyl, and —C3-C4 hydroxyalkyl.

Aspect 151. The compound of any one of Aspect 1-Aspect 136, wherein R⁵⁰ is selected from hydrogen, -C2-C10 alkyl, -C2-C10 aminoalkyl, and —C2-C10 hydroxyalkyl.

Aspect 152. The compound of any one of Aspect 1-Aspect 136, wherein R⁵⁰ is selected from hydrogen, -C2-C8 alkyl, -C2-C8 aminoalkyl, and -C2-C8 hydroxyalkyl.

Aspect 153. The compound of any one of Aspect 1-Aspect 136, wherein R⁵⁰ is selected from hydrogen, —C2-C6 alkyl, -C3-C6 aminoalkyl, and —C2-C6 hydroxyalkyl.

Aspect 154. The compound of any one of Aspect 1-Aspect 136, wherein R⁵⁰ is selected from hydrogen, —C2-C5 alkyl, -C2-C5 aminoalkyl, and —C2-C5 hydroxyalkyl.

Aspect 155. The compound of any one of Aspect 1-Aspect 136, wherein R⁵⁰ is selected from hydrogen, —C2-C4 alkyl, —C2-C4 aminoalkyl, and —C2-C4 hydroxyalkyl.

Aspect 156. The compound of any one of Aspect 1-Aspect 136, wherein R⁵⁰ is selected from hydrogen, —C2-C3 alkyl, —C2-C3 aminoalkyl, and —C2-C3 hydroxyalkyl.

Aspect 157. The compound of any one of Aspect 1-Aspect 156, wherein R⁶⁰ is selected from hydrogen, -C6-C10 alkyl, -C6-C10 aminoalkyl, and -C6-C10 hydroxyalkyl.

Aspect 158. The compound of any one of Aspect 1-Aspect 156, wherein R⁶⁰ is selected from hydrogen, -C6-C8 alkyl, -C6-C8 aminoalkyl, and -C6-C8 hydroxyalkyl.

Aspect 159. The compound of any one of Aspect 1-Aspect 156, wherein R⁶⁰ is selected from hydrogen, -C5-C10 alkyl, -C5-C10 aminoalkyl, and -C5-C10 hydroxyalkyl.

Aspect 160. The compound of any one of Aspect 1-Aspect 156, wherein R⁶⁰ is selected from hydrogen, -C5-C8 alkyl, -C5-C8 aminoalkyl, and -C5-C8 hydroxyalkyl.

Aspect 161. The compound of any one of Aspect 1-Aspect 156, wherein R⁶⁰ is selected from hydrogen, -C5-C6 alkyl, -C5-C6 aminoalkyl, and -C5-C6 hydroxyalkyl.

Aspect 162. The compound of any one of Aspect 1-Aspect 156, wherein R⁶⁰ is selected from hydrogen, -C4-C10 alkyl, —C4-C10 aminoalkyl, and —C4-C10 hydroxyalkyl.

Aspect 163. The compound of any one of Aspect 1-Aspect 156, wherein R⁶⁰ is selected from hydrogen, -C4-C8 alkyl, -C4-C8 aminoalkyl, and —C4-C8 hydroxyalkyl.

Aspect 164. The compound of any one of Aspect 1-Aspect 156, wherein R⁶⁰ is selected from hydrogen, -C4-C6 alkyl, -C4-C6 aminoalkyl, and —C4-C6 hydroxyalkyl.

Aspect 165. The compound of any one of Aspect 1-Aspect 156, wherein R⁶⁰ is selected from hydrogen, -C4-C5 alkyl, -C4-C5 aminoalkyl, and —C4-C5 hydroxyalkyl.

Aspect 166. The compound of any one of Aspect 1-Aspect 156, wherein R⁶⁰ is selected from hydrogen, -C3-C10 alkyl, -C3-C10 aminoalkyl, and -C3-C10 hydroxyalkyl.

Aspect 167. The compound of any one of Aspect 1-Aspect 136, wherein R⁶⁰ is selected from hydrogen, -C3-C8 alkyl, -C3-C8 aminoalkyl, and -C3-C8 hydroxyalkyl.

Aspect 168. The compound of any one of Aspect 1-Aspect 156, wherein R⁶⁰ is selected from hydrogen, -C3-C6 alkyl, -C3-C6 aminoalkyl, and -C3-C6 hydroxyalkyl.

Aspect 169. The compound of any one of Aspect 1-Aspect 156, wherein R⁶⁰ is selected from hydrogen, -C3-C5 alkyl, -C3-C5 aminoalkyl, and —C3-C5 hydroxyalkyl.

Aspect 170. The compound of any one of Aspect 1-Aspect 156, wherein R⁶⁰ is selected from hydrogen, -C3-C4 alkyl, -C3-C4 aminoalkyl, and —C3-C4 hydroxyalkyl.

Aspect 171. The compound of any one of Aspect 1-Aspect 156, wherein R⁶⁰ is selected from hydrogen, -C2-C10 alkyl, -C2-C10 aminoalkyl, and -C2-C10 hydroxyalkyl.

Aspect 172. The compound of any one of Aspect 1-Aspect 156, wherein R⁶⁰ is selected from hydrogen, —C2-C8 alkyl, —C2-C8 aminoalkyl, and —C2-C8 hydroxyalkyl.

Aspect 173. The compound of any one of Aspect 1-Aspect 156, wherein R⁶⁰ is selected from hydrogen, -C2-C6 alkyl, -C3-C6 aminoalkyl, and -C2-C6 hydroxyalkyl.

Aspect 174. The compound of any one of Aspect 1-Aspect 156, wherein R⁶⁰ is selected from hydrogen, —C2-C5 alkyl, -C2-C5 aminoalkyl, and —C2-C5 hydroxyalkyl.

Aspect 175. The compound of any one of Aspect 1-Aspect 156, wherein R⁶⁰ is selected from hydrogen, —C2-C4 alkyl, —C2-C4 aminoalkyl, and —C2-C4 hydroxyalkyl.

Aspect 176. The compound of any one of Aspect 1-Aspect 156, wherein R⁶⁰ is selected from hydrogen, —C2-C3 alkyl, —C2-C3 aminoalkyl, and —C2-C3 hydroxyalkyl.

Aspect 177. The compound of any one of Aspect 1-Aspect 176, wherein R⁷⁰ is selected from hydrogen, -C6-C10 alkyl, -C6-C10 aminoalkyl, and -C6-C10 hydroxyalkyl.

Aspect 178. The compound of any one of Aspect 1-Aspect 176, wherein R⁷⁰ is selected from hydrogen, -C6-C8 alkyl, -C6-C8 aminoalkyl, and -C6-C8 hydroxyalkyl.

Aspect 179. The compound of any one of Aspect 1-Aspect 176, wherein R⁷⁰ is selected from hydrogen, -C5-C10 alkyl, -C5-C10 aminoalkyl, and -C5-C10 hydroxyalkyl.

Aspect 180. The compound of any one of Aspect 1-Aspect 176, wherein R⁷⁰ is selected from hydrogen, -C5-C8 alkyl, -C5-C8 aminoalkyl, and -C5-C8 hydroxyalkyl.

Aspect 181. The compound of any one of Aspect 1-Aspect 176, wherein R⁷⁰ is selected from hydrogen, -C5-C6 alkyl, -C5-C6 aminoalkyl, and -C5-C6 hydroxyalkyl.

Aspect 182. The compound of any one of Aspect 1-Aspect 176, wherein R⁷⁰ is selected from hydrogen, -C4-C10 alkyl, -C4-C10 aminoalkyl, and -C4-C10 hydroxyalkyl.

Aspect 183. The compound of any one of Aspect 1-Aspect 176, wherein R⁷⁰ is selected from hydrogen, -C4-C8 alkyl, -C4-C8 aminoalkyl, and —C4-C8 hydroxyalkyl.

Aspect 184. The compound of any one of Aspect 1-Aspect 176, wherein R⁷⁰ is selected from hydrogen, -C4-C6 alkyl, -C4-C6 aminoalkyl, and —C4-C6 hydroxyalkyl.

Aspect 185. The compound of any one of Aspect 1-Aspect 176, wherein R⁷⁰ is selected from hydrogen, -C4-C5 alkyl, -C4-C5 aminoalkyl, and —C4-C5 hydroxyalkyl.

Aspect 186. The compound of any one of Aspect 1-Aspect 176, wherein R⁷⁰ is selected from hydrogen, -C3-C10 alkyl, -C3-C10 aminoalkyl, and -C3-C10 hydroxyalkyl.

Aspect 187. The compound of any one of Aspect 1-Aspect 176, wherein R⁷⁰ is selected from hydrogen, -C3-C8 alkyl, -C3-C8 aminoalkyl, and -C3-C8 hydroxyalkyl.

Aspect 188. The compound of any one of Aspect 1-Aspect 176, wherein R⁷⁰ is selected from hydrogen, -C3-C6 alkyl, -C3-C6 aminoalkyl, and —C3-C6 hydroxyalkyl.

Aspect 189. The compound of any one of Aspect 1-Aspect 176, wherein R⁷⁰ is selected from hydrogen, -C3-C5 alkyl, -C3-C5 aminoalkyl, and —C3-C5 hydroxyalkyl.

Aspect 190. The compound of any one of Aspect 1-Aspect 176, wherein R⁷⁰ is selected from hydrogen, -C3-C4 alkyl, -C3-C4 aminoalkyl, and —C3-C4 hydroxyalkyl.

Aspect 191. The compound of any one of Aspect 1-Aspect 176, wherein R⁷⁰ is selected from hydrogen, -C2-C10 alkyl, -C2-C10 aminoalkyl, and -C2-C10 hydroxyalkyl.

Aspect 192. The compound of any one of Aspect 1-Aspect 176, wherein R⁷⁰ is selected from hydrogen, —C2-C8 alkyl, —C2-C8 aminoalkyl, and —C2-C8 hydroxyalkyl.

Aspect 193. The compound of any one of Aspect 1-Aspect 176, wherein R⁷⁰ is selected from hydrogen, -C2-C6 alkyl, -C3-C6 aminoalkyl, and -C2-C6 hydroxyalkyl.

Aspect 194. The compound of any one of Aspect 1-Aspect 176, wherein R⁷⁰ is selected from hydrogen, —C2-C5 alkyl, -C2-C5 aminoalkyl, and —C2-C5 hydroxyalkyl.

Aspect 195. The compound of any one of Aspect 1-Aspect 176, wherein R⁷⁰ is selected from hydrogen, —C2-C4 alkyl, —C2-C4 aminoalkyl, and —C2-C4 hydroxyalkyl.

Aspect 196. The compound of any one of Aspect 1-Aspect 176, wherein R⁷⁰ is selected from hydrogen, -C2-C3 alkyl, -C2-C3 aminoalkyl, and -C2-C3 hydroxyalkyl.

Aspect 197. The compound of any one of Aspect 1-Aspect 196, wherein A¹ is selected from —O—, —NH—, —NCH₃—, —NCH₂CH₃—, —N(CH2)2CH₃—, —NCH(CH₃)2—, —N(CH2)3CH₃—, and —N(CH2)4CH₃—.

Aspect 198. The compound of Aspect 197, wherein A¹ is selected from —O—, —NH—, —NCH₃—, and —NCH₂CH₃—.

Aspect 199. The compound of Aspect 197, wherein A¹ is —O—.

Aspect 200. The compound of Aspect 197, wherein A¹ is —NH—.

Aspect 201. The compound of Aspect 197, wherein A¹ is —NCH₃—.

Aspect 202. The compound of Aspect 197, wherein A¹ is —NCH₂CH₃—.

Aspect 203. The compound of any one of Aspect 1-Aspect 202, wherein A² is selected from —O—, —NH—, —NCH₃—, —NCH₂CH₃—, —N(CH2)2CH₃—, —NCH(CH₃)2—, —N(CH2)3CH₃—, and —N(CH2)4CH₃—.

Aspect 204. The compound of Aspect 203, wherein A² is selected from —O—, —NH—, —NCH₃—, and —NCH₂CH₃—.

Aspect 205. The compound of Aspect 203, wherein A² is —O—.

Aspect 206. The compound of Aspect 203, wherein A² is —NH—.

Aspect 207. The compound of Aspect 203, wherein A² is —NCH₃—.

Aspect 208. The compound of Aspect 203, wherein A² is —NCH₂CH₃—.

Aspect 209. The compound of any one of Aspect 1-Aspect 208, wherein A³ is selected from —O—, —NH—, —NCH₃—, —NCH₂CH₃—, —N(CH2)2CH₃—, —NCH(CH₃)2—, —N(CH2)3CH₃—, and —N(CH2)4CH₃—.

Aspect 210. The compound of Aspect 209, wherein A³ is selected from —O—, —NH—, —NCH₃—, and —NCH₂CH₃—.

Aspect 211. The compound of Aspect 209, wherein A³ is —O—.

Aspect 212. The compound of Aspect 209, wherein A³ is —NH—.

Aspect 213. The compound of Aspect 209, wherein A³ is —NCH₃—.

Aspect 214. The compound of Aspect 209, wherein A³ is —NCH₂CH₃—.

Aspect 215. The compound of any one of Aspect 1-Aspect 214, wherein Ar¹ is an unsubstituted phenyl group.

Aspect 216. The compound of any one of Aspect 1-Aspect 214, wherein Ar¹ is a phenyl group substituted with one group selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl.

Aspect 217. The compound of Aspect 216, wherein Ar¹ is a phenyl group substituted with one group selected from halogen, —SF₅, —CN, —N₃, —OH, —NH₂, —OCH₃, —NHCH₃, —N(CH₃)₂, —CH₂OH, —CH₃, —CH₂Cl, —CHCl₂, —CCl₃, —CHF₂, —CH₂F, and —CF₃.

Aspect 218. The compound of Aspect 216, wherein Ar¹ is a phenyl group substituted with one group selected from halogen, —SF₅, —CN, —N₃, —OH, —NH₂, —CHF₂, —CH₂F, and —CF₃.

Aspect 219. The compound of Aspect 216, wherein Ar¹ is a phenyl group substituted with one group selected from —F, —Cl, —SF₅, —CN, —N₃, —OH, —NH₂, —CHF₂, —CH₂F, and —CF₃.

Aspect 220. The compound of any one of Aspect 1-Aspect 214, wherein Ar¹ is a phenyl group substituted with two groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl.

Aspect 221. The compound of Aspect 220, wherein Ar¹ is a phenyl group substituted with two groups independently selected from halogen, —SF₅, —CN, —N₃, —OH, —NH₂, —OCH₃, —NHCH₃, —N(CH₃)₂, —CH₂OH, —CH₃, —CH₂Cl, —CHCl₂, —CCl₃, —CHF₂, —CH₂F, and —CF₃.

Aspect 222. The compound of Aspect 220, wherein Ar¹ is a phenyl group substituted with two groups independently selected halogen, —SF₅, —CN, —N₃, —OH, —NH₂, —CHF₂, —CH₂F, and —CF₃.

Aspect 223. The compound of Aspect 220, wherein Ar¹ is a phenyl group substituted two groups independently selected from —F, —Cl, —SF₅, —CN, —N₃, —OH, —NH₂, —CHF₂, —CH₂F, and —CF₃.

Aspect 224. The compound of any one of Aspect 1-Aspect 214, wherein Ar¹ is a phenyl group substituted with three groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl.

Aspect 225. The compound of Aspect 224, wherein Ar¹ is a phenyl group substituted with three groups independently selected from halogen, —SF₅, —CN, —N₃, —OH, —NH₂, —OCH₃, —NHCH₃, —N(CH₃)₂, —CH₂OH, —CH₃, —CH₂Cl, —CHCl₂, —CCl₃, —CHF₂, —CH₂F, and —CF₃.

Aspect 226. The compound of Aspect 224, wherein Ar¹ is a phenyl group substituted with three groups independently selected halogen, —SF₅, —CN, —N₃, —OH, —NH₂, —CHF₂, —CH₂F, and —CF₃.

Aspect 227. The compound of Aspect 224, wherein Ar¹ is a phenyl group substituted three groups independently selected from —F, —Cl, —SF₅, —CN, —N₃, —OH, —NH₂, —CHF₂, —CH₂F, and —CF₃.

Aspect 228. The compound of Aspect 1, having a structure represented by a formula:

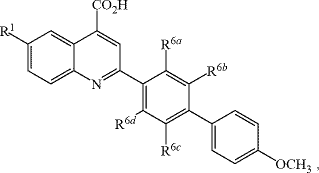

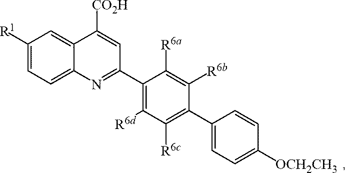

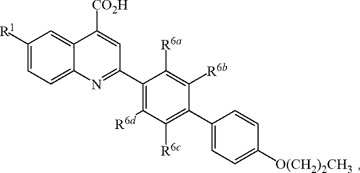

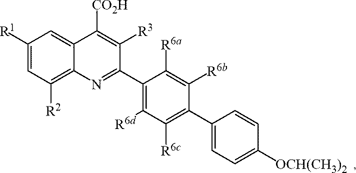

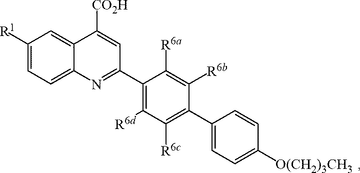

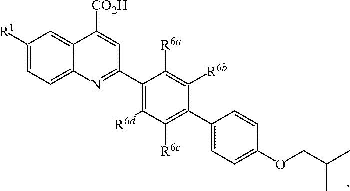

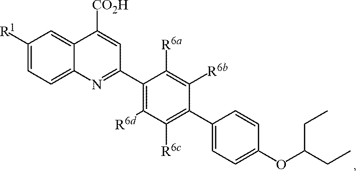

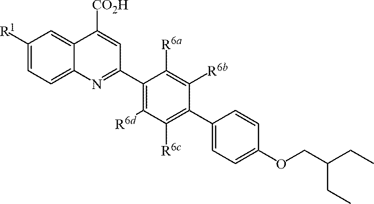

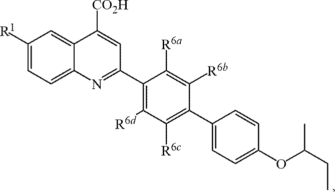

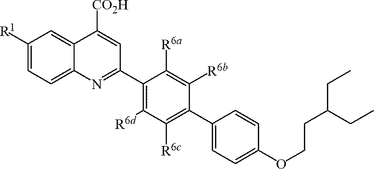

or combinations thereof.

Aspect 229. The compound of Aspect 1, having a structure represented by a formula:

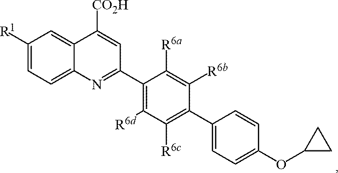

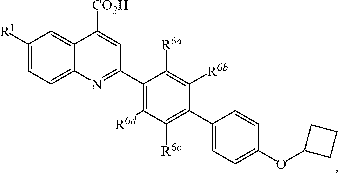

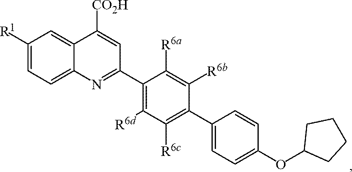

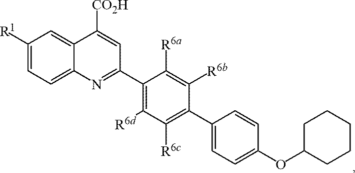

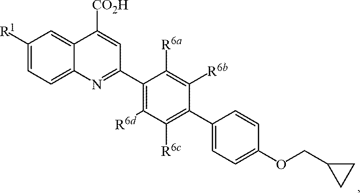

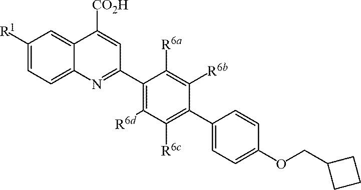

or combinations thereof.

Aspect 230. The compound of Aspect 1, having a structure represented by a formula:

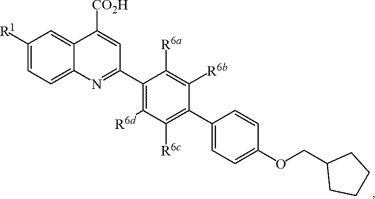

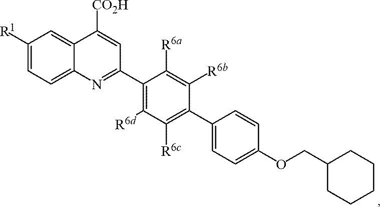

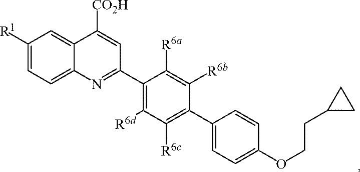

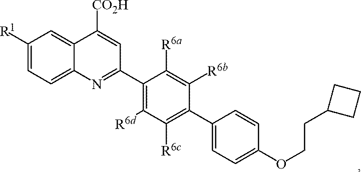

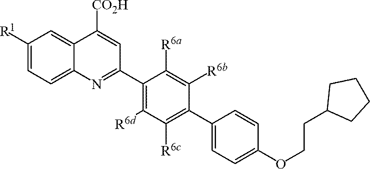

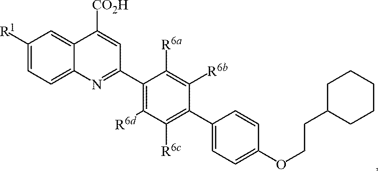

or combinations thereof.

Aspect 231. The compound of Aspect 1, having a structure represented by a formula:

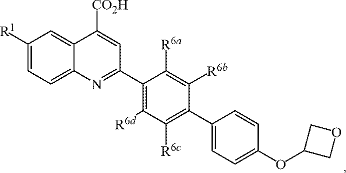

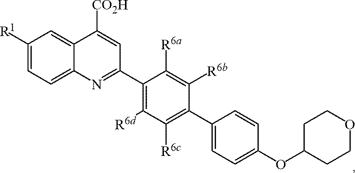

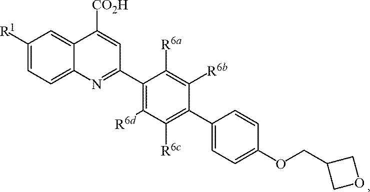

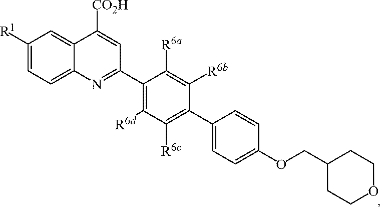

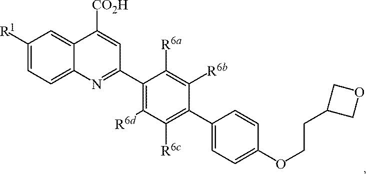

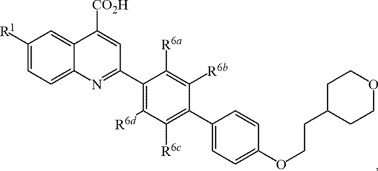

or combinations thereof.

Aspect 232. The compound of Aspect 1, having a structure represented by a formula:

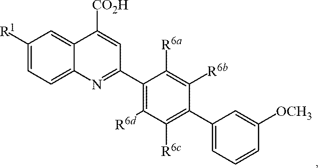

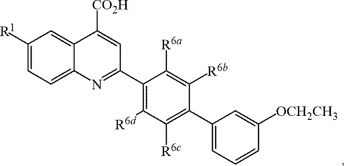

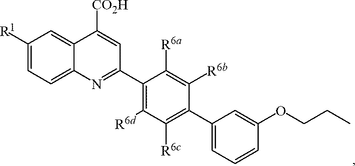

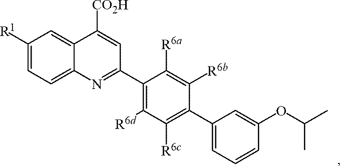

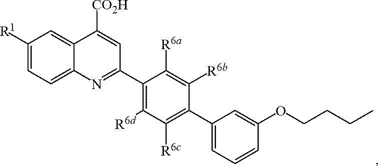

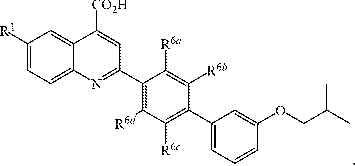

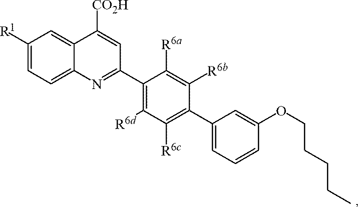

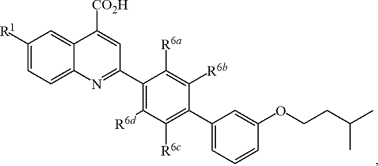

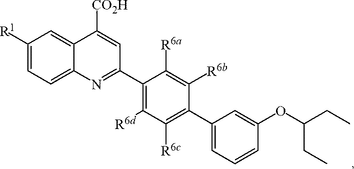

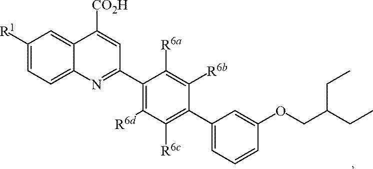

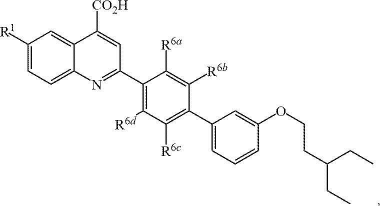

or combinations thereof.

Aspect 233. The compound of Aspect 1, having a structure represented by a formula:

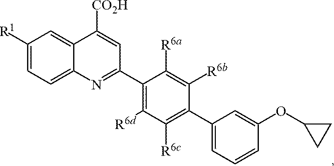

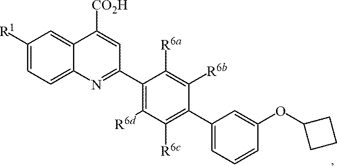

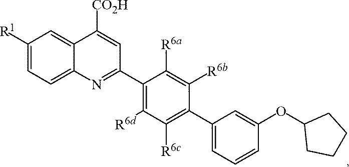

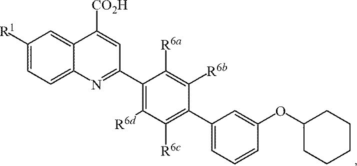

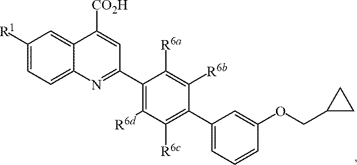

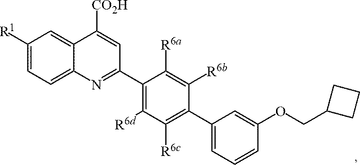

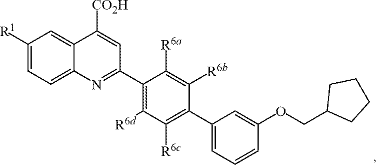

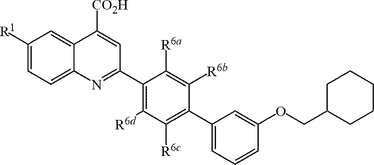

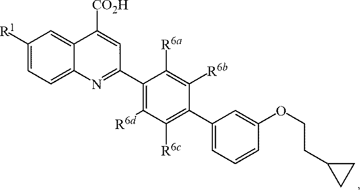

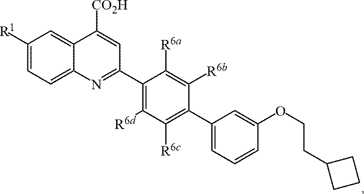

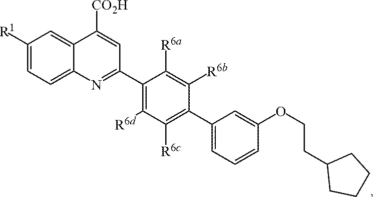

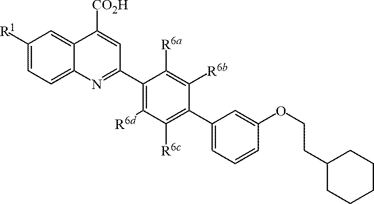

or combinations thereof.

Aspect 234. The compound of Aspect 1, having a structure represented by a formula:

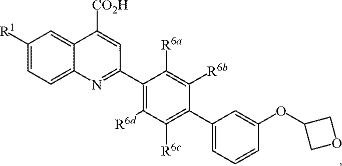

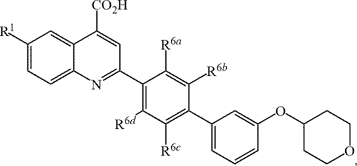

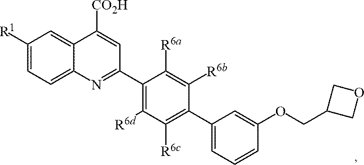

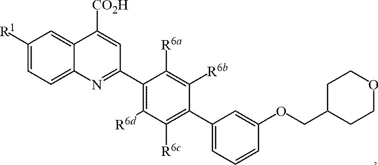

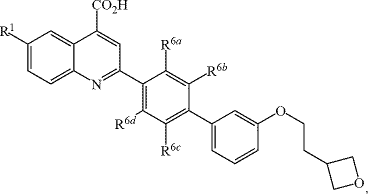

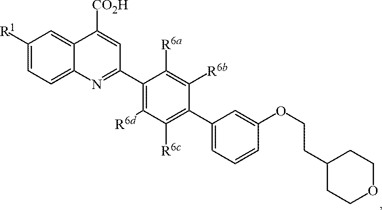

or combinations thereof.

Aspect 235. The compound of Aspect 1, having a structure represented by a formula:

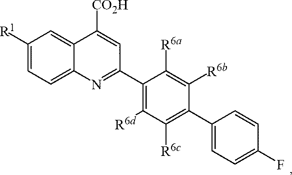

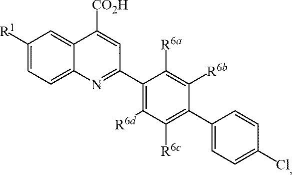

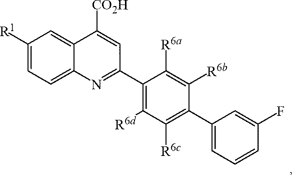

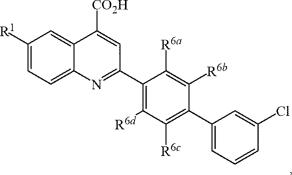

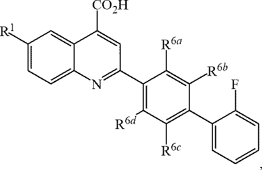

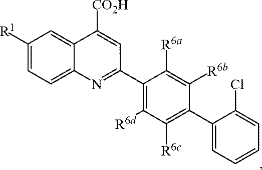

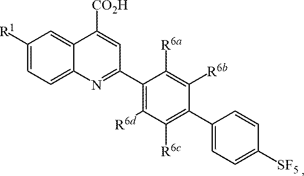

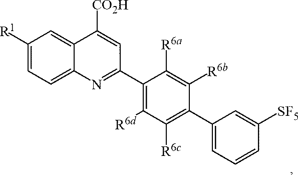

or combinations thereof.

Aspect 236. The compound of Aspect 1, having a structure represented by a formula:

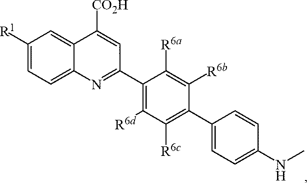

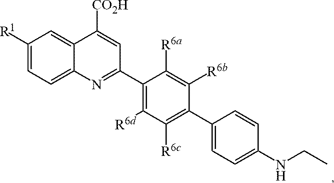

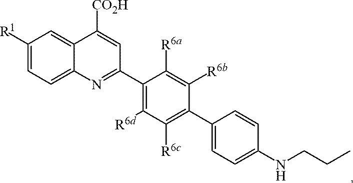

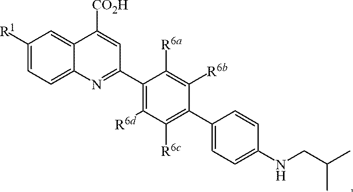

or combinations thereof.

Aspect 237. The compound of Aspect 1, having a structure represented by a formula:

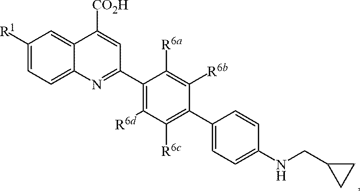

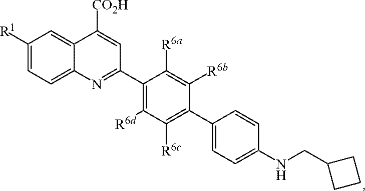

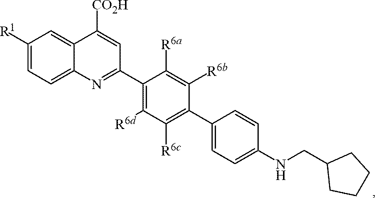

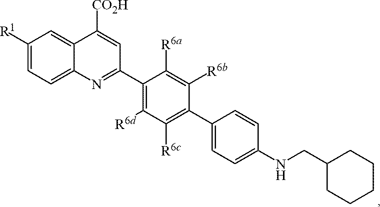

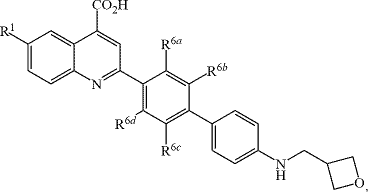

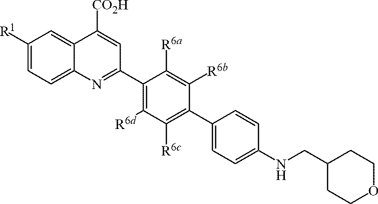

or combinations thereof.

Aspect 238. The compound of any one of Aspect 228-Aspect 237, wherein R¹ is selected from halogen, —SF₅, —CF₃, and —CF₂CF₃.

Aspect 239. The compound of Aspect 238, wherein R¹ is halogen or —SF₅.

Aspect 240. The compound of Aspect 238, wherein R¹ is —F or —Cl.

Aspect 241. The compound of Aspect 238, wherein R¹ is —F.

Aspect 242. The compound of Aspect 238, wherein R¹ is—Cl.

Aspect 243. The compound of Aspect 238, wherein R¹ is —SF₅.

Aspect 244. The compound of Aspect 238, wherein R¹ is selected-CF₃ and —CF₂CF₃.

Aspect 245. The compound of any one of Aspect 228-Aspect 237, wherein each of R^{6a}, R^{6b}, R^{6c}, and R^{6d} is independently selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl, provided that at least one of R^{6a}, R^{6b}, R^{6c}, and R^{6d} is not hydrogen.

Aspect 246. The compound of Aspect 245, wherein R^{6a} and R^{6b} are independently selected from hydrogen, halogen, —SF₅, —CN, —N₃, —OH, —NH₂, —CHF₂, —CH₂F, and —CF₃.

Aspect 247. The compound of Aspect 246, wherein R^{6a} and R^{6b} are independently selected from halogen, —SF₅, —CN, —N₃, —OH, —NH₂, —CHF₂, —CH₂F, and —CF₃.

Aspect 248. The compound of Aspect 247, wherein R^{6a} and R^{6b} are independently selected from —F, —Cl, —SF₅, —CN, —N₃, —OH, —NH₂, —CHF₂, —CH₂F, and —CF₃.

Aspect 249. The compound of Aspect 245, wherein R^{6a} and R^{6c} are independently selected from hydrogen, halogen, —SF₅, —CN, —N₃, —OH, —NH₂, —CHF₂, —CH₂F, and —CF₃.

Aspect 250. The compound of Aspect 249, wherein R^{6a} and R^{6c} are independently selected from halogen, —SF₅, —CN, —N₃, —OH, —NH₂, —CHF₂, —CH₂F, and —CF₃.

Aspect 251. The compound of Aspect 250, wherein R^{6a} and R^{6c} are independently selected from —F, —Cl, —SF₅, —CN, —N₃, —OH, —NH₂, —CHF₂, —CH₂F, and —CF₃.

Aspect 252. The compound of Aspect 245, wherein R^{6a} and R^{6d} are independently selected from hydrogen, halogen, —SF₅, —CN, —N₃, —OH, —NH₂, —CHF₂, —CH₂F, and —CF₃.

Aspect 253. The compound of Aspect 252, wherein R^{6a} and R^{6d} are independently selected from halogen, —SF₅, —CN, —N₃, —OH, —NH₂, —CHF₂, —CH₂F, and —CF₃.

Aspect 254. The compound of Aspect 253, wherein R^{6a} and R^{6d} are independently selected from —F, —Cl, —SF₅, —CN, —N₃, —OH, —NH₂, —CHF₂, —CH₂F, and —CF₃.

Aspect 255. The compound of Aspect 245, wherein R^{6a} is selected from —F, —Cl, —SF₅, —CN, —N₃, —OH, and —NH₂.

Aspect 256. The compound of Aspect 245, wherein R^{6a} is selected from —F, —SF₅, —CN, —N₃, —OH, and —NH₂.

Aspect 257. The compound of Aspect 245, wherein R^{6b} is selected from —F, —Cl, —SF₅, —CN, —N₃, —OH, and —NH₂.

Aspect 258. The compound of Aspect 245, wherein R^{6a} is selected from —F, —SF₅, —CN, —N₃, —OH, and —NH₂.

Aspect 259. The compound of any of Aspect 245-Aspect 259, wherein each of R^{6c} and R^{6d} are hydrogen.

Aspect 260. The compound of any one of Aspect 228-Aspect 237, wherein R^{6a} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b}, R^{6c}, and R^{6d} is hydrogen.

Aspect 261. The compound of Aspect 260, wherein R^{6a} is selected from halogen, —SF₅, —CN, —N₃, —OH, —NH₂, —CHF₂, —CH₂F, and —CF₃.

Aspect 262. The compound of Aspect 261, wherein R^{6a} is selected from —F, —Cl, —SF₅, —CN, —N₃, —OH, —NH₂, —CHF₂, —CH₂F, and —CF₃.

Aspect 263. The compound of Aspect 262, wherein R^{6a} is —F.

Aspect 264. The compound of any one of Aspect 228-Aspect 237, wherein R^{6b} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a}, R^{6c}, and R^{6d} is hydrogen.

Aspect 265. The compound of Aspect 264, wherein R^{6b} is selected from halogen, —SF₅, —CN, —N₃, —OH, —NH₂, —CHF₂, —CH₂F, and —CF₃.

Aspect 266. The compound of Aspect 265, wherein R^{6b} is selected from —F, —Cl, —SF₅, —CN, —N₃, —OH, —NH₂, —CHF₂, —CH₂F, and —CF₃.

Aspect 267. The compound of Aspect 266, wherein R^{6b} is —F.

Aspect 268. The compound of any one of Aspect 228-Aspect 237, wherein each of R^{6a} and R^{6b} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6c} and R^{6d} is hydrogen.

Aspect 269. The compound of Aspect 268, wherein each of R^{6a} and R^{6b} is independently selected from halogen, —SF₅, —CN, —N₃, —OH, —NH₂, —CHF₂, —CH₂F, and —CF₃.

Aspect 270. The compound of Aspect 269, wherein each of R^{6a} and R^{6b} is independently selected from —F, —Cl, —SF₅, —CN, —N₃, —OH, —NH₂, —CHF₂, —CH₂F, and —CF₃.

Aspect 271. The compound of Aspect 270, wherein each of R^{6a} and R^{6b} is —F.

Aspect 272. The compound of any one of Aspect 228-Aspect 237, wherein each of R^{6a} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6d} is hydrogen.

Aspect 273. The compound of Aspect 272, wherein each of R^{6a} and R^{6c} is independently selected from halogen, —SF₅, —CN, —N₃, —OH, —NH₂, —CHF₂, —CH₂F, and —CF₃.

Aspect 274. The compound of Aspect 273, wherein each of R^{6a} and R^{6c} is independently selected from —F, —Cl, —SF₅, —CN, —N₃, —OH, —NH₂, —CHF₂, —CH₂F, and —CF₃.

Aspect 275. The compound of Aspect 274, wherein each of R^{6a} and R^{6c} is —F.

Aspect 276. The compound of any one of Aspect 228-Aspect 237, wherein each of R^{6a} and R^{6d} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6c} is hydrogen.

Aspect 277. The compound of Aspect 276, wherein each of R^{6a} and R^{6d} b is independently selected from halogen, —SF₅, —CN, —N₃, —OH, —NH₂, —CHF₂, —CH₂F, and —CF₃.

Aspect 278. The compound of Aspect 277, wherein each of R^{6a} and R^{6d} is independently selected from —F, —Cl, —SF₅, —CN, —N₃, —OH, —NH₂, —CHF₂, —CH₂F, and —CF₃.

Aspect 279. The compound of Aspect 278, wherein each of R^{6a} and R^{6d} —F.

Aspect 280. The compound of any one of Aspect 228-Aspect 237, wherein each of R^{6b} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a} and R^{6d} is hydrogen.

Aspect 281. The compound of Aspect 280, wherein each of R^{6b} and R^{6c} is independently selected from halogen, —SF₅, —CN, —N₃, —OH, —NH₂, —CHF₂, —CH₂F, and —CF₃.

Aspect 282. The compound of Aspect 281, wherein each of R^{6b} and R^{6c} is independently selected from —F, —Cl, —SF₅, —CN, —N₃, —OH, —NH₂, —CHF₂, —CH₂F, and —CF₃.

Aspect 283. The compound of Aspect 282, wherein each of R^{6b} and R^{6c} is —F.

Aspect 284. The compound of 1, having a structure represented by a formula:

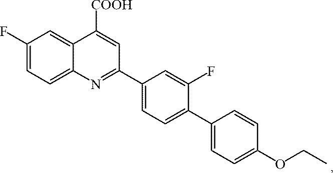

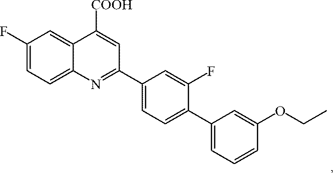

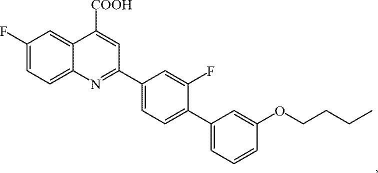

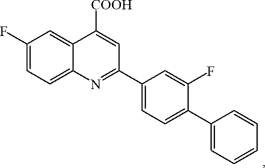

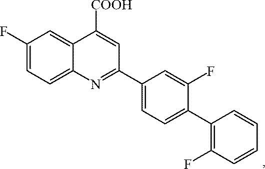

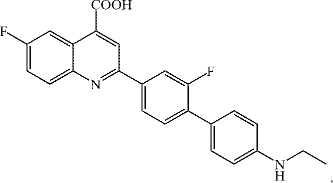

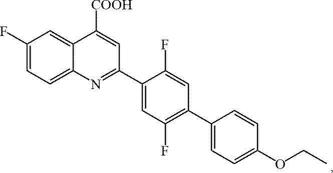

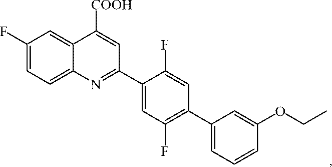

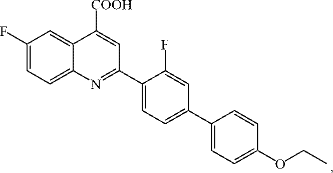

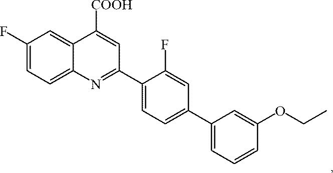

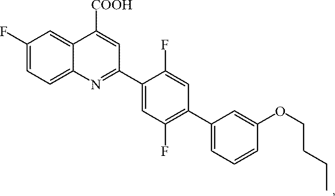

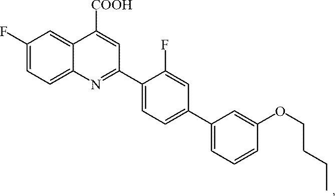

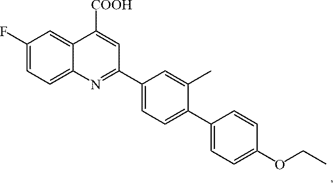

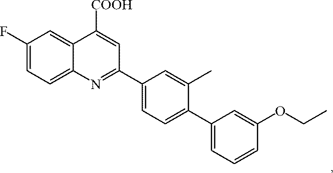

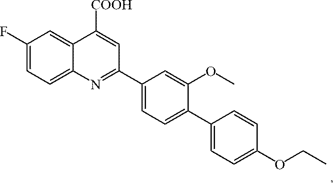

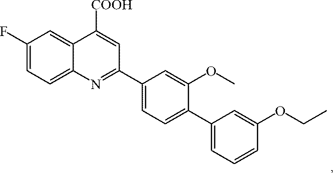

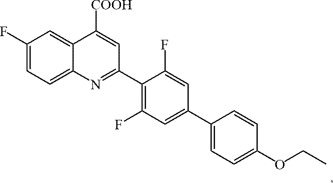

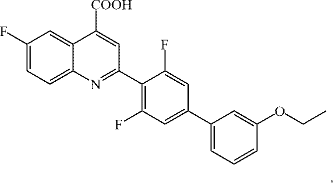

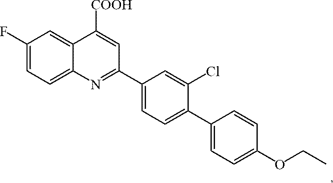

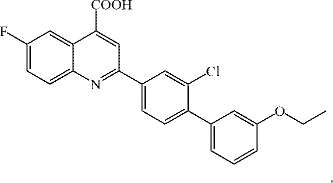

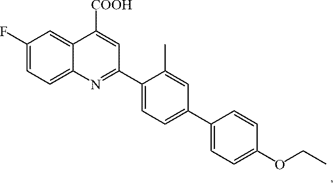

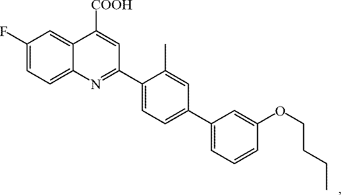

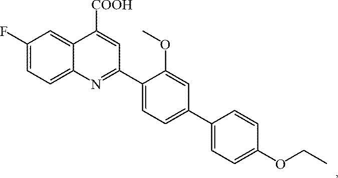

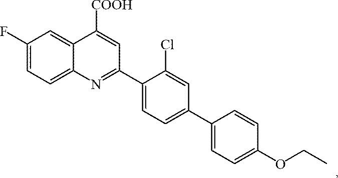

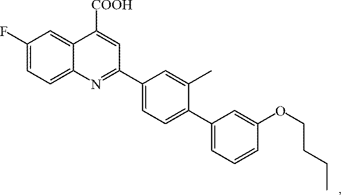

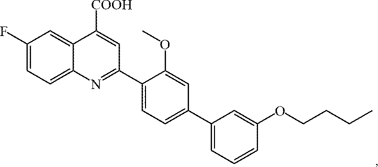

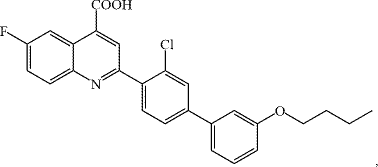

or combinations thereof.

Aspect 285. The compound of 1, present as:

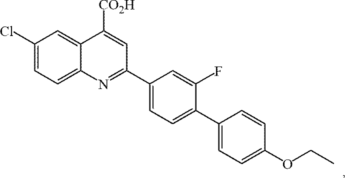

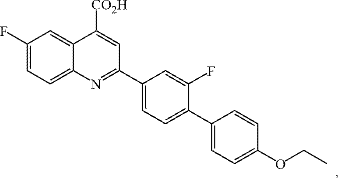

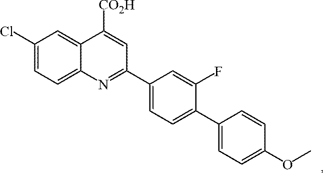

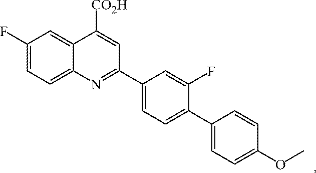

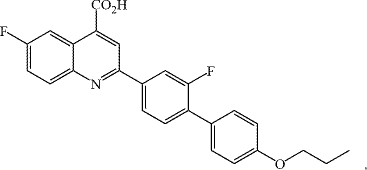

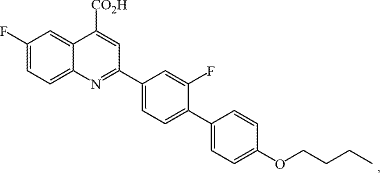

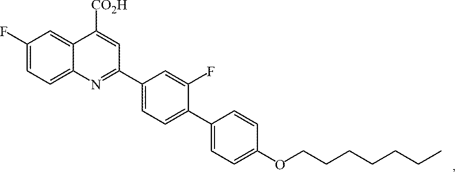

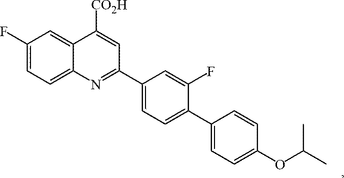

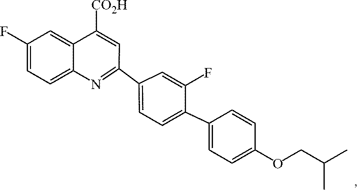

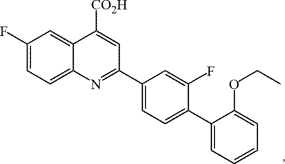

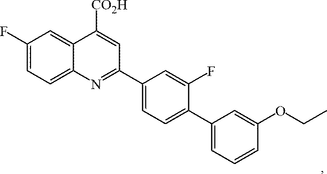

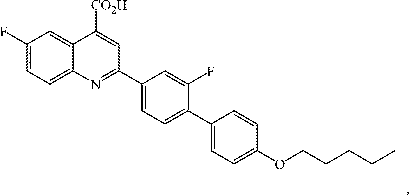

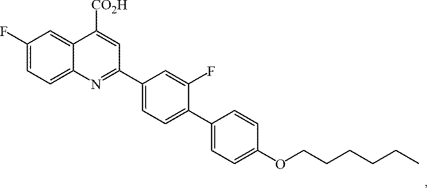

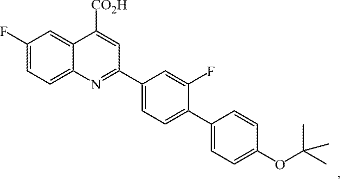

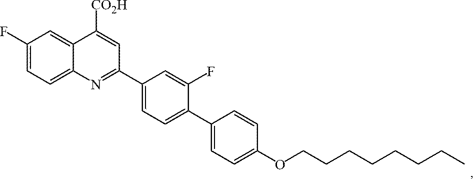

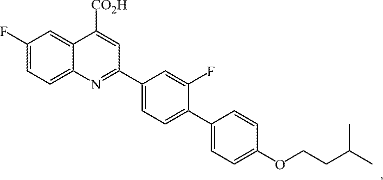

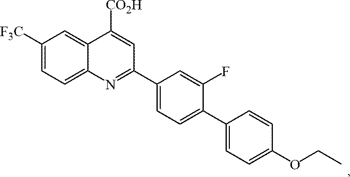

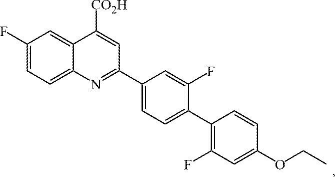

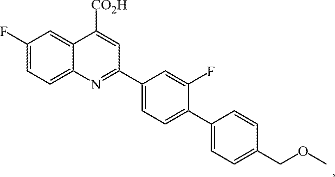

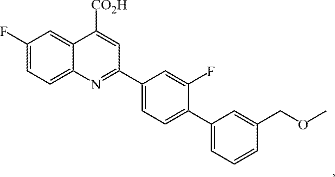

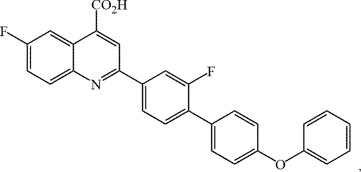

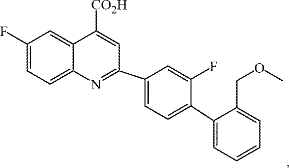

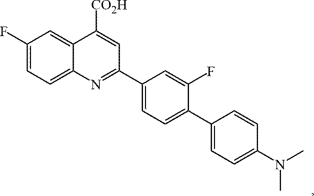

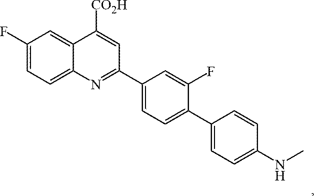

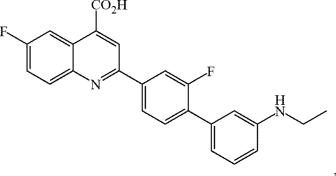

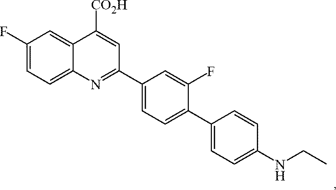

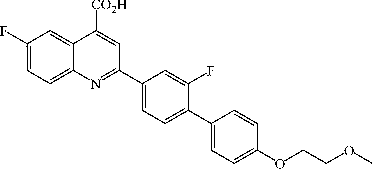 [SMILES: COCCOc1ccc(-c2ccc(-c3cc(C(=O)O)c4cc(F)ccc4n3)cc2F)cc1]

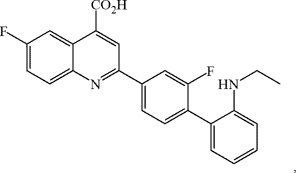

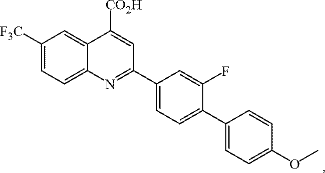

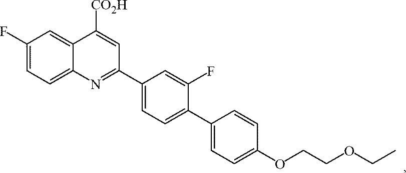

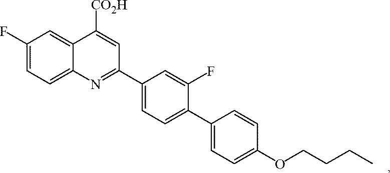

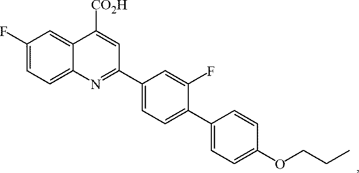

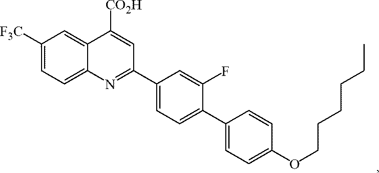

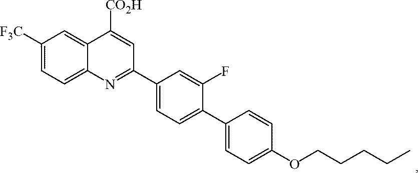

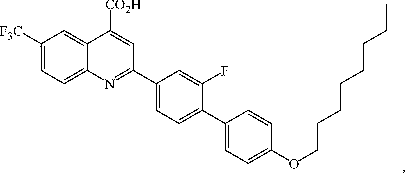

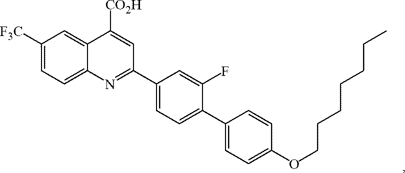

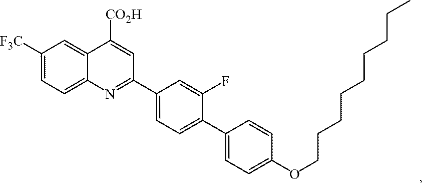

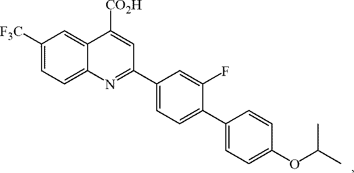

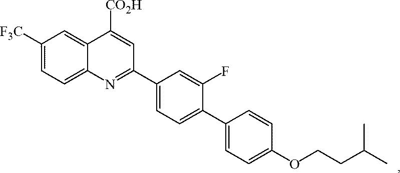

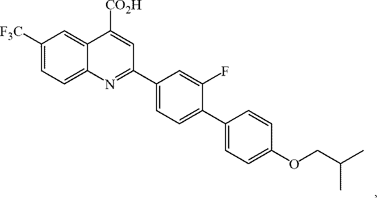

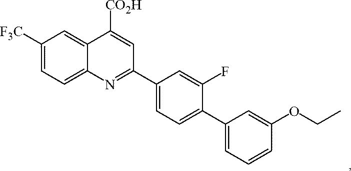

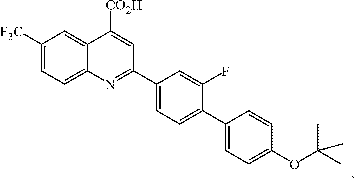

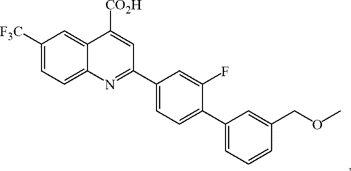

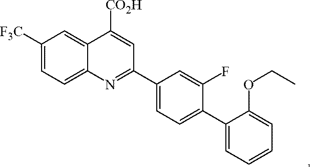

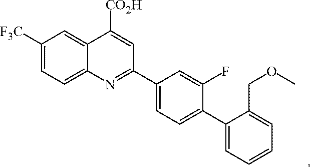

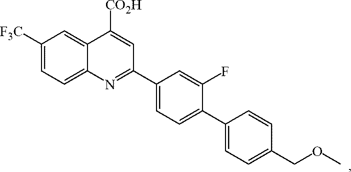

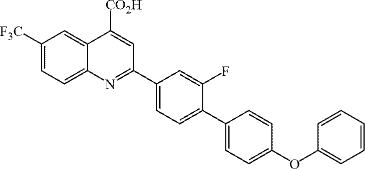 [SMILES: O=C(O)c1cc(-c2ccc(-c3ccc(Oc4ccccc4)cc3)c(F)c2)nc2ccc(C(F)(F)F)cc12]

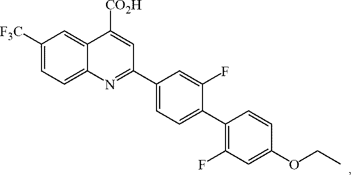

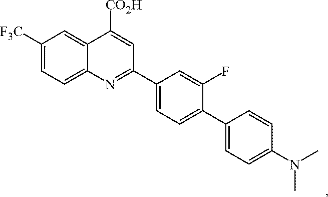

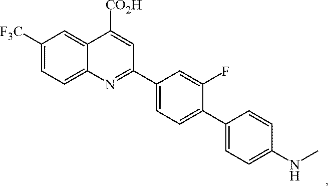

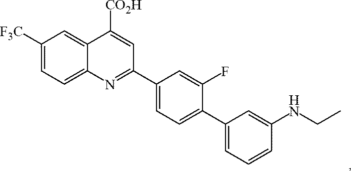

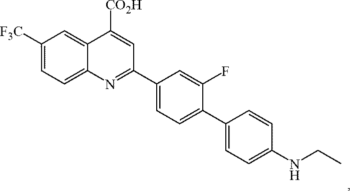

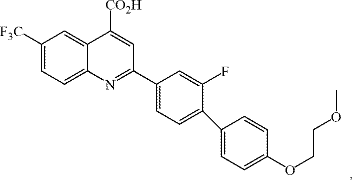

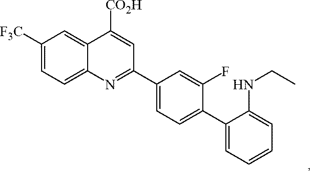

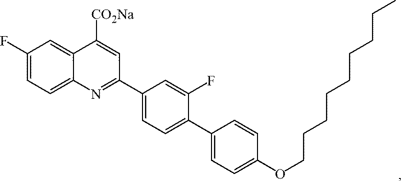

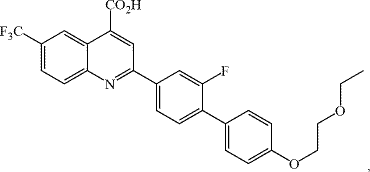

or a subgroup thereof.

Aspect 286. The compound of 1, present as:

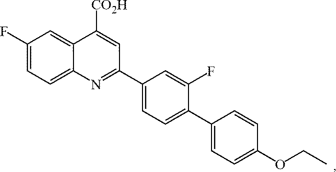

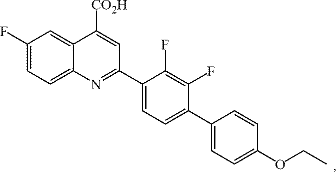

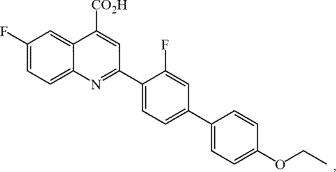

or a combination thereof.

Aspect 287. The compound of anyone of 1-Aspect 286, wherein the compound is a pharmaceutically acceptable salt thereof comprising the conjugate base form of the compound, and a counter ion selected from Li+, K+, Na+, ammonium, tetramethylammonium, tetraethylammonium, Fe⁺², Cu⁺², Zn⁺², Mg⁺², Ca⁺², Al⁺³, Fe⁺³, and combinations thereof.

Aspect 288. The compound of 0, wherein the counter ion is Na⁺.

### DHODH Inhibitor Compounds - Group V

A disclosed DHODH inhibitor can be other DHODH inhibitors as disclosed herein below and referred to as DHODH Inhibitor Compounds - Group V.

In various aspects, an exemplary DHODH inhibitor of DHODH Inhibitor Compounds -Group V disclosed herein is selected from the group consisting of brequinar, leflunomide, redoxal, vidofludimas, S-2678, 2-(3,5-difluoro-3′methoxybiphenyl-4-ylamino)nicotinic acid (also known as ASLAN003), BAY-2402234 (-N-(2-chloro-6-fluorophenyl)-4-(4-ethyl-3-(hydroxymethyl)-5-oxo-4,5-dihydro-1H-1,2,4-triazol-1-yl)-5-fluoro-2-((1,1,1-trifluoropropan-2-yl)oxy)benzamide), AG-636 (1-methyl-5-(2′-methyl-[1,1′-biphenyl]-4-yl)-1H-benzo[d][1,2,3]triazole-7-carboxylic acid), PTC-299 (4-chlorophenyl (S)-6-chloro-1-(4-methoxyphenyl)-1,3,4,9-tetrahydro-2H-pyrido[3,4-b]indole-2-carboxylate), JNJ-74856665, Meds433, RP7214, ML390, Laflunimus, Tenovin-1, Tenovin-6, hDHODH-IN-4, DHODH-IN-11, and teriflunomide.

In various aspects, an exemplary DHODH inhibitor of DHODH Inhibitor Compounds -Group V disclosed herein is selected from the group consisting of teriflunomide, leflunomide a compound of formula (II) (disclosed in WO2008/077639 incorporated herein by reference):

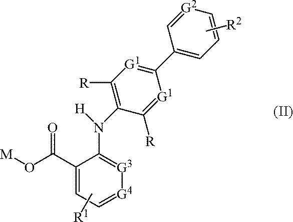

,wherein:
one of the groups G¹ represents a nitrogen atom or a group CR^{c} and the other group represents CR^{c};
G² represents a nitrogen atom or a group CR^{d};
R¹ represents a group selected from hydrogen, halogen, C₁₋₄ alkyl which may be optionally substituted with 1, 2 or 3 substituents selected from the group comprising halogen, hydroxy, and C₃₋₈ cycloalkyl which may be optionally substituted with 1, 2 or 3 substituents selected from halogen and hydroxyl;
R² represents a group selected from hydrogen, halogen, hydroxyl, C₁₋₄ alkyl which may be optionally substituted by 1, 2 or 3 substituents selected from the group comprising halogen, hydroxy, C₃₋₈ alkyl which may be optionally substituted with 1, 2, or 3 substituents selected from halogen and hydroxyl;
R^{a}, R^{b} and R^{c} independently represent a radical selected from the group comprising hydrogen, halogen, C₁₋₄ alkyl optionally substituted by 1, 2 or 3 substituents selected from the group comprising halogen, hydroxy and C₁₋₄alkoxy;
R^{d} represents a group selected from hydrogen, halogen, hydroxyl, C₁₋₄ alkyl which may be substituted by 1, 2 or 3 substituents selected from the group comprising halogen, hydroxyl, C₁₋₄ alkoxy which may be optionally substituted with 1, 2 or 3 substituent selected from the group comprising halogen, hydroxy, and C₃₋₈ cycloalkoxy which may be optionally substituted with 1, 2 or 3 substitutents selected from halogen and hydroxyl;
G³ & G⁴ one is a nitrogen atom and the other is a CH;
M is hydrogen or a pharmaceutically acceptable cation.

In a further aspect, the compound of formula (II) has the proviso that, when at least one of the groups R^{a} and R^{b} represents a hydrogen atom and G² is a group CR^{d}, then R^{d} represents a group selected from C₁₋₄ alkoxy which may be optionally substituted with 1, 2 or 3 substituents selected from halogen, hydroxy, C₃₋₈ cycloalkoxy which may be optionally substituted with 1, 2 or 3 substituents selected from halogen and hydroxyl.

In a further aspect, an exemplary DHODH inhibitor of DHODH Inhibitor Compounds -Group V can be 2-(3, 5-difluoro-3′-methoxybiphenyl-4-ylamino) nicotinic acid (referred to herein as ASLAN003) or a pharmaceutically acceptable salt thereof, in particular:

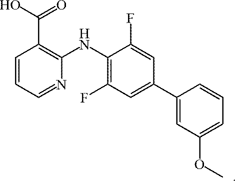

In a further aspect, an exemplary DHODH inhibitor of DHODH Inhibitor Compounds -Group V which may be employed in a method or pharmaceutical combination of the present disclosure include:
teriflunomide which has the following structure:

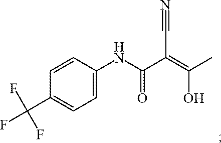

the compounds disclosed in WO97/34600 incorporated herein by reference;
leflunomide which has the following structure:

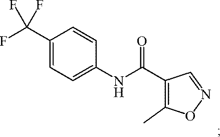

the DHODH inhibitors of formula (1) disclosed in WO99/45926 incorporated herein by reference;
compounds of formula (I) disclosed in WO2003/006425 incorporated herein by reference;
the DHODH inhibitors of formula (I) disclosed in WO2004/056746 incorporated herein by reference;
compounds of formula (I) disclosed in WO2006/022442 incorporated herein by reference; and
DHODH inhibitors disclosed in WO2009/021696 incorporated herein by reference.
Suitable salts of DHODH inhibitors include those disclosed in WO2010/102826, WO2010/10225 and WO2010/102824 each incorporated herein by reference.

In a further aspect, an exemplary DHODH inhibitor of DHODH Inhibitor Compounds -Group V can be represented by a structure:

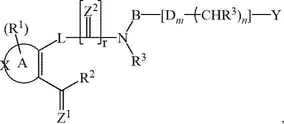

A is an aromatic or non-aromatic 5- or 6-membered hydrocarbon ring wherein optionally one or more of the carbon atoms are replaced by a group X, wherein X is independently selected from the group consisting of S, O, N, NR⁴, SO₂ and SO; L is a single bond or NH; D is O, S, SO₂, NR⁴, or CH₂: Z¹ is O, S, or NR⁵; Z² is O, S, or NR⁵; R¹ independently represents H, halogen, haloalkanyl, haloalkenyl, haloalkynyl, haloalkanyloxy, haloalkenyloxy, haloalkynyloxy, —CO₂R″. —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″— CO₂—R′, —NR″—CO—R*, —NR″—SO₂—R′, —O—CO—R*, —O—CO₂—R*. —O—CO—NR*R″, cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl; R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl; R′ independently represents H, —CO₂R″, —CONR″R‴, —CR″O, —SO₂NR″, — NR″—CO-haloalkanyl, haloalkenyl, haloalkynyl, —NO₂, —NR″—SO₂—haloalkanyl, haloalkenyl, haloalkynyl, —NR″—SO₂—alkanyl, —NR″—SO₂—alkenyl, —NR″—SO₂—alkynyl, —SO₂—alkanyl, —SO₂—alkenyl, —SO₂—alkynyl, —NR″—CO—alkanyl. —NR″—CO—alkenyl, —NR″—CO—alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl; R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl; R‴ independently represents H or alkanyl; R² is H or OR⁶, NHR⁷, NR⁷OR⁷; or R² together with the nitrogen atom which is attached to R⁸ forms a 5 to 7 membered, preferably 5 or 6 membered heterocyclic ring wherein R² is —[CH₂]ₛ and R⁸ is absent; R³ is H, alkanyl, alkenyl, alkynyl, cycloalkyl, heterocycloalkyl, aryl, alkanyloxy, alkenyloxy, alkynyloxy, —O—aryl; —O—cycloalkyl, —O—heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S—aryl; —S—cycloalkyl, —S—heterocycloalkyl, aralkyl, haloalkanyl, haloalkenyl or haloalkynyl; R⁴ is H, alkanyl, alkenyl, alkynyl, cycloalkyl, heterocycloalkyl, aryl or heteroaryl; R⁵ is H, OH, alkanyloxy, alkenyloxy, alkynyloxy, O-aryl, alkanyl, alkenyl, alkynyl or aryl; R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylalkanyl, (acyloxy)alkanyl, (acyloxy)alkenyl, (acyloxy)alkynyl acyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate; R⁷ is H, OH, alkanyl, alkenyl, alkynyl, aryl, alkanyloxy, alkenyloxy, alkynyloxy, —O—aryl, cycloalkyl, heterocycloalkyl, —O—cycloalkyl, or —O—heterocycloalkyl; R⁸ is H, alkanyl, alkenyl or alkynyl; E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′; Y is H, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is

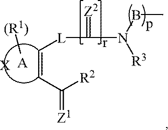

m is 0 or 1; n is 0 or 1; p is 0 or 1; q is 0 or 1; r is 0 or 1; s is 0 to 2; and t is 0 to 3, as disclosed in U.S. Pat. Publ. No. 2019/0025313, which is incorporated herein in its entirety.

For use in the present disclosure, inhibitors of DHODH include known inhibitors as well as compounds that are identified herein as inhibitors. Known inhibitors of DHODH include the immunomodulatory drugs teriflunomide and leflunomide. Other inhibitors include, without limitation, those disclosed in, for example Baumgartner et al. (2006) J. Med. Chem. 49(4):1239-1247; Lolli et al. (2012) Eur. J. Med. Chem. 49:102-109; Lucas-Hourani et al. (2015) J. Med. Chem. 58(14):5579-5598.

Known compounds that were previously not known to be inhibitors of DHODH include compounds disclosed in International Pat. Publ. No. WO 2006/118607, herein specifically incorporated by reference. Included in such compositions are GSK983, a tetrahydrocarbazole that inhibits the replication of a variety of unrelated viruses in vitro with EC50 values of 5-20 nM (see Harvey et al. (2009) Antiviral Res. 82(1):1-11) and analogs thereof. Such compounds may have the structure:

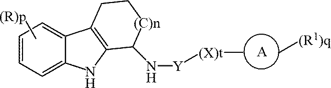

wherein: n is 0, 1,or 2; t is 0 or 1; X is —NH—, —O—, —R¹⁰—, —OR¹⁰—, —R¹⁵O—, -R¹⁰OR¹⁰-, -NR¹⁰-, -R¹⁰N-, -R¹⁰NR¹⁰-, -R¹⁰S(O)ₘ-, or -R¹⁰S(O)ₘR¹⁰-; Y is — C(O)— or —S(O)ₘ—; each R is the same or different and is independently selected from the group consisting of halogen, haloalkyl, akkyl, akenyl, alkynyl, cycloalkyl, cycloakenyl, -R¹⁰cycloalkyl, Ay, —NHR¹⁰Ay, Het, —NHHet, —NHR¹⁰Het, —OR², —OAy, —OHet, —R¹⁰OR², —NR²R³, —NR²AXy, —R¹⁰NR²R³, —R¹⁰NR²Ay, —R¹⁰C(O)R², —C(O)R², —CO₂R², -R¹⁰CO₂R², —C(O)NR²R³, —C(O)Ay, —C(O)NR²Ay, —C(O)Het, —C(O)NHR¹⁰Het, -R¹⁰C(O)NR²R³, —C(S)NR²R³, —R¹⁰C(S)NR²R³, —R¹⁰NHC(NH)NR²R³, —C(NH)NR²R³, -R¹⁰C(NH)NR²R³, —S(O)₂NR²R³, —S(O)₂NR²Ay, —R¹⁰SO₂NHCOR², —R¹⁰SO₂NR²R³, - R¹⁰SO₂R², —S(O)ₘR², —S(O)ₘAy, cyano, nitro, or azido; each R¹ is the same or different and is independently selected from the group consisting of halogen, haloalkyl, alkyl, alkenyl, alkynyl, cycloakyl, cycloakenyl, —R¹⁰cycloakyl, Ay, —NHR¹⁰Ay, Met, —NHHet, —NHR¹⁰Het, —OR², —OAy, —OHet, —R¹⁰OR², —NR²R³, —NR²Ay, —R¹⁰NR²R³, —R¹⁰NR²Ay, -R¹⁰C(O)R², —C(O)R², —CO₂R², —C(O)NR2R3, —C(O)Ay, —C(O)NR²Ay, —C(O)Het, -C(O)NHR¹⁰Het, —R¹⁰C(O)NR²R³, —C(S)NR²R³, —R¹⁰C(S)NR²R³, —R¹⁰NHC(NH)NR³R³, C(NH)NR²R³, -R¹⁰C(NH)NR²R³. —S(O)₂NR²R³, —S(O)₂NR²Ay, —R¹⁰SO₂NHCOR², -R¹⁰NR²T³, —R¹⁰SO₂R², —S(O)ₘR². —S(O)ₘAy, cyano, nitro, or azido; each m independently is 0, 1, or 2; each R¹⁰ is the same or different and b Independently selected from alkylene, cycloalkylene, alkenylene, cycloalkenylene, and alkynylene; p and q are each independently selected from 0, 1, 2, 3, 4, or 5; each of R² and R³ are the same or different and are independently selected from the group consisting of H, alkyl, alkenyl, alkenyl, cycloakyl, cycloalkenyl, —R¹⁰cycloakyl, —R¹⁰OH, -R¹⁰(OR¹⁰)_{w}, and —R¹⁰NR⁴R⁵; w is 1-10; each of R⁴ and R⁶ are the same or different and are independently selected Horn the group consisting of alkyl, cycloakyl, alkenyl, cycloakenyl, and alkynyl; Ay represents an aryl group; Het represents a 5- or 6-membered heterocyclyl or heteroaryl group; ring A is aryl or heteroaryl; provided that when the A ring is aryl, t is 0, and Y is SO₂, then p is not 0; inducting salts, solvates and physiologically functional derivatives thereof.

In some aspects the inhibitor of DHODH is GSK983 or an analog thereof, including without limitation 6Br-pF, 6Br-oTol, and GSK984, which compounds have the following structures:

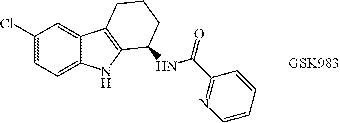 [SMILES: O=C(N[C@@H]1CCCc2c1[nH]c1ccc(Cl)cc21)c1ccccn1]

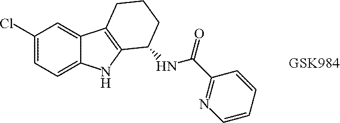 [SMILES: O=C(N[C@H]1CCCc2c1[nH]c1ccc(Cl)cc21)c1ccccn1]

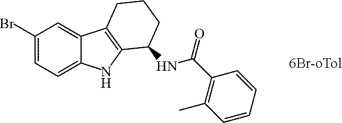 [SMILES: Cc1ccccc1C(=O)N[C@@H]1CCCc2c1[nH]c1ccc(Br)cc21]

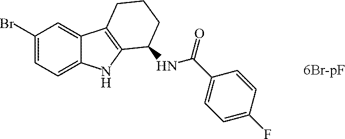 [SMILES: O=C(N[C@@H]1CCCc2c1[nH]c1ccc(Br)cc21)c1ccc(F)cc1]

A disclosed DHODH inhibitor can be a compound known to inhibit DHODH that has already been approved by a drug regulatory authority or is in pre-clinical or clinical development. Exemplary other DHODH inhibitors include ASLAN-003, brequinar, BAY-2402234, AG-636, PTC-299, teriflunomide, leflunomide, DSM-265, olorofim (F-901318), vidofludimus (IMU-838), PP-001, IMU-935, laflunimus (AP-325), RP-7214, 4SC-302, DSM-421, LAS-187247, ABR-224050, FK-778, or combinations thereof.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one aspect, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure. Certain isotopically-labelled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present disclosure and prodrugs thereof can generally be prepared by carrying out the procedures below, by substituting a readily available isotopically labelled reagent for a non- isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the disclosure to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The disclosed compounds can be conviently utilized as a component of a degrader molecule. Accordingly, in various aspects, a disclosed compound can be used as a ligand, a linker, or an adjoining chemical structure within a proteolysis targeting complex or targeted protein degrader complex. For example, Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Bondeson and Crews, Annu Rev Pharmacol Toxicol. 2017 Jan 6; 57: 107-123; Lai et al. Angew Chem Int Ed Engl. 2016 Jan 11; 55(2): 807-810; and PCT Appl. No. PCT/US2018/061573).

In a further aspect, the disclosed compounds can further comprise linkage to a PROteolysis-TArgeting Chimera (PROTAC), thereby providing interaction with the intracellular ubiquitin-proteasome system to selectively degrade target protein. For example, in some instances, any one or more compounds can be utilized to form a composition, chimera, fusion, or complex having a protein degrading function. Some exemplary complexes can include a proteolysis-targeting chimaera (PROTAC) or a degronimid. As understood by a skilled artisan, such a complex is capable of uniting or combining cellular processes related to protein degradation to a specific target protein, wherein the cellular machinery and the target protein are complexed by a ligand, a linker, or an adjoining chemical structure.

DHODH inhibitors for use in the present disclosure can alternatively be based on antisense oligonucleotide constructs. Anti-sense oligonucleotides, including anti-sense RNA molecules and anti-sense DNA molecules, would act to directly block the translation of DHODH mRNA by binding thereto and thus preventing protein translation or increasing mRNA degradation, thus decreasing the level DHODH protein, and thus activity, in a cell. For example, antisense oligonucleotides of at least about 15 bases and complementary to unique regions of the mRNA transcript sequence encoding DHODH can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors for use in the present disclosure. DHODH gene expression can be reduced by contacting the tumor, subject or cell with a small double stranded RNA (dsRNA), or a vector or construct causing the production of a small double stranded RNA, such that expression of DHODH is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as DHODH inhibitors for use in the present disclosure. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present disclosure. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the disclosure can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

### Methods of Making the Compounds

In one aspect, the present disclosure relates to methods of making compounds useful as inhibitors of dihydroorotate dehydrogenase (DHODH), which can be useful in the treatment of clinical conditions, diseases, and disorders associated with DHODH dysfunction and other diseases in which DHODH is involved. In one aspect, the disclosure relates to the disclosed synthetic manipulations. In a further aspect, the disclosed compounds comprise the products of the synthetic methods described herein. In a further aspect, the disclosed compounds comprise a compound produced by a synthetic method described herein. In a still further aspect, the disclosure comprises a pharmaceutical composition comprising a therapeutically effective amount of the product of the disclosed methods and a pharmaceutically acceptable carrier. In a still further aspect, the disclosure comprises a method for manufacturing a medicament comprising combining at least one compound of any of disclosed compounds or at least one product of the disclosed methods with a pharmaceutically acceptable carrier or diluent.

The compounds of this disclosure can be prepared by employing reactions as shown in the disclosed schemes, in addition to other standard manipulations that are known in the literature, exemplified in the experimental sections or clear to one skilled in the art. The following examples are provided so that the disclosure might be more fully understood, are illustrative only, and should not be construed as limiting. For clarity, examples having a fewer substituent can be shown where multiple substituents are allowed under the definitions disclosed herein.

It is contemplated that each disclosed method can further comprise additional steps, manipulations, and/or components. It is also contemplated that any one or more step, manipulation, and/or component can be optionally omitted from the disclosure. It is understood that a disclosed method can be used to provide the disclosed compounds. It is also understood that the products of the disclosed methods can be employed in the disclosed compositions, kits, and uses.

In one aspect, substituted 6-substituted-2-(phenylheteroaryl)quinoline-4-carboxylic acid analogs of the present disclosure can be prepared generically by the synthetic scheme as shown below.

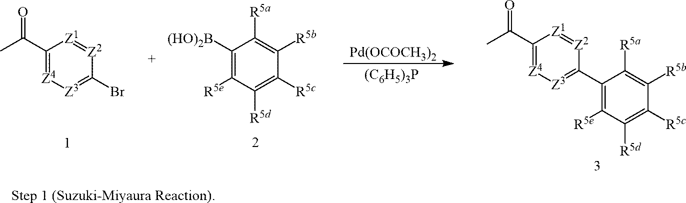

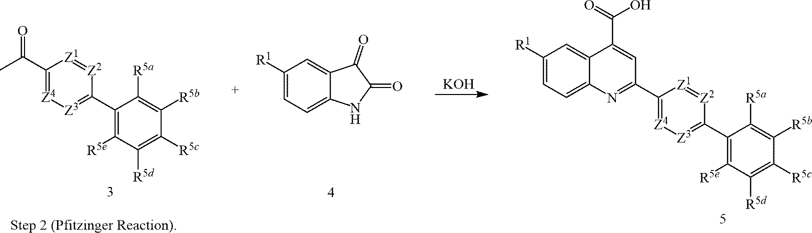

Compounds are represented in generic form, with substituents as noted in compound descriptions elsewhere herein. A more specific example is set forth below.

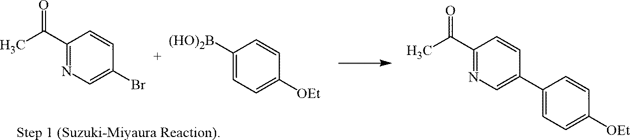

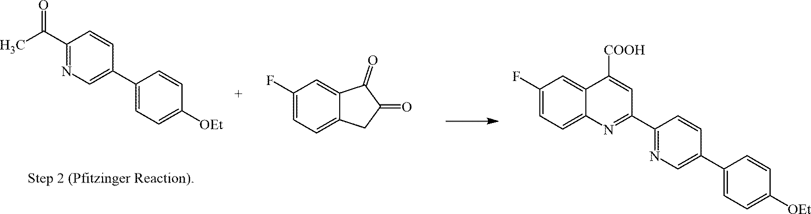

In one aspect, compounds of the present disclosure, e.g. compounds of Formula 5 can be prepared in a two-step reaction as shown above. Briefly, the synthesis of compound of Formula 5 begins in Step 1 with reaction of compounds of Formulas 1 and 2 to yield compounds of Formula 3. Compounds of Formula 1, i.e., 4-halosubstituted heteroaryl ethanone analogs, e.g., 1-(5-bromopyridin-2-yl)ethan-1-one, and Formula 2, i.e., appropriately substituted phenylboronic acids, e.g., 4-ethoxyphenylboronic acid, can be obtained from commercial sources or can be readily prepared by one skilled in the art according to methods described in the literature. For example, both 1-(5-bromopyridin-2-yl)ethan-1-one and 4-ethoxyphenylboronic acid are available commercially. The reaction of reaction of compounds of Formulas 1 and 2 is typically carried at a molar ratio of Formula 1 compound to Formula 2 compound of about 25:1 to about 1:1 out in a suitable solvent, e.g., 1-propanol, in the presence of palladium acetate and triphenylphosphine, at a suitable temperature, e.g. about 75° C. to about 200° C., for a suitable period of time, e.g. about 10 minutes to about 2 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art.

In Step 2, the compound of Formula 3, isolated from Step 1, is reacted with compounds of Formula 4 to yield the desired disclosed compound of Formula 5 as shown above. Briefly, a mixture of the appropriate isatin, i.e., a compound of Formula 4, e.g., 5-fluoroisatin (5-fluoroindoline-2,3-dione), and a suitable base, e.g., aqueous potassium hydroxide solution (33%), are stirred and heated gently. To this solution, the slurry of a compound of Formula 3, e.g., 1-(5-(4-ethoxyphenyl)pyridin-2-yl)ethan-1-one, in an amount of about equimolar to the compound of Formula 4, and a suitable solvent is used to prepare the slurry, e.g., ethanol. The reaction mixture is then heated to a suitable temperature, e.g., reflux or about 70° C. to about 200° C., for a suitable period of time, e.g., about 10 minutes to about 3 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art.

In one aspect, substituted 6-substituted-2-(phenylheteroaryl)quinoline-4-carboxylic acid analogs of the present disclosure can be prepared generically by the synthetic scheme as shown below.

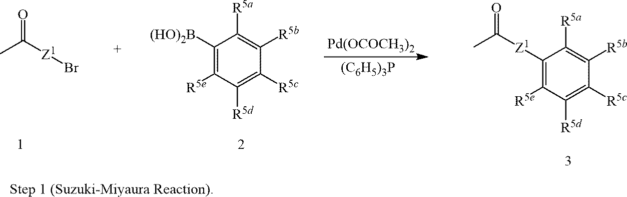

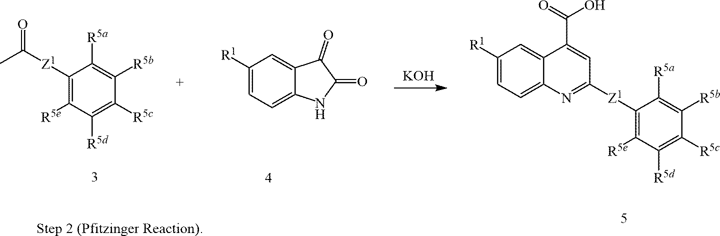

Compounds are represented in generic form, with substituents as noted in compound descriptions elsewhere herein. A more specific example is set forth below.

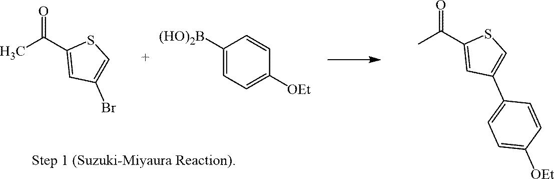

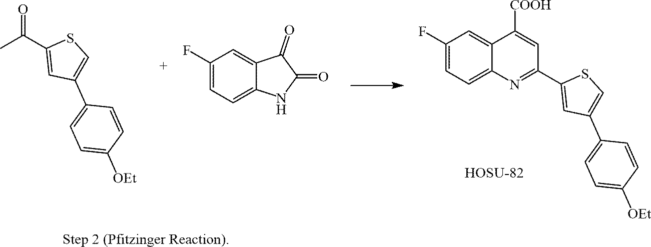

In one aspect, compounds of the present disclosure, e.g. compounds of Formula 5 can be prepared in a two-step reaction as shown above. Briefly, the synthesis of compound of Formula 5 begins in Step 1 with reaction of compounds of Formulas 1 and 2 to yield compounds of Formula 3. Compounds of Formula 1, i.e., halosubstituted heteroaryl ethanone analogs, e.g., 1-(4-bromothiophen-2-yl)ethan-1-one, and Formula 2, i.e., appropriately substituted phenylboronic acids, e.g., 4-ethoxyphenylboronic acid, can be obtained from commercial sources or can be readily prepared by one skilled in the art according to methods described in the literature. For example, both 1-(4-bromothiophen-2-yl)ethan-1-one and 4-ethoxyphenylboronic acid are available commercially. The reaction of reaction of compounds of Formulas 1 and 2 is typically carried at a molar ratio of Formula 1 compound to Formula 2 compound of about 25:1 to about 1:1 out in a suitable solvent, e.g., 1-propanol, in the presence of palladium acetate and triphenylphosphine, at a suitable temperature, e.g. about 75° C. to about 200° C., for a suitable period of time, e.g. about 10 minutes to about 2 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art.

In Step 2, the compound of Formula 3, isolated from Step 1, is reacted with compounds of Formula 4 to yield the desired disclosed compound of Formula 5 as shown above. Briefly, a mixture of the appropriate isatin, i.e., a compound of Formula 4, e.g., 5-fluoroisatin (5-fluoroindoline-2,3-dione), and a suitable base, e.g., aqueous potassium hydroxide solution (33%), are stirred and heated gently. To this solution, the slurry of a compound of Formula 3, e.g., 1-(4-(4-ethoxyphenyl)thiophen-2-yl)ethan-1-one, in an amount of about equimolar to the compound of Formula 4, and a suitable solvent is used to prepare the slurry, e.g., ethanol. The reaction mixture is then heated to a suitable temperature, e.g., reflux or about 70° C. to about 200° C., for a suitable period of time, e.g., about 10 minutes to about 3 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art. The product may also be further purified if residual solvent is present, e.g., as described herein below in the Examples.

In one aspect, substituted 6-substituted-2-([1,1′-biphenyl]-4-yl)quinoline-4-carboxylic acid analogs of the present disclosure can be prepared generically by the synthetic scheme as shown below.

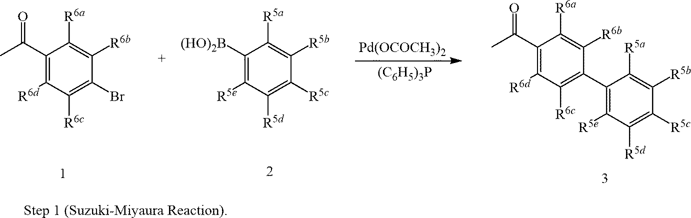

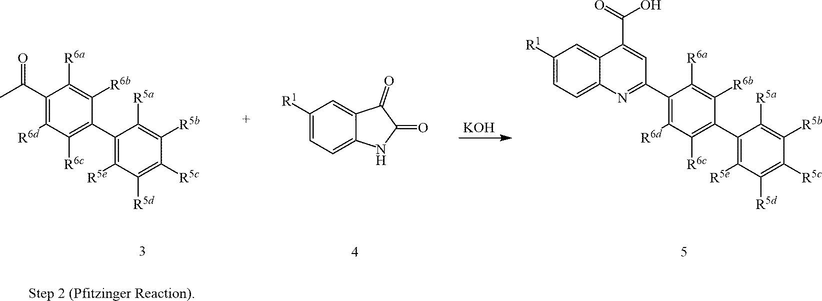

Compounds are represented in generic form, with substituents as noted in compound descriptions elsewhere herein. A more specific example is set forth below.

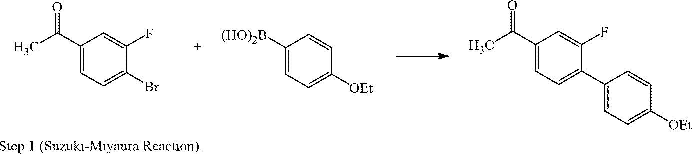

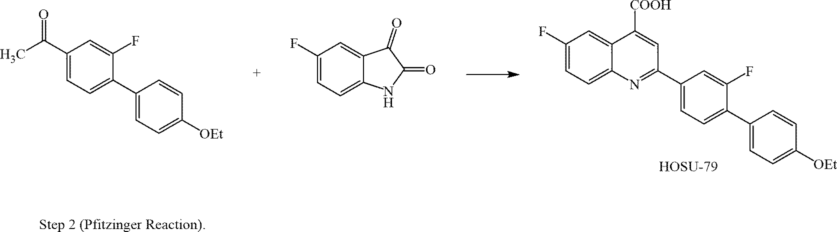

In one aspect, compounds of the present disclosure, e.g. compounds of Formula 5 can be prepared in a two-step reaction as shown above. Briefly, the synthesis of compound of Formula 5 begin in Step 1 with reaction of compounds of Formulas 1 and 2 to yield compounds of Formula 3. Compounds of Formula 1, i.e., 4-halosubstituted phenone analogs, e.g., 3-fluoro-4-bromoacetophenone, and Formula 2, i.e., appropriately substituted phenylboronic acids, e.g., 4-ethoxyphenylboronic acid, can be obtained from commercial sources or can be readily prepared by skilled in the art according to methods described in the literature. For example, both 3-fluoro-4-bromophenone and 4-ethoxyphenylboronic acid are available commercially. The reaction of reaction of compounds of Formulas 1 and 2 is typically carried at a molar ratio of Formula 1 compound to Formula 2 compound of about 25:1 to about 1:1 in a suitable solvent, e.g., 1-propanol, in the presence of palladium acetate and triphenylphosphine, at a suitable temperature, e.g. about 75° C. to about 200° C., for a suitable period of time, e.g. about 10 minutes to about 2 hours, in order to ensure that the reaction is complete. The reaction is then cooled to a suitable temperature, e.g., room temperature, and then can be further cooled, e.g., to about 0° C. to obtain suitable crystals, which can be collected by filtration. Other suitable methods of isolating the product will be apparent to one skilled in the art.

In Step 2, the compound of Formula 3, isolated from Step 1, is reacted with compounds of Formula 4 to yield the desired disclosed compound of Formula 5 as shown above. Briefly, a mixture of the appropriate isatin, i.e., a compound of Formula 4, e.g., 5-fluoroisatin (5-fluoroindoline-2,3-dione), and a suitable base, e.g., aqueous potassium hydroxide solution (33%), are stirred and heated gently. To this solution, the slurry of a compound of Formula 3, e.g., 1-(4′-ethoxy-[1,1′-biphenyl]-4-yl)ethan-1-one, in an amount of about equimolar to the compound of Formula 4, and a suitable solvent is used to prepare the slurry, e.g., ethanol. The reaction mixture is then heated to a suitable temperature, e.g., reflux or about 70° C. to about 200° C., for a suitable period of time, e.g., about 10 minutes to about 3 hours, in order to ensure that the reaction is complete. The reaction is then cooled to a suitable temperature, e.g., room temperature, and then can be further cooled, e.g., to about 0° C. to obtain suitable crystals, which can be collected by filtration. Other suitable methods of isolating the product will be apparent to one skilled in the art. The product may also be further purified if residual solvent is present, e.g., by methods known in the art.

In one aspect, substituted 3,4,6,8-substituted-2-([1,1′-biphenyl]-4-yl)quinoline analogs of the present disclosure can be prepared generically by the synthetic scheme as shown below.

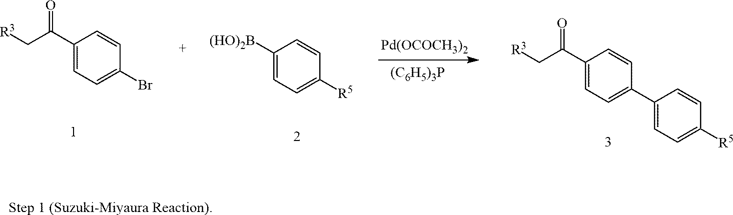

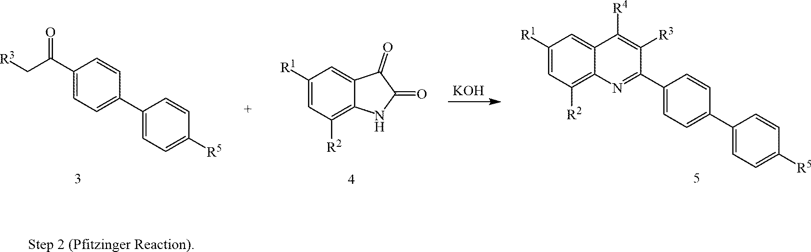

Compounds are represented in generic form, with substituents as noted in compound descriptions elsewhere herein. A more specific example is set forth below.

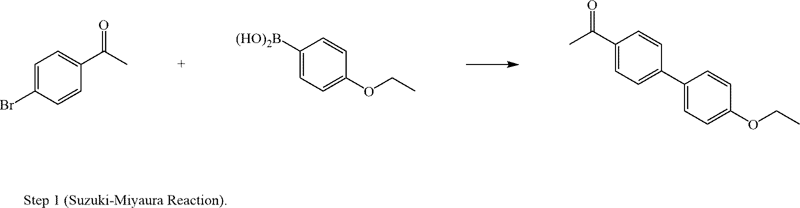

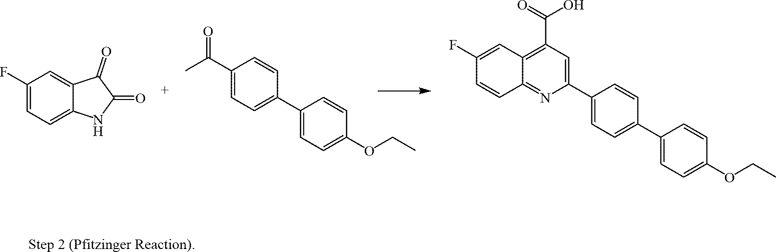

In one aspect, compounds of the present disclosure, e.g. compounds of Formula 5 can be prepared in a two-step reaction as shown above. Briefly, the synthesis of compound of Formula 5 begin in Step 1 with reaction of compounds of Formulas 1 and 2 to yield compounds of Formula 3. Compounds of Formula 1, i.e., 4-halosubstituted phenone analogs, e.g., 4-bromoacetophenone, and Formula 2, i.e., appropriately substituted phenylboronic acids, e.g., 4-ethoxyphenylboronic acid, can be obtained from commercial sources or can be readily prepared by skilled in the art according to methods described in the literature. For example, both 4-bromophenone and 4-ethoxyphenylboronic acid are available commercially. The reaction of reaction of compounds of Formulas 1 and 2 is typically carried at a molar ratio of Formula 1 compound to Formula 2 compound of about 5-25:1 out in a suitable solvent, e.g., 1-propanol, in the presence of palladium acetate and triphenylphosphine, at a suitable temperature, e.g. about 75° C. to about 200° C., for a suitable period of time, e.g. about 10 minutes to about 2 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art.

In Step 2, the compound of Formula 3, isolated from Step 1, is reacted with compounds of Formula 4 to yield the desired disclosed compound of Formula 5 as shown above. Briefly, a mixture of the appropriate isatin, i.e., a compound of Formula 4, e.g., 5-fluoroisatin (5-fluoroindoline-2,3-dione), and a suitable base, e.g., aqueous potassium hydroxide solution (33%), are stirred and heated gently. To this solution, the slurry of a compound of Formula 3, e.g., 1-(4′-ethoxy-[1,1′-biphenyl]-4-yl)ethan-1-one, in an amount of about equimolar to the compound of Formula 4, and a suitable solvent is used to prepare the slurry, e.g., ethanol. The reaction mixture is then heated to a suitable temperature, e.g., reflux or about 70° C. to about 200° C., for a suitable period of time, e.g., about 10 minutes to about 3 hours, in order to ensure that the reaction is complete. The reaction is then cooled to a suitable temperature, e.g., room temperature, and then can be further cooled, e.g., to about 0° C. to obtain suitable crystals, which can collected by filtration. Other suitable methods of isolating the product will be apparent to one skilled in the art. The product may also be further purified if residual solvent is present.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington’s Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine; and the like.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and dibutylsebacate, dibutylsuccinate, dibutyltartrate; diethylene glycol dipropionate; ethyleneglycol diacetate, dibutyrate, dipropionate; tributyl phosphate, tributyrin; polyethylene glycol sorbitan monooleate (polysorbates such as Polysorbate 50); sorbitan monooleate; and the like.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 mole ethylene oxide per 1 mole glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylenediaminetetracetic acid, nitrilotriacetic acid, diethylenetriaminepentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer’s dextrose, dextrose and sodium chloride, lactated Ringer’s and fixed oils. Intravenous vehicles include fluid and nutrient replenishers, electrolyte replenishers such as those based on Ringer’s dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present disclosure may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt% to about 10 wt% of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present disclosure include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present disclosure therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present disclosure include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the disclosure formulated in a pharmaceutically acceptable carrier may also be prepared, placed in an appropriate container, and labeled for treatment of an indicated condition.

Another patch system configuration which can be used by the present disclosure is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present disclosure is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require of inhibition dihydroorotate dehydrogenase activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating dihydroorotate dehydrogenase activity (e.g., treatment of one or more disorders, such as a cancer or a graft-versus-host-disease, that can be treated via inhibition of dihydroorotate dehydrogenase dysfunction activity) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and a therapeutic agent that can be used to treat autoimmune diseases, immune and inflammatory diseases, destructive bone disorders, malignant neoplastic diseases, angiogenic-related disorders, viral diseases, and infectious diseases. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Methods of Using the Compounds

In a further aspect, the present disclosure provides methods of treatment comprising administration of a therapeutically effective amount of a disclosed compound or pharmaceutical composition as disclosed herein above to a subject in need thereof. In particular, the disclosed compounds and disclosed pharmaceutical compositions can be used in methods of treating a disease or disorder that are associated with increased, aberrant, or dysfunctional levels of dihydroorotate dehydrogenase (DHODH) activity in a cell, tissue, or organism. That is, the disclosed compounds and disclosed pharmaceutical compositions can be used to inhibit DHODH activity in a cell, tissue or organism to provide a clinical or therapeutic benefit to a subject which has been determined to or been diagnosed to have with increased, aberrant, or dysfunctional levels of dihydroorotate dehydrogenase (DHODH) activity.

In some aspects of the disclosed methods, the subject has been diagnosed with a need for treatment prior to the administering step. In some aspects of the disclosed method, the subject has been diagnosed with a disorder treatable by inhibition of DHODH and/or a need for inhibition of DHODH prior to the administering step. In some aspects of the disclosed method, the subject has been diagnosed with a cancer, a disorder associated with T-cell proliferation, or a may be at risk for graft-versus-host disease or organ rejection following transplantation prior to the administering step. In some aspects of the disclosed methods, the subject has been identified with a need for treatment prior to the administering step.

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

DHODH is an enzyme that catalyzes the fourth step in the de novo biosynthesis of pyrimidine. It converts dihydroorotate (DHO) to orotate (ORO). Human DHODH is a ubiquitous flavine mononucleotide (FMN) moiety flavoprotein. In a mammalian cell, DHODH is anchored at the inner mitochondrial leaflet and catalyzes the conversion of DHO to ORO, which represents the rate limiting step in the de novo pyrimidine biosynthesis. Kinetic studies indicate a sequential ping-pong mechanism for the conversion of DHO to ORO (e.g., see Knecht et al., Chem. Biol. Interact. 2000, 124, 61-76). The first half-reaction comprises the reduction of DHO to ORO. Electrons are transferred to the FMN which becomes oxidized to dihydroflavin mononucleotide (FMNH2). After dissociation of ORO from the enzyme, FMNH2 is regenerated by a ubiquinone molecule, which is recruited from the inner mitochondrial membrane. Kinetic and structural studies revealed two distinct binding sites for DHO/ORO and ubiquinone, respectively.

Human DHODH is composed of two domains, a large C-terminal domain (Met78-Arg396) and a smaller N-terminal domain (Met30-Leu68), connected by an extended loop. The large C-terminal domain can be best described as an α/β-barrel fold with a central barrel of eight parallel β strands surrounded by eight α helices. The redox site, formed by the substrate binding pocket and the site that binds the cofactor FMN, is located on this large C-terminal domain. The small N-terminal domain, on the other hand, consists of two α helices (labeled α1 and α2), both connected by a short loop. This small N-terminal domain harbors the binding site for the cofactor ubiquinone. The helices α1 and α2 span a slot of about 10×20 Å2 in the so-called hydrophobic patch, with the short α1-α2 loop at the narrow end of that slot. The slot forms the entrance to a tunnel that ends at the FMN cavity nearby the α1-α2 loop. This tunnel narrows toward the proximal redox site and ends with several charged or polar side chains (Gln47, Tyr356, Thr360, and Arg136). Structural clues, as discussed above, along with kinetic studies suggest that ubiquinone, which can easily diffuse into the mitochondrial inner membrane, uses this tunnel to approach the FMN cofactor for the redox reaction (e.g., see Baumgartner et al., J. Med. Chem. 2006, 49, 1239-1247).

In an organism, DHODH catalyzes the synthesis of pyrimidines, which are necessary for cell growth. An inhibition of DHODH inhibits the growth of (pathologically) fast proliferating cells, whereas cells which grow at normal speed may obtain their required pyrimidine bases from the normal metabolic cycle. The most important types of cells for the immune response, the lymphocytes, use exclusively the synthesis of pyrimidines for their growth and react particularly sensitively to DHODH inhibition.

DHODH inhibition results in decreased cellular levels of ribonucleotide uridine monophosphate (rUMP), thus arresting proliferating cells in the GI phase of the cell cycle. The inhibition of de novo pyrimidine nucleotide synthesis is of great interest in view of the observations that lymphocytes seem not to be able to undergo clonal expansion when this pathway is blocked. Substances that inhibit the growth of lymphocytes are important medicaments for the treatment of auto-immune diseases.

During homeostatic proliferation, the salvage pathway which is independent of DHODH seems sufficient for the cellular supply with pyrimidine bases. Only, cells with a high turnover and particularly T and B lymphocytes need the de novo pathway to proliferate. In these cells, DHODH inhibition stops the cell cycle progression suppressing DNA synthesis and consequently cell proliferation.

Therefore, inhibitors of DHODH show beneficial immunosuppressant and antiproliferative effects in human diseases characterized by abnormal and uncontrollable cell proliferation causing chronic inflammation and tissue destruction. The human enzyme dihydroorotate dehydrogenase (DHODH) represents a well-characterized target for small molecular weight Disease Modifying Antirheumatic Drugs (DMARDs).

Accordingly, in various aspects, the present disclosure pertains to methods of treating a variety of diseases or disorders, including, but not limited to, autoimmune diseases, immune and inflammatory diseases, destructive bone disorders, cancers and malignant neoplastic diseases, angiogenic-related disorders, viral diseases, and infectious diseases.

In a further aspect, the present disclosure pertains to a methods for treating an immunological disorder, inflammatory disorder, cancer or other proliferative disease via inhibition of DHODH by administering to a subject in need of such treatment an effective amount of at least one disclosed compound or at least one disclosed pharmaceutical composition.

In a further aspect, the present disclosure pertains to method for treating an immunological disorder, inflammatory disorder, cancer or other proliferative disease via inhibition of DHODH by administering to a patient in need of such treatment an effective amount of at least one disclosed compound or at least one disclosed pharmaceutical composition in combination (simultaneously or sequentially) with at least one other antiinflammatory, immunomodulator or anti-cancer agent.

In various aspects, an autoimmune disorder or disease that can be treated by the disclosed compounds or disclosed pharmaceutical compositions include, but are not limited, one selected from lupus, rheumatoid arthritis, ankylosing spondylitis, glomerulonephritis, minimal change disease, ulcerative colitis, crohns disease, addison’s disease, adult Still’s disease, alopecia areata, autoimmune hepatitis, autoimmune angioedema, Bechet’s disease, pemphigoid and variants, celiac disease, chronic inflammatory demyelinating polyneuropathy, churg-Straus syndrome, Crest syndrome, dermatomyositis, neuromyelitis optica, discoid lupus, fibromyalgia, giant cell arteritis, giant cell myocarditis, Goodpasteur’s disease, evan’s syndrome, autoimmune hemolytic anemia, immune thrombocytopenia, Henoch-Schonlein purpura, IgA nephropathy, IgG4 related sclerosing disease, juvenile arthritis, juvenile diabetes, Kawasaki disease, Leukocytoclastic vasculitis, mixed connective disease, multiple sclerosis, multifocal motor neuropathy, myasthenia gravis, autoimmune neutropenia, optic neuritis, peripheral neuropathy, POEMS syndrome, polymyositis, primary biliary cirrhosis, non-alcoholic hepatosteotosis and associated cirrhosis, psoriasis, scleroderma, sarcoidosis, temporal arteritis, vasculitis, and uveitis.

In a further aspect, autoimmune diseases that can be treated by the disclosed compounds or disclosed pharmaceutical compositions include, but are not limited, to rheumatoid arthritis, psoriatic arthritis, systemic lupus erythematosus, multiple sclerosis, psoriasis, ankylosing spondilytis, Wegener’s granulomatosis, polyarticular juvenile idiopathic arthritis, inflammatory bowel disease such as ulcerative colitis and Crohn’s disease, Reiter’s syndrome, fibromyalgia and type-1 diabetes.

Immune and inflammatory diseases that can be treated by the disclosed compounds or disclosed pharmaceutical compositions include, but are not limited, to asthma, COPD, respiratory distress syndrome, acute or chronic pancreatitis, graft versus-host disease, chronic sarcoidosis, transplant rejection, contact dermatitis, atopic dermatitis allergic rhinitis, allergic conjunctivitis, Behçet’s syndrome, inflammatory eye conditions such as conjunctivitis and uveitis.

In various aspects, the present disclosure pertains to methods for treating organ rejection diseases or ameliorating and/or preventing organ rejection diseases in patients predisposed to organ rejection by administering to a patient in need of such treatment an effective amount of at least one disclosed compound or disclosed pharmaceutical composition. In a further aspect, the patient has received an organ transplant or is diagnosed as requiring an organ transplant. In a still further aspect, the organ transplant can include, but is not limited to, a transplanted organ of the kidney, liver, skin, heart, pancreas, lung, or combinations thereof.

In various aspects, the present disclosure pertains to methods for treating EBV viral lymphoproliferation in the setting of tumor immunosuppression. In a further aspect, the method of treating EBV viral lymphoproliferation can be to provide both continued organ transplantation preservation and also treatment of the underlying EBV lymphoproliferation.

Destructive bone disorders that can be treated by the disclosed compounds or disclosed pharmaceutical compositions include, but are not limited, to osteoporosis, osteoarthritis and multiple myeloma-related bone disorder.

Cancers and malignant neoplastic that can be treated by the disclosed compounds or disclosed pharmaceutical compositions include, but are not limited, to prostate, ovarian and brain cancer. Carcinoma, including that of the bladder, breast, colon, kidney, liver, lung, including small cell lung cancer, esophagus, gall bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin’s lymphoma, non-Hodgkins lymphoma, hairy cell lymphoma and Burkett’s lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including melanoma, seminoma, teratocarcinoma, osteosarcoma, xenoderoma pigmentosum, keratoctanthoma, thyroid follicular cancer and Kaposi’s sarcoma.

Angiogenesis-related disorders that can be treated by the disclosed compounds or disclosed pharmaceutical compositions include, but are not limited, to hemangiomas, ocular neovascularization, macular degeneration or diabetic retinopathy.

Viral diseases that can be treated by the disclosed compounds or disclosed pharmaceutical compositions include, but are not limited, to HIV infection, hepatitis and cytomegalovirus infection.

Infectious diseases that can be treated by the disclosed compounds or disclosed pharmaceutical compositions include, but are not limited, to sepsis, septic shock, endotoxic shock, Gram negative sepsis, toxic shock syndrome, Shigellosis and other protozoal infestations such as malaria.

In further aspects, the disclosed compounds or disclosed pharmaceutical compositions can act as modulators of apoptosis, and accordingly, can be useful in the treatment of cancer (including but not limited to those types mentioned herein above), viral infections (including but not limited to herpes virus, poxvirus, Epstein-Barr virus, Sindbis virus and adenovirus), prevention of AIDS development in HIV-infected individuals, autoimmune diseases (including but not limited to systemic lupus, erythematosus, autoimmune mediated glomerulonephritis, rheumatoid arthritis, psoriasis, inflammatory bowel disease, and autoimmune diabetes mellitus), neurodegenerative disorders (including but not limited to Alzheimer’s disease, AIDS-related dementia, Parkinson’s disease, amyotrophic lateral sclerosis, retinitis pigmentosa, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis) aspirin-sensitive rhinosinusitis, cystic fibrosis, multiple sclerosis, kidney diseases and cancer pain.

In further aspects, the disclosed compounds or disclosed pharmaceutical compositions can act to modulate the level of cellular RNA and DNA synthesis. Accordingly, the disclosed compounds and disclosed pharmaceutical compositions can be used in the treatment of viral infections (including but not limited to HIV, human papilloma virus, herpesvirus, poxvirus, Epstein-Barr virus, Sindbis virus and adenovirus).

In further aspects, the disclosed compounds or disclosed pharmaceutical compositions can be used in the chemoprevention of cancer. Chemoprevention is understood to be a clinical intervention to inhibit the development of invasive cancer by either blocking the initiating mutagenic event or by blocking the progression of pre-malignant cells that have already suffered an insult or inhibiting tumor relapse. Accordingly, the disclosed compounds and disclosed pharmaceutical compositions can be used in inhibiting tumor angiogenesis and metastasis.

In further aspects, the disclosed compounds and disclosed pharmaceutical compositions can also be combined with other active compounds in the treatment of diseases wherein the inhibition of DHODH is known to show beneficial effect.

In various aspects, the diseases, conditions or disorders that can benefit from inhibition of DHODH include, but are not limited to, an immune system-related disease (e.g., an autoimmune disease), a disease or disorder involving inflammation (e.g., asthma, chronic obstructive pulmonary disease, rheumatoid arthritis, inflammatory bowel disease, glomerulonephritis, neuroinflammatory diseases, multiple sclerosis, uveitis and disorders of the immune system), cancer or other proliferative disease, hepatic diseases or disorders, renal diseases or disorders.

In a further aspect, the disclosed compounds and disclosed pharmaceutical compositions can be used as immunosuppressants to prevent transplant graft rejections, allogeneic or xenogeneic transplantation rejection (organ, bone marrow, stem cells, other cells and tissues), and graft-versus-host disease. In other aspects, transplant graft rejections result from tissue or organ transplants. In further aspects, graft-versus-host disease results from bone marrow or stem cell transplantation.

In a further aspect, the disclosed compounds and disclosed pharmaceutical compositions can be used in the treatment of a variety of inflammatory diseases including, but not limited to, inflammation, glomerulonephritis, uveitis, hepatic diseases or disorders, renal diseases or disorders, chronic obstructive pulmonary disease, rheumatoid arthritis, inflammatory bowel disease, vasculitis, dermatitis, osteoarthritis, inflammatory muscle disease, allergic rhinitis, vaginitis, interstitial cystitis, scleroderma, osteoporosis, eczema, allogeneic or xenogeneic transplantation, graft rejection, graft-versus-host disease, corneal transplant rejection, lupus erythematosus, systemic lupus erythematosus, proliferative lupus nephritis, type I diabetes, pulmonary fibrosis, dermatomyositis, thyroiditis, myasthenia gravis, autoimmune hemolytic anemia, cystic fibrosis, chronic relapsing hepatitis, primary biliary cirrhosis, allergic conjunctivitis, hepatitis and atopic dermatitis, asthma and Sjogren’s syndrome.

In a further aspect, the disclosed compounds and disclosed pharmaceutical compositions can be used in the treatment of a variety of diseases including Felty’s syndrome, Wegener’s granulomatosis, Crohn’s disease, sarcoidosis, Still’s disease, pemphigoid, Takayasu arteritis, systemic sclerosis, relapsing polychondritis, refractory IgA nephropathy, SAPHO2 syndrome (SAS), cytomegalovirus infection including rhinitis or cyst, psoriasis, IGG4 disease, and multiple myeloma.

In a further aspect, the disclosed compounds and disclosed pharmaceutical compositions can be used in combination (administered together or sequentially) with known anti-cancer treatments such as radiation therapy or with cytostatic or cytotoxic or anticancer agents, such as for example, but not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and antimetabolites, such as methotrexate, other tyrosine kinase inhibitors such as Iressa and OSI-774; angiogenesis inhibitors; BTK inhibitors, SYK inhibitors, ITK inhibitors, Pl3-kinase inhibitors, FLT3 inhibitors, EGF inhibitors; PAK inhibitors, VEGF inhibitors; CDK inhibitors; SRC inhibitors; c-Kit inhibitors; Her½ inhibitors and monoclonal antibodies directed against growth factor receptors such as erbitux (EGF) and herceptin (Her2) and other protein kinase modulators as well. These agents can be used in combination with differentiation agents such as ATRA, EZH2 inhibitors, DNMT inhibitors, corticosteroids, IDH1 inhibitors, IDH2 inhibitors, and Vitamin C. These agents can be used in combination with small molecules that enhance DNA damage killing in cancer cells including PARP inhibitors, MDM2 inhibitors, NAMPT inhibitors, and HSP90 inhibitors. These agents can be used in combination with antibodies that target cell surface molecules on immune or cancer cells including but not limited to CD33, CD37, CD19, CD20, CD3, CD123, CD70, BAFFR, CD4, CD8, CD56, and CD38. These agents can be used in combination with antibodies or peptides which neutralize cytokines including, but not limited to IL1Beta, IL6, IL10, IL21, TNFA, TNFB, and IFN. These agents can be used in combination with cellular CAR-T cells to diminish cellular proliferation in the setting of significant cytokine release syndrome and neurotoxicity. These agents can be used to diminish T-cell proliferation, cytokine production, and neurotoxicity in combination with bi-specific antibodies or peptide molecules that target in a dual manner T-cells and immune/tumor cell antigens such as, but not limited to CD19, CD20 CD33, CD123, CD38, and CD37. These agents can be used to diminish T-cell proliferation and tissue damage caused by immune check point inhibitor antibodies to targets such as, but not limited to PD1, PDL1, CTLA4, and LAG3.

In a further aspect, diseases, disorders or conditions that can be treated or prevented using the disclosed compounds and disclosed pharmaceutical compositions are capable of inhibiting DHODH, and accordingly, useful in the treatment of diseases, conditions or disorders involving inflammation and/or that are related to the immune system. These diseases include, but are not limited, to asthma, chronic obstructive pulmonary disease, rheumatoid arthritis, inflammatory bowel disease, glomerulonephritis, neuroinflammatory diseases such as multiple sclerosis, and disorders of the immune system.

In a further aspect, the disclosed compounds and disclosed pharmaceutical compositions can be used for treating immune and immune-related disorders, including, for example, chronic immune diseases/disorders, acute immune diseases/disorders, autoimmune and immunodeficiency diseases/disorders, diseases/disorders involving inflammation, organ transplant graft rejections and graft-versus-host disease and altered (e.g., hyperactive) immune responses. In a still further aspect, other exemplary immune disorders that can be treated using the disclosed compounds and disclosed pharmaceutical compositions include psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, osteoporosis, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjogren’s syndrome, thyroiditis (e.g., Hashimoto’s and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

Chronic graft-versus-host disease (cGVHD) is a primary cause of nonrelapse mortality after allogeneic hematopoietic stem cell transplantation (HSCT) (Baird K, Pavletic SZ. Curr Opin Hematol. 2006; 13(6):426-435; Lee SJ, Vogelsang G, Flowers ME. Biol Blood Marrow Transplant. 2003; 9(4):215-233; Pidala J, et al. Blood. 2011; 117(17):4651-4657; and Arai S, et al. Blood. 2011; 118(15):4242-4249). Drug therapy for cGVHD has been predominantly limited to steroids and calcineurin inhibitors, which are incompletely effective and associated with infections as well as long-term risks of toxicity (Holler, E. Best Pract Res Clin Haematol. 2007; 20(2):281-294). The disclosed compounds can be used for the treatment of cGVHD.

### Kits

In various aspects, the present disclosure pertains to kits comprising a therapeutically effective amount of at least one disclosed compound, a disclosed product of the methods of making a disclosed compound, or a pharmaceutically acceptable salt thereof, or a disclosed pharmaceutical composition; and: at least one agent known to treat a cancer, a host-versus-graft-disease, and/or a disorder associated with T-cell proliferation; and instructions for treating a cancer, a host-versus-graft-disease, and/or a disorder associated with T-cell proliferation.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present disclosure also features such kits further containing instructions for use.

In a further aspect, the present disclosure also provides a pharmaceutical pack or kit comprising one or more containers filled with one or more of the ingredients of the pharmaceutical compositions of the disclosure. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### Research Tools

The disclosed compounds and pharmaceutical compositions have activity as inhibitors of DHODH activity or inhibitors of cell proliferation. As such, the disclosed compounds are also useful as research tools. Accordingly, one aspect of the present disclosure relates to a method of using a compound of the disclosure as a research tool, the method comprising conducting a biological assay using a compound of the disclosure. Compounds of the disclosure can also be used to evaluate new chemical compounds. Thus another aspect of the disclosure relates to a method of evaluating a test compound in a biological assay, comprising: (a) conducting a biological assay with a test compound to provide a first assay value; (b) conducting the biological assay with a compound of the disclosure to provide a second assay value; wherein step (a) is conducted either before, after or concurrently with step (b); and (c) comparing the first assay value from step (a) with the second assay value from step (b). Exemplary biological assays include an in vitro DHODH enzymatic assay or in a cell culture-based assay measuring cell proliferation. Methods suitable for carrying out such assays are described herein. Still another aspect of the disclosure relates to a method of studying a biological system, e.g., a model animal for a clinical condition, or biological sample comprising a DHODH protein, the method comprising: (a) contacting the biological system or sample with a compound of the disclosure; and (b) determining the effects caused by the compound on the biological system or sample.

### Aspects

The following listing of exemplary aspects supports and is supported by the disclosure provided herein as listed in the following:
Aspect 1. A pharmaceutical composition comprising at least one anti-CD38 therapeutic agent as disclosed herein and at least one DHODH inhibitor compound as disclosed herein, a pharmaceutically acceptable salt thereof, and a pharmaceutically acceptable carrier.
Aspect 2. The pharmaceutical composition of Aspect 1, wherein the anti-CD38 therapeutic agent comprises an antibody recognizing CD38.
Aspect 3. The pharmaceutical composition of Aspect 2, wherein the antibody recognizing CD38 is capable of killing a CD38+ cell by antibody dependent cell-mediated phagocytosis (ADCP), cellular fratricide, apoptosis, antibody-dependent cell-mediated cytotoxicity (ADCC) and/or complement-dependent cytotoxicity (CDC).
Aspect 4. The pharmaceutical composition of Aspect 2 or Aspect 3, wherein the antibody recognizing CD38 is a chimeric or humanized antibody, an antibody fragment, an antibody-drug conjugate, a radioimmune therapy antibody conjugate, a nanobody, a bispecific antibody, a trispecific antibody, a single variable-domain antibody, or combinations thereof.
Aspect 5. The pharmaceutical composition of any one of Aspect 2- Aspect 4, wherein the antibody recognizing CD38 is selected from daratumumab, isatuximab (SAR650984), felzartamab, ISB-1342, Y-150, ISB-1908, KPMW-101, AMG-424, XmAb-13243, XmAb-13551, MOR202 (MorphoSys AG), TAK-079, TAK-169, KP-1196, BM38, TJ202, and combinations thereof.
Aspect 6. The pharmaceutical composition of Aspect 1, wherein the anti-CD38 therapeutic agent comprises an antigen-specific adoptive cell therapy.
Aspect 7. The pharmaceutical composition of Aspect 6, wherein the antigen-specific adoptive cell therapy comprises a CAR T cell directed to recognize a CD38 epitope or antigen.
Aspect 9. The pharmaceutical composition of Aspect 8, wherein the DHODH inhibitor compound is a compound having a formula represented by a structure:

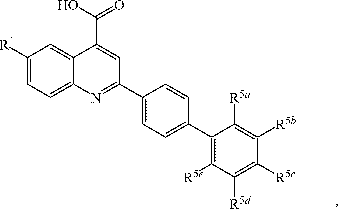

wherein R¹ is selected from halogen, —SF₅, —CN, —N₃, —OH, —NH₂, —CF₃, and —CF₂CF₃; wherein each of R^{5b} and R^{5c} is independently selected from —R²⁰, hydrogen, halogen, —SF₅, —CN, —N₃, —OH, —NH₂, —CF₃, and —CF₂CF₃; wherein R²⁰ is selected from -C1-C10 alkylamino and -C1-C10 alkoxy; provided that one of R^{5b} and R^{5c} is -R²⁰; and wherein each R^{5a}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.
Aspect 10. The pharmaceutical composition of Aspect 9, wherein R^{5b} is —R²⁰; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.
Aspect 11. The pharmaceutical composition of Aspect 7, wherein R²⁰ is —C2-C7 alkylamino.
Aspect 12. The pharmaceutical composition of Aspect 7, wherein R²⁰ is —C2-C7 alkoxy.
Aspect 13. The pharmaceutical composition of Aspect 7, wherein each of R^{5a}, R^{5c}, R^{5d}, and R^{5e} is selected from halogen and hydrogen.
Aspect 14. The pharmaceutical composition of Aspect 7, wherein each of R^{5a}, R^{5c}, R^{5d}, and R^{5e} is hydrogen.
Aspect 15. The pharmaceutical composition of Aspect 7, wherein R¹ is halogen.
Aspect 16. The pharmaceutical composition of Aspect 15, wherein R¹ is fluoro.
Aspect 17. The pharmaceutical composition of 6, wherein R^{5c} is —R²⁰; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.
Aspect 18. The pharmaceutical composition of Aspect 17, wherein R²⁰ is —C2-C7 alkylamino.
Aspect 19. The pharmaceutical composition of Aspect 17, wherein R²⁰ is —C2-C7 alkoxy.
Aspect 20. The pharmaceutical composition of Aspect 17, wherein each of R^{5a}, R^{5b}, R^{5d}, and R^{5e} is selected from halogen and hydrogen.
Aspect 21. The pharmaceutical composition of Aspect 17, wherein each of R^{5a}, R^{5b}, R^{5d}, and R^{5e} is hydrogen.
Aspect 22. The pharmaceutical composition of Aspect 17, wherein R¹ is halogen.
Aspect 23. The pharmaceutical composition of Aspect 22, wherein R¹ is fluoro.
Aspect 24. The pharmaceutical composition of Aspect 8, wherein the DHODH inhibitor compound is present as:

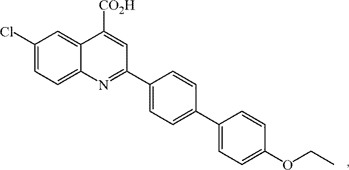

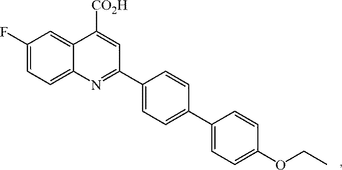

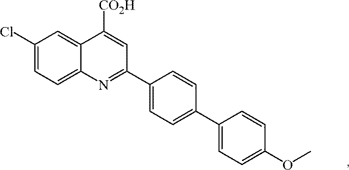

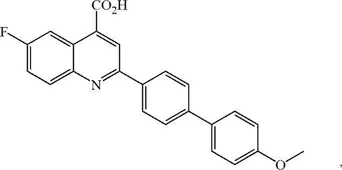

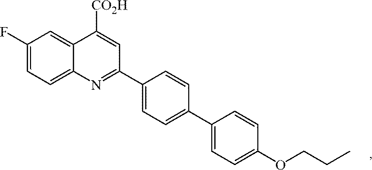

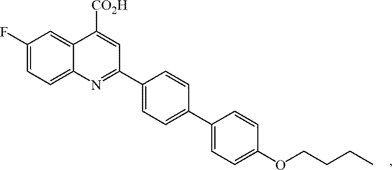

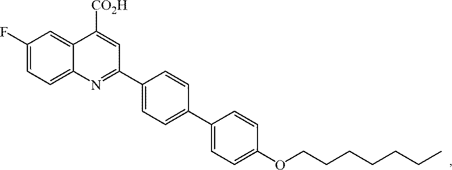

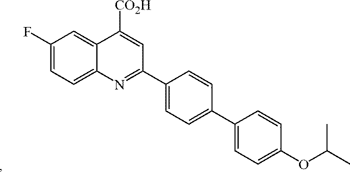

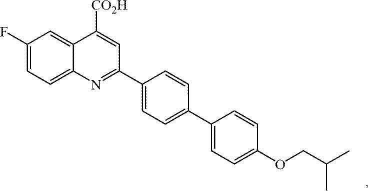

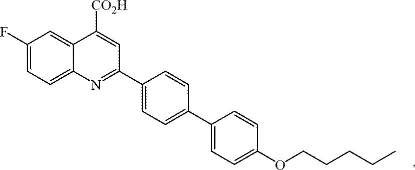

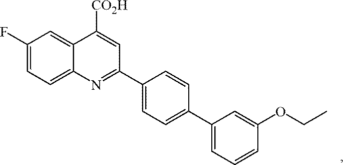

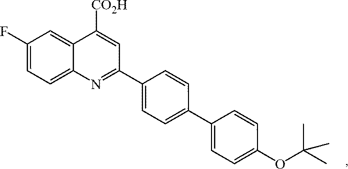

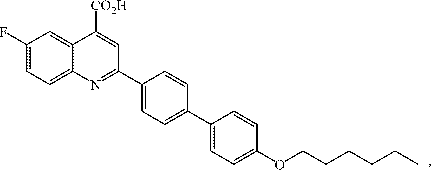

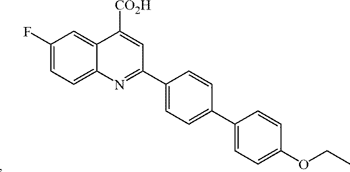

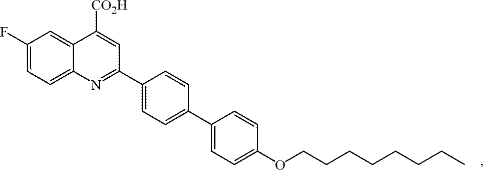

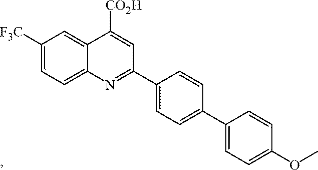

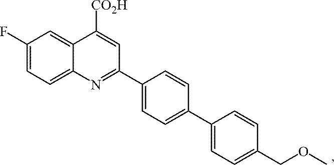

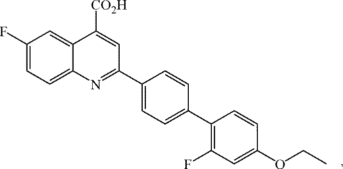

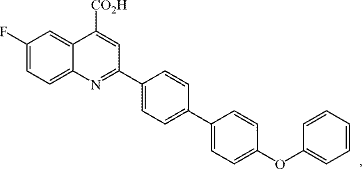

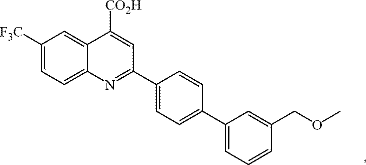

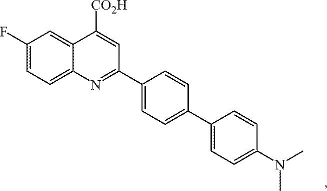

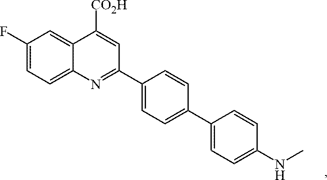

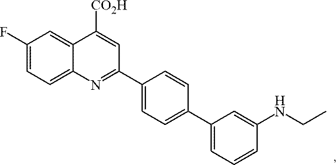

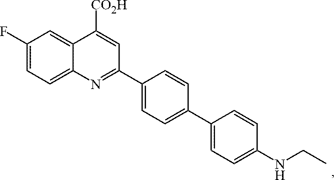

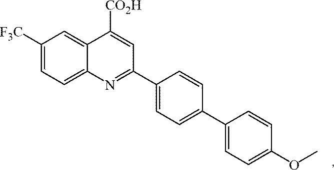

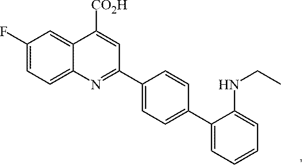

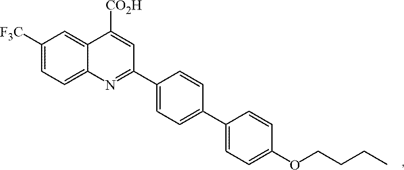

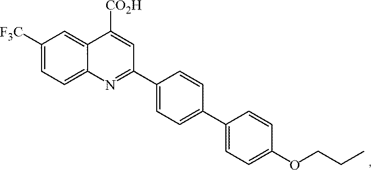

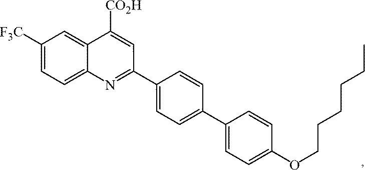

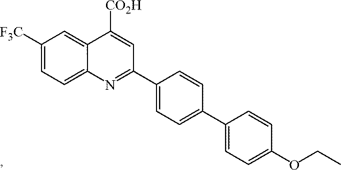

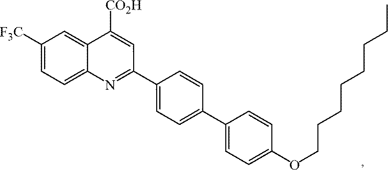

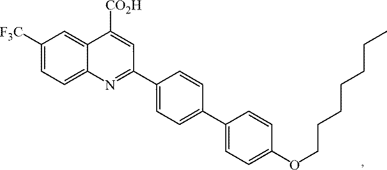

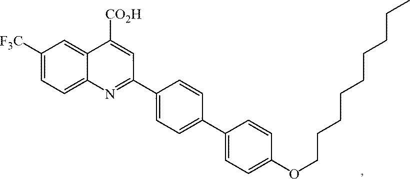

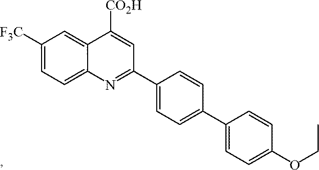

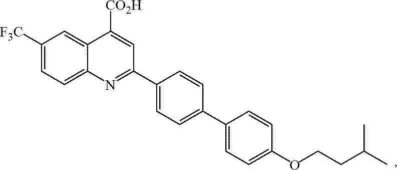

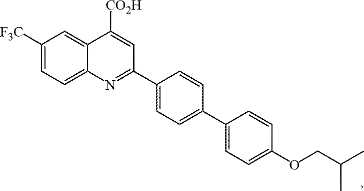

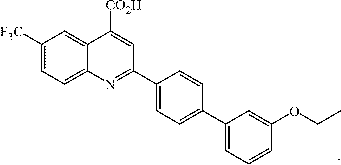

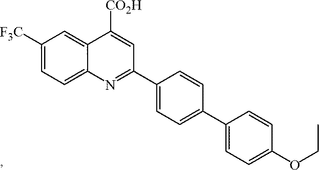

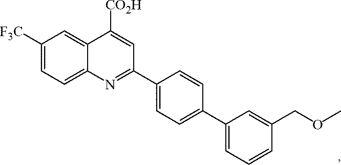

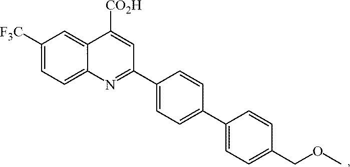

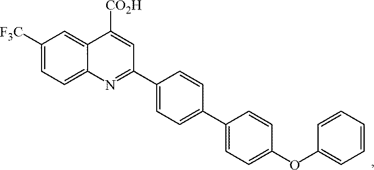

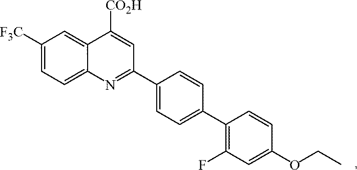

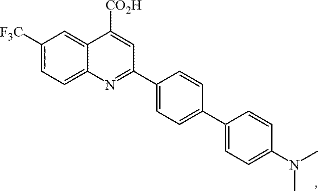

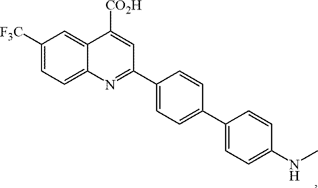

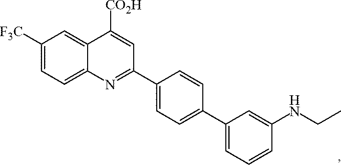

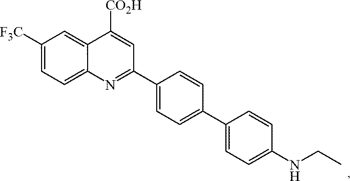

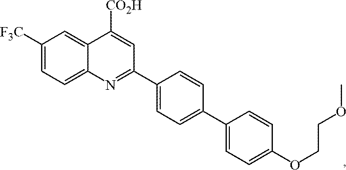

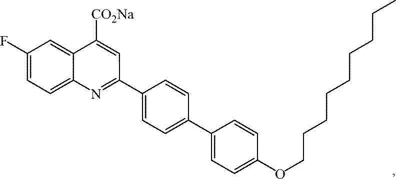

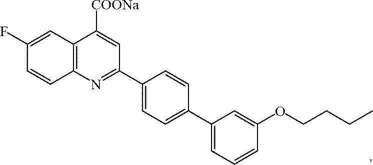

or a subgroup thereof.
Aspect 25. The pharmaceutical composition of any one of Aspect 1-Aspect 7, wherein the DHODH inhibitor compound is a compound of DHODH inhibitor compound Group II as disclosed herein.
Aspect 26. The pharmaceutical composition of Aspect 25, wherein the DHODH inhibitor compound is a compound having a formula represented by a structure:

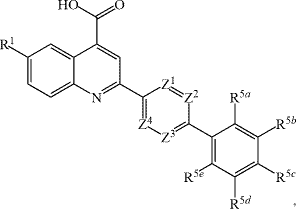

wherein each of Z¹, Z², Z³, and Z⁴ is independently selected from CH and N; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, -R³⁰-A¹-R⁴⁰, -A¹-R⁴⁰, -A¹-R³⁰-A²-R⁴⁰, or —A¹—R³⁰—A²—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, and -C1-C10 alkoxy; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; or a pharmaceutically acceptable salt thereof.
Aspect 27. The pharmaceutical composition of Aspect 26, wherein the DHODH inhibitor compound has a structure represented by a formula:

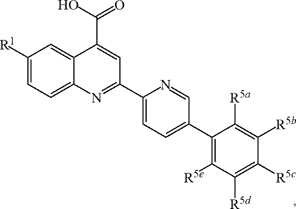

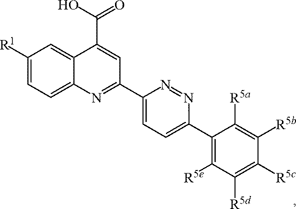

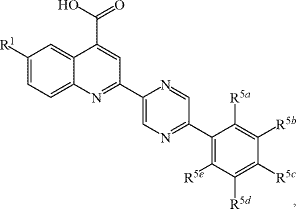

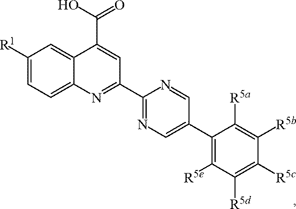

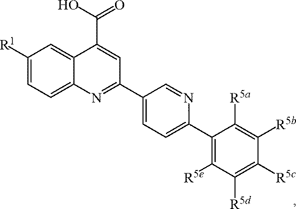

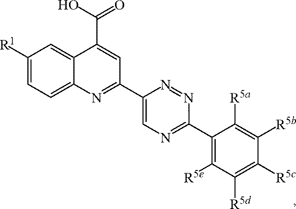

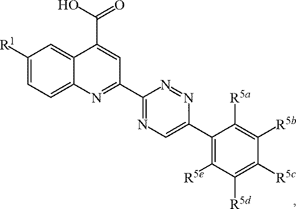

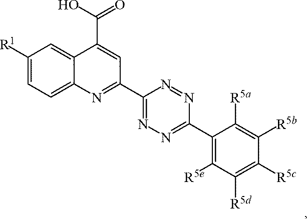

or a subgroup thereof.
Aspect 28. The pharmaceutical composition of Aspect 26, wherein the DHODH inhibitor compound has a structure represented by a formula:

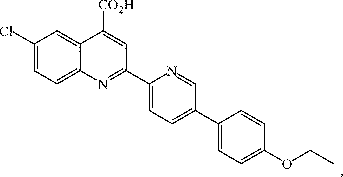

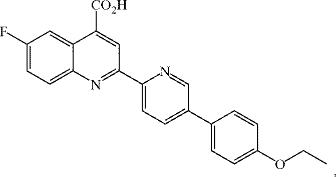

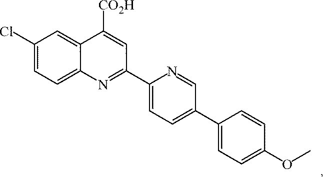

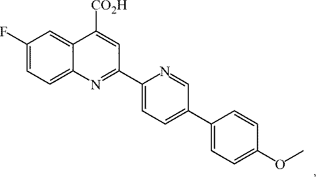

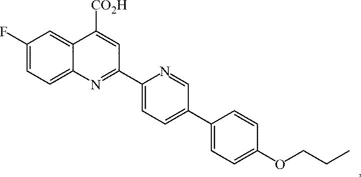

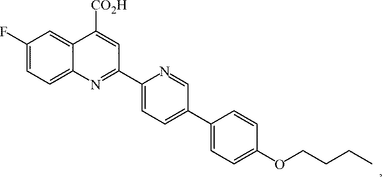

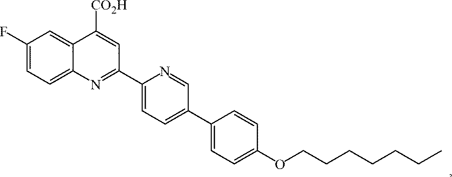

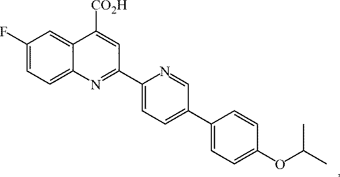

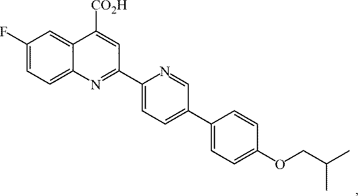

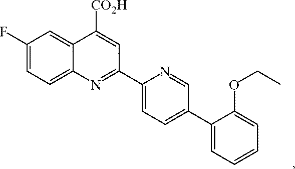

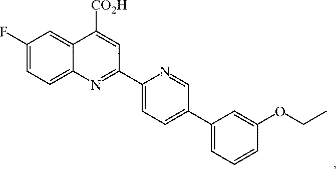

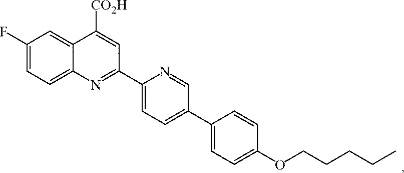

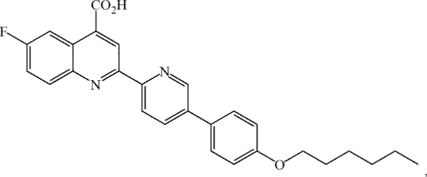

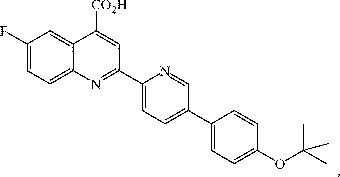

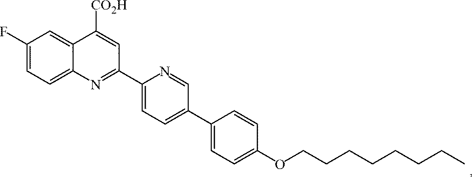

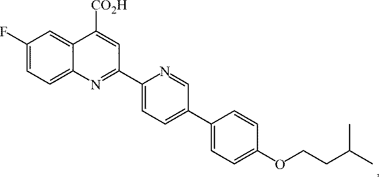

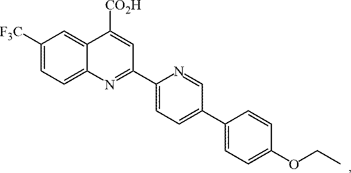

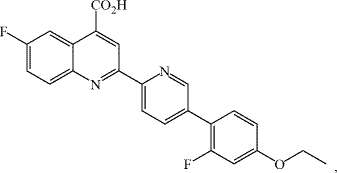

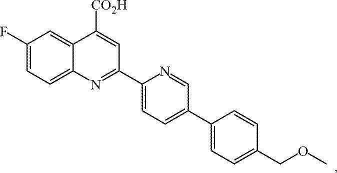

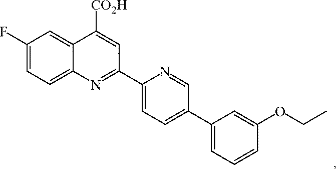

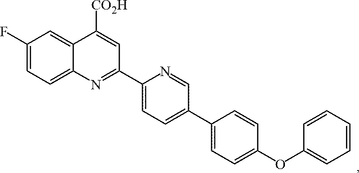

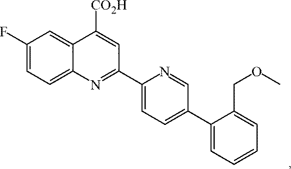

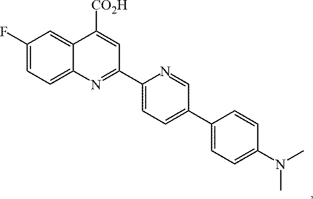

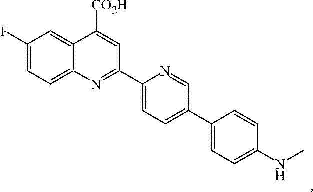

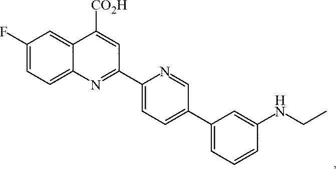

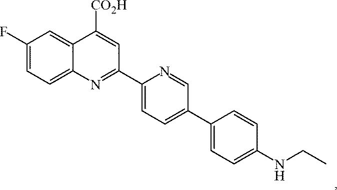

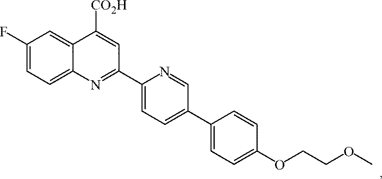

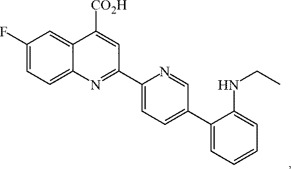

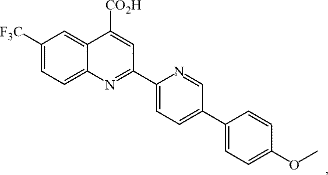

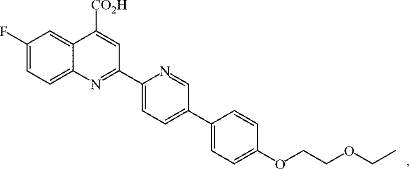

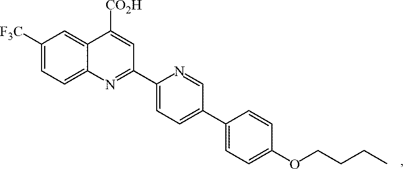

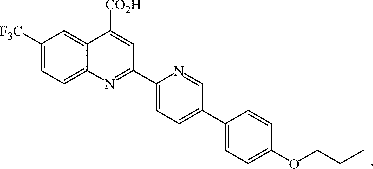

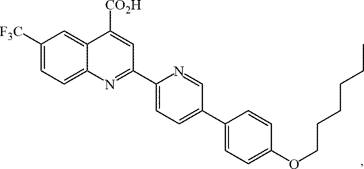

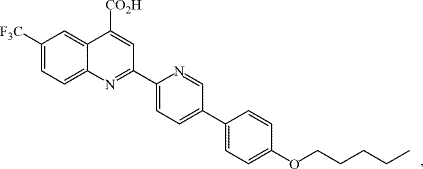

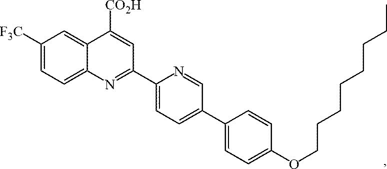

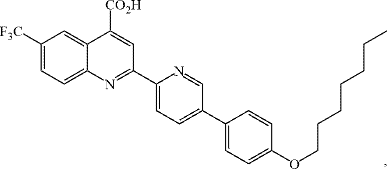

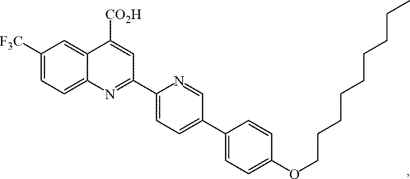

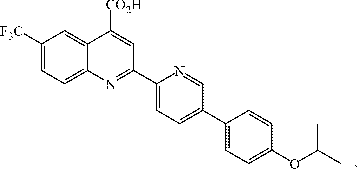

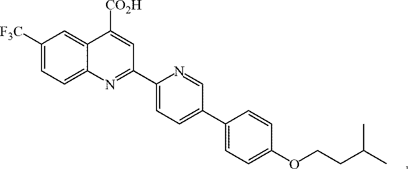

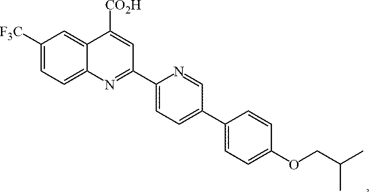

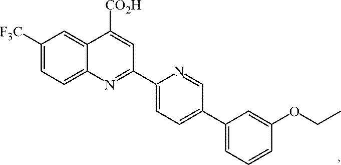

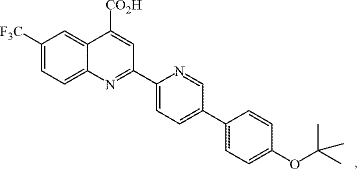

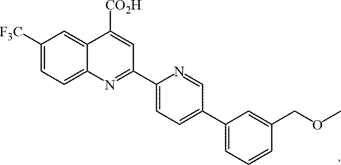

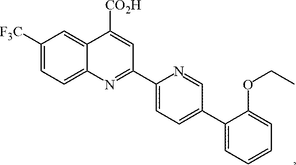

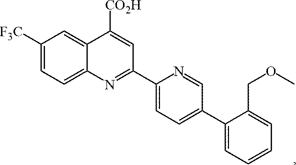

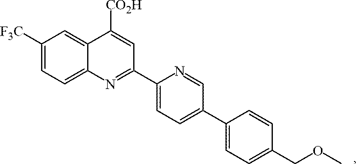

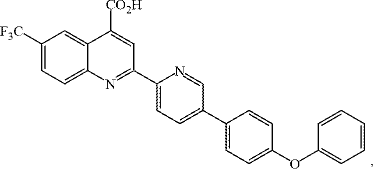

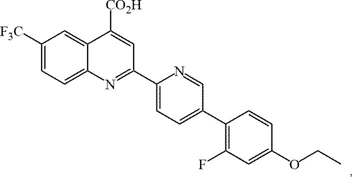

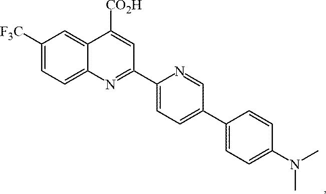

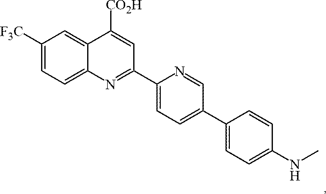

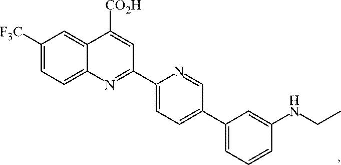

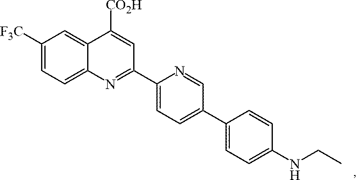

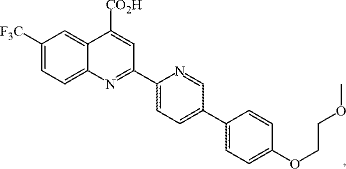

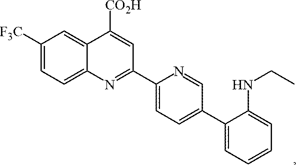

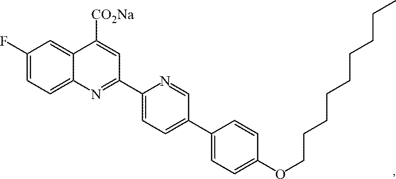

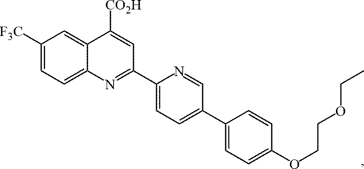

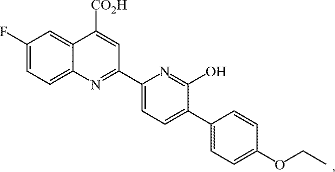

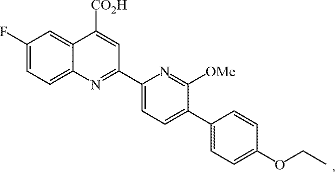

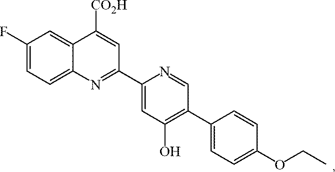

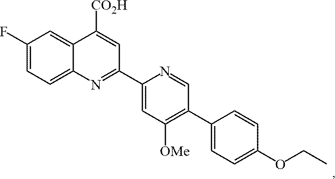

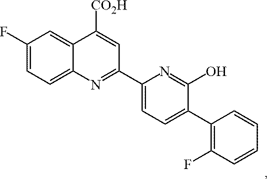

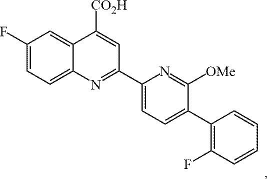

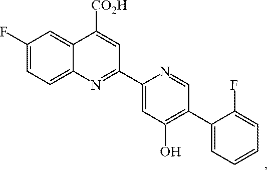

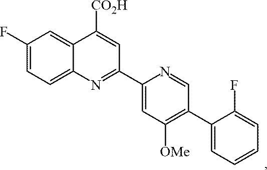

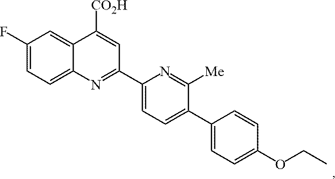

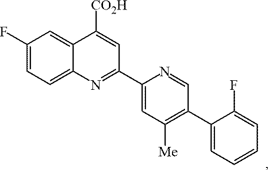

or a subgroup thereof.
Aspect 29. The pharmaceutical composition of any one of Aspect 1-Aspect 7, wherein the DHODH inhibitor compound is a compound of DHODH inhibitor compound Group III as disclosed herein.
Aspect 30. The pharmaceutical composition of Aspect 29, wherein the DHODH inhibitor compound is a compound having a formula represented by a structure:

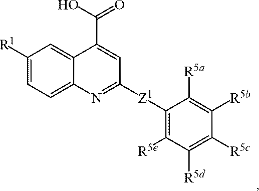

wherein Z¹ is a five-membered heterocyclic diyl; wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, —R³⁰—A¹—R⁴⁰, -A¹-R⁴⁰, —A¹—R³⁰—A²—R⁴⁰, or -A¹-R³⁰-A²-R³¹-A³-R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from -O- and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 alkylamino and -C1-C10 alkoxy; wherein each of R³⁰ and R³¹ is independently selected from -C1-C10 alkanediyl, -C1-C10 aminoalkanediyl, and -C1-C10 hydroxyalkanediyl; and wherein R⁴⁰ is selected from -C1-C10 alkyl, -C1-C10 aminoalkyl, -C1-C10 hydroxyalkyl, and —(CH₂)ₙAr¹; wherein n is an integer selected from 1, 2, and 3; and wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃; or a pharmaceutically acceptable salt thereof.
Aspect 31. The pharmaceutical composition of Aspect 30, wherein Z¹ has a formula represented by a structure:

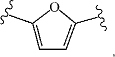

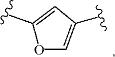

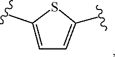

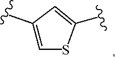

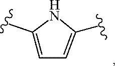

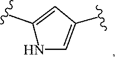

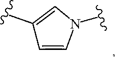

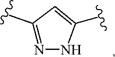

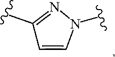

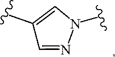

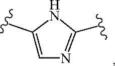

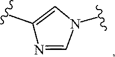

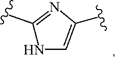

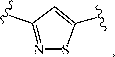

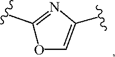

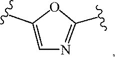

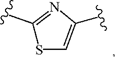

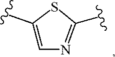

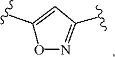

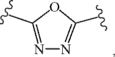

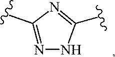

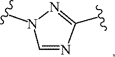

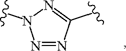

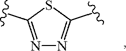

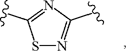

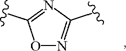

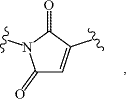

or subgroups thereof.
Aspect 33. The pharmaceutical composition of any one of 1-Aspect 7, wherein the DHODH inhibitor compound is a compound of DHODH inhibitor compound Group IV as disclosed herein.
Aspect 34. The pharmaceutical composition of Aspect 33, wherein the DHODH inhibitor compound is a compound having a formula represented by a structure:

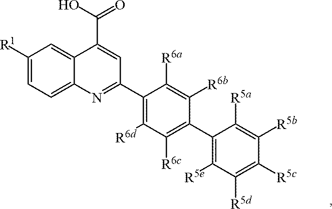

wherein R¹ is selected from hydrogen, halogen, —SF₅, —CN, —N₃, —OH, —NH₂, —CF₃, and —CF₂CF₃; wherein one of R^{5a}, R^{5b}, R^{5c}, R^{5d}, and R^{5e} is selected from a group having formula represented by a structure: —R²⁰, -R³⁰-A¹-R⁴⁰, -A¹-R⁴⁰, -A¹-R³⁰-A²-R⁴⁰, or —A¹—R³⁰—A2—R³¹—A³—R⁴⁰; wherein A¹ is selected from —O— and —NR⁵⁰—; wherein R⁵⁰ is selected from hydrogen, -C1-C10 alkyl, -C1-C10 aminoalkyl, and —C1-C10 hydroxyalkyl; wherein A² is selected from —O— and —NR⁶⁰—; wherein R⁶⁰ is selected from hydrogen, -C1-C10 alkyl, -C1-C10 aminoalkyl, and —C1-C10 hydroxyalkyl; wherein A³ is selected from —O— and —NR⁷⁰—; wherein R⁷⁰ is selected from hydrogen, -C1-C10 alkyl, -C1-C10 aminoalkyl, and -C1-C10 hydroxyalkyl; wherein R²⁰ is selected from halogen, -C1-C10 alkyl, -C1-C10 haloalkyl, -C1-C10 hydroxyalkyl, -C1-C10 alkylamino, -C1-C10 alkoxy, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and wherein R⁴⁰ is selected from -C1-C10 alkyl, -C1-C10 haloalkyl, -C1-C10 aminoalkyl, -C1-C10 hydroxyalkyl, —(CH₂)ₙCy¹, and —(CH₂)ₙAr¹; wherein n is an integer selected from 1, 2, and 3; and wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl; and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃; wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen; or a pharmaceutically acceptable salt thereof.
Aspect 35. The pharmaceutical composition of Aspect 34, wherein the DHODH inhibitor compound is a compound having a formula represented by a structure:

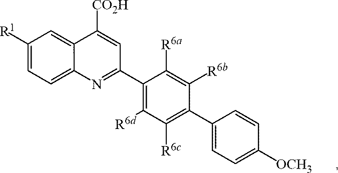

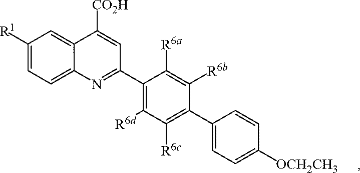

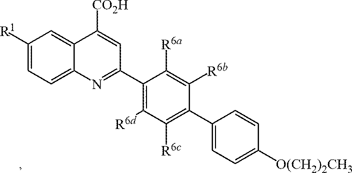

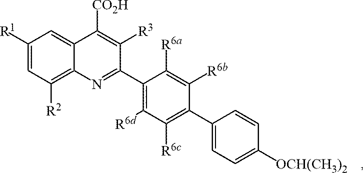

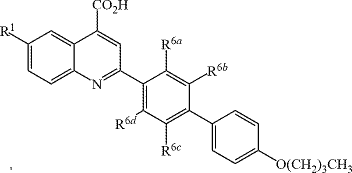

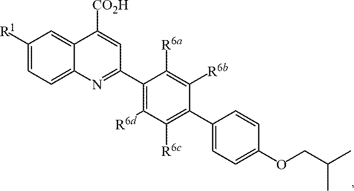

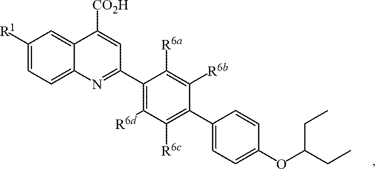

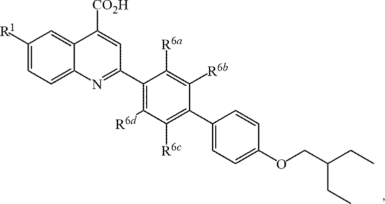

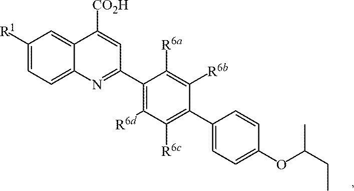

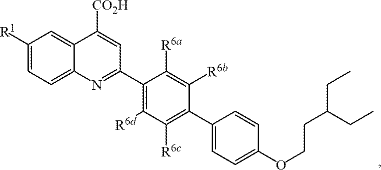

or combinations thereof.
Aspect 36. The pharmaceutical composition of Aspect 34, wherein the DHODH inhibitor compound is a compound having a formula represented by a structure:

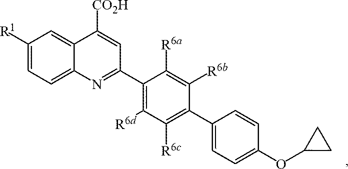

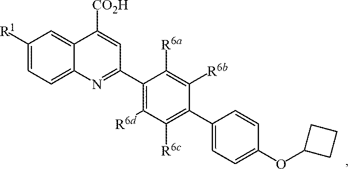

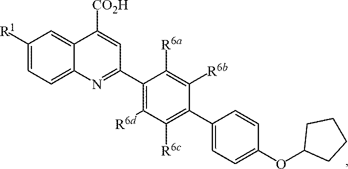

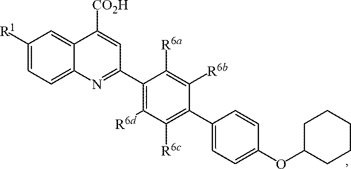

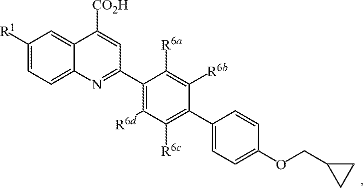

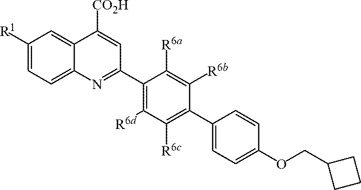

or combinations thereof.
Aspect 37. The pharmaceutical composition of Aspect 34, wherein the DHODH inhibitor compound is a compound having a formula represented by a structure:

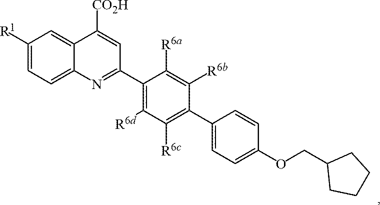

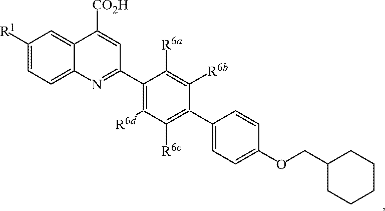

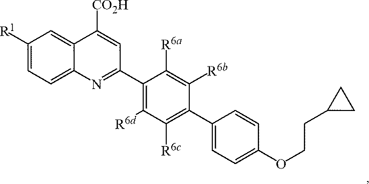

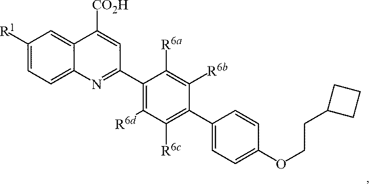

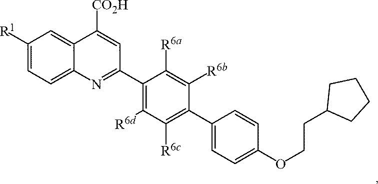

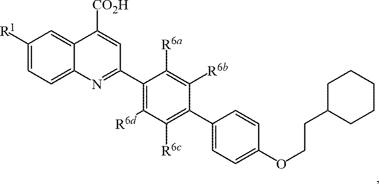

or combinations thereof.
Aspect 38. The pharmaceutical composition of Aspect 34, wherein the DHODH inhibitor compound is a compound having a formula represented by a structure:

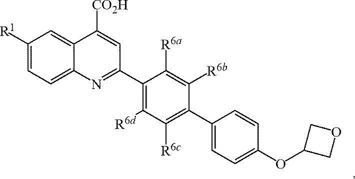

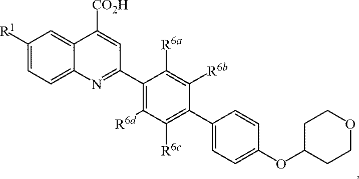

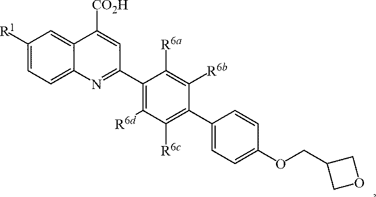

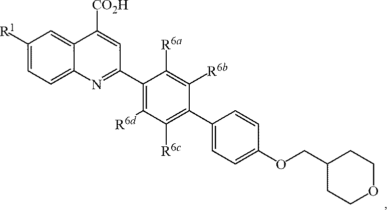

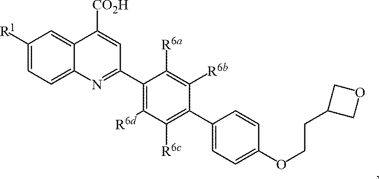

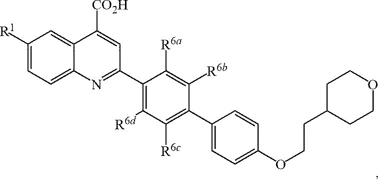

or combinations thereof.
Aspect 39. The pharmaceutical composition of Aspect 34, wherein the DHODH inhibitor compound is a compound having a formula represented by a structure:

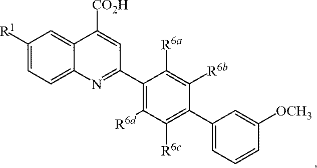

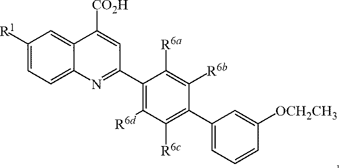

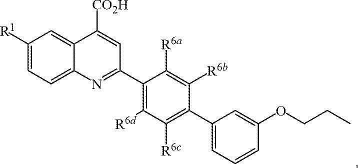

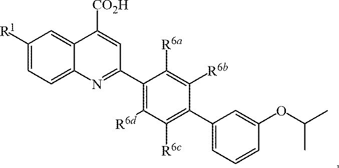

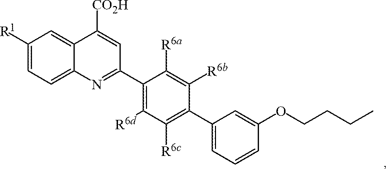

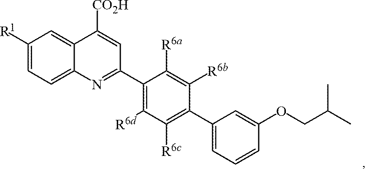

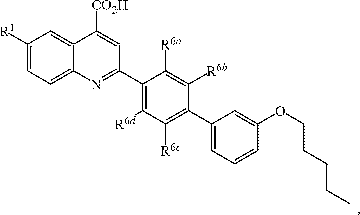

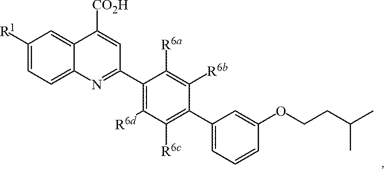

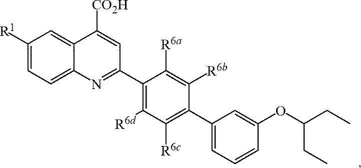

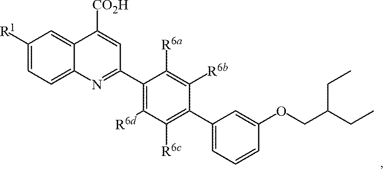

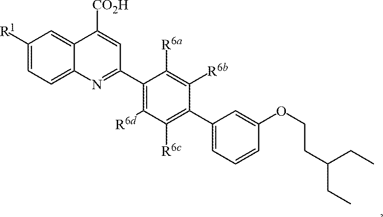

or combinations thereof.
Aspect 40. The pharmaceutical composition of Aspect 34, wherein the DHODH inhibitor compound is a compound having a formula represented by a structure:

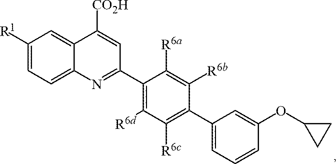

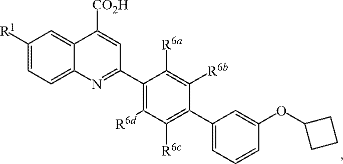

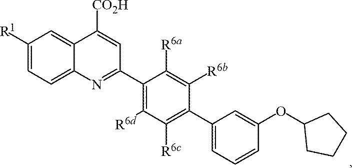

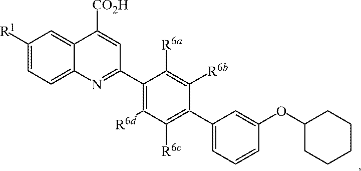

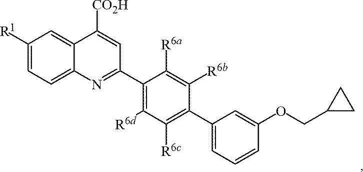

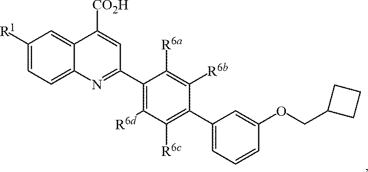

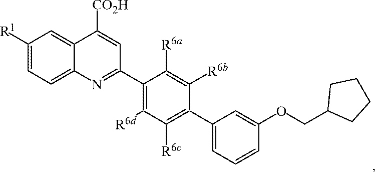

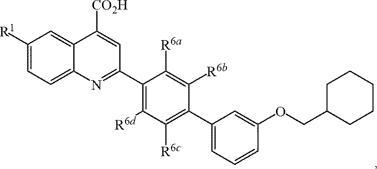

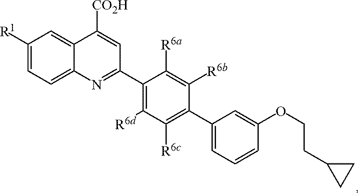

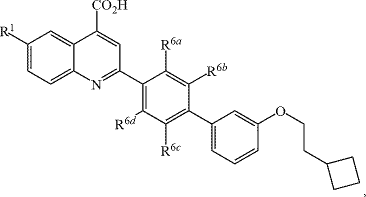

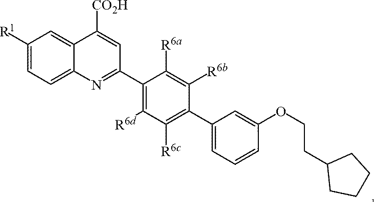

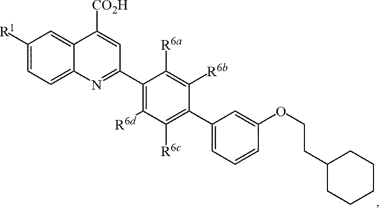

or combinations thereof.
Aspect 41. The pharmaceutical composition of Aspect 34, wherein the DHODH inhibitor compound is a compound having a formula represented by a structure:

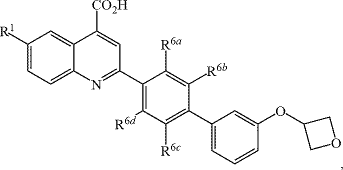

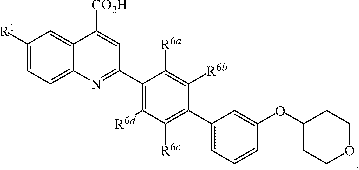

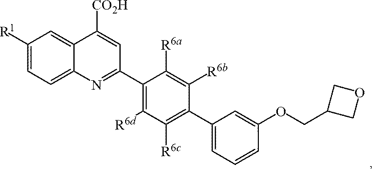

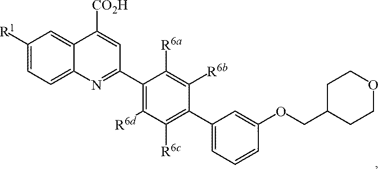

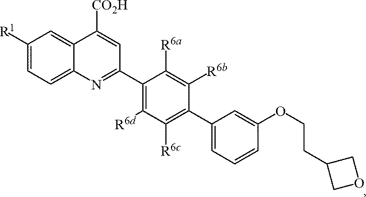

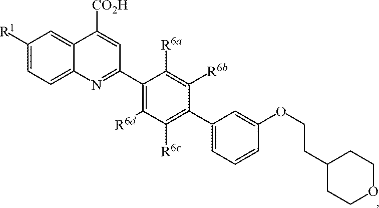

or combinations thereof.
Aspect 42. The pharmaceutical composition of Aspect 34, wherein the DHODH inhibitor compound is a compound having a formula represented by a structure:

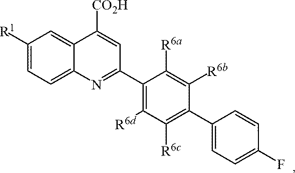

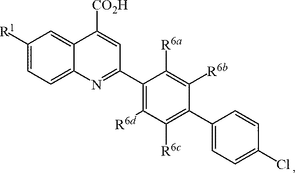

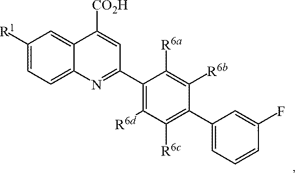

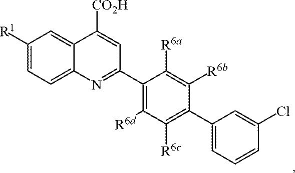

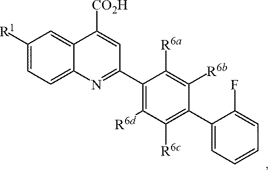

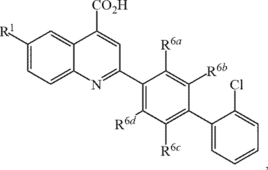

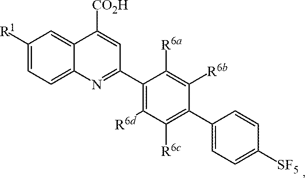

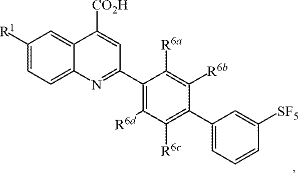

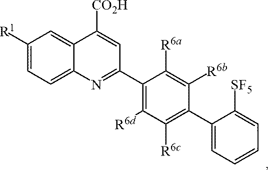

or combinations thereof.
Aspect 43. The pharmaceutical composition of Aspect 34, wherein the DHODH inhibitor compound is a compound having a formula represented by a structure:

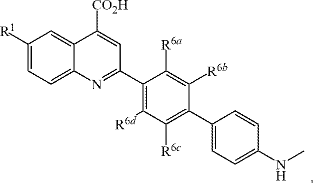

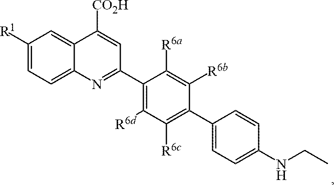

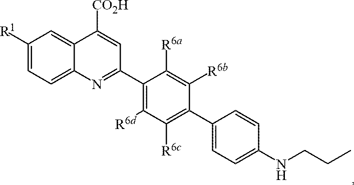

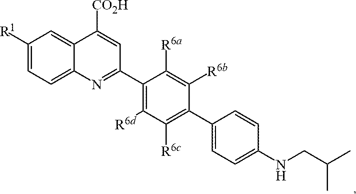

or combinations thereof.
Aspect 44. The pharmaceutical composition of Aspect 34, wherein the DHODH inhibitor compound is a compound having a formula represented by a structure:

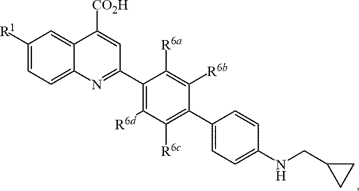

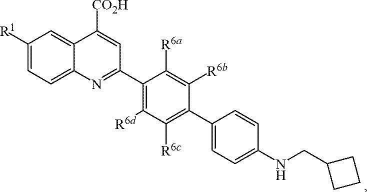

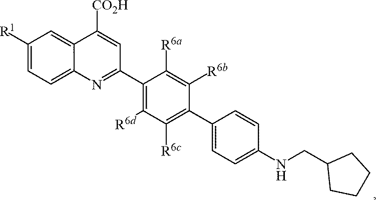

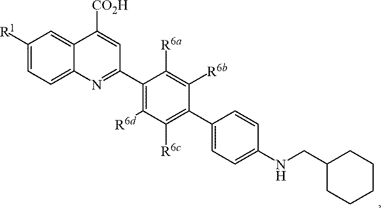

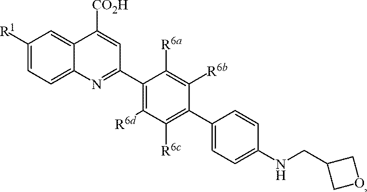

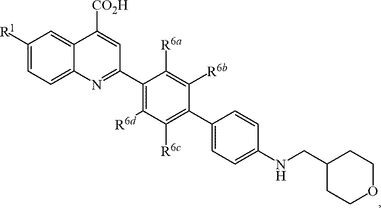

or combinations thereof.
Aspect 45. The pharmaceutical composition of any one of Aspect 34-Aspect 44, wherein R¹ is selected from halogen, —SF₅, —CF₃, and —CF₂CF₃.
Aspect 46. The pharmaceutical composition of Aspect 45, wherein R¹ is halogen or —SF₅.
Aspect 47. The pharmaceutical composition of Aspect 45, wherein R¹ is —F or —Cl.
Aspect 48. The pharmaceutical composition of Aspect 45, wherein R¹ is —F.
Aspect 49. The pharmaceutical composition of Aspect 45, wherein R¹ is—Cl.
Aspect 50. The pharmaceutical composition of Aspect 45, wherein R¹ is —SF₅.
Aspect 51. The pharmaceutical composition of Aspect 45, wherein R¹ is selected—CF₃ and —CF₂CF₃.
Aspect 52. The pharmaceutical composition of any one of Aspect 34-Aspect 51, wherein each of R^{6a}, R^{6b}, R^{6c}, and R^{6d} is independently selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl, provided that at least one of R^{6a}, R^{6b}, R^{6c}, and R^{6d} is not hydrogen.
Aspect 53. The pharmaceutical composition of Aspect 52, wherein R^{6a} and R^{6b} are independently selected from hydrogen, halogen, —SF₅, —CN, —N₃, —OH, —NH₂, —CHF₂, —CH₂F, and —CF₃.
Aspect 54. The pharmaceutical composition of Aspect 53, wherein R^{6a} and R^{6b} are independently selected from halogen, —SF₅, —CN, —N₃, —OH, —NH₂, —CHF₂, —CH₂F, and —CF₃.
Aspect 55. The pharmaceutical composition of Aspect 54, wherein R^{6a} and R^{6b} are independently selected from —F, —Cl, —SF₅, —CN, —N₃, —OH, —NH₂, —CHF₂, —CH₂F, and —CF₃.
Aspect 56. The pharmaceutical composition of Aspect 52, wherein R^{6a} and R^{6c} are independently selected from hydrogen, halogen, —SF₅, —CN, —N₃, —OH, —NH₂, —CHF₂, —CH₂F, and —CF₃.
Aspect 57. The pharmaceutical composition of Aspect 56, wherein R^{6a} and R^{6c} are independently selected from halogen, —SF₅, —CN, —N₃, —OH, —NH₂, —CHF₂, —CH₂F, and —CF₃.
Aspect 58. The pharmaceutical composition of Aspect 57, wherein R^{6a} and R^{6c} are independently selected from —F, —Cl, —SF₅, —CN, —N₃, —OH, —NH₂, —CHF₂, —CH₂F, and —CF₃.
Aspect 59. The pharmaceutical composition of Aspect 52, wherein R^{6a} and R^{6d} are independently selected from hydrogen, halogen, —SF₅, —CN, —N₃, —OH, —NH₂, —CHF₂, —CH₂F, and —CF₃.
Aspect 60. The pharmaceutical composition of Aspect 59, wherein R^{6a} and R^{6d} are independently selected from halogen, —SF₅, —CN, —N₃, —OH, —NH₂, —CHF₂, —CH₂F, and —CF₃.
Aspect 61. The pharmaceutical composition of Aspect 60, wherein R^{6a} and R^{6d} are independently selected from —F, —Cl, —SF₅, —CN, —N₃, —OH, —NH₂, —CHF₂, —CH₂F, and —CF₃.
Aspect 62. The pharmaceutical composition of Aspect 52, wherein R^{6a} is selected from —F, —Cl, —SF₅, —CN, —N₃, —OH, and —NH₂.
Aspect 63. The pharmaceutical composition of Aspect 52, wherein R^{6a} is selected from —F, —SF₅, —CN, —N₃, —OH, and —NH₂.
Aspect 64. The pharmaceutical composition of Aspect 52, wherein R^{6b} is selected from —F, —Cl, —SF₅, —CN, —N₃, —OH, and —NH₂.
Aspect 65. The pharmaceutical composition of Aspect 52, wherein R^{6a} is selected from —F, —SF₅, —CN, —N₃, —OH, and —NH₂.
Aspect 66. The pharmaceutical composition of Aspect 52-Aspect 65, wherein each of R^{6c} and R^{6d} are hydrogen.
Aspect 67. The pharmaceutical composition of any one of Aspect 35-Aspect 51, wherein R^{6a} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b}, R^{6c}, and R^{6d} is hydrogen.
Aspect 68. The pharmaceutical composition of Aspect 67, wherein R^{6a} is selected from halogen, —SF₅, —CN, —N₃, —OH, —NH₂, —CHF₂, —CH₂F, and —CF₃.
Aspect 69. The pharmaceutical composition of Aspect 68, wherein R^{6a} is selected from —F, —Cl, —SF₅, —CN, —N₃, —OH, —NH₂, —CHF₂, —CH₂F, and —CF₃.
Aspect 70. The pharmaceutical composition of Aspect 69, wherein R^{6a} is —F.
Aspect 71. The pharmaceutical composition of any one of Aspect 35-Aspect 51, wherein R^{6b} is selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a}, R^{6c}, and R^{6d} is hydrogen.
Aspect 72. The compound of Aspect 71, wherein R^{6b} is selected from halogen, —SF₅, —CN, —N₃, —OH, —NH₂, —CHF₂, —CH₂F, and —CF₃.
Aspect 73. The pharmaceutical composition of Aspect 72, wherein R^{6b} is selected from —F, —Cl, —SF₅, —CN, —N₃, —OH, —NH₂, —CHF₂, —CH₂F, and —CF₃.
Aspect 74. The pharmaceutical composition of Aspect 73, wherein R^{6b} is —F.
Aspect 75. The pharmaceutical composition of any one of Aspect 35-Aspect 51, wherein each of R^{6a} and R^{6b} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6c} and R^{6d} is hydrogen.
Aspect 76. The pharmaceutical composition of Aspect 75, wherein each of R^{6a} and R^{6b} is independently selected from halogen, —SF₅, —CN, —N₃, —OH, —NH₂, —CHF₂, —CH₂F, and —CF₃.
Aspect 77. The pharmaceutical composition of Aspect 76, wherein each of R^{6a} and R^{6b} is independently selected from —F, —Cl, —SF₅, —CN, —N₃, —OH, —NH₂, —CHF₂, —CH₂F, and —CF₃.
Aspect 78. The pharmaceutical composition of Aspect 77, wherein each of R^{6a} and R^{6b} is —F.
Aspect 79. The pharmaceutical composition of any one of Aspect 35-Aspect 51, wherein each of R^{6a} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6d} is hydrogen.
Aspect 80. The pharmaceutical composition of Aspect 79, wherein each of R^{6a} and R^{6c} is independently selected from halogen, —SF₅, —CN, —N₃, —OH, —NH₂, —CHF₂, —CH₂F, and —CF₃.
Aspect 81. The pharmaceutical composition of Aspect 80, wherein each of R^{6a} and R^{6c} is independently selected from —F, —Cl, —SF₅, —CN, —N₃, —OH, —NH₂, —CHF₂, —CH₂F, and —CF₃.
Aspect 82. The pharmaceutical composition of Aspect 81, wherein each of R^{6a} and R^{6c} is —F.
Aspect 83. The pharmaceutical composition of any one of Aspect 35-Aspect 51, wherein each of R^{6a} and R^{6d} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6b} and R^{6c} is hydrogen.
Aspect 84. The pharmaceutical composition of Aspect 83, wherein each of R^{6a} and R^{6d} is independently selected from halogen, —SF₅, —CN, —N₃, —OH, —NH₂, —CHF₂, —CH₂F, and —CF₃.
Aspect 85. The pharmaceutical composition of Aspect 84, wherein each of R^{6a} and R^{6d} is independently selected from —F, —Cl, —SF₅, —CN, —N₃, —OH, —NH₂, —CHF₂, —CH₂F, and —CF₃.
Aspect 86. The pharmaceutical composition of Aspect 85, wherein each of R^{6a} and R^{6d} —F.
Aspect 87. The pharmaceutical composition of any one of Aspect 35-Aspect 51, wherein each of R^{6b} and R^{6c} is independently selected from halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl; and wherein each of R^{6a} and R^{6d} is hydrogen.
Aspect 88. The pharmaceutical composition of Aspect 87, wherein each of R^{6b} and R^{6c} is independently selected from halogen, —SF₅, —CN, —N₃, —OH, —NH₂, —CHF₂, —CH₂F, and —CF₃.
Aspect 89. The pharmaceutical composition of Aspect 88, wherein each of R^{6b} and R^{6c} is independently selected from —F, —Cl, —SF₅, —CN, —N₃, —OH, —NH₂, —CHF₂, —CH₂F, and —CF₃.
Aspect 90. The pharmaceutical composition of Aspect 89, wherein each of R^{6b} and R^{6c} is —F.
Aspect 91. The pharmaceutical composition of Aspect 34, wherein the DHODH inhibitor compound is a compound having a formula represented by a structure:

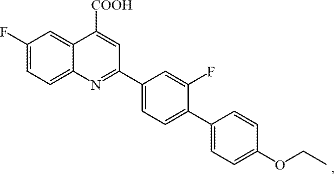

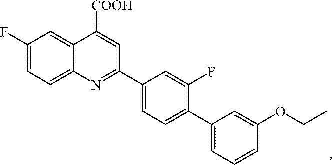

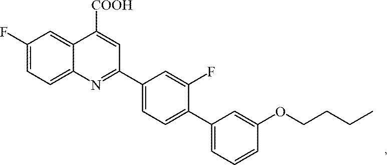

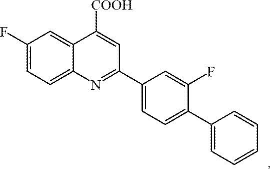

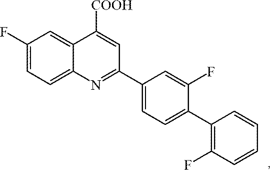

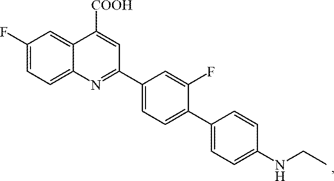

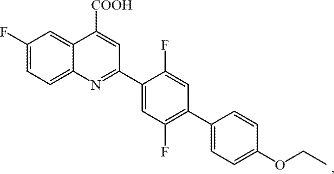

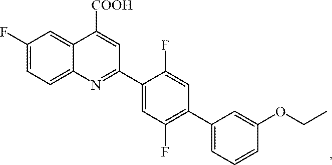

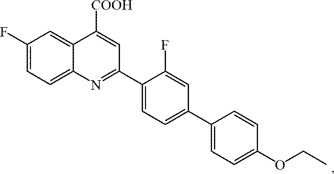

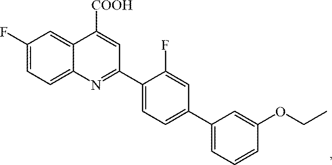

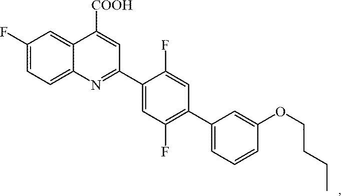

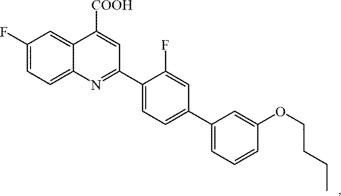

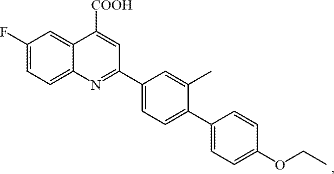

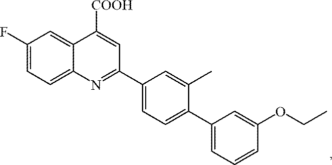

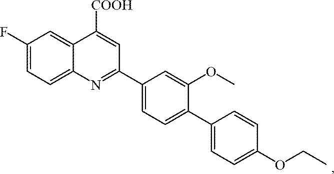

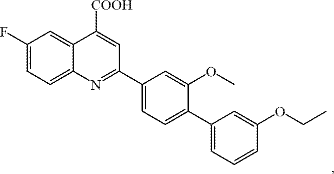

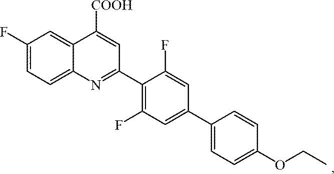

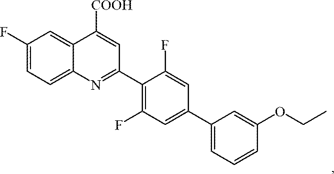

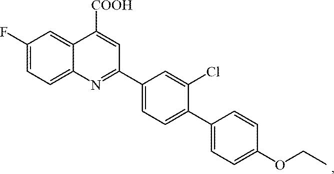

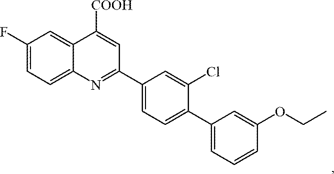

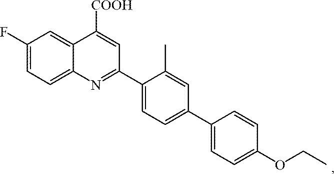

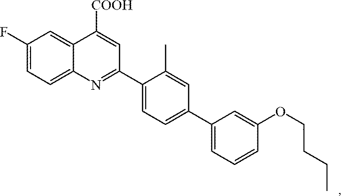

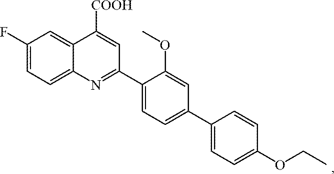

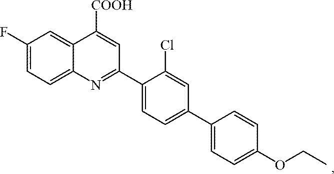

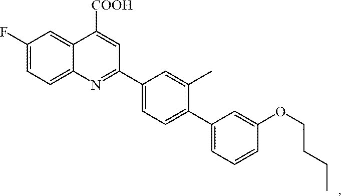

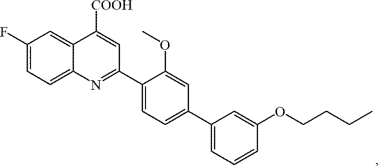

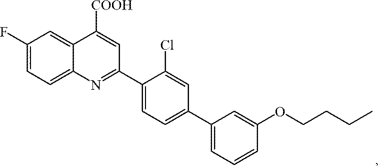

or combinations thereof.
Aspect 92. The pharmaceutical composition of Aspect 34, wherein the DHODH inhibitor compound is a compound having a formula represented by a structure:

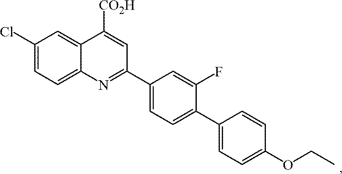

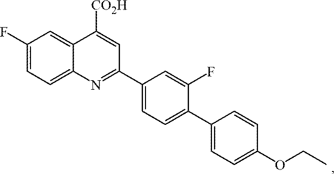

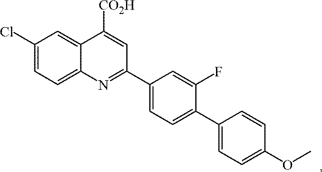

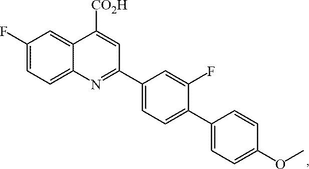

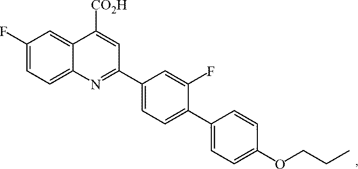

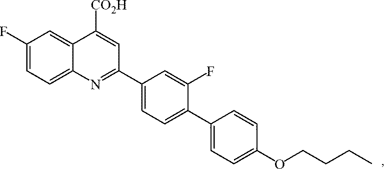

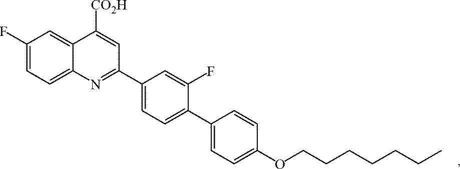

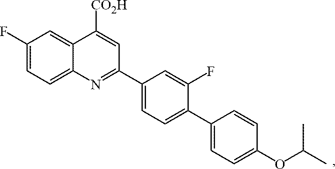

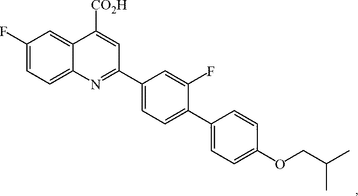

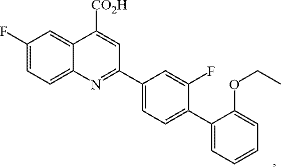

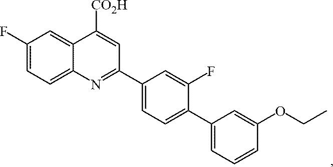

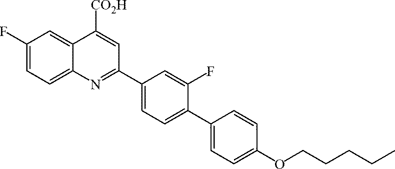 [SMILES: CCCCCOc1ccc(-c2ccc(-c3cc(C(=O)O)c4cc(F)ccc4n3)cc2F)cc1]

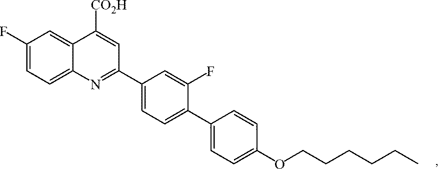

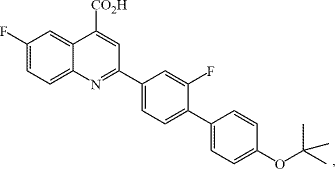

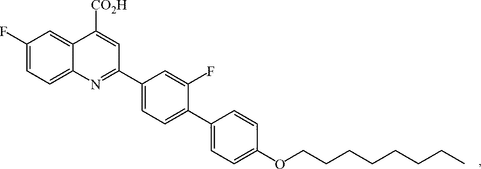

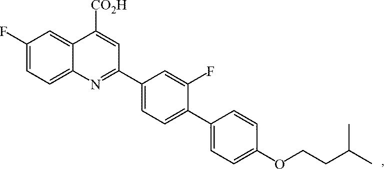

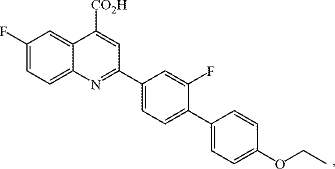

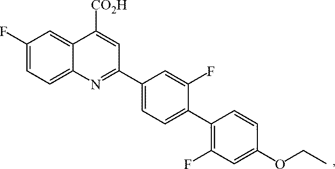

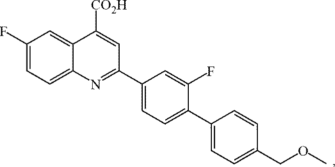

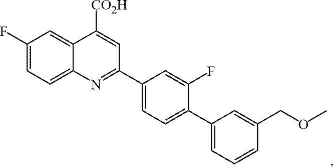

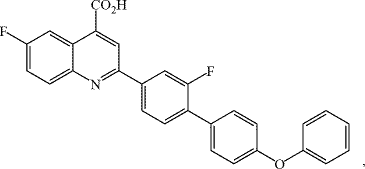

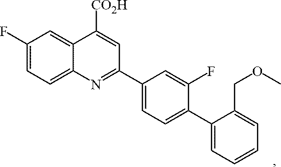

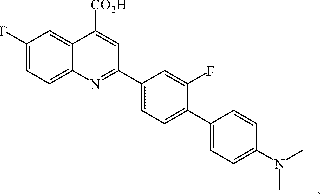

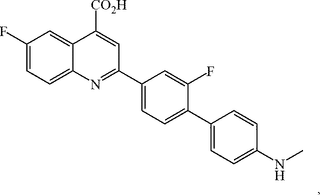

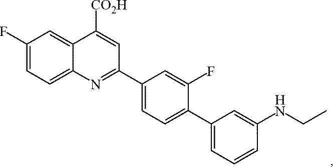

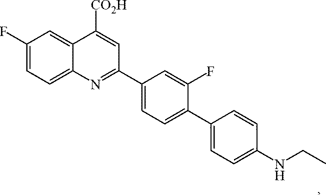

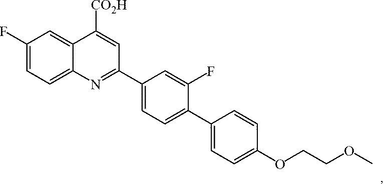

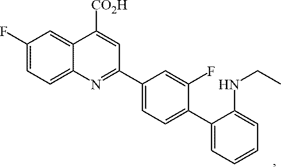

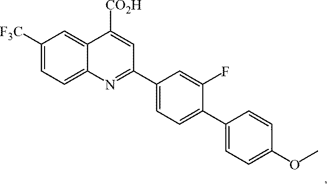

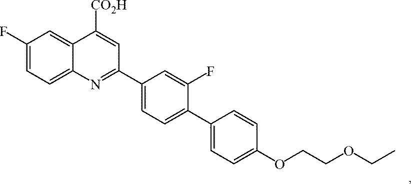

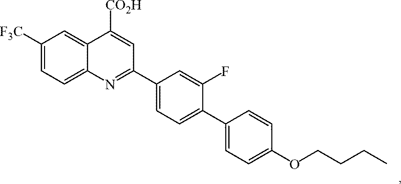

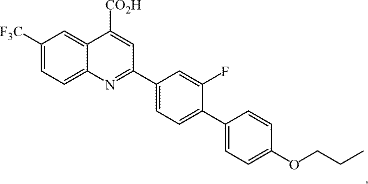

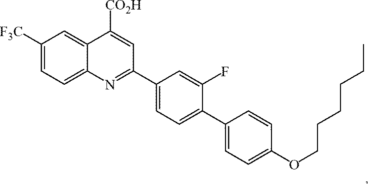

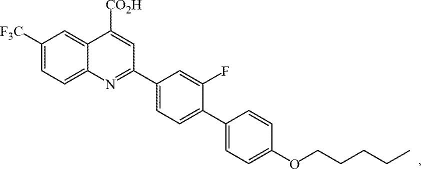

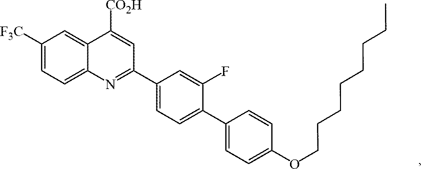

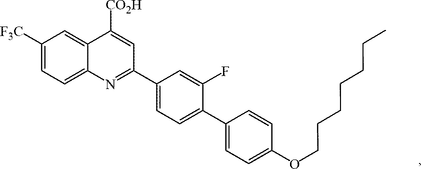

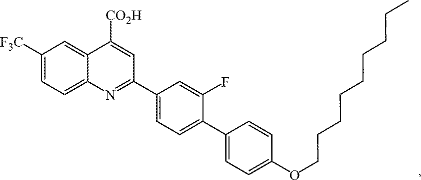

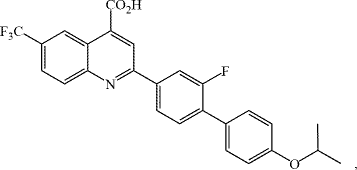

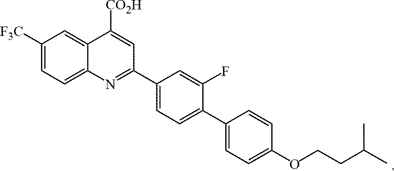

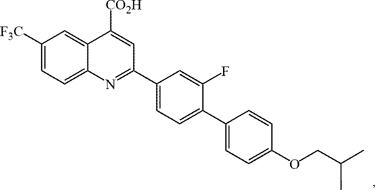

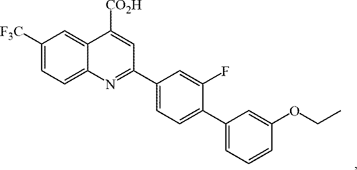

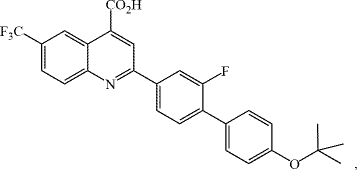

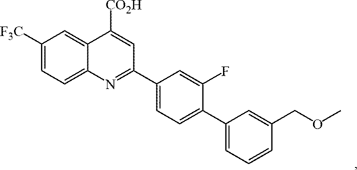

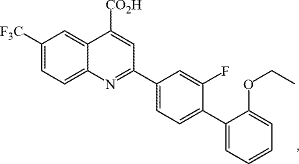

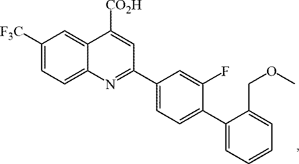

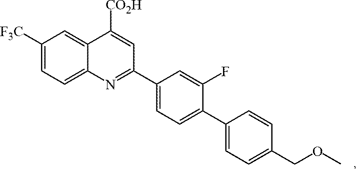

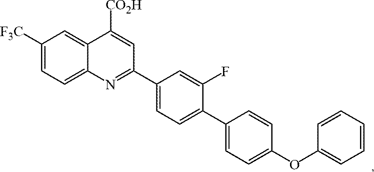

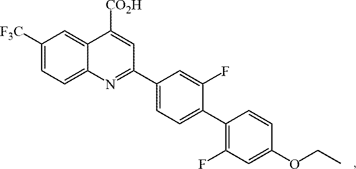

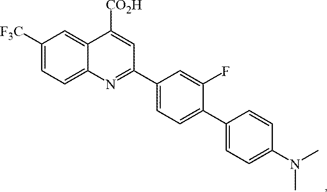

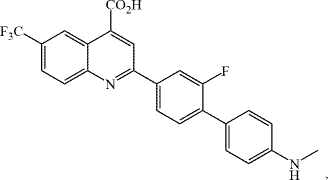

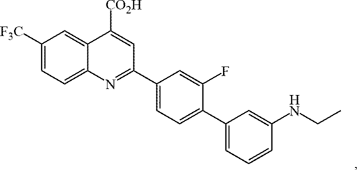

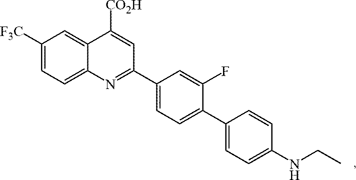

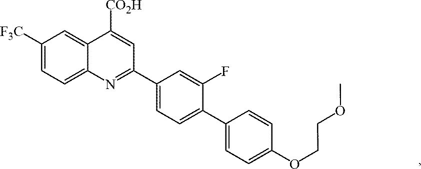

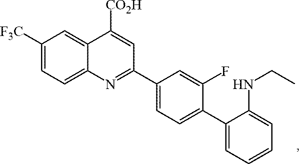

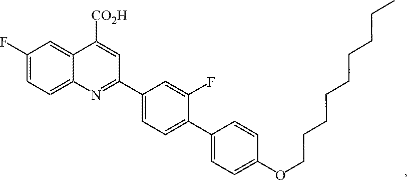

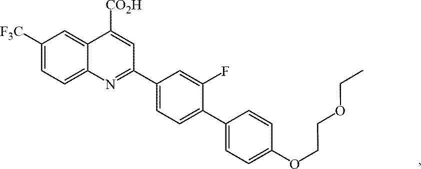

or a subgroup thereof.
Aspect 93. The pharmaceutical composition of Aspect 34, wherein the DHODH inhibitor compound is a compound having a formula represented by a structure:

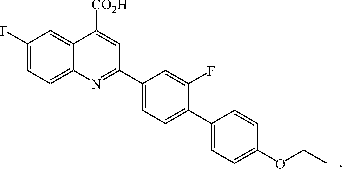

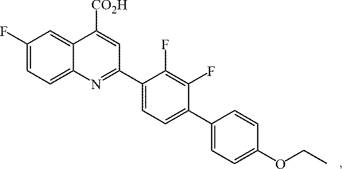

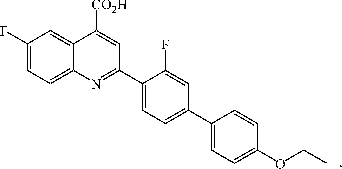

or a combination thereof.
Aspect 94. The pharmaceutical composition of any one of Aspect 6- Aspect 87, wherein the the DHODH inhibitor compound is a pharmaceutically acceptable salt thereof comprising the conjugate base form of the compound, and a counter ion selected from Li⁺, K⁺, Na⁺, ammonium, tetramethylammonium, tetraethylammonium, Fe⁺², Cu⁺², Zn⁺², Mg⁺², Ca⁺², Al⁺³, Fe⁺³, and combinations thereof.
Aspect 95. The pharmaceutical composition of Aspect 88, wherein the counter ion is Na⁺.
Aspect 96. The pharmaceutical composition of any one of Aspect 1-Aspect 7, wherein the DHODH inhibitor compound is a compound of DHODH inhibitor compound Group V as disclosed herein.
Aspect 97. The pharmaceutical composition of Aspect 96, wherein the DHODH inhibitor compound is selected from the group consisting of brequinar, leflunomide, redoxal, vidofludimas, S-2678, 2-(3,5-difluoro-3 ′ methoxybiphenyl-4-ylamino)nicotinic acid (also known as ASLAN003), BAY-2402234 (-N-(2-chloro-6-fluorophenyl)-4-(4-ethyl-3-(hydroxymethyl)-5-oxo-4,5-dihydro-1H-1,2,4-triazol-1-yl)-5-fluoro-2-((1,1,1-trifluoropropan-2-yl)oxy)benzamide), AG-636 (1-methyl-5-(2′-methyl-[1,1′-biphenyl]-4-yl)-1H-benzo[d][1,2,3]triazole-7-carboxylic acid), PTC-299 (4-chlorophenyl (S)-6-chloro-1-(4-methoxyphenyl)-1,3,4,9-tetrahydro-2H-pyrido[3,4-b]indole-2-carboxylate), JNJ-74856665, Meds433, RP7214, ML390, Laflunimus, Tenovin-1, Tenovin-6, hDHODH-IN-4, DHODH-IN-11, and teriflunomide.
Aspect 98. The pharmaceutical combination of any one of Aspect 1-Aspect 97, further comprising at least one agent known to treat a cancer.
Aspect 99. The pharmaceutical combination of Aspect 98, wherein the at least one agent is a DNA methyltransferase inhibitor, an HDAC-inhibitor, a glucocorticoid, an mTOR inhibitor, a cytotoxic agent, or combinations thereof.
Aspect 100. The pharmaceutical combination of Aspect 99, wherein the DNA methyltransferase inhibitor is 5-aza-2 ′ -deoxycytidine, 5-azacytidine, zebularin, epigallocatechin-3-gallate, procaine, or combinations thereof.
Aspect 101. The pharmaceutical combination of Aspect 99, wherein the HDAC-inhibitor is vorinostat, entinostat, panbinostat, trichostatin A, mocetinostat, belinostat, dacinostat, givinostat, tubastatin A, pracinostat, droxinostat, quisinostat, romidepsin, valproic acid, AR-42 (OSU-HDAC42), tacedinaline, rocilinostat, apicidin, or combinations thereof.
Aspect 102. The pharmaceutical combination of Aspect 99, wherein the glucocorticoid is dexamethasone, prednisolone, methylprednisolone, betamethasone, triamicinolone, fludrocortisone, beclomethasone, or combinations thereof.
Aspect 103. The pharmaceutical combination of Aspect 99, wherein the mTor inhibitor is BEZ235, everolimus, temsirolimus, rapamycin, AZD8055, or cobminations thereof.
Aspect 104. The pharmaceutical combination of Aspect 99, wherein the cytotoxic agent is an alkylating agent, an antimetabolite agent, an antineoplastic antibiotic agent, a mitotic inhibitor agent, a mTor inhibitor agent or other chemotherapeutic agent.
Aspect 105. The pharmaceutical combination of Aspect 104, wherein the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.
Aspect 106. The pharmaceutical combination of Aspect 104, wherein the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.
Aspect 107. The pharmaceutical combination of Aspect 104, wherein the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.
Aspect 108. The pharmaceutical combination of Aspect 104, wherein the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.
Aspect 109. The pharmaceutical combination of Aspect 104, wherein the mTor inhibitor is everolimus, sirolimus, temsirolimus, or combinations thereof.
Aspect 110. The pharmaceutical combination of Aspect 104, wherein the other chemotherapeutic agent is an anthracycline, cytarabine, a purine analog, sorafenib, gemtuzumab ozogamicin, rituximab, or combinations thereof.
Aspect 111. The pharmaceutical combination of Aspect 110, wherein the anthracycline is daunorubicin, idarubicin, or combinations thereof.
Aspect 112. The pharmaceutical combination of Aspect 110, wherein the purine analog is cladribine, fludarabine, clofarabine, or combinations thereof.
Aspect 113. The pharmaceutical combination of any one of Aspect 1-Aspect 97, further comprising at least one agent known to treat GVHD.
Aspect 114. The pharmaceutical combination of Aspect 113, wherein the least one agent known to treat GVHD is a steroid, an mTor inhibitor, a tyrosine kinase inhibitor, or other agent known to treat GVHD.
Aspect 115. The pharmaceutical combination of Aspect 114, wherein the steroid is dexamethasone, prednisolone, methylprednisolone, betamethasone, triamicinolone, fludrocortisone, beclomethasone, or combinations thereof.
Aspect 116. The pharmaceutical combination of Aspect 114, wherein tyrosine kinase inhibitor is imatinib, ruxolitinib, or a combination thereof.
Aspect 117. The pharmaceutical combination of Aspect 114, wherein the mTor inhibitor is everolimus, sirolimus, temsirolimus, or combinations thereof.
Aspect 118. The pharmaceutical combination of Aspect 114, wherein the other agent known to treat GVHD is tacrolimus, clofazimine, psoralen, cyclosporine, alemtuzumab, infliximab, rituximab, etanercept, antithymocyte globulin, thalidomide, mycophenolate mofetil, pentostatin, methotrexate, halofuginone, hydroxychloroquine, or combinations thereof.
Aspect 119. The pharmaceutical combination of any one of 1-Aspect 97, further comprising at least one agent known to treat an autoimmune disorder or disease.
Aspect 120. The pharmaceutical combination of Aspect 119, wherein the at least one agent known to treat an autoimmune disorder or disease is selected from the group consisting of: (a) disease modifying antirheumatic drugs; (b) nonsteroidal anitinflammatory drugs; (c) COX-2 selective inhibitors; (d) COX-1 inhibitors; (e) immunosuppressive drugs, including p70S6 kinase inhibitors; and inosine monophosphate dehydrogenase inhibitors; (f) steroids; (g) biological response modifiers; and (h) other agents useful for the treatment of autoimmune disorders.
Aspect 121. The pharmaceutical combination of Aspect 120, wherein the disease modifying antirheumatic drug is selected from methotrexate, gold salts, D-penicillamine, hydroxychloroquine, auranofin, sulfasalazine, and combinations thereof.
Aspect 122. The pharmaceutical combination of Aspect 120, wherein the nonsteroidal anitinflammatory drug is selected from indomethacin, naproxen, diclofenac, ibuprofen, aspirin and aspirin analogs, acetaminophen, and combinations thereof.
Aspect 123. The pharmaceutical combination of Aspect 120, wherein the COX-2 selective inhibitor is selected from celecoxib, rofecoxib, etoricoxib, valdecoxib, lumiracoxib, and combinations thereof.
Aspect 124. The pharmaceutical combination of Aspect 120, wherein the immunosuppressive drug is selected from a calcineurin inhibitor such as cyclosporin and FK506;a p70S6 kinase inhibitor such as sirolimus and rapamycin; an inosine monophosphate dehydrogenase inhibitor such as mycophenolate; leflunomide, cyclophosphamide, azathioprine, and combinations thereof.
Aspect 125. The pharmaceutical combination of Aspect 120, wherein the steroid is selected from prednisone, betamethasone, budesonide and dexamethasone, and combinations thereof.
Aspect 126. The pharmaceutical combination of Aspect 120, wherein the biological response modifier is selected from TNFα antagonists such as infliximab, adalimmab and etanercept; IL-1 receptor antagonists such as anakinra; humanized or chimeric antibodies or fusion proteins such as alefacept, efalizumab, daclizumab; anti-chemokine antibodies; anti-interleukin antibodies; and combinations thereof.
Aspect 127. The pharmaceutical combination of Aspect 120, wherein the other agent useful for the treatment of autoimmune disorder is selected from hemokine receptor antagonists or modulators, cannabinoid receptor antagonists or modulators, inhibitors of matrix metalloproteinases, TNFα-converting enzymes, nitric oxide synthetases or phosphodiesterase IV, such as roflumilast or cilomilast; inhibitors of p38 MAP-kinase, the NF-kappaβ, pathway or IL-1 receptor associated kinase or inhibitors of interactions involving adhesion molecules such as LFA-1, VLA-4, ICAM-1, VCAM-1, α₄β₇, MAdCAM-1, and αᵥβ₃; and combinations thereof.
Aspect 128. A method for the treatment of a disease or disorder in a mammal comprising the step of administering to the mammal either: (a) a therapeutically effective amount of a pharmaceutical combination of any of Apsect 1-Aspect 127; or (b) co-administering an anti-CD38 therapeutic agent as disclosed herein and at least one DHODH inhibitor compound as disclosed herein.
Aspect 129. The method of Aspect 128, wherein the mammal is a human.
Aspect 130. The method of Aspect 128, wherein the mammal has been diagnosed with a need for treatment of the disorder prior to the administering step.
Aspect 131. The method of Aspect 130, wherein the disorder or disease is associated with abnormal, increased, or aberrant dihydroorotate dehydrogenase (DHODH) activity.
Aspect 132. The method of Aspect 131, wherein the disorder or disease can be treated by inhibition of dihydroorotate dehydrogenase (DHODH) activity.
Aspect 133. The method of any one of Aspect 128-Aspect 132, further comprising the step of identifying a mammal in need of treatment of the disorder or disease.
Aspect 134. The method of Aspect 133, wherein the disorder or disease is associated with abnormal, increased, or aberrant dihydroorotate dehydrogenase (DHODH) activity.
Aspect 135. The method of Aspect 134, wherein the disorder or disease can be treated by inhibition of dihydroorotate dehydrogenase (DHODH) activity.
Aspect 136. The method of any one of Aspect 128-Aspect 135, wherein the disorder is selected from aplastic anemia, depletion of malignant myeloid derived suppressor cells, Immunoglobulin light chain amyloidosis (AL), and combinations thereof.
Aspect 137. The method of any one of Aspect 128-Aspect 135, wherein the disorder is selected from chronic lymphocytic leukemia, MGUS/multiple myeloma, extranodal natural killer (NK)/T-cell lymphoma, large cell lymphoma, nasal type (ENKTL-N), myelodysplasia, treatment related myeloid malignancies, acute myeloid leukemia, chronic myelomonocytic leukemia, T-lymphoblastic lymphoma/leukemia, B-lymphoblastic lymphoma/leukemia, Burkitt’s leukemia/lymphoma, primary effusion lymphoma, Philadelphia-positive acute lymphoblastic leukemia, immunomodulation for solid tumors, and combinations thereof.
Aspect 138. The method of any one of Aspect 128-Aspect 135, wherein the disorder is a cancer.
Aspect 139. The method of Aspect 138, wherein the cancer is selected from breast cancer, renal cancer, gastric cancer, colorectal cancer, ovarian cancer, prostate cancer, pancreatic cancer, brain cancer, genitourinary tract cancer, lymphatic system cancer, stomach cancer, larynx cancer, lung cancer, pancreatic cancer, breast cancer, and malignant melanoma.
Aspect 140. The method of Aspect 138, wherein the cancer is a hematological cancer.
Aspect 141. The method of Aspect 140, wherein the hematological cancer is leukemia, lymphoma, myeloma, myelodysplastic syndrome, or myeloproliferative neoplasm.
Aspect 142. The method of Aspect 141, wherein the hematological cancer is chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin’s lymphoma, non-Hodgkin’s lymphoma, hairy cell lymphoma, Burkett’s lymphoma, Hodgkin lymphoma, and non-Hodgkin lymphoma.
Aspect 143. The method of Aspect 142, wherein the hematological cancer is chronic myeloid leukemia (CML) or acute myeloid leukemia (AML).
Aspect 144. The method of any one of Aspect 128-Aspect 143, further comprising the step of administering a therapeutically effective amount of at least one agent known to treat a cancer.
Aspect 145. The method of Aspect 144, wherein the at least one agent is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, and vasostatin.
Aspect 146. The method of Aspect 144, wherein the at least one agent is a DNA methyltransferase inhibitor, an HDAC-inhibitor, a glucocorticoid, an mTOR inhibitor, a cytotoxic agent, or combinations thereof.
Aspect 147. The method of Aspect 146, wherein the DNA methyltransferase inhibitor is 5-aza-2 ′ -deoxycytidine, 5-azacytidine, zebularin, epigallocatechin-3-gallate, procaine, or combinations thereof.
Aspect 148. The method of Aspect 146, wherein the HDAC-inhibitor is vorinostat, entinostat, panbinostat, trichostatin A, mocetinostat, belinostat, dacinostat, givinostat, tubastatin A, pracinostat, droxinostat, quisinostat, romidepsin, valproic acid, AR-42 (OSU-HDAC42), tacedinaline, rocilinostat, apicidin, or combinations thereof.
Aspect 149. The method of Aspect 146, wherein the glucocorticoid is dexamethasone, prednisolone, methylprednisolone, betamethasone, triamicinolone, fludrocortisone, beclomethasone, or combinations thereof.
Aspect 150. The method of Aspect 146, wherein the mTor inhibitor is BEZ235, everolimus, temsirolimus, rapamycin, AZD8055, or cobminations thereof.
Aspect 151. The method of Aspect 146, wherein the cytotoxic agent is an alkylating agent, an antimetabolite agent, an antineoplastic antibiotic agent, a mitotic inhibitor agent, a mTor inhibitor agent or other chemotherapeutic agent.
Aspect 152. The method of Aspect 151, wherein the antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt, hydrate, solvate, or polymorph thereof.
Aspect 153. The method of Aspect 151, wherein the antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt, hydrate, solvate, or polymorph thereof.
Aspect 154. The method of Aspect 151, wherein the alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt, hydrate, solvate, or polymorph thereof.
Aspect 155. The method of Aspect 151, wherein the mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt, hydrate, solvate, or polymorph thereof.
Aspect 156. The method of Aspect 151, wherein the mTor inhibitor is everolimus, sirolimus, temsirolimus, or combinations thereof.
Aspect 157. The method of Aspect 151, wherein the other chemotherapeutic agent is an anthracycline, cytarabine, a purine analog, sorafenib, gemtuzumab ozogamicin, rituximab, or combinations thereof.
Aspect 158. The method of Aspect 157, wherein the anthracycline is daunorubicin, idarubicin, or combinations thereof.
Aspect 159. The method of Aspect 157, wherein the purine analog is cladribine, fludarabine, clofarabine, or combinations thereof.
Aspect 160. The method of any one of Aspect 128-Aspect 159, wherein the at least one compound and the at least one agent are administered sequentially.
Aspect 161. The method of any one of Aspect 128-Aspect 159, wherein the at least one compound and the at least one agent are administered simultaneously.
Aspect 162. The method of any one of Aspect 128-Aspect 159, wherein the at least one compound and the at least one agent are co-formulated.
Aspect 163. The method of any one of Aspect 128-Aspect 159, wherein the at least one compound and the at least one agent are co-packaged.
Aspect 164. The method of any one of Aspect 128-Aspect 133, wherein the disorder is mediated by T-cell proliferation.
Aspect 165. The method of Aspect 164, wherein the disorder is psoriasis.
Aspect 166. The method of Aspect 164, wherein the disorder is graft-versus-host disease (GVHD).
Aspect 167. The method of Aspect 166, wherein the GVHD is associated with an organ transplant, an allograft, a xenograft, or a hematopoietic stem cell transplantation.
Aspect 168. The method of Aspect 166 or Aspect 167, wherein the GVHD is acute GVHD.
Aspect 169. The method of Aspect 166 or Aspect 167, wherein the GVHD is chronic GVHD.
Aspect 170. The method of any one of Aspect 166- Aspect 169, further comprising the step of administering a therapeutically effective amount of at least one agent known to treat GVHD.
Aspect 171. The method of Aspect 170, wherein the least one agent known to treat GVHD is a steroid, an mTor inhibitor, a tyrosine kinase inhibitor, or other agent known to treat GVHD.
Aspect 172. The method of Aspect 171, wherein the steroid is dexamethasone, prednisolone, methylprednisolone, betamethasone, triamicinolone, fludrocortisone, beclomethasone, or combinations thereof.
Aspect 173. The method of Aspect 171, wherein tyrosine kinase inhibitor is imatinib, ruxolitinib, or a combination thereof.
Aspect 174. The method of Aspect 171, wherein the mTor inhibitor is everolimus, sirolimus, temsirolimus, or combinations thereof.
Aspect 175. The method of Aspect 171, wherein the other agent known to treat GVHD is tacrolimus, clofazimine, psoralen, cyclosporine, alemtuzumab, infliximab, rituximab, etanercept, antithymocyte globulin, thalidomide, mycophenolate mofetil, pentostatin, methotrexate, halofuginone, hydroxychloroquine, or combinations thereof.
Aspect 176. The method of any one of Aspect 128-Aspect 133, wherein the disorder is associated with T-cell proliferation.
Aspect 177. The method of any one of Aspect 128-Aspect 133, wherein the disorder is an autoimmune disorder or disease.
Aspect 178. The method of Aspect 177, wherein the autoimmune disorder or disease is selected from lupus, rheumatoid arthritis, ankylosing spondylitis, glomerulonephritis, minimal change disease, ulcerative colitis, crohns disease, addison’s disease, adult Still’s disease, alopecia areata, autoimmune hepatitis, autoimmune angioedema, Bechet’s disease, pemphigoid and variants, celiac disease, chronic inflammatory demyelinating polyneuropathy, churg-Straus syndrome, Crest syndrome, dermatomyositis, neuromyelitis optica, discoid lupus, fibromyalgia, giant cell arteritis, giant cell myocarditis, Goodpasteur’s disease, evan’s syndrome, autoimmune hemolytic anemia, immune thrombocytopenia, Henoch-Schonlein purpura, IgA nephropathy, IgG4 related sclerosing disease, juvenile arthritis, juvenile diabetes, Kawasaki disease, Leukocytoclastic vasculitis, mixed connective disease, multiple sclerosis, multifocal motor neuropathy, myasthenia gravis, autoimmune neutropenia, optic neuritis, peripheral neuropathy, POEMS syndrome, polymyositis, primary biliary cirrhosis, non-alcoholic hepatosteotosis and associated cirrhosis, psoriasis, scleroderma, sarcoidosis, temporal arteritis, vasculitis, and uveitis.
Aspect 179. The method of Aspect 177 or Aspect 178, further comprising the step of administering a therapeutically effective amount of at least one agent known to treat an autoimmune disorder or disease.
Aspect 180. The method of Aspect 179, wherein the at least one agent known to treat an autoimmune disorder or disease is selected from the group consisting of: (a) disease modifying antirheumatic drugs; (b) nonsteroidal anitinflammatory drugs; (c) COX-2 selective inhibitors; (d) COX-1 inhibitors; (e) immunosuppressive drugs, including p70S6 kinase inhibitors; and inosine monophosphate dehydrogenase inhibitors; (f) steroids; (g) biological response modifiers; and (h) other agents useful for the treatment of autoimmune disorders.
Aspect 181. The method of Aspect 180, wherein the disease modifying antirheumatic drug is selected from methotrexate, gold salts, D-penicillamine, hydroxychloroquine, auranofin, sulfasalazine, and combinations thereof.
Aspect 182. The method of Aspect 180, wherein the nonsteroidal anitinflammatory drug is selected from indomethacin, naproxen, diclofenac, ibuprofen, aspirin and aspirin analogs, acetaminophen, and combinations thereof.
Aspect 183. The method of Aspect 180, wherein the COX-2 selective inhibitor is selected from celecoxib, rofecoxib, etoricoxib, valdecoxib, lumiracoxib, and combinations thereof.
Aspect 184. The method of Aspect 180, wherein the immunosuppressive drug is selected from a calcineurin inhibitor such as cyclosporin and FK506;a p70S6 kinase inhibitor such as sirolimus and rapamycin; an inosine monophosphate dehydrogenase inhibitor such as mycophenolate; leflunomide, cyclophosphamide, azathioprine, and combinations thereof.
Aspect 185. The method of Aspect 180, wherein the steroid is selected from prednisone, betamethasone, budesonide and dexamethasone, and combinations thereof.
Aspect 186. The method of Aspect 180, wherein the biological response modifier is selected from TNFα antagonists such as infliximab, adalimmab and etanercept; IL-1 receptor antagonists such as anakinra; humanized or chimeric antibodies or fusion proteins such as alefacept, efalizumab, daclizumab; anti-chemokine antibodies; anti-interleukin antibodies; and combinations thereof.
Aspect 187. The method of Aspect 180, wherein the other agent useful for the treatment of autoimmune disorder is selected from hemokine receptor antagonists or modulators, cannabinoid receptor antagonists or modulators, inhibitors of matrix metalloproteinases, TNFα-converting enzymes, nitric oxide synthetases or phosphodiesterase IV, such as roflumilast or cilomilast; inhibitors of p38 MAP-kinase, the NF-kappaβ, pathway or IL-1 receptor associated kinase or inhibitors of interactions involving adhesion molecules such as LFA-1, VLA-4, ICAM-1, VCAM-1, α₄β₇, MAdCAM-1, and αᵥβ₃; and combinations thereof.
Aspect 188. The method of any one of Aspect 128-Aspect 187, wherein the administering is administering the therapeutically effective amount of a pharmaceutical combination of any of 1-Aspect 127.
Aspect 189. The method of any one of Aspect 128-Aspect 187, wherein the administering is administering the co-administering the at least one anti-CD38 therapeutic agent as disclosed herein and the least one DHODH inhibitor compound as disclosed herein.
Aspect 190. The method of Aspect 189, wherein the co-administering comprises sequential administration of the at least one anti-CD38 therapeutic agent as disclosed herein and the least one DHODH inhibitor compound as disclosed herein.
Aspect 191. The method of Aspect 189, wherein the co-administering comprises simultaneous administration of the at least one anti-CD38 therapeutic agent as disclosed herein and the least one DHODH inhibitor compound as disclosed herein.
Aspect 192. The method of Aspect 189, wherein the co-administering comprises administration of the anti-CD38 therapeutic agent as disclosed herein on a first dose administration schedule and administration of the least one DHODH inhibitor compound as disclosed herein on a second dose administration schedule.
Aspect 193. A method for inhibiting dihydroorotate dehydrogenase activity in at least one cell, comprising the step of contacting the at least one cell with either: (a) a pharmaceutical combination of any of 1-Aspect 127; or (b) at least one anti-CD38 therapeutic agent as disclosed herein and at least one DHODH inhibitor compound as disclosed herein.
Aspect 194. The method of Aspect 193, wherein the cell is mammalian.
Aspect 195. The method of Aspect 194, wherein the cell is human.
Aspect 196. The method of any one of Aspect 193-Aspect 195, wherein the cell has been isolated from a mammal prior to the contacting step.
Aspect 197. The method of any one of Aspect 193-Aspect 195, wherein contacting is via administration to a mammal.
Aspect 198. The method of Aspect 197, wherein the mammal has been diagnosed with a need for inhibiting dihydroorotate dehydrogenase activity prior to the administering step.
Aspect 199. The method of Aspect 198, wherein the mammal has been diagnosed with a need for treatment of a disorder related to dihydroorotate dehydrogenase activity prior to the administering step.
Aspect 200. The method of any one of Aspect 193-Aspect 199, wherein the compound exhibits inhibition of dihydroorotate dehydrogenase with an IC₅₀ of less than about 1,000 nM using a cell-free enzymatic assay.
Aspect 201. The method of Aspect 200, exhibits inhibition of dihydroorotate dehydrogenase with an IC₅₀ of less than about 500 nM.
Aspect 202. The method of Aspect 200, exhibits inhibition of dihydroorotate dehydrogenase with an IC₅₀ of less than about 250 nM.
Aspect 203. The method of Aspect 200, exhibits inhibition of dihydroorotate dehydrogenase with an IC₅₀ of less than about 100 nM.
Aspect 204. The method of Aspect 200, exhibits inhibition of dihydroorotate dehydrogenase with an IC₅₀ of less than about 50 nM.
Aspect 205. The method of any one of Aspect 193-Aspect 204, wherein the contacting is contacting the cell with a pharmaceutical combination of any of 1-Aspect 127.
Aspect 206. The method of any one of Aspect 193-Aspect 204, wherein the contacting is co-contacting the cell with the at least one anti-CD38 therapeutic agent as disclosed herein and the least one DHODH inhibitor compound as disclosed herein.
Aspect 207. The method of Aspect 206, wherein the co-contacting comprises sequential contacting of the cell with the at least one anti-CD38 therapeutic agent as disclosed herein and the least one DHODH inhibitor compound as disclosed herein.
Aspect 208. The method of Aspect 206, wherein the co-contacting comprises simultaneous contacting of the cell with the at least one anti-CD38 therapeutic agent as disclosed herein and the least one DHODH inhibitor compound as disclosed herein.
Aspect 209. The method of Aspect 206, wherein the co-contacting comprises contacting of the cell with the anti-CD38 therapeutic agent as disclosed herein on a first dose contacting schedule and contacting of the least one DHODH inhibitor compound as disclosed herein on a second dose contacting schedule.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### 1 Example 1: Synthesis of Exemplary Disclosed Compounds

Synthesis of 2-(4′-ethoxy-[1,1′-biphenyl]-4-yl)-6-fluoroquinoline-4-carboxylic acid (Cpd3). A representative DHODH inhibitor compound was used in the studies described herein. In the figures it is designated as “Cpd3”, which is 2-(4′-ethoxy-[1,1′-biphenyl]-4-yl)-6-fluoroquinoline-4-carboxylic acid. A chemical structure for Cpd3 is shown immediately below. Cpd3 was prepared as described in Intl. Pat. Appl. No. PCT/US19/38622, which is incorporated herein by reference.

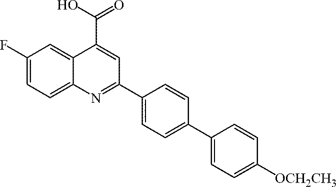 [SMILES: CCOc1ccc(-c2ccc(-c3cc(C(=O)O)c4cc(F)ccc4n3)cc2)cc1]

2-(4′-ethoxy-[1,1′-biphenyl]-4-yl)-6-fluoroquinoline-4-carboxylic acid (Cpd3).

2-(3′-butoxy-[1,1′-biphenyl]-4-yl)-6-fluoroquinoline-4-carboxylic acid (Cpd4). The procedures described herein below were used to prepare 2-(3′-butoxy-[1,1′-biphenyl]-4-yl)-6-fluoroquinoline-4-carboxylic acid (Cpd4) from compounds C1 and C2, and the sodium salt form of Cpd4, i.e., sodium 2-(3′-butoxy-[1,1′-biphenyl]-4-yl)-6-fluoroquinoline-4-carboxylate (Cpd4Na).

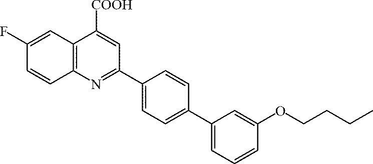 [SMILES: CCCCOc1cccc(-c2ccc(-c3cc(C(=O)O)c4cc(F)ccc4n3)cc2)c1]

2-(3′-butoxy-[1,1′-biphenyl]-4-yl)-6-fluoroquinoline-4-carboxylic acid (Cpd4).

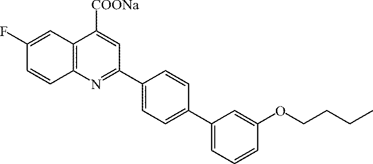

Sodium 2-(3′-butoxy-[1,1′-biphenyl]-4-yl)-6-fluoroquinoline-4-carboxylate (Cpd4Na).

General procedure for preparation of compound C1. The procedures described herein below were used to prepare compound C1, which was used in the preparation of 2-(3′-butoxy-[1,1′-biphenyl]-4-yl)-6-fluoroquinoline-4-carboxylic acid (Cpd4).

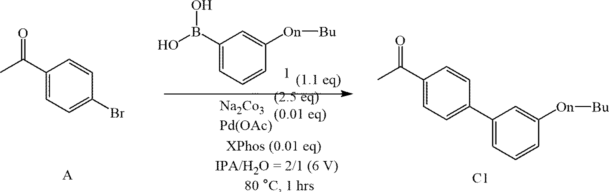

The mixture of compound A (40.0 g, 201 mmol, 1.00 eq) in IPA (200 mL) and H₂O (100 mL), compound 1 (42.9 g, 221 mmol, 1.10 eq), Na₂CO₃ (53.3 g, 502 mmol, 2.50 eq), Pd(OAc)₂ (451 mg, 2.01 mmol, 0.01 eq) and XPhos (958 mg, 2.01 mmol, 0.01 eq) was added to the mixture. The mixture was degassed and purged with N₂ for 3 times, and then the mixture was stirred at 80° C. for 1 hr under N₂ atmosphere. TLC (petroleum ether / ethyl acetate = 20 / 1, compound A: R_{f} = 0.61, compound C1: R_{f} = 0.43) indicated the compound A was consumed completely, and one major new spot with larger polarity was detected. The reaction mixture was diluted with H₂O (800 mL) and extracted with ethyl acetate (400 mL, 300 mL, 200 mL). The combined organic layers were washed with brine (600 mL), dried over Na₂SO₄, filtered and concentrated. The crude product was triturated with petroleum ether at 25° C. for 4 hrs. The mixture was filtered and the filter cake was wash with petroleum ether (30.0 mL) and dry under reduced pressure. Compound C1 (30.0 g, 109 mmol, 54.5% yield, 98% purity) was obtained as a off-white solid.

General procedure for preparation of compound Cpd4. The procedures described herein below were used to prepare 2-(3′-butoxy-[1,1′-biphenyl]-4-yl)-6-fluoroquinoline-4-carboxylic acid (Cpd4) from compound C1.

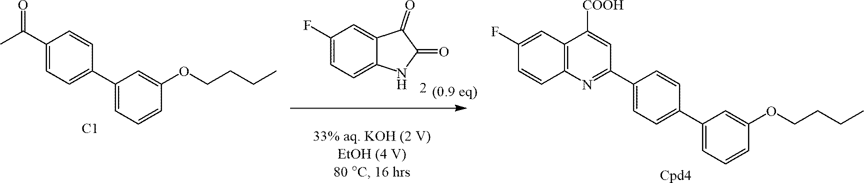

The mixture of compound C1 (30.0 g, 112 mmol, 1.00 eq) in KOH (60.0 mL, 33% purity) was stirred and heated to 35° C. until clear yellow solution formed. Compound 2 (16.6 g, 101 mmol, 0.90 eq) and EtOH (120 mL) was added to this solution. The reaction mixture was stirring at 80° C. for 12 hrs. TLC (petroleum ether / ethyl acetate = 1 / 1, compound C1: R_{f} = 0.57, compound C2: R_{f} = 0.35) indicated compound C1 was consumed completely, and one major new spot with larger polarity was detected. The reaction mixture was cooled to 25° C. The pH was adjusted to 4 with aq. HCI (6 M). The mixture filtered under reduced pressure to give a residue. The crude product was triturated with petroleum ether / ethyl acetate = 3 / 1 (150 mL) at 20° C. for 12 hrs. The mixture was filtered and the filter cake was wash with petroleum ether (30.0 mL) and dry under reduced pressure. Compound C2 (20.0 g, 48.1 mmol, 43.1% yield, 97% purity) was obtained as a yellow solid.

General procedure for preparation of compound Cpd4Na. The procedures described herein below were used to prepare sodium 2-(3′-butoxy-[1,1′-biphenyl]-4-yl)-6-fluoroquinoline-4-carboxylate (Cpd4Na) from compound Cpd4.

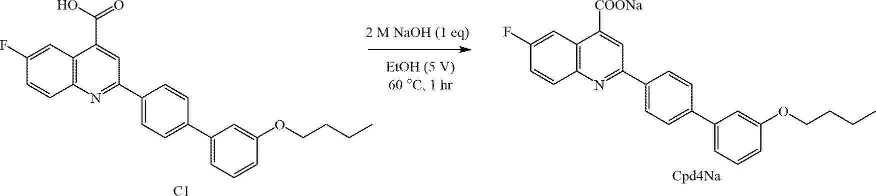

To a solution of compound Cpd4 (18.0 g, 43.3 mmol, 1.00 eq) in EtOH (120 mL) was added aqueous NaOH (2 M, 21.7 mL, 1.00 eq). The mixture was degassed and purged with N₂ for 3 times, and then the mixture was stirred at 60° C. for 1 hr under N₂ atmosphere. The reaction mixture was concentrated under vacuum to removed EtOH. H₂O (250 mL) was added to the residue and the mixture was freeze-dried to give the product. Cpd4 (16.8 g, 38.4 mmol, 88.6% yield) was obtained as a yellow solid. MS (M+1)⁺: calcd. m/z = 415.17, found m/z = 416.1.

### 2. Example 2: Expression of Cd38 After Treatment of AML Cells With a Dhodh Inhibitor

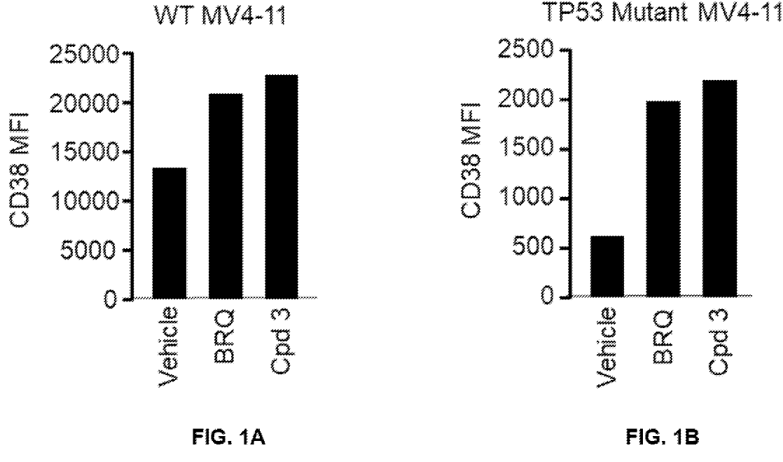

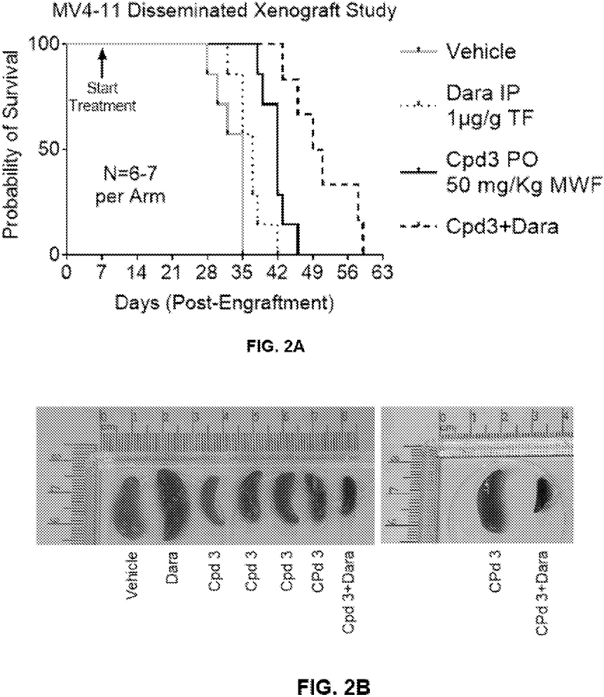

MV-411 cells with wild type (WT) p53 or mutant p53 were treated with brequinar (BRQ, 1 µM), or Cpd3 (1 µM) for 72 hours. CD38 surface expression was determined by flow cytometry. The CD38 mean fluorescent intensity (MFI) is calculated based on the X-median expression. FIG. 1A show data obtained with MV-411 having wild type p53. These data show that these cells have a high baseline of relative expression of CD38 (see vehicle treated data), which is upregulated following 72 hour exposure to either brequinar or Cpd3, both which are DHODH inhibitors. FIG. 1B show the results obtained with MV-411 having a mutant p53. These data show that these cells have a very low baseline of relative expression of CD38, which is highly upregulated following 72 hour exposure to either brequinar or Cpd3. The data show that AML cells having different genetic backgrounds can respond to exposure to a DHODH inhibitor with upregulation of CD38 on the cell plasma membrane.

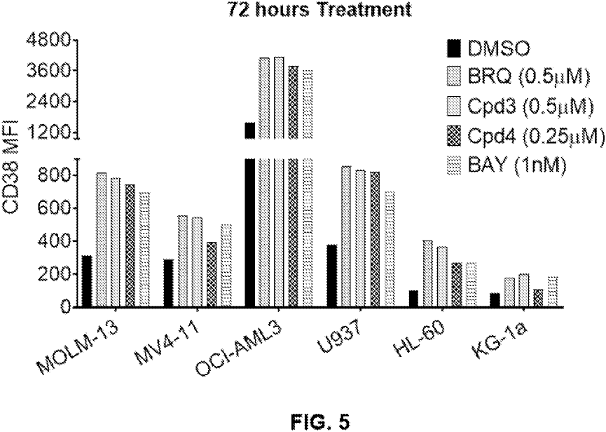

Data were also obtained for six AML cell lines spanning different mutation background were treated for three days with DHODH inhibitors (Brequinar, Cpd3, and Cpd4). Flow cytometry was used to measure CD38 surface expression using mean fluorescence intensity as described above. The data are shown in FIG. 5.

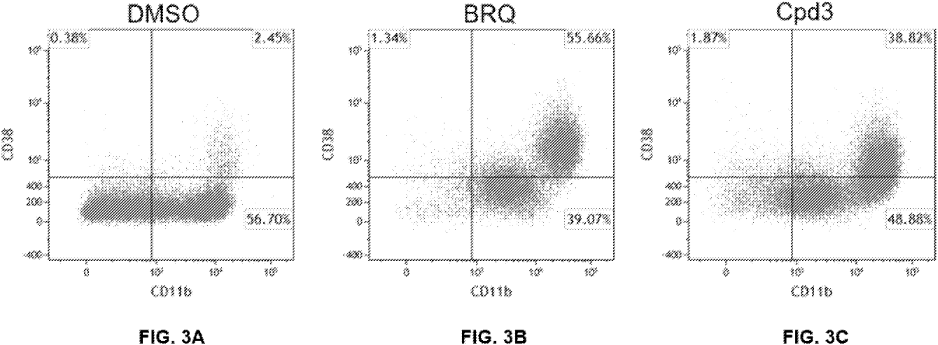

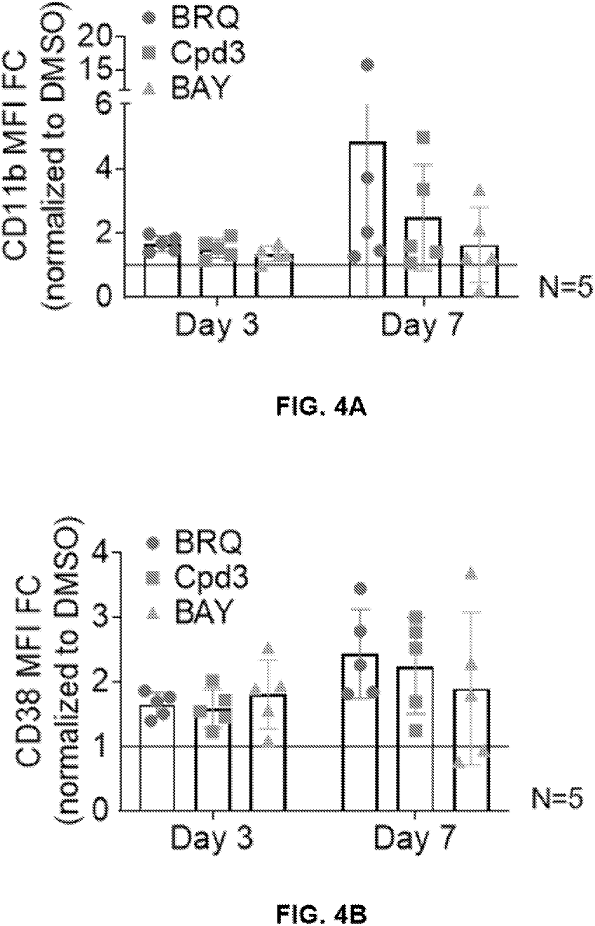

Primary AML cells were cultured on collagen coated plates in StemSpan™ SFEM II (STEMCELL Technologies) media supplemented with cytokines for up to seven days. Flow cytometry was used to measure the surface expression level of CD38 and CD11b on day three and day seven. Flow cytometry data obtained following treatment of primary AML cells with vehicle (DMSO), Brequinar (BRQ), and Cpd3 are shown in FIGS. 3A-3C, respectively. Mean fluorescence intensity plots are normalized to vehicle (DMSO), and data are plotted in FIGS. 4A-4B. It should be noted that CD11b is indicative of cellular differentiation towards the myeloid lineage, whereas the data herein show that CD38 is a useful biomarker for the disclosed pharmaceutical compositions and methods.

### 3 Example 3: Xenograft Studies Using Mv4-11 Cells

Xenograft Model Methodology. Animal studies were approved by the Institutional Laboratory Animal Care and Use Committee (IACUC) at the Ohio State University. 1E7 M4-11 cells were engrafted intravenously into NSG mice (males, 12-week old, strain NOD.Cg-Prkdcscid ll2rgtm1Wjl/SzJ). These mice were allowed to develop a fatal leukemia, at which point splenocytes containing leukemia cells were collected and cryopreserved. Subsequently, 0.3E6 of these splenocytes collected from previously MV4-11 engrafted mice were injected intravenously into 28 NSG mice (secondary engraftment). Generally, these mice develop leukemia (AML) in two weeks after the injection of spleen MNC from adapted MV4-11 cells of NSG mice (Ranganathan et. al., (2012) Blood (2012) 120 (9): 1765-1773) and their lifespan was around 3 \~4 weeks. One-week post engraftment, mice were randomly enrolled to receive: 1) vehicle, 2) 50 mg/kg Cpd3 orally on Monday, Wednesday, and Friday (MWF), 3) 1 µg/g daratumumab via intraperitoneal injection on Tuesday and Friday (TF), or 4) Cpd3 (MWF) and daratumumab (TF) combination therapy. Two additional mice were kept as non-engrafted controls. Mice were considered at early/end removal criteria (ERC) and removed from the study upon: 20% weight loss (based on weight at study initiation), paralysis, inability to stand, scruffy appearance, uncontrolled shivering or unwillingness to eat or drink. Overall survival was calculated using Kapler Meyer analysis. All the experiments were conducted in accordance with the institutional guidelines for animal care and use. Each arm included 6-7 mice and treatment began a week post engraftment.

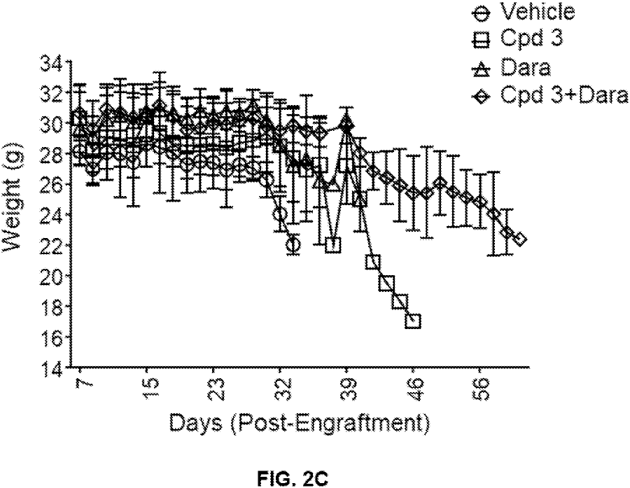

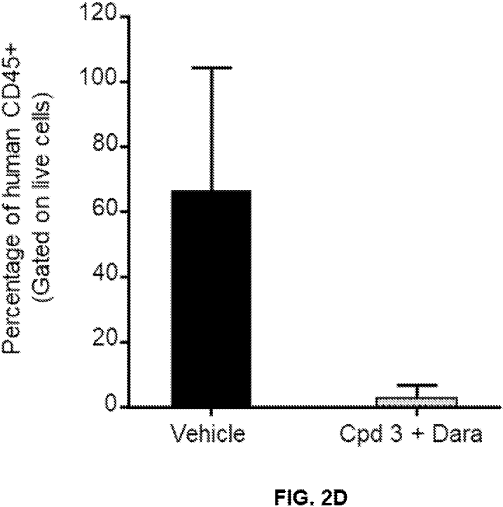

Study Results. Representative survival data are shown in FIG. 2A, and the median survival data are summarized in Tables 4 and 5 below. The data show that in this model, using the treatment protocol described above, that treatment with an anti-CD38 antibody, daratumumab, alone provided a marginal improviement in median survival (37 days compared to 35 days for vehicle treatment). Monotherapy with a DHODH inhibitor showed a slightly better improvement in median survival (42 days compared to 35 days for vehicle treatment). Surprisingly, the combination of a DHODH inhibitor with an anti-CD38 antibody provided a profound synergistic effect in the improvement of median survival (51 days compared to 35 days for vehicle treatment).

TABLE 4

| Group | N | N died | Estimated median survival time (days PE) | 95 %Cl |
|---|---|---|---|---|
| Vehicle | 7 | 7 | 35 | (28, --) |
| Cpd3 | 7 | 7 | 42 | (38, 43) |
| Dara | 7 | 7 | 37 | (32, 38) |
| Cpd3/Dara | 7 | 6 | 51 | (43, --) |

TABLE 5

| | Comparison | Log-rank p-value |
|---|---|---|
| Primary | Cpd3 vs. Cpd3/Dara | 0.001 |
| Secondary* | Cpd3 vs. Vehicle | 0.001 |
| | Dara vs. Vehicle | 0.026 |
| | Cpd3/Dara vs. Vehicle | 0.001 |
| | Cpd3/Dara vs. Dara | 0.001 |

In addition to a synergistic improvement in median survival, it was determined that the spleens of animals receiving the combination therapy were considerably smaller compared to vehicle treatment or treatment with anti-CD38 antibody alone (see FIG. 2B). It is believed that the smaller spleen size correlates with a decreased tumor burden in these animals. The positive foregoing results demonstrating a surprising beneficial effect of the combination therapy, the weight of test animals was monitored over the course of the study since weight loss is typically associated with leukemia progression. The data show (see FIG. 2C) that animals receiving the combination therapy had a pronounced improvement in maintenance of body weight compared to the other treatment groups. Finally, spleens were isolated from the animals from each treatment group as they reached ERC and the percentage of human CD45+ cells in the spleen were determined by flow cytometry. The data show (see FIG. 2D) an almost complete ablation of the presence of human CD45+ cells in the spleens of animals receiving the combination therapy compared to vehicle treated animals.

The foregoing studies demonstrate that treatment of AML cells with a DHODH inhibitor can result in the upregulation of CD38. It was determined in a xenograft model that a combination therapy comprising a DHODH inhibitor and an anti-CD38 antibody provided a surprising improvement in survival and tumor burden, and importantly, the effect was strongly synergistic when viewed against the results obtained for treatment with either agent alone.

### 4 Example 4: Xenograft Studies Using Mv4-11 Cells

Xenograft Model Methodology. Animal studies were performed at Charles River Laboratories (CRL). 1E5 MOLM-13 cells were engrafted intravenously into NCG mice (males, 8-12-week old). 4 days post engraftment, mice were randomly enrolled to receive:
1. Vehicle
2. 10 mg/kg Cpd 53 orally (daily)
3. 10 mg/kg Cpd 53 orally (TIWK, Monday, Wednesday, and Friday)
4. 4 mg/kg BAY2402234 orally (daily)
5. 1 µg/g Daratumumab via intraperitoneal injection (BIWK, Tuesday and Friday)
6. 2.5 µg/g Isatuximab via intraperitoneal injection (BIWK, Tuesday and Friday)
7. Cpd4 (daily) and Daratumumab (BIWK) combination
8. Cpd4 (TIWK) and Daratumumab (BIWK) combination
9. BAY2402234 (daily) and Daratumumab (BIWK) combination
10. Cpd4 (daily) and Isatuximab (BIWK) combination
11. Cpd4 (TIWK) and Isatuximab (BIWK) combination
12. BAY2402234 (daily) and Isatuximab (BIWK) combination

Further details on dosing groups, dosing levels, frequency of dosing, and route of administration are provided below in Table 6.

TABLE 6

| Gr. | N | Agent | Formulation dose | Active dose | Route | Schedule |
|---|---|---|---|---|---|---|
| 1^{#} | 8 | vehicle 1 | - | - | po | qd to end |
| 2 | 8 | vehicle 1 // vehicle 2 | na // na | na // na | po // ip | qd to end // qd to end |
| 3 | 8 | Cpd4 | 10.53 mg/kq | 10 mg/kg | po | qd to end |
| 4 | 8 | Cpd4 | 10.53 mg/kg | 10 mg/kg | po | tiwk to end |
| 5 | 8 | BAY2402234 | 4 mg/kg | 4 mg/kg | po | qd to end |
| 6 | 8 | daratumumab | 1 mg/kg | 1 mg/kg | ip | biwk to end |
| 7 | 8 | isatuximab | 2.5 mg/kg | 2.5 mg/kg | ip | biwk to end |
| 8 | 8 | Cpd4 // daratumumab | 10.53 mg/kg // 1 mg/kq | 10 mg/kg // 1 mg/kg | po // ip | qd to end // biwk to end |
| 9 | 8 | Cpd4 // daratumumab | 10.53 mg/kg // 1 mg/kq | 10 mg/kg // 1 mg/kg | po // ip | tiwk to end // biwk to end |
| 10 | 8 | BAY2402234 // daratumumab | 4 mg/kg // 1 mg/kg | 4 mg/kg // 1 mg/kg | po // ip | qd to end // biwk to end |
| 11 | 8 | Cpd4 // isatuximab | 10.53 mg/kg // 2.5 mg/kq | 10 mg/kg // 2.5 mg/kg | po // ip | qd to end // biwk to end |
| 12 | 8 | Cpd4 // isatuximab | 10.53 mg/kg // 2.5 mg/kq | 10 mg/kg // 2.5 mg/kg | po // ip | tiwk to end // biwk to end |
| 13 | 8 | BAY2402234 // isatuximab | 4 mg/kg // 2.5 mg/kq | 4 mg/kg // 2.5 mg/kg | po // ip | qd to end // biwk to end |

Procedures used in the study are as follows:
Mice with 1×10⁵ MOLM-13 tumor cells in 0% Matrigel iv tail vein.
Study start is 4 days post implant
Animals were randomized into treatment groups based on Day 1 bodyweight.
Cell Injection Volume was 0.2 mL/mouse.
Age at Start Date: 8 to 12 weeks.
Body Weight: qd x 5 then biwk to end
Any individual animal with a single observation of > than 30% body weight loss or three consecutive measurements of >25% body weight loss was euthanized.
Any group with a mean body weight loss of >20 % or >10% mortality ceased dosing. The group was not euthanized and recovery is allowed. Within a group with >20% weight loss, individuals hitting the individual body weight loss endpoint was euthanized. If the group treatment related body weight loss is recovered to within 10% of the original weights, dosing resumed at a lower dose or less frequent dosing schedule. Exceptions to non-treatment body weight % recovery may be allowed on a case-by-case basis.
Endpoint: Moribundity. Animals were to be monitored individually. The endpoint of the experiment was Moribundity or 75 days, whichever comes first. When the endpoint is reached, the animals were euthanized.
Clinical signs associated with progression of tumor include impairment of hind limb function, ocular proptosis, and weight loss. Full hindlimb paralysis, ocular proptosis, or moribundity will be considered sufficient for euthanasia.

Dosing preparation and formulation details were as follows:
Compounds in Salt form: Cpd4 is a sodium salt (Formula weight = 437.45, Molecular weight = 415.46, Correction factor = 1.053)
Dosing solutions:
   BAY2402234
      10%Ethanol/90%PEG400
   Daratumumab in PBS, store at 4° C., protect from light, prep once per study
   Cpd4 40% HPBCD in water, protect from light
      All doses represent total substance dosed by weight of compound (including salt weight). The amount of active compound dosed is as follows:
      Dose 10.53 mg/kg = 10 mg/kg Active Compound
   Isatuximab = isatuximab in PBS, prep once per study
   Vehicle 1 = vehicle 1 40% HPBCD in water
   Vehicle 2 = vehicle 2 in PBS
Dosing volume = 10 mL/kg (0.200 mL/20 g mouse). Adjust volume accordingly for body weight.

Mice were considered at early/end removal criteria (ERC) and removed from the study upon: 20% weight loss (based on weight at study initiation), paralysis, inability to stand, scruffy appearance, uncontrolled shivering or unwillingness to eat or drink. Overall survival was calculated using Kapler Meyer analysis. Median survival data are summarized in tabular format below. All the experiments were conducted in accordance with the relevant institutional guidelines for animal care and use. Each arm included 6-7 mice and treatment began a week post engraftment

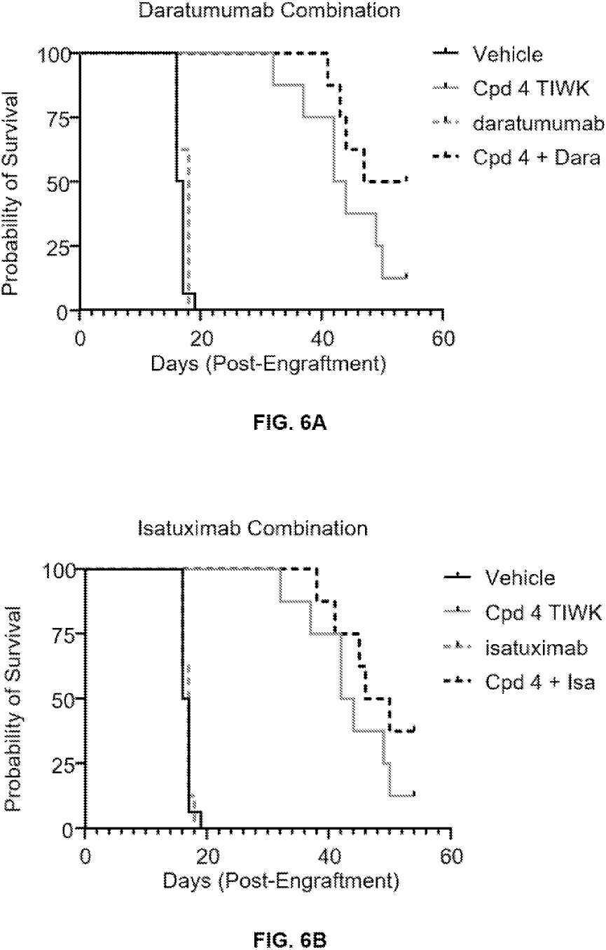

Study Results. The study was carried out as described above, with the following specific study details. 1e5 MOLM-13 cells were engrafted intravenously into NCG mice (males, 8-12 week-old). Four days post engraftment, mice were randomly enrolled to receive vehicle, 10 mg/kg Cpd4 (PO) MWF, 1 µg/g Daratumumab (IP) OR 2.5 mg/kg Isatuximab on Tuesday and Friday (TF), or Cpd4 and Daratumumab OR Isatuximab combination. Representative survival data are shown in FIGS. 6A-6B, and the median survival data are summarized in Table 7 below for treatment with Cpd4 alone and in combination with either daratumumab or Isatuximab using the dosing regiment of TIWK (MWF) Cpd4 treatment. The data show that in this model, using the treatment protocol described above, that the combination of a DHODH inhibitor, Cpd4, with an anti-CD38 antibody provided a synergistic effect in the improvement of median survival compared to one of these agents alone (48 or 48.5 days compared to 16.5 days for vehicle treatment, 17 or 18 days for anti-CD38 antibody alone, or 43 days for Cpd4 alone) using the dosing regiment of TIWK (MWF) Cpd4 treatment.

TABLE 7

| Treatment | N | Median survival (days) |
|---|---|---|
| Vehicle | 8 | 16.5 |
| Daratumumab (Dara) | 8 | 18 |
| Isatuximab (ISA) | 8 | 17 |
| Cpd4 | 8 | 43 |
| Cpd4 + Dara | 8 | 48.5 |
| Cpd4 + Isa | 8 | 48 |

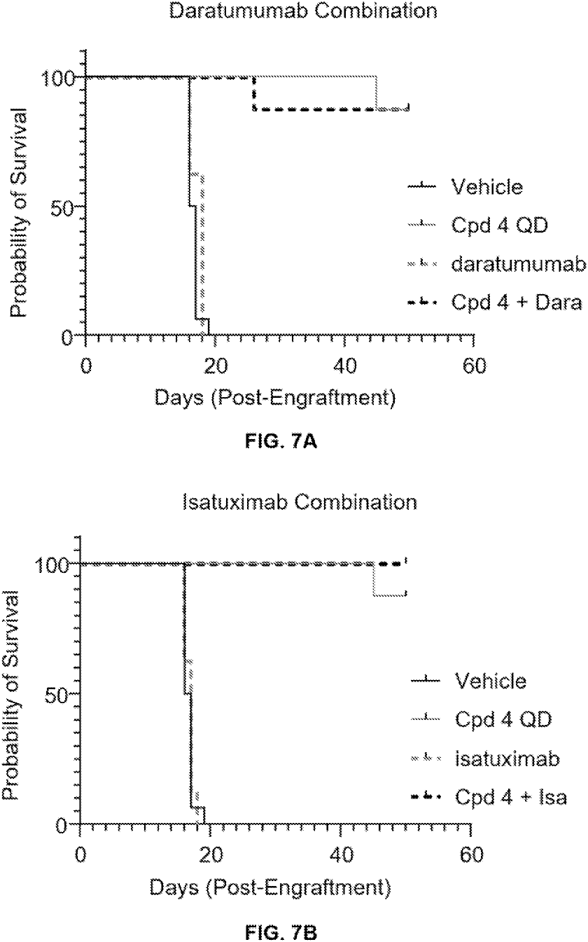

Study Results. The study was carried out as described above, with the following specific study details. 1e5 MOLM-13 cells were engrafted intravenously into NCG mice (males, 8-12-week-old). Four days post engraftment, mice were randomly enrolled to receive vehicle, 10 mg/kg Cpd 53 (daily PO), 1 µg/g Daratumumab (IP) OR 2.5 mg/kg Isatuximab on Tuesday and Friday (TF), or Cpd4 and Daratumumab OR Isatuximab combination. Representative survival data are shown in FIGS. 7A-7B, and the median survival data are summarized in Table 8 below for treatment with Cpd4 alone and in combination with either daratumumab or Isatuximab using the dosing regiment of daily Cpd4 treatment. The data show that in this model, using the treatment protocol described above, that the combination of a DHODH inhibitor, Cpd4, with an anti-CD38 antibody appears to provide provided a synergistic effect in the improvement of median survival compared to one of these agents alone using the dosing regiment of daily Cpd4 treatment.

TABLE 8

| Treatment | N | Median survival (days) |
|---|---|---|
| Vehicle | 8 | 16.5 |
| Daratumumab (Dara) | 8 | 18 |
| Isatuximab (Isa) | 8 | 17 |
| Cpd4 | 8 | NR* |
| Cpd4 + Dara | 8 | NR |
| Cpd4 + Isa | 8 | NR |

* “NR” indicates that median survival not reached.

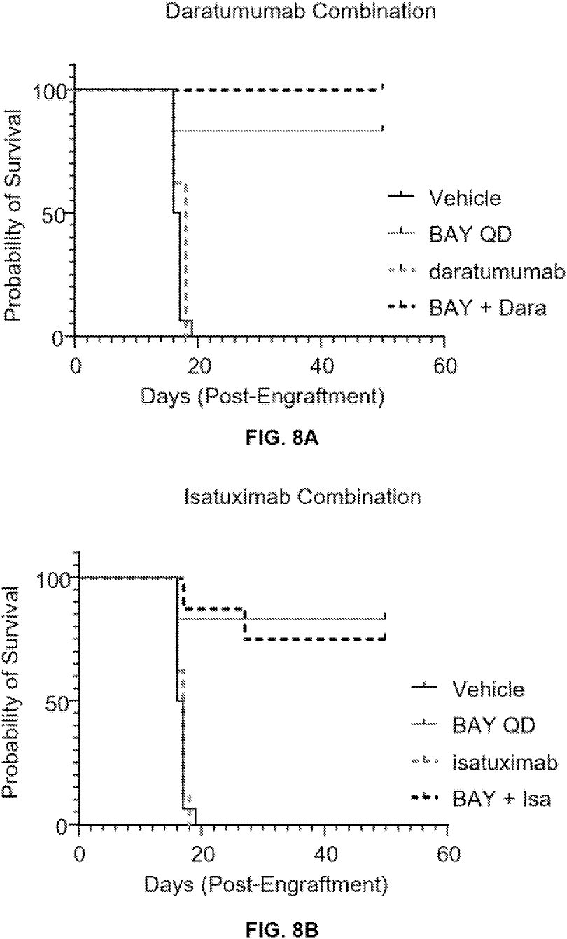

Study Results. The study was carried out as described above, with the following specific study details. 1e5 MOLM-13 cells were engrafted intravenously into NCG mice (males, 8-12-week old). Four days post engraftment, mice were randomly enrolled to receive vehicle, 4 mg/kg BAY2402234 (“BAY”; daily PO), 1 µg/g Daratumumab (IP) OR 2.5 mg/kg Isatuximab on Tuesday and Friday (TF), or BAY2402234 and Daratumumab OR Isatuximab combination. Representative survival data are shown in FIGS. 8A-8B, and the median survival data are summarized in Table 9 below for treatment with BAY alone and in combination with either daratumumab or Isatuximab using the dosing regiment of daily Cpd4 treatment. The data show that in this model, using the treatment protocol described above, that the combination of a DHODH inhibitor, BAY, with an anti-CD38 antibody appears to provide provided a synergistic effect in the improvement of median survival compared to one of these agents alone using the dosing regiment of daily BAY treatment.

TABLE 9

| Treatment | N | Median survival (days) |
|---|---|---|
| Vehicle | 8 | 16.5 |
| Daratumumab (Dara) | 8 | 18 |
| Isatuximab (Isa) | 8 | 17 |
| BAY2402234 (BAY) | 8 | NR* |
| BAY + Dara | 8 | NR |
| BAY + Isa | 8 | NR |

* “NR” indicates that median survival not reached.

### 5 Example 5: Prospective Determination of Mutational Subsets

Studies can be carried to determine whether there are specific mutational subsets of AML cells more or less responsive to upregulation of CD38 following DHODH inhibition. For example, primary AML samples (bone marrow, apheresis, or blood) will be used to evaluate CD38 upregulation upon DHODH inhibition. Cells are cultured in StemSpan (STEMCELL Technologies) in the presence of 20 ng/ml of FLT3L, SCF, GM-CSF, IL3, G-CSF, IL6, TPO cytokines and 10 ng/ml of EPO cytokine. RNA will be collected one, three, and seven days after treatment with 0.5 µM of brequinar (BRQ) or Cpd3 to measure transcriptional upregulation of CD38. Similarly, flow cytometry analysis will be done to measure the protein expression level of CD38 in addition to myeloid differentiation markers, CD11b and CD14. The collected RNA will be submitted for sequencing to identify mutational subsets demonstrating CD38 upregulation in response to DHODH inhibition.

### 6. Example 6: Prospective Analysis of Mechanism of Combination Therapy Synergism

It has previously been observed that all-trans retinoic acid (ATRA) induced CD38 expression in MV4-11 cell line can cause AML fratricide with daratumumab (Dara) treatment. (Buteyn NJ, et al. Anti-leukemic effects of all-trans retinoic acid in combination with Daratumumab in acute myeloid leukemia. Int Immunol. 2018;30(8):375-383). Studies can be understaken to assess if DHODH inhibitors, e.g., brequinar (BRQ) and/or Cpd3, in combination with Daratumumab can induce AML fratricide using Non-Radioactive Cytotoxicity Assay (lactate dehydrogenase, LDH) and Conjugate formation assay. Briefly, LDH can be assayed as follows: 0.5E6 MV4-11 cells will be treated with vehicle, 1 µM BRQ, or Cpd3 with or without 20 µg/ml Dara. Every 24 hours, supernatant will be collected and used for lactate dehydrogenase (LDH) assay using colorimetric analysis to calculate Percent Cytotoxicity using the equation: [Experimental LDH release OD490/Maximum LDH release OD490] × 100 normalized to untreated (vehicle) or single treatment conditions. A conjugate formation assay can be carried out as follows: 1E6 MV4-11 cells will be plated and treated with vehicle, 1 µM BRQ, or Cpd3 with or without 20 µg/ml Dara for 24 hours. Cells will be collected and washed with PBS to be fixed with 4% paraformaldehyde for 10 min. Following fixation, cells will be stained with Rhodamine Phalloidin F-actin for 30 min in the dark then washed three times with PBS to remove excess stain. Cells will be mounted on slides (cytospin) to count the number of conjugated cells in blinded fashion using fluorescent microscopy. Conjugation index will be calculated as the number of cells with at least one conjugate per 100 cells.

A second approach that be used to assess the mechanism of synergism is using an assay of cell-mediated antibody-dependent cell-mediated cytotoxicity (“ADCC”). ADCC will be determined using the standard four-hour ⁵¹Cr-release assay. MV4-11 cells will be pretreated with vehicle, Brequinar or Cpd3 for 24 hours. Pretreated MV4-11 target cells will be labelled with ⁵¹Cr (0.1 mCi ⁵¹Cr per million cells) then washed followed by 30 min incubation with Daratumumab then plated in a 96-well plate. Healthy donor natural killer (NK) cells (effector cells) will be isolated and added to the wells at different E:T ratios, 25:1, 12:1, 6:1, 3:1, 1.5:1, 0.75:1, and 0:1. After four hour incubation, supernatant is removed and counted using a gamma counter to measure specific cell lysis percentage calculated using equation: 100 × (ER - SR)/ (MR - SR). ER = experimental release, SR = spontaneous release, MR = maximum release. NK cells express CD38 and therefore Daratumumab treatment may cause NK fratricide. If that appears to occur, we will generate CD38 knockout NK cells using CRISPR-Cas9 as described to avoid Daratumumab induced NK fratricide (see Naeimi Kararoudi, M. et al., Generation of Knock-out Primary and Expanded Human NK Cells Using Cas9 Ribonucleoproteins. J. Vis. Exp. (136), e58237, doi:10.3791/58237 (2018); and Nagai, Y. et al., CD38 Knockout Primary NK Cells to Prevent “Fratricide” and Boost Daratumumab Activity, Oral Presentation, The American Society of Hematology Annual meeting, December 6-10, 2019, Orlando, FL).

It will be apparent to those skilled in the art that various modifications and variations can be made in the present disclosure without departing from the scope or spirit of the disclosure. Other aspects and aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A pharmaceutical composition comprising at least one anti-CD38 therapeutic agent and at least one DHODH inhibitor compound, a pharmaceutically acceptable salt thereof, and a pharmaceutically acceptable carrier.

2. The pharmaceutical composition of claim 1, wherein the at least one anti-CD38 therapeutic agent comprises an antibody recognizing CD38.

3. The pharmaceutical composition of claim 2, wherein the antibody recognizing CD38 is capable of killing a CD38+ cell by antibody dependent cell-mediated phagocytosis (ADCP), cellular fratricide, apoptosis, antibody-dependent cell-mediated cytotoxicity (ADCC) and/or complement-dependent cytotoxicity (CDC).

4. The pharmaceutical composition of claim 2, wherein the antibody recognizing CD38 comprises a chimeric or humanized antibody, an antibody fragment, an antibody-drug conjugate, a nanobody, a bispecific antibody, a trispecific antibody, a single variable-domain antibody, or combinations thereof.

5. The pharmaceutical composition of claim 2, wherein the antibody recognizing CD38 is selected from daratumumab, isatuximab (SAR650984), felzartamab, ISB-1342, Y-150, ISB-1908, KPMW-101, AMG-424, XmAb-13243, XmAb-13551, MOR202 (MorphoSys AG), TAK-079, TAK-169, KP-1196, BM38, TJ202, and combinations thereof.

6. The pharmaceutical composition of claim 1, wherein the DHODH inhibitor compound is a compound having a formula represented by a structure: 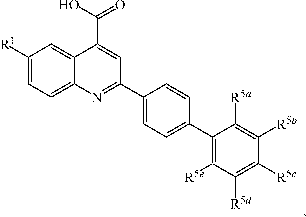
wherein R¹ is selected from halogen, —SF₅, —CN, —N₃, —OH, —NH₂, —CF₃, and —CF₂CF₃;
wherein each of R^{5b} and R^{5c} is independently selected from —R²⁰, hydrogen, halogen, —SF₅, —CN, —N₃, —OH, —NH₂, —CF₃, and —CF₂CF₃; wherein R²⁰ is selected from -C1-C10 alkylamino and -C1-C10 alkoxy;
provided that one of R^{5b} and R^{5c} is —R²⁰; and
wherein each R^{5a}, R^{5d}, and R^{5e} is independently selected from hydrogen, halogen, —SF₅, —CN, —N₃, —OH, —NH₂, —CF₃, and —CF₂CF₃;
or a pharmaceutically acceptable salt thereof.

7. The pharmaceutical composition of claim 6, wherein R^{5b} is —R²⁰; and wherein each of R^{5a}, R^{5c}, R^{5d}, and R^{5e} is independently selected from hydrogen, halogen, —SF₅, —CN, —N₃, —OH, —NH₂, —CF₃, and —CF₂CF₃.

8-13. (canceled)

14. The pharmaceutical composition of claim 6, wherein R^{5c} is -R²⁰; and wherein each of R^{5a}, R^{5b}, R^{5d}, and R^{5e} is independently selected from hydrogen, halogen, —SF₅, —CN, —N₃, —OH, —NH₂, —CF₃, and —CF₂CF₃.

15. The pharmaceutical composition of claim 1, wherein R²⁰ is selected from -C2-C7 alkylamino and —C2-C7 alkoxy.

16-21. (canceled)

22. The pharmaceutical composition of claim 1, wherein the DHODH inhibitor compound is a compound having a formula represented by a structure: 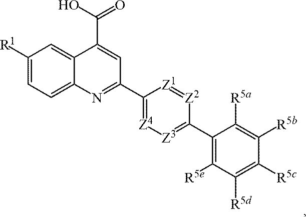
wherein each of Z¹, Z², Z³, and Z⁴ is independently selected from CH and N;
wherein R¹ is selected from hydrogen, halogen, —SF₅, —CN, —N₃, —OH, —NH₂, —CF₃, and —CF₂CF₃;
wherein one of R^{5a}, R^{5b}, R^{5c}, R^{5d}, and R^{5e} is selected from a group having formula represented by a structure:
—R²⁰, -R³⁰-A¹-R⁴⁰, -A¹-R⁴⁰, -A¹-R³⁰-A²-R⁴⁰, or -A¹-R³⁰-A²-R³¹-A³-R⁴⁰;
wherein A¹ is selected from —O— and —NR⁵⁰—;
wherein R⁵⁰ is selected from hydrogen, -C1-C10 alkyl, -C1-C10 aminoalkyl, and —C1-C10 hydroxyalkyl;
wherein A² is selected from —O— and —NR⁶⁰—;
wherein R⁶⁰ is selected from hydrogen, -C1-C10 alkyl, -C1-C10 aminoalkyl, and —C1-C10 hydroxyalkyl;
wherein A³ is selected from —O— and —NR⁷⁰—;
wherein R⁷⁰ is selected from hydrogen, -C1-C10 alkyl, -C1-C10 aminoalkyl, and -C1-C10 hydroxyalkyl;
wherein R²⁰ is selected from halogen, -C1-C10 alkyl, -C1-C10 haloalkyl, -C1-C10 hydroxyalkyl, -C1-C10 alkylamino, and -C1-C10 alkoxy;
wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and
wherein R⁴⁰ is selected from -C1-C10 alkyl, -C1-C10 haloalkyl, -C1-C10 aminoalkyl, -C1-C10 hydroxyalkyl, and —(CH₂)ₙAr¹;
wherein n is an integer selected from 1, 2, and 3; and
wherein Ar¹ is a phenyl group substituted with 0,1, 2, 3, 4, or 5 groups independently selected from halogen, —SF₅, —CN, —N₃, —OH, —NH₂, -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃;
or a pharmaceutically acceptable salt thereof.

23-24. (canceled)

25. The pharmaceutical composition of claim 1, wherein the DHODH inhibitor compound is a compound having a formula represented by a structure: 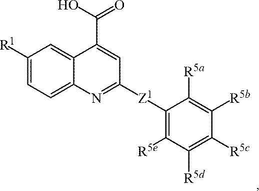
wherein Z¹ is a five-membered heterocyclic diyl;
wherein R¹ is selected from hydrogen, halogen, —SF₅, —CN, —N₃, —OH, —NH₂, —CF₃, and —CF₂CF₃;
wherein one of R^{5a}, R^{5b}, R^{5c}, R^{5d}, and R^{5e} is selected from a group having formula represented by a structure:
-R²⁰, —R³⁰—A¹—R⁴⁰, -A¹-R⁴⁰, -A¹-R³⁰-A²-R⁴⁰, or -A¹-R³⁰-A²-R³¹-A³-R⁴⁰;
wherein A¹ is selected from —O— and -NR⁵⁰-;
wherein R⁵⁰ is selected from hydrogen, -C1-C10 alkyl, -C1-C10 aminoalkyl, and -C1-C10 hydroxyalkyl;
wherein A² is selected from —O— and —NR⁶⁰—;
wherein R⁶⁰ is selected from hydrogen, -C1-C10 alkyl, -C1-C10 aminoalkyl, and —C1-C10 hydroxyalkyl;
wherein A³ is selected from —O— and —NR⁷⁰—;
wherein R⁷⁰ is selected from hydrogen, -C1-C10 alkyl, -C1-C10 aminoalkyl, and —C1-C10 hydroxyalkyl;
wherein R²⁰ is selected from halogen, -C1-C10 alkyl, -C1-C10 alkylamino and -C1-C10 alkoxy;
wherein each of R³⁰ and R³¹ is independently selected from -C1-C10 alkanediyl, -C1-C10 aminoalkanediyl, and -C1-C10 hydroxyalkanediyl; and
wherein R⁴⁰ is selected from -C1-C10 alkyl, -C1-C10 aminoalkyl, -C1-C10 hydroxyalkyl, and —(CH₂)ₙAr¹;
wherein n is an integer selected from 1, 2, and 3; and
wherein Ar¹ is a phenyl group substituted with 0,1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
and wherein four of R^{5a}, R^{5b}, R^{5c}, R^{5d}, and R^{5e} is independently selected from hydrogen, halogen, —SF₅, —CN, —N₃, —OH, —NH₂, —CF₃, and —CF₂CF₃;
or a pharmaceutically acceptable salt thereof.

26-27. (canceled)

28. The pharmaceutical composition of claim 1, wherein the DHODH inhibitor compound is a compound having a formula represented by a structure: 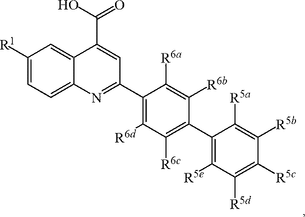
wherein R¹ is selected from hydrogen, halogen, —SF₅, —CN, —N₃, —OH, —NH₂, —CF₃, and —CF₂CF₃;
wherein one of R^{5a}, R^{5b}, R^{5c}, R^{5d}, and R^{5e} is selected from a group having formula represented by a structure:
—R²⁰, -R³⁰-A¹-R⁴⁰, —A¹—R⁴⁰, —A¹—R³⁰—A²—R⁴⁰, or -A¹-R³⁰-A²-R³¹-A³-R⁴⁰;
wherein A¹ is selected from —O— and —NR⁵⁰—;
wherein R⁵⁰ is selected from hydrogen, -C1-C10 alkyl, -C1-C10 aminoalkyl, and -C1-C10 hydroxyalkyl;
wherein A² is selected from —O— and —NR⁶⁰—;
wherein R⁶⁰ is selected from hydrogen, -C1-C10 alkyl, -C1-C10 aminoalkyl, and —C1-C10 hydroxyalkyl;
wherein A³ is selected from —O— and —NR⁷⁰—;
wherein R⁷⁰ is selected from hydrogen, -C1-C10 alkyl, -C1-C10 aminoalkyl, and —C1-C10 hydroxyalkyl;
wherein R²⁰ is selected from halogen, -C1-C10 alkyl, -C1-C10 haloalkyl, -C1-C10 hydroxyalkyl, -C1-C10 alkylamino, -C1-C10 alkoxy, —(CH₂)ₙCy¹, and —(CH₂)ₙAr¹;
wherein n is an integer selected from 1, 2, and 3; and
wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
wherein each of R³⁰ and R³¹ is independently selected from -C1-C10 alkanediyl, -C1-C10 haloalkanediyl, -C1-C10 aminoalkanediyl, and -C1-C10 hydroxyalkanediyl; and
wherein R⁴⁰ is selected from -C1-C10 alkyl, -C1-C10 haloalkyl, -C1-C10 aminoalkyl, -C1-C10 hydroxyalkyl, —(CH₂)ₙCy¹, and —(CH₂)ₙAr¹;
wherein n is an integer selected from 1, 2, and 3; and
wherein Cy¹ is a C3-C10 cycloalkyl group or a C2-C9 heterocycloalkyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
wherein Ar¹ is a phenyl group substituted with 0, 1, 2, 3, 4, or 5 groups independently selected from halogen, —SF₅, —CN, —N₃, —OH, —NH₂, from -C1-C4 alkyl, -C1-C4 alkoxy, -C1-C4 haloalkyl, -C1-C4 aminoalkyl, -C1-C4 alkylamino, -C1-C4 haloalkylamino, -C1-C4 hydroxyalkyl, -C1-C4 halohydroxyalkyl, cycloalkyl, and heterocycloalkyl;
and wherein four of R^{5a}, R^{5b}, R^{5c}, R^{5d}, and R^{5e} are independently selected from hydrogen, halogen, —SF₅, —CN, —N₃, —OH, —NH₂, —CF₃, and —CF₂CF₃;
wherein each of R^{6a}, R^{6b}, R^{6c}, and R^{6d} is independently selected from hydrogen, halogen, —SF₅, —CN, —N₃, —OH, —NH₂, C1-C10 alkyl, C1-C10 alkoxy, C1-C10 haloalkyl, C1-C10 aminoalkyl, and C1-C10 hydroxyalkyl, provided that at least one of R^{6a}, R^{6b}, R^{6c}, and R^{6d} is not hydrogen;
or a pharmaceutically acceptable salt thereof.

29. The pharmaceutical composition of claim 28, wherein the DHODH inhibitor compound is a compound having a formula represented by a structure: 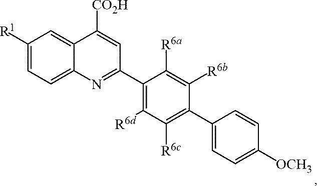 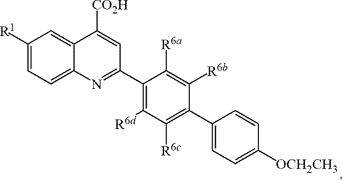 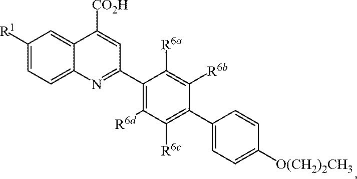 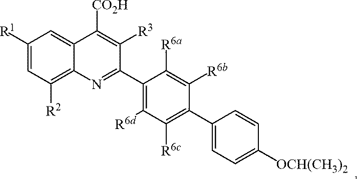 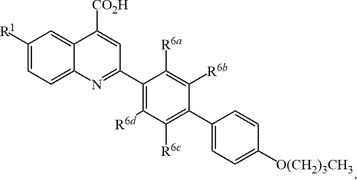 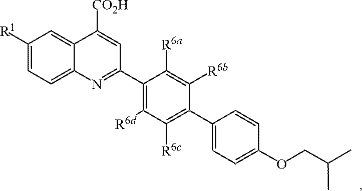 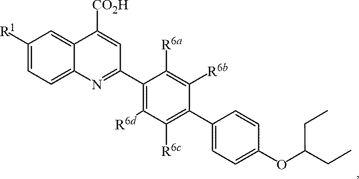 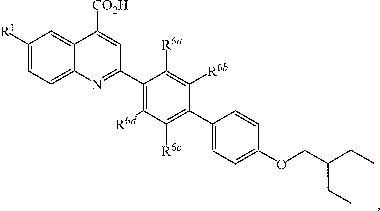 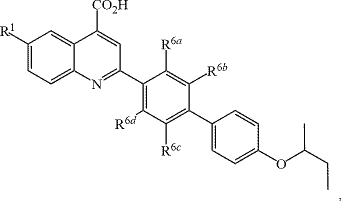 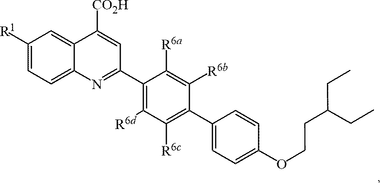
or combinations thereof.

30-36. (canceled)

37. The pharmaceutical composition of claim 28, wherein the DHODH inhibitor compound is a compound having a formula represented by a structure: 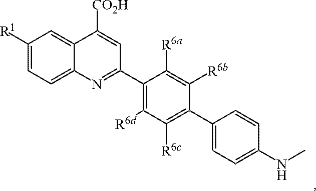 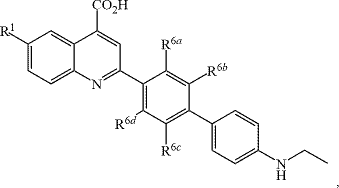 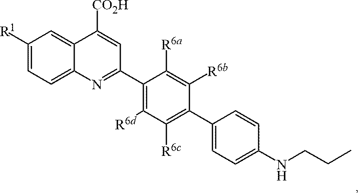 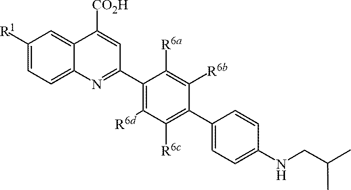
or combinations thereof.

38. (canceled)

39. The pharmaceutical composition of claim 1, wherein R¹ is selected from halogen, —SF₅, —CF₃, and —CF₂CF₃.

40-41. (canceled)

42. The compound of claim 39, wherein R¹ is —F.

43-45. (canceled)

46. The pharmaceutical composition of claim 1, wherein each of R^{6a}, R^{6b}, R^{6c}, and R^{6d} is independently selected from hydrogen, halogen, —SF₅, —CN, —N₃, —OH, —NH₂, C1-C3 alkyl, C1-C3 alkoxy, C1-C3 haloalkyl, C1-C3 aminoalkyl, and C1-C3 hydroxyalkyl, provided that at least one of R^{6a}, R^{6b}, R^{6c}, and R^{6d} is not hydrogen.

47-89. (canceled)

90. The pharmaceutical composition of claim 1, wherein the DHODH inhibitor compound is selected from the group consisting of brequinar, leflunomide, redoxal, vidofludimas, S-2678, 2-(3,5-difluoro-3′methoxybiphenyl-4-ylamino)nicotinic acid (also known as ASLAN003), BAY-2402234 (-N-(2-chloro-6-fluorophenyl)-4-(4-ethyl-3-(hydroxymethyl)-5-oxo-4,5-dihydro-1H-1,2,4-triazol-1-yl)-5-fluoro-2-((1,1,1-trifluoropropan-2-yl)oxy)benzamide), AG-636 (1-methyl-5-(2′-methyl-[1,1′-biphenyl]-4-yl)-1H-benzo[d][1,2,3]triazole-7-carboxylic acid), PTC-299 (4-chlorophenyl (S)-6-chloro-1-(4-methoxyphenyl)-1,3,4,9-tetrahydro-2H-pyrido[3,4-b]indole-2-carboxylate), JNJ-74856665, Meds433, RP7214, ML390, Laflunimus, Tenovin-1, Tenovin-6, hDHODH-IN-4, DHODH-IN-11, and teriflunomide.

91-94. (canceled)

95. A method for the treatment of a disease or disorder in a mammal comprising the step of administering to the mammal a therapeutically effective amount of a pharmaceutical composition of claim 1.

96-101. (canceled)

96. The method of claim 95, wherein the disorder is a cancer.

103-121. (canceled)
